(19) United States
(12) Patent Application Publication
(10) Pub. No.: US 2024/0400577 A1
(43) Pub. Date: Dec. 5, 2024

(54) MODULATORS OF PROTEIN KINASES

(71) Applicant: VIBLIOME THERAPEUTICS, LLC, Bozeman, MT (US)

(72) Inventors: Gary A. Flynn, Oro Valley, AZ (US); Paul Galatsis, Bozeman, MT (US); Andrew C. Huntsman, Bozeman, MT (US); Khoi Huynh, Bozeman, MT (US); Doug Werner, Bozeman, MT (US)

(21) Appl. No.: 18/577,481

(22) PCT Filed: Jul. 7, 2022
(86) PCT No.: PCT/US2022/036403

§ 371 Date: Jan. 8, 2024

Related U.S. Application Data
(60) Provisional application No. 63/219,459, filed on Jul. 8, 2021.

(51) Int. Cl. C07D 495/04; A61K 31/4184; A61K 31/437; A61K 31/4375; A61K 31/4439; A61K 31/4709; A61K 31/5025; A61K 31/506; A61K 31/517; A61K 31/52; A61K 31/5377; C07D 401/12; C07D 401/14; C07D 403/12; C07D 413/12; C07D 413/14; C07D 417/14; C07D 471/04; C07D 473/00; C07D 487/04

(52) U.S. Cl. CPC C07D 495/04; A61K 31/4184; A61K 31/437; A61K 31/4375; A61K 31/4439; A61K 31/4709; A61K 31/5025; A61K 31/506; A61K 31/517; A61K 31/52; A61K 31/5377; C07D 401/12; C07D 401/14; C07D 403/12; C07D 413/12; C07D 413/14; C07D 417/14; C07D 471/04; C07D 473/00; C07D 487/04

(57) ABSTRACT

Provided herein are small molecule protein kinase modulators, pharmaceutical compositions comprising such, and their uses in treating one or more conditions.

### RELATED APPLICATIONS

This application claims the benefit of priority to U.S. Provisional Application No. 63/219,459, filed Jul. 8, 2021, the entire contents of which are incorporated herein by reference.

### BACKGROUND

The more than 523 typical and atypical kinases in the human kinome represent a constellation of enzymes that catalyze the transfer of a phosphate group from ATP to a variety of amino acid residues, such as tyrosine, serine, and threonine. By so doing, these enzymes and their interrelated networks are effectors of cellular signal transduction. In particular, receptor tyrosine kinases (RTKs) coupled with their downstream intracellular kinases and phosphatases mediated cascades and feedback loops establish critical conduits for the transfer and regulation of signals from the cell exterior into the nucleus where transcriptional regulation takes place. Phosphate transfer to specific sites on proteins results in enzyme activation or inactivation, changes in conformation, increased or decreased affinity for other proteins, appropriate localization, and in some cases targeting of proteins for degradation by the proteosome. Kinase inhibitors, design strategies, and various mechanisms of inhibition have been extensively reviewed [Zhang J., et. al. Nature Reviews Cancer (2009) 9: 28-39; Blanc J. et. al., Anti-Cancer Agents in Med. Chem. (2013) 13, 17 pages; Gross S. et. al., J. Clin. Invest. (2015) 125(5); 1780-9; Cosgarea I. et. al., J. der Deutsch. Dermatol. Gesellschaft, (2017) 887-93, DOI: 10.1111/ddg.13321]. In addition, mechanistically similar lipid kinases, such as PI3Ks and SPK1, also contribute to the regulatory process (Brown J. R., et. al., BMC Evolutionary Biology (2011) 11(4): 1471-2148; Alvarez S. E., et. al., Nature (2010) 465: 1084-1088).

Because these processes regulate essential functions in cell growth, proliferation, differentiation and development, division, adhesion, angiogenesis, stress responses, cell-cell or cell-matrix interactions, short range contact-mediated axional guidance and mitogenesis, the activities of RTKs and their downstream kinase partners in signal transduction are tightly regulated and balanced through control of external receptor ligands as well as expression of receptors, receptor antagonists, decoy receptors, and through redundancies or crosstalk between signaling pathways. Therefore, the aberrant expression of kinases or activating mutations in kinases, inactivating mutations in negative regulators, and alterations in phosphatase expression or activity, are known to participate in a variety of diseases, including many cancers.

### SUMMARY

Provided herein are compounds having the Formula I.

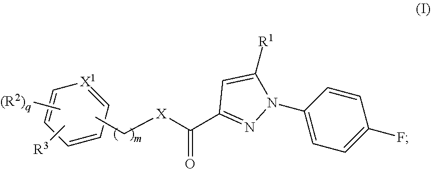

and pharmaceutically acceptable salts thereof, wherein R¹, R², R³, X, m, X¹ and q are as defined herein. These compounds act as modulators of protein kinase (e.g., kinase inhibitors) and are useful in treating conditions responsive to the inhibition of protein kinase (e.g., cancer). See e.g., Table 1 and 2.

In one aspect, the (4-fluorophenyl)pyrazolyl group on compounds of Formula I was found to be important for type II kinase inhibitors. It is contemplated that such compounds provide an alternative binding mode, compared to traditional type II inhibitors, which provides an alternative method for addressing kinase selectivity.

Also provided are pharmaceutically acceptable compositions comprising the disclosed protein kinase inhibitors.

### DETAILED DESCRIPTION

### 1. General Description of Compounds

In a first embodiment, provided is a compound having the Formula I:

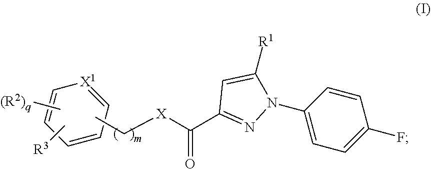

or a pharmaceutically acceptable salt thereof, wherein
R¹ is hydrogen, —SO₂R^{a}, —SOR^{a}, or —SR^{a};
X is —NR^{b} or 0;
m is 0, 1, or 2;
X¹ is CH or N;
R² is halogen, hydroxyl, (C₁-C₆)alkyl, cyano, (C₁-C₆)alkoxy, halo(C₁-C₆)alkyl, —NR^{b}R^{d}, and halo(C₁-C₆)alkoxy;
R³ is —OR⁴, —NHR⁵, —N(C₁-C₆)alkylR⁵, —CCHR⁶, —NHCOR⁷, —N(C₁-C₆)alkylCOR⁷, —C(O)R⁷, phenyl, —(C₁-C₆)alkyl[phenyl], heteroaryl, —(C₁-C₆)alkyl[heteroaryl], heterocyclyl, or —(C₁-C₆)alkyl[heterocyclyl] wherein said phenyl, heteroaryl and heterocyclyl alone or part of —(C₁-C₆)alkyl[phenyl], —(C₁-C₆)alkyl[heteroaryl], and —(C₁-C₆)alkyl[heterocyclyl] are each optionally substituted with 1 to 3 groups selected from R⁸; or R³ is taken together with one R² to form a 4- to 6-membered heteroaryl optionally substituted with a heteroaryl which is optionally substituted with 1 to 3 groups selected from R⁹;
R⁴ is heteroaryl optionally substituted with 1 to 3 groups selected from R¹⁰;
R⁵ is heteroaryl or heterocyclyl, each of which are optionally substituted with 1 to 3 groups selected from R¹²;
R⁶ and R⁷ are each independently heteroaryl optionally substituted with 1 to 3 groups selected from R¹²;
R⁸ is selected from halo, (C₁-C₆)alkyl, halo(C₁-C₆)alkyl, (C₁-C₆)alkoxy, halo(C₁-C₆)alkoxy, oxo, 4- to 6-membered heterocyclyl, —O(C₁-C₆)hydroxyalkyl, —NR^{b}C(O)R^{b}, and NR^{b}R^{c};
R⁹ is selected from halo, (C₁-C₆)alkyl, halo(C₁-C₆)alkyl, (C₁-C₆)alkoxy, and halo(C₁-C₆)alkoxy;
R¹⁰ is selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, halo, (C₁-C₆)alkoxy, halo(C₁-C₆)alkoxy, (C₁-C₆)alkyl, halo(C₁-C₆)alkyl, heterocyclyl, and heteroaryl, wherein said heterocyclyl and heteroaryl group are each optionally substituted with 1 to 3 groups selected from oxo, halo, (C₁-C₆)alkyl halo(C₁-C₆)alkyl, (C₁-C₆)alkoxy, halo(C₁-C₆)alkoxy, —C(O)NR^{b}R^{c}, —NR^{b}R^{c}, —C(O)ONR^{b}R^{c}, —C(O)R^{b}, —C(O)OR^{b}, —NR^{b}C(O)R^{b}, and —(C₁-C₆)alkylNH(C₁-C₆)hydroxyalkyl;
R¹¹ is selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, (C₁-C₆)alkoxy, halo(C₁-C₆)alkoxy, oxo, halo, (C₁-C₆)alkyl, halo(C₁-C₆)alkyl, heterocyclyl, and heteroaryl wherein said heterocyclyl and heteroaryl are each optionally substituted with 1 to 3 groups selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, (C₁-C₆)alkoxy, halo(C₁-C₆)alkoxy, oxo, halo, (C₁-C₆)alkyl, and halo(C₁-C₆)alkyl;
R¹² is selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, (C₁-C₆)alkoxy, halo(C₁-C₆)alkoxy, oxo, halo, (C₁-C₆)alkyl, and halo(C₁-C₆)alkyl;
q is 0, 1, 2, or 3;
R^{a} is (C₁-C₆)alkyl;
R^{b} and R^{d} are each independently hydrogen or (C₁-C₆)alkyl; and
R^{c} is selected from hydrogen, (C₁-C₆)alkyl, and 4- to 6-membered heterocyclyl.

Alternatively, as part of a first embodiment, provided is a compound having the Formula I:

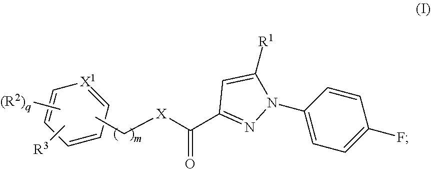

or a pharmaceutically acceptable salt thereof, wherein R³ is —OR⁴, —NHR⁵, —N(C₁-C₆)alkylR⁵, —CCHR⁶, —NHCOR⁷, —N(C₁-C₆)alkylCOR⁷, —C(O)R⁷, phenyl, heteroaryl, or heterocyclyl, wherein said phenyl, heteroaryl and heterocyclyl are each optionally substituted with 1 to 3 groups selected from R⁸; or R³ is taken together with one R² to form a 4- to 6-membered heteroaryl optionally substituted with a heteroaryl which is optionally substituted with 1 to 3 groups selected from R⁹; and q is 0, 1, or 2, wherein the remaining variables are as described in the preceding paragraph for Formula I.

### 2. Definitions

When used in connection to describe a chemical group that may have multiple points of attachment, a hyphen (-) designates the point of attachment of that group to the variable to which it is defined. For example, —NR^{b}R^{c} means that the point of attachment for this group occurs on the nitrogen atom.

The terms “halo” and “halogen” refer to an atom selected from fluorine (fluoro, —F), chlorine (chloro, —Cl), bromine (bromo, —Br), and iodine (iodo, —I).

The term “alkyl” when used alone or as part of a larger moiety, such as “haloalkyl”, and the like, means saturated straight-chain or branched monovalent hydrocarbon radical. Unless otherwise specified, an alkyl group typically has 1-4 carbon atoms, i.e., (C₁-C₄)alkyl.

“Alkoxy” means an alkyl radical attached through an oxygen linking atom, represented by —O-alkyl. For example, “(C₁-C₄)alkoxy” includes methoxy, ethoxy, propoxy, and butoxy.

The term “haloalkyl” includes mono, poly, and perhaloalkyl groups where the halogens are independently selected from fluorine, chlorine, bromine, and iodine (e.g., —CF₃, —CHF₂, etc.

“Haloalkoxy” is a haloalkyl group which is attached to another moiety via an oxygen atom such as, e.g., but are not limited to —OCHF₂ or —OCF₃.

The term “heteroaryl” used alone or as part of a larger moiety refers to a 5- to 12-membered (e.g., a 5- to 7-membered or 5- to 6-membered) aromatic radical containing 1-4 heteroatoms selected from N, O, and S. A heteroaryl group may be mono- or bi-cyclic. Monocyclic heteroaryl includes, for example, thienyl, furanyl, pyrrolyl, imidazolyl, pyrazolyl, triazolyl, tetrazolyl, oxazolyl, isoxazolyl, triazinyl, tetrazinyl, oxadiazolyl, thiazolyl, isothiazolyl, thiadiazolyl, pyridyl, pyridazinyl, pyrimidinyl, pyrazinyl, etc. Bi-cyclic heteroaryls include groups in which a monocyclic heteroaryl ring is fused to one or more aryl or heteroaryl rings. Nonlimiting examples include indolyl, imidazopyridinyl, benzooxazolyl, benzoxadiazolyl, indazolyl, benzimidazolyl, benzthiazolyl, quinolyl, quinazolinyl, quinoxalinyl, pyrrolopyridinyl, pyrrolopyrimidinyl, pyrazolopyridinyl, thienopyridinyl, thienopyrimidinyl, indolizinyl, purinyl, naphthyridinyl, and pteridinyl. It will be understood that when specified, optional substituents on a heteroaryl group may be present on any substitutable position and, include, e.g., the position at which the heteroaryl is attached.

The term “heterocyclyl” means a 4- to 12-membered (e.g., a 4- to 7-membered or 4- to 6-membered) saturated or partially unsaturated heterocyclic ring containing 1 to 4 heteroatoms independently selected from N, O, and S. It can be monocyclic, bicyclic (e.g., a bridged, fused, or spiro bicyclic ring), or tricyclic. A heterocyclyl ring can be attached to its pendant group at any heteroatom or carbon atom that results in a stable structure. Examples of such saturated or partially unsaturated heterocyclic radicals include, without limitation, tetrahydrofuranyl, tetrahydrothienyl, tetrahydropyranyl, pyrrolidinyl, pyridinonyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, oxetanyl, azetidinyl and tetrahydropyrimidinyl. A heterocyclyl group may be mono- or bicyclic. The term “heterocyclyl” also includes, e.g., unsaturated heterocyclic radicals fused to another unsaturated heterocyclic radical or aryl or heteroaryl ring, such as for example, tetrahydronaphthyridine, indolinone, dihydropyrrolotriazole, imidazopyrimidine, quinolinone, dioxaspirodecane. It will also be understood that when specified, optional substituents on a heterocyclyl group may be present on any substitutable position and, include, e.g., the position at which the heterocyclyl is attached.

The term “spiro” refers to two rings that shares one ring atom (e.g., carbon).

The term “fused” refers to two rings that share two adjacent ring atoms with one another.

The term “bridged” refers to two rings that share three ring atoms with one another.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

It is to be understood that, when a disclosed compound has at least one chiral center, the present invention encompasses one enantiomer free from the corresponding optical isomer, racemic mixture of the compound and mixtures enriched in one enantiomer relative to its corresponding optical isomer. When a mixture is enriched in one enantiomer relative to its optical isomers, the mixture contains, for example, an enantiomeric excess of at least 50%, 75%, 90%, 95% 99% or 99.5%.

The enantiomers of the present invention may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. Where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a disclosed compound has at least two chiral centers, the present invention encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s). When a mixture is enriched in one diastereomer or diastereomeric pair(s) relative to the other diastereomers or diastereomeric pair(s), the mixture is enriched with the depicted or referenced diastereomer or diastereomeric pair(s) relative to other diastereomers or diastereomeric pair(s) for the compound, for example, by a molar excess of at least 50%, 75%, 90%, 95%, 99% or 99.5%.

The diastereoisomeric pairs may be separated by methods known to those skilled in the art, for example chromatography or crystallization and the individual enantiomers within each pair may be separated as described above. Specific procedures for chromatographically separating diastereomeric pairs of precursors used in the preparation of compounds disclosed herein are provided the examples herein.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

As used herein, the terms “treatment,” “treat,” and “treating” refer to reversing, alleviating, delaying the onset of, or inhibiting the progress of a disease or disorder, or one or more symptoms thereof, as described herein. In some aspects, treatment may be administered after one or more symptoms have developed, i.e., therapeutic treatment. In other aspects, treatment may be administered in the absence of symptoms. For example, treatment may be administered to a susceptible individual prior to the onset of symptoms (e.g., in light of a history of symptoms and/or in light of exposure to a particular organism, or other susceptibility factors), i.e., prophylactic treatment. Treatment may also be continued after symptoms have resolved, for example to delay their recurrence.

The term “pharmaceutically acceptable carrier” refers to a non-toxic carrier, adjuvant, or vehicle that does not destroy the pharmacological activity of the compound with which it is formulated. Pharmaceutically acceptable carriers, adjuvants or vehicles that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

For use in medicines, the salts of the compounds described herein refer to non-toxic “pharmaceutically acceptable salts.” Pharmaceutically acceptable salt forms include pharmaceutically acceptable acidic/anionic or basic/cationic salts. Suitable pharmaceutically acceptable acid addition salts of the compounds described herein include e.g. salts of inorganic acids (such as hydrochloric acid, hydrobromic, phosphoric, nitric, and sulfuric acids) and of organic acids (such as, acetic acid, benzenesulfonic, benzoic, methanesulfonic, and p-toluenesulfonic acids). Compounds of the present teachings with acidic groups such as carboxylic acids can form pharmaceutically acceptable salts with pharmaceutically acceptable base(s). Suitable pharmaceutically acceptable basic salts include e.g., ammonium salts, alkali metal salts (such as sodium and potassium salts) and alkaline earth metal salts (such as magnesium and calcium salts). Compounds with a quaternary ammonium group also contain a counteranion such as chloride, bromide, iodide, acetate, perchlorate and the like. Other examples of such salts include hydrochlorides, hydrobromides, sulfates, methanesulfonates, nitrates, benzoates and salts with amino acids such as glutamic acid.

The term “effective amount” or “therapeutically effective amount” refers to an amount of a compound described herein that will elicit a desired or beneficial biological or medical response of a subject e.g., a dosage of between 0.01-100 mg/kg body weight/day.

### 3. Description of Exemplary Compounds

In a second embodiment, the compound of Formula I is of the Formula II:

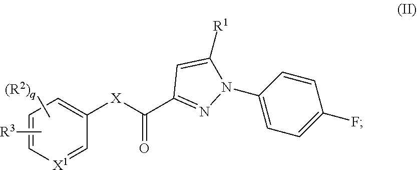

or a pharmaceutically acceptable salt thereof, wherein the variables are as described above for Formula I.

In a third embodiment, the compound of Formula I is of the Formula III:

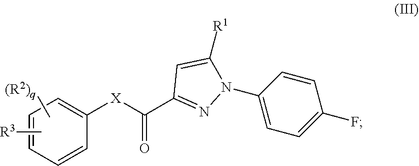

or a pharmaceutically acceptable salt thereof, wherein the variables are as described above for Formula I.

In a fourth embodiment, the compound of Formula I is of the Formula IV:

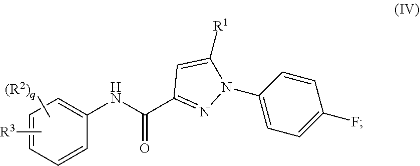

or a pharmaceutically acceptable salt thereof, wherein the variables are as described above for Formula I.

In a fifth embodiment, R² in the compound of any one of Formulae I to IV, or a pharmaceutically acceptable salt thereof, is —SO₂R^{a}, —SOR^{a}, or —SR^{a}, wherein the remaining variables are as described above for Formula I. Alternatively, as part of a fifth embodiment, R² in the compound of any one of Formulae I to IV, or a pharmaceutically acceptable salt thereof, is —SO₂R^{a}, —SOR^{a}, or —SR^{a} and R^{a} is (C₁-C₃)alkyl, wherein the remaining variables are as described above for Formula I. In another alternative, as part of a fifth embodiment, R² in the compound of any one of Formulae I to IV, or a pharmaceutically acceptable salt thereof, is —SO₂CH₃, —SCH₃, or —SOCH₃.

In a sixth embodiment, R² in the compound of any one of Formulae I to IV, or a pharmaceutically acceptable salt thereof, is halogen, hydroxyl, —NR^{b}R^{d}, or (C₁-C₃)alkyl, wherein the remaining variables are as described above for Formula I or the fifth embodiment. Alternatively, as part of a sixth embodiment, R² in the compound of any one of Formulae I to IV, or a pharmaceutically acceptable salt thereof, is hydroxyl, fluoro, bromo, methyl or NH₂, wherein the remaining variables are as described above for Formula I or the fifth embodiment.

In a seventh embodiment, q in the compound of any one of Formulae I to IV, or a pharmaceutically acceptable salt thereof, is 1 or 2, wherein the remaining variables are as described above for Formula I or the fifth or sixth embodiment. In a seventh embodiment, q in the compound of any one of Formulae I to IV, or a pharmaceutically acceptable salt thereof, is 1 or 2, wherein the remaining variables are as described above for Formula I or the fifth or sixth embodiment.

In an eighth embodiment, R³ in the compound of any one of Formulae I to IV, or a pharmaceutically acceptable salt thereof, is —OR⁴, NHR⁵, —CCHR⁶, —NHCOR⁷, —C(O)R⁷, phenyl, heteroaryl, or heterocyclyl, wherein said phenyl, heteroaryl and heterocyclyl are each optionally substituted with 1 to 3 groups selected from R⁸; or R³ is taken together with one R² to form a 4- to 6-membered heteroaryl optionally substituted with a 9- or 10-membered fused-bicyclic heteroaryl which is optionally substituted with 1 to 3 groups selected from R⁹, wherein the remaining variables are as described above for Formula I or the fifth, sixth, or seventh embodiment. Alternatively, as part of an eighth embodiment, R³ in the compound of any one of Formulae I to IV, or a pharmaceutically acceptable salt thereof, is —OR⁴, NHR⁵, —CCHR⁶, —NHCOR⁷, —C(O)R⁷, phenyl, 5- to 6-membered heteroaryl, 9- to 10-membered fused bicyclic heteroaryl, 5- to 6-membered heterocyclyl, or 9- to 10-membered fused bicyclic heterocyclyl, wherein said phenyl, 5- to 6-membered heteroaryl, 9- to 10-membered fused bicyclic heteroaryl, 5- to 6-membered heterocyclyl, and 9- to 10-membered fused bicyclic heterocyclyl are each optionally substituted with 1 to 3 groups selected from R⁸; or R³ is taken together with one R² to form a 5-membered heteroaryl optionally substituted with a 9- or 10-membered fused-bicyclic heteroaryl which is optionally substituted with 1 to 3 groups selected from R⁹, wherein the remaining variables are as described above for Formula I or the fifth, sixth, or seventh embodiment. In another alternative, as part of an eighth embodiment, R³ in the compound of any one of Formulae I to IV, or a pharmaceutically acceptable salt thereof, is —OR⁴, NHR⁵, —CCHR⁶, —NHCOR⁷, —C(O)R⁷, (C₁-C₄)alkylpyrrolopyridinyl, phenyl, pyridinyl, thienopyrimidinyl, dihydropyridopyrimidinyl, dihydrobenzoimidazolyl imidazopyridinyl, pyridopyrimidinyl, or dihydropyridinyl, wherein said phenyl, pyridinyl, thienopyrimidinyl, dihydropyridopyrimidinyl, dihydrobenzoimidazolyl imidazopyridinyl, pyridopyrimidinyl, dihydropyridinyl, and pyrrolopyridinyl on the (C₁-C₄)alkylpyrrolopyridinyl are each optionally substituted with 1 to 3 groups selected from R⁸; or R³ is taken together with one R² to form a furanyl optionally substituted with imidazopyridazinyl which is optionally substituted with 1 to 3 groups selected from R⁹, wherein the remaining variables are as described above for Formula I or the fifth, sixth, or seventh embodiment. In another alternative, as part of an eighth embodiment, R³ in the compound of any one of Formulae I to IV, or a pharmaceutically acceptable salt thereof, is —OR⁴, NHR⁵, —CCHR⁶, —NHCOR⁷, —C(O)R⁷, phenyl, pyridinyl, thienopyrimidinyl, dihydrobenzoimidazolyl imidazopyridinyl, pyridopyrimidinyl, or dihydropyridinyl, wherein said phenyl, pyridinyl, thienopyrimidinyl, dihydrobenzoimidazolyl imidazopyridinyl, pyridopyrimidinyl, and dihydropyridinyl are each optionally substituted with 1 to 3 groups selected from R⁸; or R³ is taken together with one R² to form a furanyl optionally substituted with imidazopyridazinyl which is optionally substituted with 1 to 3 groups selected from R⁹, wherein the remaining variables are as described above for Formula I or the fifth, sixth, or seventh embodiment.

In a ninth embodiment, R⁴ in the compound of any one of Formulae I to IV, or a pharmaceutically acceptable salt thereof, is 5- to 6-membered heteroaryl or 9- to 10-membered fused bicyclic heteroaryl, each of which are optionally substituted with 1 to 3 groups selected from R¹⁰, wherein the remaining variables are as described above for Formula I or the fifth, sixth, seventh, or eighth embodiment. Alternatively, as part of a ninth embodiment, R⁴ in the compound of any one of Formulae I to IV, or a pharmaceutically acceptable salt thereof, is pyridinyl, dihydroquinazolinyl, dihydropyridopyrimidinyl, thiazolopyridinyl, quinolinyl, pyrrolopyridinyl, or tetrahydronaphthyridinyl, each of which are optionally substituted with 1 to 3 groups selected from R¹⁰, wherein the remaining variables are as described above for Formula I or the fifth, sixth, seventh, or eighth embodiment. In another alternative, as part of a ninth embodiment, R⁴ in the compound of any one of Formulae I to IV, or a pharmaceutically acceptable salt thereof, is pyridinyl, dihydroquinazolinyl, dihydropyridopyrimidinyl, thiazolopyridinyl, quinolinyl, or tetrahydronaphthyridinyl, each of which are optionally substituted with 1 to 3 groups selected from R¹⁰, wherein the remaining variables are as described above for Formula I or the fifth, sixth, seventh, or eighth embodiment.

In a tenth embodiment, R⁵ in the compound of any one of Formulae I to IV, or a pharmaceutically acceptable salt thereof, is 5- to 6-membered heteroaryl, 9- to 10-membered fused bicyclic heteroaryl, 5- to 6-membered heterocyclyl, or 9- to 10-membered fused bicyclic heterocyclyl, each of which are optionally substituted with 1 to 3 groups selected from R¹¹, wherein the remaining variables are as described above for Formula I or any one of the fifth to ninth embodiments. Alternatively, as part of a tenth embodiment, R⁵ in the compound of any one of Formulae I to IV, or a pharmaceutically acceptable salt thereof, is pyridinyl, pyrimidinyl, dihydroquinazolinyl, thiazolyl, dihydropyridopyrimidinyl, or imidazopyridazinyl, each of which are optionally substituted with 1 to 3 groups selected from R¹¹, wherein the remaining variables are as described above for Formula I or any one of the fifth to ninth embodiments.

In an eleventh embodiment, R⁶ in the compound of any one of Formulae I to IV, or a pharmaceutically acceptable salt thereof, is a 9- to 10-membered fused bicyclic heteroaryl optionally substituted with 1 to 3 groups selected from R¹², wherein the remaining variables are as described above for Formula I or any one of the fifth to tenth embodiments. Alternatively, as part of an eleventh embodiment, R⁶ is imidazopyridazinyl or thienopyrimidinyl, each of which are optionally substituted with 1 to 3 groups selected from R¹², wherein the remaining variables are as described above for Formula I or any one of the fifth to tenth embodiments.

In a twelfth embodiment, R⁷ in the compound of any one of Formulae I to IV, or a pharmaceutically acceptable salt thereof, is a 5- to 6-membered heteroaryl or a 9- to 10-membered heteroaryl each optionally substituted with 1 to 3 groups selected from R¹², wherein the remaining variables are as described above for Formula I or any one of the fifth to eleventh embodiments. Alternatively, as part of a twelfth embodiment, R⁷ in the compound of any one of Formulae I to IV, or a pharmaceutically acceptable salt thereof, is a 5- to 6-membered heteroaryl optionally substituted with 1 to 3 groups selected from R¹², wherein the remaining variables are as described above for Formula I or any one of the fifth to eleventh embodiments. In another alternative, as part of a twelfth embodiment, R⁷ in the compound of any one of Formulae I to IV, or a pharmaceutically acceptable salt thereof, is pyrrolopyridinyl, wherein the remaining variables are as described above for Formula I or any one of the fifth to eleventh embodiments. In another alternative, as part of a twelfth embodiment, R⁷ in the compound of any one of Formulae I to IV, or a pharmaceutically acceptable salt thereof, is a 9- to 10-membered heteroaryl optionally substituted with 1 to 3 groups selected from R¹².

In a thirteenth embodiment, R⁸ in the compound of any one of Formulae I to IV, or a pharmaceutically acceptable salt thereof, is selected from (C₁-C₆)alkyl, oxo, morpholinyl, —O(C₁-C₆)hydroxyalkyl, and —NR^{b}C(O)R^{b}, NR^{b}R^{c}, wherein the remaining variables are as described above for Formula I or any one of the fifth to twelfth embodiments.

In a fourteenth embodiment, R⁹ in the compound of any one of Formulae I to IV, or a pharmaceutically acceptable salt thereof, is halo, wherein the remaining variables are as described above for Formula I or any one of the fifth to thirteenth embodiments.

In a fifteenth embodiment, R¹⁰ in the compound of any one of Formulae I to IV, or a pharmaceutically acceptable salt thereof, is —C(O)NR^{b}R^{c}, —NR^{b}R^{c}, (C₁-C₆)alkoxy, and pyridinyl, wherein said pyridinyl is optionally substituted with —(C₁-C₆)alkylNH(C₁-C₆)hydroxyalkyl, wherein the remaining variables are as described above for Formula I or any one of the fifth to fourteenth embodiments.

In a sixteenth embodiment, R¹¹ in the compound of any one of Formulae I to IV, or a pharmaceutically acceptable salt thereof, is oxo, (C₁-C₆)alkyl, and heteroaryl, wherein said heteroaryl is optionally substituted with 1 to 3 groups selected from NR^{b}R^{c}, halo, (C₁-C₆)alkyl, —NR^{b}C(O)R^{b}, and —NR^{b}C(O)OR^{b}, wherein the remaining variables are as described above for Formula I or any one of the fifth to fifteenth embodiments. Alternatively, as part of a sixteenth embodiment, R¹¹ in the compound of any one of Formulae I to IV, or a pharmaceutically acceptable salt thereof, is oxo, (C₁-C₆)alkyl, pyridinyl, thiazolyl, or purinyl, wherein said pyridinyl, thiazolyl, or purinyl are each optionally substituted with 1 to 3 groups selected from NR^{b}R^{c}, halo, (C₁-C₆)alkyl, —NR^{b}C(O)R^{b}, and —NR^{b}C(O)OR^{b}, wherein the remaining variables are as described above for Formula I or any one of the fifth to fifteenth embodiments. In another alternative, as part of a fifteenth embodiment, R¹¹ in the compound of any one of Formulae I to IV, or a pharmaceutically acceptable salt thereof, is oxo, (C₁-C₆)alkyl, pyridinyl, thiazolyl, or purinyl, wherein said pyridinyl, thiazolyl, or purinyl are each optionally substituted with 1 to 3 groups selected is optionally substituted with 1 to 3 groups selected from oxo and (C₁-C₆)alkyl, wherein the remaining variables are as described above for Formula I or any one of the fifth to fifteenth embodiments.

In a seventeenth embodiment, R¹² in the compound of any one of Formulae I to IV, or a pharmaceutically acceptable salt thereof, is NR^{b}R^{c} or halo, wherein the remaining variables are as described above for Formula I or any one of the fifth to fifteenth embodiments.

In an eighteenth embodiment, R^{c} in the compound of any one of Formulae I to IV, or a pharmaceutically acceptable salt thereof, is selected from hydrogen, (C₁-C₆)alkyl, and 4- to 6-membered heterocyclyl, wherein the remaining variables are as described above for Formula I or any one of the fifth to seventeenth embodiments.

Compounds having the disclosed formulae are further disclosed in the Exemplification and are included in the present disclosure. Pharmaceutically acceptable salts thereof as well as the neutral forms are included.

### 4. Uses, Formulation and Administration

The compounds and compositions described herein are generally useful for modulating the activity of protein kinase. In some aspects, the compounds and pharmaceutical compositions described herein inhibit the activity of protein kinase.

In some aspects, the compounds and pharmaceutical compositions described herein are useful in treating a disorder associated with protein kinase function. Thus, provided herein are methods of treating a condition associated with protein kinase function, comprising administering to a subject in need thereof, a therapeutically effective amount of a compound described herein, or a pharmaceutically acceptable salt thereof, or a pharmaceutical composition comprising a disclosed compound or pharmaceutically acceptable salt thereof. Also provided is the use of a compound described herein, or a pharmaceutically acceptable salt thereof, or a pharmaceutical composition comprising a disclosed compound or pharmaceutically acceptable salt thereof, for the manufacture of a medicament for treating a condition associated with protein kinase function. Also provided is a compound described herein, or a pharmaceutically acceptable salt thereof, or a pharmaceutical composition comprising a disclosed compound or pharmaceutically acceptable salt thereof, for use in treating a condition associated with protein kinase function.

In some aspects, the compounds and pharmaceutical compositions described herein are useful in treating a condition selected from an inflammatory disease, a neurodegenerative disease, cardiovascular disease, metabolic disease, pain, and cancer.

Examples of inflammatory disease include, but are not limited to, rheumatoid arthritis, psoriatic arthritis, inflammatory bowel disease, chronic obstructive pulmonary disease, osteo-arthritis, progression of atherosclerotic plaques, bone metastasis, asthma, interstitial cystitis, atopic dermatitis, psoriasis and systemic lupus erythematosus (SLE).

Examples of neurodegenerative disease include, but are not limited to, Alzheimers, Parkinson's disease, and multiple sclerosis.

Examples of cardiovascular disease include, but are not limited to, hypertension, coronary and cerebral vasospasm, restenosis, atherosclerosis, stroke, and heart failure

Examples of metabolic disease include, but are not limited to, type 1 diabetes, type 2 diabetes.

Examples of cancer include, but are not limited to, colon, lung, ovarian, kidney, pancreatic, thyroid, hepatocellular, renal, gastric, breast, and brain cancers.

In certain aspects, a pharmaceutical composition described herein is formulated for administration to a patient in need of such composition. Pharmaceutical compositions described herein may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the pharmaceutical compositions described herein may be aqueous or oleaginous suspension. These suspensions may be formulated according to techniques known in the art using suitable dispersing or wetting agents and suspending agents.

In some aspects, the pharmaceutical compositions are administered orally.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

### EXEMPLIFICATION

Kinase compounds disclosed herein are synthesized according to the following examples. As used below, and throughout the description of the invention, the following abbreviations, unless otherwise indicated, shall be understood to have the following meanings:
ACN: acetonitrile
° C.: degrees Celsius
δ: chemical shift in parts per million downfield from tetramethylsilane
dichloromethane (CH₂Cl₂)
DCM: dimethylformamide
DMF: dimethylsulfoxide
Et₂O: diethyl ether
EtOAc: ethyl acetate
ES⁺: electrospray ionization
Et: ethyl
g: gram(s)
Hex: hexanes
h: hour(s)
HPLC: high performance liquid chromatography
Hz: hertz
J: coupling constant (in NMR spectrometry)
LCMS: liquid chromatography mass spectrometry
p: micro
m: multiplet (spectral); meter(s); milli
M: molar
M+: parent molecular ion
Me: methyl
MeOH: methanol
MHz: megahertz
min: minute(s)
mol: mole(s); molecular (as in mol wt)
mL: milliliter
NIS: N-iodosuccinimide
MS: mass spectrometry
nm: nanometer(s)
NMR: nuclear magnetic resonance
pH: negative base 10 logarithm of hydrogen cation concentration; a measure of the acidity or basicity of an aqueous solution
PE: petroleum ether
rt: room temperature
s: singlet (spectral)
t: triplet (spectral)
T: temperature
TFA: trifluoroacetic acid
THF: tetrahydrofuran.

### General Analytical Techniques

### LCMS

Liquid Chromatography Mass Spectrometry (LCMS) was performed on a Shimadzu LCMS system consisting of Nexera XR HPLC stack (20 Series) with Nexera X2 SPD-M30A DAD and LCMS-2020 mass spectrometer using LabSolutions, v.5.89 software under the following parameters: Column temp: 45° C., Sample temp: 18° C. Gradient elution methods, mobile phase eluents, and columns are shown below.

Alternatively, Liquid Chromatography Mass Spectrometry (LCMS) was performed on a Shimadzu SCL-10AVP HPLC/PE SCIEX API 100/365 mass spectrometer under the following parameters:
Column: Agilent, Eclipse XDB-C18; Length: 50 mm; Diameter: 3 mm; pore size: 2.7 micron.
Column temp: 50° C.,
Sample temp: room temperature.
Gradient elution methods and mobile phase eluents are shown below.
Solvent A (0.1% Trifluoroacetic acid in water, pH=2.3)
Solvent B (0.1% Trifluoroacetic acid in acetonitrile)

05991008_AA0 (0.8 mL/min flow)

| Time (min) | Solvent A (%) | Solvent B (%) |
|---|---|---|
| 0.0 | 95 | 0 |
| 0.2 | 95 | 0 |
| 10.2 | 0 | 100 |
| 12.2 | 0 | 100 |
| 12.4 | 95 | 5 |
| 14.0 | 95 | 5 |

00951008_BB1 (0.8 mL/min flow)

| Time (min) | Solvent A (%) | Solvent B (%) |
|---|---|---|
| 0.0 | 100 | 0 |
| 0.2 | 100 | 0 |
| 10.2 | 5 | 95 |
| 12.5 | 5 | 95 |
| 13.0 | 100 | 0 |
| 14.5 | 100 | 0 |

05991008_BB1 (0.8 mL/min flow)—mixed mode column 1

| Time (min) | Solvent A (%) | Solvent B (%) |
|---|---|---|
| 0.0 | 95 | 5 |
| 0.2 | 95 | 5 |
| 10.2 | 0 | 100 |
| 12.2 | 0 | 100 |
| 12.4 | 95 | 5 |
| 14.5 | 95 | 5 |

05990510_AA0 (1.0 mL/min flow)—RP column 2

| Time (min) | Solvent A (%) | Solvent B (%) |
|---|---|---|
| 0.0 | 95 | 5 |
| 0.2 | 95 | 5 |
| 5.2 | 0 | 100 |
| 6.2 | 0 | 100 |
| 6.25 | 95 | 5 |
| 8.0 | 95 | 5 |

05991008_BB1HT (0.8 mL/min flow, column temp 50′Q)—mixed mode column 2

| Time (min) | Solvent A (%) | Solvent B (%) |
|---|---|---|
| 0.0 | 95 | 5 |
| 0.2 | 95 | 5 |
| 10.2 | 0 | 100 |
| 12.2 | 0 | 100 |
| 12.4 | 95 | 5 |
| 14.2 | 95 | 5 |

Method A-6

polar_6 min_100_1500 (1.5 mL/min flow)

| Time (min) | Solvent A (%) | Solvent B (%) |
|---|---|---|
| 0 | 95 | 5 |
| 0.1 | 95 | 5 |
| 3.6 | 0 | 100 |
| 4.6 | 0 | 100 |
| 4.7 | 95 | 5 |

Method B

polar_6 min_100_1500 (1.0 mL/min flow)

| Time (min) | Solvent A (%) | Solvent B (%) |
|---|---|---|
| 0 | 95 | 5 |
| 0.01 | 95 | 5 |
| 0.50 | 0 | 100 |
| 1.49 | 95 | 5 |
| 4 | 0 | 100 |

Method A-12

polar_12 min_100_1500 (1.5 mL/min flow)

| Time (min) | Solvent A (%) | Solvent B (%) |
|---|---|---|
| 0 | 95 | 5 |
| 0.01 | 95 | 5 |
| 9.6 | 0 | 100 |
| 10.6 | 0 | 100 |
| 10.7 | 95 | 5 |

### Neutral Mobile Phase

Solvent A (20 mM ammonium acetate in 10% MeOH/water, pH 7.4)
Solvent B (100% acetonitrile)

### Acidic Mobile Phase

Solvent A (0.1% formic acid in water, pH 2.3)
Solvent B (0.1% formic acid in acetonitrile)

### Columns

RP column 1: ACE EXCEL 3 C18; 3.0 um, 100×3 mm (Mac-Mod Part #EXL-111-1003U)
RP column 2: Zorbax Eclipse XDB C8; 1.8 um, 50×4.6 mm (Agilent Part #922975-906)
Mixed Mode column 1: Scherzo SM-C18; 3.0 um, 100×3 mm (Imtakt Part #SM034)
Mixed Mode column 2: Scherzo SM-C18; 3.0 um, 75×2 mm (Imtakt Part #SM023)

### Method C

Liquid Chromatography Mass Spectrometry (LCMS) was performed on a Shimadzu LCMS system consisting of consisting of a LC 20 AD prominence pump, DGU-20 A3 prominence degasser, SPD-M20A prominence DAD detector, SIL-HTC autosampler coupled with a Shimadzu LCMS (SQD) mass spectrometer using Lab Solutions, v.3.70.390 software under the following parameters: Column temp: 50° C. Gradient elution methods, mobile phase eluents, PDA detection and columns are shown below.

LCMS Gradient (1.2 mL/min flow)

| Time (min) | Solvent A (%) | Solvent B (%) |
|---|---|---|
| 0.0 | 98 | 2 |
| 2.0 | 2 | 98 |
| 3.0 | 2 | 98 |
| 3.20 | 98 | 2 |
| 4.0 | 98 | 2 |

### Acidic Mobile Phase

Solvent A: 0.05% formic acid in water (95%):ACN (5%)
Solvent B: 0.05% formic acid in acetonitrile

Injection volume: 2.0 uL

### PDA

Detection at 214 nm, 254 nm & 280 nm

### Columns

Waters X-Select CSH (3.0*50) mm, 2.5 μm

### Method D

Liquid Chromatography Mass Spectrometry (LCMS) was performed on a Shimadzu LCMS system consisting of a LC 20 AD prominence pump, DGU-20 A3 prominence degasser, SPD-M20A prominence DAD detector, SIL-HTC autosampler coupled with a Shimadzu LCMS (SQD) mass spectrometer using Lab Solutions, v.3.70.390 software under the following parameters: Column temp: 50° C. Gradient elution methods, mobile phase eluents, PDA detection and columns are shown below.

LCMS Gradient (1.2 mL/min flow)

| Time (min) | Solvent A (%) | Solvent B (%) |
|---|---|---|
| 0.0 | 100 | 0 |
| 6.0 | 2 | 98 |
| 8.0 | 2 | 98 |
| 9.0 | 100 | 0 |
| 10.0 | 100 | 0 |

### Acidic Mobile Phase

Solvent A: 0.05% formic acid in water (95%):ACN (5%)

Solvent B: 0.05% formic acid in acetonitrile

### PDA

Detection at 214 nm, 254 nm & 280 nm

Injection volume: 2.0 uL

### Columns

Waters X-Bridge CSH (3.0*50 mm) 2.5 μm.

### Method E

Liquid Chromatography Mass Spectrometry (LCMS) was performed on a Shimadzu LCMS system consisting of a LC 20 AD prominence pump, DGU-20 A3 prominence degasser, SPD-M20A prominence DAD detector, SIL-HTC autosampler coupled with a Shimadzu LCMS(SQD) mass spectrometer using Lab Solutions, v.3.70.390 software under the following parameters: Column temp: 50° C. Gradient elution methods, mobile phase eluents, PDA detection and columns are shown below.

LCMS Gradient (1.2 mL/min flow)

| Time (min) | Solvent A (%) | Solvent B (%) |
|---|---|---|
| 0.01 | 98 | 2 |
| 0.2 | 98 | 2 |
| 2.2 | 2 | 98 |
| 3.0 | 2 | 98 |
| 3.2 | 98 | 2 |
| 4.0 | 98 | 2 |

### Acidic Mobile Phase

Solvent A: 0.05% formic acid in water
Solvent B: 0.05% formic acid in acetonitrile

Injection volume: 2.0 uL

### PDA

Detection at 214 nm, 254 nm & 280 nm

### Columns

Waters XSelect-C18(3.0*50) mm, 2.5 μm.

### Method F

Liquid Chromatography Mass Spectrometry (LCMS) was performed on a Shimadzu LCMS system consisting of consisting of a LC 20 AD prominence pump, DGU-20 A3 prominence degasser, SPD-M20A prominence DAD detector, SIL-HTC autosampler coupled with a Shimadzu LCMS(SQD) mass spectrometer using Lab Solutions, v.3.70.390 software under the following parameters: Column temp: 40° C. Gradient elution methods, mobile phase eluents, PDA detection and columns are shown below.

LCMS Gradient (1.2 mL/min flow)

| Time (min) | Solvent A (%) | Solvent B (%) |
|---|---|---|
| 0.00 | 100 | 0 |
| 1.5 | 0 | 100 |
| 2.2 | 0 | 100 |
| 2.6 | 100 | 0 |
| 3.0 | 93 | 7 |

### Mobile Phase

Solvent A: 2.5 mM Ammonium Bicarbonate+5% ACN in H₂0
Solvent B: Acetonitrile.

Injection volume: 2.0 μL

### PDA

Detection at 214 nm, 254 nm & 280 nm

### Columns

X Select CSH C18 (3.0*50) mm 2.5 u

Analytical HPLC

### Method A

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (1.2 mL/min flow)

| Time (min) | Solvent A (%) | Solvent B (%) |
|---|---|---|
| 0.01 | 95 | 5 |
| 1.0 | 95 | 5 |
| 8.0 | 0 | 100 |
| 12.0 | 0 | 100 |
| 14.0 | 95 | 5 |
| 18.0 | 95 | 5 |

### Mobile Phase

Solvent A: 0.1% TFA in water
Solvent B: 0.1% TFA in acetonitrile

Inj Volume—5.0 uL

### Columns

Waters X-Select CSH C18 (4.6*150) mm 5 μm.

### Method B

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (1.0 mL/min flow)

| Time (min) | Solvent A (%) | Solvent B (%) |
|---|---|---|
| 0.01 | 90 | 10 |
| 6.0 | 10 | 90 |
| 10.0 | 0 | 100 |
| 12.0 | 0 | 100 |
| 14.0 | 90 | 10 |
| 18.0 | 90 | 10 |

### Mobile Phase

Solvent A: 0.1% Formic acid in water:Acetonitrile (95:05)
Solvent B: Acetonitrile

Inj Volume—5.0 uL

### Columns

Waters X-Select CSH C18 (4.6*150) mm 5 μm.

### Method C

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (1.0 mL/min flow)

| Time (min) | Solvent A (%) | Solvent B (%) |
|---|---|---|
| 0.01 | 90 | 10 |
| 6.0 | 10 | 90 |
| 10.0 | 0 | 100 |
| 12.0 | 0 | 100 |
| 14.0 | 90 | 10 |
| 18.0 | 90 | 10 |

### Mobile Phase

Solvent A: 5 mM Ammonium Bicarbonate in water
Solvent B: Acetonitrile

Inj Volume—5.0 uL

### Columns

Waters X-Bridge CSH C18 (4.6*150) mm 5 μm.

### Method D

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (1.2 mL/min flow)

| Time (min) | Solvent A (%) | Solvent B (%) |
|---|---|---|
| 0.01 | 95 | 5 |
| 1.0 | 95 | 5 |
| 8.0 | 0 | 100 |
| 12.0 | 0 | 100 |
| 14.0 | 95 | 5 |
| 18.0 | 95 | 5 |

### Mobile Phase

Solvent A: 0.1% TFA in water
Solvent B: Acetonitrile

Inj Volume—5.0 uL

### Columns

Waters X-Select CSH C18 (4.6*150) mm 5 μm.

### HPLC

Preparative High-performance liquid chromatography (HPLC) was performed on a Shimadzu HPLC equipped with 2× LC-10ADvp pumps, Rheodyne 7725i manual injection valve, SPD-10AVvp UV/vis detector, SCL-10Avp system controller, and FRC-10A fraction collector using LabSolutions Lite, v.6.43 SP1 software and under the following conditions: Column temp: ambient; sample temp: ambient. Elution methods and mobile phase eluents are shown below.

Preparative isocratic HPLC 1 [Method RP-2525-2080]

| Time (min) | Flow (mL/min) | Solvent A (%) | Solvent B (%) |
|---|---|---|---|
| 0.0 | 8 | 75 | 25 |
| 20.0 | 8 | 75 | 25 |
| 20.2 | 8 | 5 | 95 |
| 23.2 | 8 | 5 | 95 |
| 23.5 | 8 | 75 | 25 |
| 27.0 | 8 | 75 | 25 |

Preparative HPLC gradient [Method RP-2040-2099]

| Time (min) | Flow (mL/min) | Solvent A (%) | Solvent B (%) |
|---|---|---|---|
| 0.0 | 9.99 | 80 | 20 |
| 20.0 | 9.99 | 60 | 40 |
| 20.4 | 9.99 | 5 | 95 |
| 24.0 | 9.99 | 5 | 95 |
| 24.5 | 9.99 | 80 | 20 |
| 30.0 | 9.99 | 80 | 20 |

### Neutral Mobile Phase

Solvent A (20 mM ammonium acetate in 10% MeOH/water, pH=7.4)
Solvent B (100% acetonitrile)

### Acidic Mobile Phase

Solvent A (0.1% formic acid in water, pH=2.3)
Solvent B (100% acetonitrile)

### Columns

RP: MACCEL PREP2005; 10.0 um; 50×20 mm (Bischoff Part #B052 0F180PS100) YMC-Pack ODS-A; 5.0 um; 150×10 mm (YMC Part #AA12S05-1510WT) ACE 5 C18-PFP; 5.0 um; 150×10 mm (Avantor-ACE Part #ACE-1210-1510)
GPC: TSKgel a-2500; 7.0 um, 300×7.8 mm; (TOSOH Part #0018339

### Method A

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (25 mL/min flow)

| Time (min) | Solvent A (%) | Solvent B (%) |
|---|---|---|
| 0.01 | 95 | 5 |
| 1.0 | 95 | 5 |
| 8.0 | 0 | 100 |
| 12.0 | 0 | 100 |
| 14.0 | 95 | 5 |
| 18.0 | 95 | 5 |

### Mobile Phase

Solvent A: 0.1% TFA in water
Solvent B: Acetonitrile

Inj Volume—5.0 uL

### Columns

X-SELECT (250*30 mm), 5.0 μm.

### Method B

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (25 mL/min flow)

| Time (min) | Solvent A (%) | Solvent B (%) |
|---|---|---|
| 0 | 85 | 15 |
| 3 | 85 | 15 |
| 12 | 75 | 25 |
| 17 | 40 | 60 |
| 22 | 35 | 65 |
| 27 | 30 | 70 |
| 32 | 25 | 75 |
| 37 | 20 | 80 |
| 42 | 10 | 90 |
| 47 | 0 | 100 |
| 52 | 0 | 100 |

### Solubility

ACN: H₂O: DMSO: TFA

### Mobile Phase

Solvent A: 0.1% TFA in water
Solvent B: Acetonitrile

Inj Volume—5.0 uL

### Columns

X-select: C-18 (30×250 mm), 5 nm.

### Method C

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (25 mL/min flow)

| Time (min) | Solvent A (%) | Solvent B (%) |
|---|---|---|
| 0.01 | 95 | 5 |
| 1.0 | 95 | 5 |
| 8.0 | 0 | 100 |
| 12.0 | 0 | 100 |
| 14.0 | 95 | 5 |
| 18.0 | 95 | 5 |

### Mobile Phase

A: 5 mM ammonium bicarbonate in Water
B: acetonitrile

Inj Volume—5.0 μL

### Column:

X-SELECT (250*30 mm), 5.0 μm.

### Method D

HPLC analyses were obtained on a Shimadzu HPLC 2010CHT HPLC consisting of a LC 20 AD prominence pump, DGU-20 A3 prominence degasser, SPD-M20A prominence DAD detector, SIL-HTC autosampler using LC Solutions, v.1.25 software under the following parameters: Column temp: 35° C. Gradient elution methods, mobile phase eluents, PDA detection and columns are shown below.

LC Gradient (25 mL/min flow)

| Time (min) | Solvent A (%) | Solvent B (%) |
|---|---|---|
| 0.01 | 95 | 5 |
| 1.0 | 95 | 5 |
| 8.0 | 0 | 100 |
| 12.0 | 0 | 100 |
| 14.0 | 95 | 5 |
| 18.0 | 95 | 5 |

### Mobile Phase

A: 5 mM ammonium bicarbonate in Water
B: acetonitrile

Inj Volume—5.0 μL

### Column:

X-Bridge C18 (4.6*150) mm 5 u
¹H NMR Proton NMR was performed on the Varian Inova 500 spectrometer operating at 500 MHz in CDCl₃, DMSO-d₆, or MeOD.
or
¹H NMR Proton NMR was performed on the Varian MR 400 MHz or Bruker Avance neo 400 MHz spectrometer operating at 400 MHz in CDCl₃, DMSO-d₆, or MeOD.

Synthetic Method A: Typically used 1 equivalent of amine intermediate, 1-2 equivalents of acid intermediate, 1 to 2 equivalents of HATU, 2-4 equivalents of DIPEA, and 3 to 16 hr of reaction time in DMF. A full example is written out below for Example 4

Synthetic Method B: Typically used 1 equivalent of amine intermediate, A range of 1-2 equivalents of acid intermediate, A range 1 to 2 equivalents of HATU, A range of 2-4 equivalents of DIPEA, and reaction time range of 3 to 16 hr in DMF. Followed by a deprotection step which used with TFA or HCl for deprotection of a Boc or THP protection group. A full example is written out below for Example 8

### Synthesis of 4-(4-(1-(4-fluorophenyl)-5-(methylsulfonyl)-1H-pyrazole-3-carboxamido)-phenoxy)-N-methylpicolinamide [Example 1]

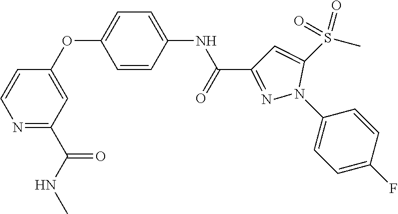 [SMILES: CNC(=O)c1cc(Oc2ccc(NC(=O)c3cc(S(C)(=O)=O)n(-c4ccc(F)cc4)n3)cc2)ccn1]

### Synthesis of [Example 1]: Followed the same procedure as Method A

### Synthesis of 1-(4-fluorophenyl)-N-(4-methyl-3-((4-(pyridin-3-yl)pyrimidin-2-yl)amino)phenyl)-5-(methylsulfonyl)-1H-pyrazole-3-carboxamide [Example 2]

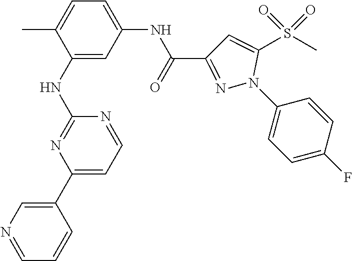 [SMILES: Cc1ccc(NC(=O)c2cc(S(C)(=O)=O)n(-c3ccc(F)cc3)n2)cc1Nc1nccc(-c2cccnc2)n1]

Synthesis of Example 2 followed the same procedure used in Synthetic Method A except used 1-(4-fluorophenyl)-5-(methylsulfonyl)-1H-pyrazole-3-carboxylic acid and 2-(5-Amino-2-methylanilino)-4-(3-pyridyl)pyrimidine. The solid was collected by filtration and dried to afford 165 mg, 0.303 mmol, 90% yield of the title compound as an off-white solid. ¹H NMR (500 MHz, DMSO-d₆) δ 10.37 (s, 1H), 9.27 (s, 1H), 8.98 (s, 1H), 8.70 (d, J=4.6 Hz, 1H), 8.51 (dd, J=6.0, 3.7 Hz, 2H), 8.10 (d, J=2.2 Hz, 1H), 7.75 (dd, J=8.8, 4.8 Hz, 2H), 7.64 (s, 1H), 7.56 (dd, J=8.1, 4.8 Hz, 1H), 7.49 (dd, J=8.3, 2.2 Hz, 1H), 7.46-7.40 (m, 3H), 7.19 (d, J=8.3 Hz, 1H), 3.32 (s, 3H), 2.21 (s, 3H); MS(ES⁺) m/z calc'd for [M+H]⁺ [C₂₇H₂₂FN₇O₃S+H]⁺: 544.1, found 544.3, t_{R}=1.89 min [Analytical Method B].

### Synthesis of 1-(4-fluorophenyl)-N-(4-methyl-3-((3-methyl-4-oxo-3,4-dihydroquinazolin-6-yl)amino)phenyl)-5-(methylthio)-1H-pyrazole-3-carboxamide [Example 3]

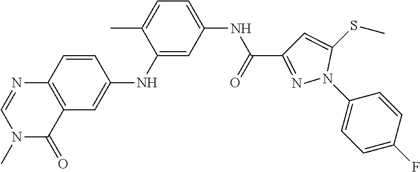 [SMILES: CSc1cc(C(=O)Nc2ccc(C)c(Nc3ccc4ncn(C)c(=O)c4c3)c2)nn1-c1ccc(F)cc1]

Synthesis of Example 3 followed the same procedure used in Synthetic Method A except used 1-(4-fluorophenyl)-5-(methylthio)-1H-pyrazole-3-carboxylic acid and 6-((5-amino-2-methylphenyl)amino)-3-methylquinazolin-4(3H)-one. The solid was collected by filtration and dried to afford 55 mg, 0.107 mmol, 17.97% yield of the title compound as an off-white solid. MS(ES⁺) m/z calc'd for [M+H]⁺ [C₂₇H₂₃FN₆O₂S+H]⁺: 515.2, found 515.2, t_{R}=2.38 min [Analytical Method B].

### Synthesis of 1-(4-fluorophenyl)-N-(4-methyl-3-((3-methyl-4-oxo-3,4-dihydroquinazolin-6-yl)amino)phenyl)-5-(methylsulfonyl)-1H-pyrazole-3-carboxamide [Example 4]

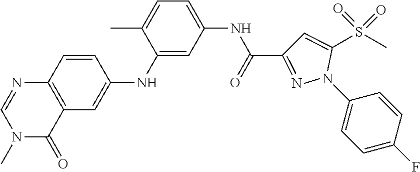 [SMILES: Cc1ccc(NC(=O)c2cc(S(C)(=O)=O)n(-c3ccc(F)cc3)n2)cc1Nc1ccc2ncn(C)c(=O)c2c1]

A solution of 6-((5-amino-2-methylphenyl)amino)-3-methylquinazolin-4(3H)-one (0.100 g, 0.357 mmol, 1.00 eq) and 1-(4-fluorophenyl)-5-(methylsulfonyl)-1H-pyrazole-3-carboxylic acid (0.12 g, 0.428 mmol, 1.20 eq) in anhydrous DMF (1 mL, 0.1784 M) was charged with diisopropylethylamine (0.19 mL, 1.07 mmol, 3.00 eq) and a solution of 1-[Bis(Dimethylamino)methylene]-1H-1,2,3-triazolo[4,5-b]pyridinium 3-oxide hexafluorophosphate (0.18 g, 0.464 mmol, 1.30 eq) in anhydrous DMF (1 mL, 0.1784 M) and stirred at rt for 2 h. The reaction mixture was charged with aqueous sat NaHCO₃ (4 mL) and stirred for 3 h. The solid was filtered through a fritted funnel and the filter cake was washed with aqueous sat. NaHCO₃ (2×15 mL), H₂O (1×15 mL), 1M LiCl (2×15 mL), H₂O (2×15 mL), and 4:1 diethyl ether/EtOAc (2×15 mL). The solid was further dried under high vac for 16 h resulting in 189.5 mg, 96.7% yield of the title compound as an off-white solid. ¹H NMR (600 MHz, DMSO-d₆) δ 10.35 (s, 1H), 8.14 (s, 1H), 7.96 (s, 1H), 7.79 (d, J=2.2 Hz, 1H), 7.75 (dd, J=8.9, 4.8 Hz, 2H), 7.62 (s, 1H), 7.53 (d, J=8.7 Hz, 1H), 7.49-7.40 (m, 5H), 7.21 (d, J=8.3 Hz, 1H), 3.45 (s, 3H), 2.18 (s, 3H); MS (ES⁺) m/z calc'd for [M+H]⁺ [C₂₇H₂₃FN₆O₄S+H]⁺: 547.58.6 found 547.3, LCMS: t_{R}=6.33 min [Analytical method:05991008_AA0_1.4KDa.lcm]

### Synthesis of 1-(4-fluorophenyl)-N-(4-methyl-3-((3-methyl-4-oxo-3,4-dihydroquinazolin-6-yl)amino)phenyl)-5-(methylsulfinyl)-1H-pyrazole-3-carboxamide [Example 5]

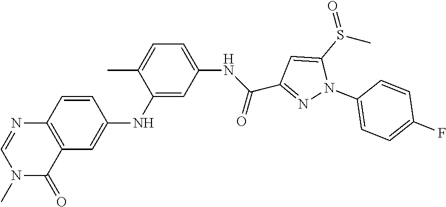 [SMILES: Cc1ccc(NC(=O)c2cc(S(C)=O)n(-c3ccc(F)cc3)n2)cc1Nc1ccc2ncn(C)c(=O)c2c1]

Synthesis of Example 5 followed the same procedure used in Synthetic Method Except used 1-(4-fluorophenyl)-5-(methylsulfinyl)-1H-pyrazole-3-carboxylic acid and 6-((5-amino-2-methylphenyl)amino)-3-methylquinazolin-4(3H)-one. Resulting 103 mg, 67.2% yield of the title compound as an off-white solid. ¹H NMR (500 MHz, DMSO-d₆) δ 10.30 (s, 1H), 10.12 (s, 1H), 8.42 (s, 1H), 8.16 (d, J=2.1 Hz, 1H), 7.81 (ddt, J=8.4, 5.5, 2.9 Hz, 2H), 7.70 (d, J=10.4 Hz, 2H), 7.65 (s, 1H), 7.55-7.44 (m, 3H), 7.20 (d, J=8.3 Hz, 1H), 6.93 (d, J=9.7 Hz, 1H), 4.12 (q, J=7.1 Hz, 2H), 3.07 (s, 3H), 2.23 (s, 3H), 1.22 (t, J=7.1 Hz, 3H); MS (ES⁺) m/z calc'd for [M+H]⁺ [C₂₇H₂₅FN₈O₄S+H]⁺: 577.61 found 577.2, LCMS: t_{R}=5.27 min [Analytical Method: 05991008_AA1.lcm]

### Synthesis of 1-(4-fluorophenyl)-N-(3-(imidazo[1,2-b]pyridazin-3-ylethynyl)-4-methylphenyl)-5-(methylsulfinyl)-1H-pyrazole-3-carboxamide [Example 6]

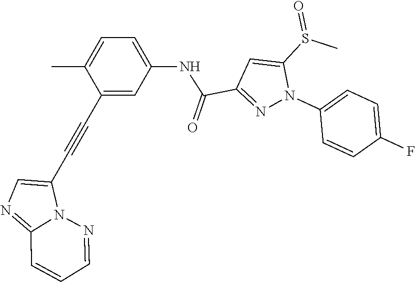 [SMILES: Cc1ccc(NC(=O)c2cc(S(C)=O)n(-c3ccc(F)cc3)n2)cc1C#Cc1cnc2cccnn12]

Synthesis of Example 6 followed the same procedure used in Synthetic Method A except used 3-(imidazo[1,2-b]pyridazin-3-ylethynyl)-4-methylaniline 2,2,2-trifluoroacetate and 1-(4-fluorophenyl)-5-(methylsulfinyl)-1H-pyrazole-3-carboxylic acid resulting in 0.062 g, 0.120 mmol, 75% yield of the title compound as a pale orange solid. ¹H NMR (600 MHz, DMSO-d₆) δ 10.45 (s, 1H), 8.71 (dd, J=4.4, 1.5 Hz, 1H), 8.25 (dd, J=9.2, 1.5 Hz, 1H), 8.22 (s, 1H), 8.11 (d, J=2.2 Hz, 1H), 7.84-7.79 (m, 2H), 7.76 (dd, J=8.3, 2.2 Hz, 1H), 7.70 (s, 1H), 7.53-7.48 (m, 2H), 7.38 (dd, J=9.2, 4.4 Hz, 1H), 7.34 (d, J=8.5 Hz, 1H), 3.08 (s, 3H), 2.49 (s, 3H); MS (ES⁺) m/z calc'd for [M+H]⁺[C₂₆H₁₉FN₆O₂S+H]⁺: 499.1 found 499.1, LCMS: t_{R}=6.42 min [method:05991008_AA0_1.4KDa.lcm]

### Synthesis of 4-(4-fluoro-3-(1-(4-fluorophenyl)-5-(methylsulfinyl)-1H-pyrazole-3-carboxamido)phenoxy)-N-methylpicolinamide[Example 7]

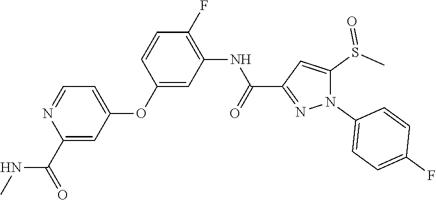 [SMILES: CNC(=O)c1cc(Oc2ccc(F)c(NC(=O)c3cc(S(C)=O)n(-c4ccc(F)cc4)n3)c2)ccn1]

Synthesis of Example 7 followed the same procedure used in Synthetic Method A except used 4-(3-amino-4-fluorophenoxy)-N-methylpicolinamide and 1-(4-fluorophenyl)-5-(methylsulfinyl)-1H-pyrazole-3-carboxylic acid resulting in 0.033 g, 0.065 mmol, 37% yield of the title compound as a pale orange solid. ¹H NMR (600 MHz, CDCl₃) δ 9.01 (d, J=2.7 Hz, 1H), 8.40 (d, J=5.6 Hz, 1H), 8.36 (dd, J=6.5, 2.9 Hz, 1H), 8.01-7.97 (m, 1H), 7.72-7.66 (m, 2H), 7.51 (s, 1H), 7.31-7.27 (m, 2H), 7.19 (dd, J=10.3, 8.9 Hz, 1H), 7.02 (dd, J=5.6, 2.6 Hz, 1H), 6.86-6.81 (m, 1H), 3.01 (d, J=5.1 Hz, 3H), 2.92 (s, 3H); MS (ES⁺) m/z calc'd for [M+H]⁺ [C₂₄H₁₉F₂N₅O₄S+H]⁺: 512.1 found 512.1, LCMS: t_{R}=6.18 min [method:05991008_AA0_1.4KDa.lcm]

Synthesis of N-(azetidin-3-yl)-4-(4-fluoro-3-(1-(4-fluorophenyl)-5-(methylthio)-1H-pyrazole-3-carboxamido)phenoxy)picolinamide [Example 8]

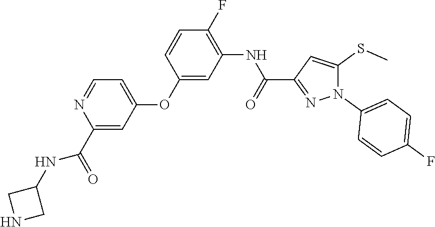 [SMILES: CSc1cc(C(=O)Nc2cc(Oc3ccnc(C(=O)NC4CNC4)c3)ccc2F)nn1-c1ccc(F)cc1]

A solution tert-butyl 3-(4-(3-amino-4-fluorophenoxy)picolinamido)azetidine-1-carboxylate (0.15 g, 0.37 mmol), 1-(4-fluorophenyl)-5-(methylthio)-1H-pyrazole-3-carboxylic acid (0.100 g, 0.41 mmol), and HATU (0.209 g, 0.55 mmol) in DMF (3.73 mL) was added DIEA (0.129 mL, 0.74 mmol) dropwise at rt. Then the mixture was stirred at rt for 16 h. At the end of the period water was added and the solid separated was collected and washed with sat. NaHCO₃ solution followed by water. The crude was chromatographed over silica gel using gradient of EtOAc in DCM to afford title product. Yield: 0.124 g

To a solution of tert-butyl 3-[[4-[2-fluoro-4-[[5-(4-fluorophenyl)-2-methylsulfanyl-oxazole-4-carbonyl]amino]phenoxy]pyridine-2-carbonyl]amino]azetidine-1-carboxylate (0.18 g, 0.282 mmol, 1.00 eq) in EtOAc (5 mL) was added 4M HCl in dioxane (3 mL) rt room temperature and stirring continued for 16 h. At the end of this period, the solvent was evaporated to dryness and the crude was chromatographed over SiO2 using 0-20% MeOH(10% NH4OH) in DCM to afford title product (0.045 g, 29.65%). ¹H NMR (600 MHz, DMSO-d₆) δ 10.50 (s, 1H), 9.13 (dd, J=43.5, 7.9 Hz, 1H), 8.58 (d, J=5.6 Hz, 1H), 8.29-8.14 (m, 2H), 8.04 (dd, J=13.0, 2.5 Hz, 1H), 7.78 (ddd, J=8.8, 2.5, 1.2 Hz, 1H), 7.53-7.30 (m, 3H), 7.25 (dd, J=5.6, 2.7 Hz, 1H), 4.68 (q, J=8.6, 8.0 Hz, 1H), 3.74-3.47 (m, 4H), 2.83 (s, 3H); MS (ES⁺) m/z calc'd for [M+H]⁺[C₂₆H₂₂F₂N₆O₃S+H]⁺: 537.56, found 537.2, LCMS: t_{R}=6.26 [05991008_AA0.lcm]

### Synthesis of 1-(4-fluorophenyl)-N-(3-(2-(2-hydroxyethoxy)-6-morpholinopyridin-4-yl)-4-methylphenyl)-5-(methylsulfonyl)-1H-pyrazole-3-carboxamide [Example 9]

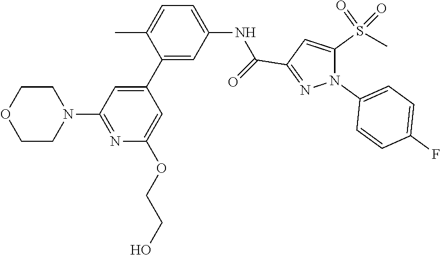 [SMILES: Cc1ccc(NC(=O)c2cc(S(C)(=O)=O)n(-c3ccc(F)cc3)n2)cc1-c1cc(OCCO)nc(N2CCOCC2)c1]

Synthesis of Example 9 followed the same procedure used in Synthetic Method A except used 4-methyl-3-(2-morpholino-6-(2-((tetrahydro-2H-pyran-2-yl)oxy)ethoxy)pyridin-4-yl)aniline and 1-(4-fluorophenyl)-5-(methylsulfonyl)-1H-pyrazole-3-carboxylic acid resulting in 63 mg, 0.105 mmol, 38.06% yield of the title compound as a white solid. 1H NMR (600 MHz, DMSO) δ 10.40 (s, 1H), 7.78-7.72 (m, 3H), 7.68 (d, J=2.2 Hz, 1H), 7.64 (s, 1H), 7.45 (t, J=8.7 Hz, 2H), 7.25 (d, J=8.5 Hz, 1H), 6.23 (s, 1H), 6.03-6.01 (m, 1H), 4.78 (t, J=5.6 Hz, 2H), 4.27-4.23 (m, 2H), 3.73-3.67 (m, 5H), 3.47-3.43 (m, 4H), 3.31 (s, 3H), 2.21 (s, 3H); MS (ES⁺) m/z calc'd for [M+H]⁺[C₂₉H₃₀FN₅O₆S+H]⁺: 597.64, found 596.2, LCMS: t_{R}=7.10 [: 05991008_AA1.lcm]

### Synthesis of 1-(4-fluorophenyl)-N-(3-(2-(2-hydroxyethoxy)-6-morpholinopyridin-4-yl)-4-methylphenyl)-5-(methylsulfinyl)-1H-pyrazole-3-carboxamide [Example 10]

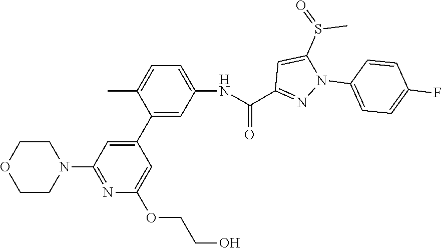 [SMILES: Cc1ccc(NC(=O)c2cc(S(C)=O)n(-c3ccc(F)cc3)n2)cc1-c1cc(OCCO)nc(N2CCOCC2)c1]

Synthesis of Example 10 followed the same procedure used in Synthetic Method B except used 1-(4-fluorophenyl)-5-(methylsulfinyl)-1H-pyrazole-3-carboxylic acid and 2-((4-(5-amino-2-methylphenyl)-6-morpholinopyridin-2-yl)oxy)ethan-1-ol. The crude material was purified by chromatography over silica gel, ISCO, CombiFlash, 4 g cartridge eluting with 80-100% EtOAc in hexanes to afford 110 mg, 33% yield of the title compound as a white solid. ¹H NMR (500 MHz, DMSO-d₆) δ=10.31 (s, 1H), 7.83-7.77 (m, 2H), 7.75 (dd, J=8.3, 2.3 Hz, 1H), 7.70-7.64 (m, 2H), 7.53-7.44 (m, 2H), 7.25 (d, J=8.4 Hz, 1H), 6.23 (d, J=1.0 Hz, 1H), 6.02 (d, J=0.9 Hz, 1H), 4.78 (t, J=5.5, 5.5 Hz, 1H), 4.24 (dd, J=5.8, 4.8 Hz, 2H), 3.69 (td, J=6.9, 6.0, 3.6 Hz, 6H), 3.45 (t, J=4.9, 4.9 Hz, 4H), 3.06 (s, 3H), 2.21 (s, 3H); MS (ES⁺): m/z=580.3 [M+H]⁺; LCMS: t_{R}=2.43 min [Analytical Method A-6].

### Synthesis of 1-(4-fluorophenyl)-N-(4-methyl-3-(1-methyl-5-morpholino-6-oxo-1,6-dihydropyridin-3-yl)phenyl)-5-(methylsulfinyl)-1H-pyrazole-3-carboxamide [Example 11]

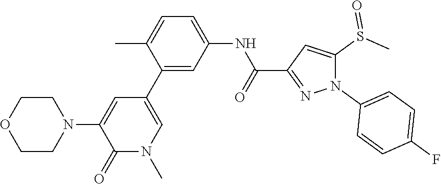 [SMILES: Cc1ccc(NC(=O)c2cc(S(C)=O)n(-c3ccc(F)cc3)n2)cc1-c1cc(N2CCOCC2)c(=O)n(C)c1]

Synthesis of Example 11 followed the same procedure as Synthetic Method A except used 5-(5-amino-2-methyl-phenyl)-1-methyl-3-morpholino-pyridin-2-one and 1-(4-fluorophenyl)-5-(methylsulfinyl)-1H-pyrazole-3-carboxylic acid. The crude was chromatographed over silica gel using 0-20% MeOH in DCM to give the title compound. ¹H NMR (600 MHz, DMSO-d₆) δ 10.29 (s, 1H), 7.88-7.77 (m, 2H), 7.77-7.64 (m, 3H), 7.55-7.45 (m, 2H), 7.45-7.35 (m, 1H), 7.25 (d, J=8.4 Hz, 1H), 6.70 (d, J=2.3 Hz, 1H), 3.72 (t, J=4.6 Hz, 4H), 3.50 (s, 3H), 3.09 (d, J=19.0 Hz, 7H), 2.26 (s, 3H); MS (ES⁺) m/z calc'd for [M+H]⁺[C₂₈H₂₈FN₅O₄S+H]⁺: 550.62, found 550.2, LCMS: t_{R}=5.91 [05991008_AA1.lcm]

### Synthesis of 1-(4-fluorophenyl)-N-(4-methyl-3-(1-methyl-5-morpholino-6-oxo-1,6-dihydropyridin-3-yl)phenyl)-5-(methylsulfonyl)-1H-pyrazole-3-carboxamide [Example 12]

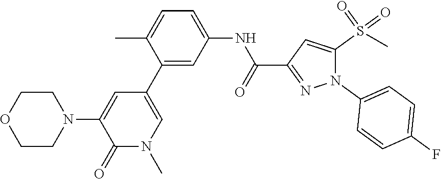 [SMILES: Cc1ccc(NC(=O)c2cc(S(C)(=O)=O)n(-c3ccc(F)cc3)n2)cc1-c1cc(N2CCOCC2)c(=O)n(C)c1]

Synthesis of Example 12 followed the same procedure used Synthetic Method A except used 1-(4-fluorophenyl)-5-(methylsulfonyl)-1H-pyrazole-3-carboxylic acid and 5-(5-amino-2-methyl-phenyl)-1-methyl-3-morpholino-pyridin-2-one. The crude was chromatographed over silica gel using 0-15% MeOH in DCM to afford 0.060 g, 72% yield of the title compound. 1H NMR (600 MHz, DMSO-d6) δ 10.38 (s, 1H), 7.80-7.73 (m, 2H), 7.72 (dd, J=8.3, 2.3 Hz, 1H), 7.70-7.62 (m, 2H), 7.49-7.42 (m, 2H), 7.39 (d, J=2.2 Hz, 1H), 7.25 (d, J=8.4 Hz, 1H), 6.70 (d, J=2.3 Hz, 1H), 3.72 (t, J=4.6 Hz, 4H), 3.50 (s, 3H), 3.33 (d, J=13.3 Hz, 5H), 3.11 (t, J=4.7 Hz, 4H), 2.26 (s, 3H); MS (ES⁺) m/z calc'd for [M+H]⁺ [C₂₈H₂₈FN₅O₅S+H]⁺: 566.62 found 566.2, LCMS: t_{R}=6.55 min [Analytical Method: 05991008_AA1.lcm]

### Synthesis of N-(3-((4-(2-aminopyridin-3-yl)pyrimidin-2-yl)amino)-4-methylphenyl)-1-(4-fluorophenyl)-5-(methylsulfonyl)-1H-pyrazole-3-carboxamide [Example 13]

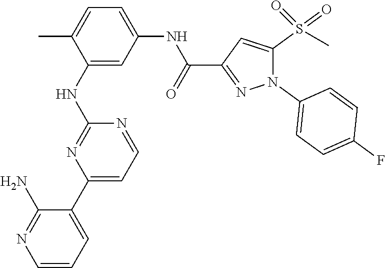 [SMILES: Cc1ccc(NC(=O)c2cc(S(C)(=O)=O)n(-c3ccc(F)cc3)n2)cc1Nc1nccc(-c2cccnc2N)n1]

Synthesis of Example 13 followed the same procedure as Synthetic Method A except used N3-[4-(2-amino-3-pyridyl)pyrimidin-2-yl]-4-methyl-benzene-1,3-diamine; hydrochloride and 1-(4-fluorophenyl)-5-(methylsulfonyl)-1H-pyrazole-3-carboxylic acid. The crude was purified by column chromatography (dry load; silica gel; 75-100% EtOAc in Hex) followed by another trituration (4:1 Et₂O:EtOAc) to afford 0.030 g, 0.0505 mmol, 17.29% yield of the title compound as a pale yellow solid. ¹H NMR (600 MHz, DMSO) δ 10.34 (s, 1H), 9.12 (s, 1H), 8.36 (d, J=5.4 Hz, 1H), 8.15-8.12 (m, 1H), 8.06 (dd, J=4.6, 1.8 Hz, 1H), 7.90 (d, J=2.1 Hz, 1H), 7.78-7.73 (m, 2H), 7.65 (s, 1H), 7.58-7.53 (m, 1H), 7.55-7.46 (m, 2H), 7.46-7.42 (m, 2H), 7.28 (d, J=5.6 Hz, 1H), 7.21 (d, J=8.4 Hz, 1H), 6.63 (dd, J=7.8, 4.7 Hz, 1H), 3.33 (s, 3H), 2.19 (s, 3H); MS (ES⁺) m/z calc'd for [M+H]⁺ [C₂₇H₂₃FN₈O₃S+H]⁺: 559.59 found 559.3, LCMS: t_{R}=5.26 min [Analytical method: 05991008_AA1.lcm]

### Synthesis of 1-(4-fluorophenyl)-N-(3-(imidazo[1,2-b]pyridazin-3-ylethynyl)-4-methylphenyl)-5-(methylsulfonyl)-1H-pyrazole-3-carboxamide [Example 14]

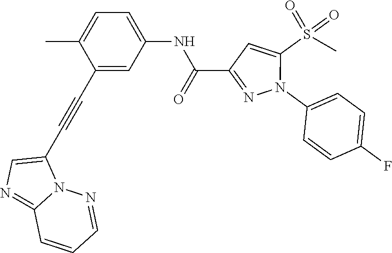 [SMILES: Cc1ccc(NC(=O)c2cc(S(C)(=O)=O)n(-c3ccc(F)cc3)n2)cc1C#Cc1cnc2cccnn12]

Synthesis of Example 14 followed the same procedure as Synthetic Method A except used 1-(4-fluorophenyl)-5-(methylsulfonyl)-1H-pyrazole-3-carboxylic acid and 3-(2-imidazo[1,2-b]pyridazin-3-ylethynyl)-4-methyl-aniline. The crude was purified with 90 EtOAc:Hex to afford 38 mg, 0.0733 mmol, 25.49% yield of the title compound. ¹H NMR (600 MHz, DMSO) δ 10.47 (s, 1H), 8.64 (dd, J=4.4, 1.4 Hz, 1H), 8.23-8.13 (m, 2H), 8.04 (d, J=2.3 Hz, 1H), 7.73-7.69 (m, 2H), 7.67 (dd, J=8.4, 2.3 Hz, 1H), 7.60 (s, 1H), 7.45-7.38 (m, 2H), 7.31 (dd, J=9.2, 4.4 Hz, 1H), 7.27 (d, J=8.5 Hz, 1H), 3.27 (s, 3H), 2.42 (s, 3H).MS (ES⁺) m/z calc'd for [M+H]⁺ [C₂₆H₁₉FN₆O₃S+H]⁺: 515.53 found 515.1, LCMS: t_{R}=7.30 min [method: 05991008_AA1.lcm]

### Synthesis of ethyl (6-((5-(1-(4-fluorophenyl)-5-(methylsulfonyl)-1H-pyrazole-3-carboxamido)-2-methylphenyl)amino)imidazo[1,2-b]pyridazin-2-yl)carbamate [Example 15]

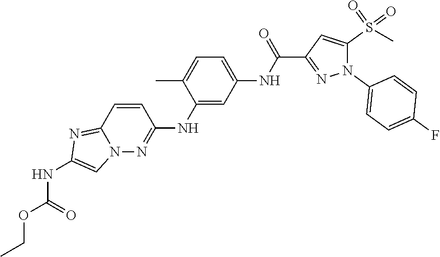 [SMILES: CCOC(=O)Nc1cn2nc(Nc3cc(NC(=O)c4cc(S(C)(=O)=O)n(-c5ccc(F)cc5)n4)ccc3C)ccc2n1]

Synthesis of Example 15 followed the same procedure as Synthetic Method A except used 1-(4-fluorophenyl)-5-(methylsulfonyl)-1H-pyrazole-3-carboxylic acid and ethyl N-[6-(5-amino-2-methyl-anilino)imidazo[1,2-b]pyridazin-2-yl]carbamate; 2,2,2-trifluoroacetic acid the crude was triturated resulting in 59 mg, 37% yield of the title compound as a light tan solid. ¹H NMR (600 MHz, DMSO-d₆) δ 10.37 (s, 1H), 10.13 (s, 1H), 8.38 (s, 1H), 8.16 (d, J=2.2 Hz, 1H), 7.79-7.74 (m, 2H), 7.70 (d, J=9.6 Hz, 1H), 7.65 (s, 2H), 7.50-7.42 (m, 3H), 7.19 (d, J=8.4 Hz, 1H), 6.91 (d, J=9.6 Hz, 1H), 4.12 (d, J=7.1 Hz, 2H), 3.33 (s, 3H), 2.22 (s, 3H), 1.23 (t, J=7.1 Hz, 3H); MS (ES⁺) m/z calc'd for [M+H]⁺ [C₂₇H₂₅FN₈O₅S+H]⁺: 593.61 found 593.1, LCMS: t_{R}=6.40 min [Analytical method: 05991008_AA1.lcm]

### Synthesis of 4-(3-fluoro-4-(1-(4-fluorophenyl)-5-(methylsulfinyl)-1H-pyrazole-3-carboxamido)phenoxy)-N-methylpicolinamide [Example 16]

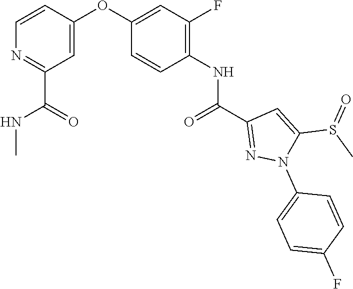 [SMILES: CNC(=O)c1cc(Oc2ccc(NC(=O)c3cc(S(C)=O)n(-c4ccc(F)cc4)n3)c(F)c2)ccn1]

Synthesis of Example 16 followed the same procedure as Synthetic Method A except used 4-(4-amino-3-fluoro-phenoxy)-N-methyl-pyridine-2-carboxamide and 1-(4-fluorophenyl)-3-(methylsulfinyl)-1H-pyrazole-5-carboxylic acid. The crude was chromatographed over silica gel using 0-15% MeOH in DCM to afford the title compound.

¹H NMR (600 MHz, DMSO-d₆) δ 10.21 (s, 1H), 8.80 (q, J=4.8 Hz, 1H), 8.56 (d, J=5.6 Hz, 1H), 7.85-7.80 (m, 2H), 7.77 (t, J=8.7 Hz, 1H), 7.72 (s, 1H), 7.55-7.49 (m, 2H), 7.46 (d, J=2.6 Hz, 1H), 7.38 (dd, J=10.9, 2.7 Hz, 1H), 7.24 (dd, J=5.6, 2.6 Hz, 1H), 7.14 (ddd, J=8.7, 2.7, 1.0 Hz, 1H), 3.09 (s, 3H), 2.80 (d, J=4.8 Hz, 3H); MS (ES⁺) m/z calc'd for [M+H]⁺[C₂₄H₁₉F₂N₅O₄S+H]⁺: 512.5 found 512.1, LCMS: t_{R}=5.97 min [Analytical method:: 05991008_AA1.lcm]

### Synthesis of 4-(3-fluoro-4-(1-(4-fluorophenyl)-5-(methylsulfonyl)-1H-pyrazole-3-carboxamido)phenoxy)-N-methylpicolinamide [Example 17]

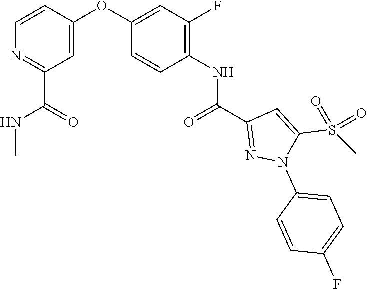 [SMILES: CNC(=O)c1cc(Oc2ccc(NC(=O)c3cc(S(C)(=O)=O)n(-c4ccc(F)cc4)n3)c(F)c2)ccn1]

Synthesis of Example 17 followed the same procedure as Synthetic Method A except used 4-(4-amino-3-fluoro-phenoxy)-N-methyl-pyridine-2-carboxamide and 1-(4-fluorophenyl)-5-(methylsulfonyl)-1H-pyrazole-3-carboxylic acid. The crude was chromatographed over silica gel using 0-15% MeOH in DCM to afford the title compound.

¹H NMR (600 MHz, DMSO-d₆) δ 10.33 (s, 1H), 8.80 (q, J=4.8 Hz, 1H), 8.56 (d, J=5.6 Hz, 1H), 7.84-7.73 (m, 2H), 7.77-7.64 (m, 2H), 7.51-7.42 (m, 2H), 7.37 (dd, J=10.9, 2.7 Hz, 1H), 7.24 (dd, J=5.6, 2.6 Hz, 1H), 7.13 (dd, J=8.7, 2.6 Hz, 1H), 3.36 (s, 2H), 2.80 (d, J=4.8 Hz, 2H); MS (ES⁺) m/z calc'd for [M+H]⁺ [C₂₄H₁₉F₂N₅O₅S+H]⁺: 528.5 found 528.1, LCMS: t_{R}=6.61 min [Analytical method: 05991008_AA1.lcm]

### Synthesis of ethyl (6-((5-(1-(4-fluorophenyl)-5-(methylsulfinyl)-1H-pyrazole-3-carboxamido)-2-methylphenyl)amino)imidazo[1,2-b]pyridazin-2-yl)carbamate [Example 18]

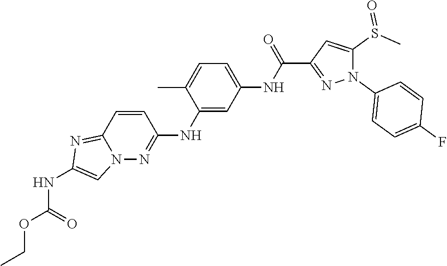 [SMILES: CCOC(=O)Nc1cn2nc(Nc3cc(NC(=O)c4cc(S(C)=O)n(-c5ccc(F)cc5)n4)ccc3C)ccc2n1]

Synthesis of Example 18 followed the same procedure as Synthetic Method A except used 1-(4-fluorophenyl)-5-(methylsulfinyl)-1H-pyrazole-3-carboxylic acid and ethyl N-[6-(5-amino-2-methyl-anilino)imidazo[1,2-b]pyridazin-2-yl]carbamate; 2,2,2-trifluoroacetic acid. The crude was triturated resulting in 103 mg, 67.2% yield of the title compound as an off-white solid. ¹H NMR (500 MHz, DMSO-d₆) δ 10.30 (s, 1H), 10.12 (s, 1H), 8.42 (s, 1H), 8.16 (d, J=2.1 Hz, 1H), 7.81 (ddt, J=8.4, 5.5, 2.9 Hz, 2H), 7.70 (d, J=10.4 Hz, 2H), 7.65 (s, 1H), 7.55-7.44 (m, 3H), 7.20 (d, J=8.3 Hz, 1H), 6.93 (d, J=9.7 Hz, 1H), 4.12 (q, J=7.1 Hz, 2H), 3.07 (s, 3H), 2.23 (s, 3H), 1.22 (t, J=7.1 Hz, 3H); MS (ES⁺) m/z calc'd for [M+H]⁺ [C₂₇H₂₅FN₈O₄S+H]⁺: 577.61 found 577.2, LCMS: t_{R}=5.27 min [Analytical method: 05991008_AA1.lcm]

### Synthesis of 1-(4-fluorophenyl)-N-(4-methyl-3-((4-(pyridin-3-yl)pyrimidin-2-yl)amino)phenyl)-5-(methylsulfinyl)-1H-pyrazole-3-carboxamide [Example 19]

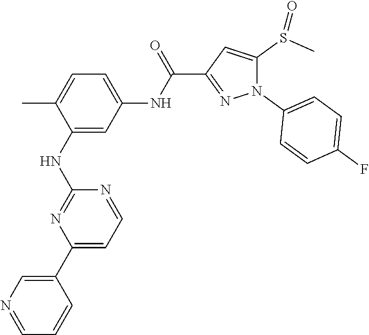 [SMILES: Cc1ccc(NC(=O)c2cc(S(C)=O)n(-c3ccc(F)cc3)n2)cc1Nc1nccc(-c2cccnc2)n1]

Synthesis of Example 19 Followed the same procedure as Synthetic Method A except used 1-(4-fluorophenyl)-5-(methylsulfinyl)-1H-pyrazole-3-carboxylic acid and 6-methyl-N¹-(4-(pyridin-3-yl)pyrimidin-2-yl)benzene-1,3-diamine resulting in 73 mg, 50% yield of the title compound as a pinkish-white solid. ¹H NMR (500 MHz, DMSO-d₆) δ 10.28 (s, 1H), 9.26 (s, 1H), 8.97 (s, 1H), 8.67 (s, 1H), 8.50 (s, 1H), 8.45 (d, J=7.9 Hz, 1H), 8.10 (s, 1H), 7.80 (s, 2H), 7.67 (s, 1H), 7.49 (dd, J=16.1, 8.2 Hz, 4H), 7.44-7.39 (m, 1H), 7.20 (d, J=8.3 Hz, 1H), 3.06 (s, 3H), 2.21 (s, 3H); MS (ES⁺): m/z=calc'd for [M+H]⁺; [C₂₉H₃0FN₅O₆S+H]⁺: 596.2 found 596.4, LCMS: t_{R}=²0.55 min [Analytical Method A-6].

### Synthesis of N-(3-((3-(9H-purin-6-yl)pyridin-2-yl)amino)-4-methylphenyl)-1-(4-fluorophenyl)-5-(methylsulfonyl)-1H-pyrazole-3-carboxamide [Example 20]

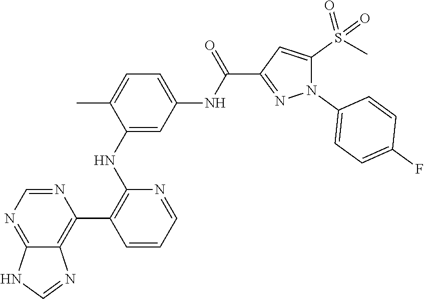 [SMILES: Cc1ccc(NC(=O)c2cc(S(C)(=O)=O)n(-c3ccc(F)cc3)n2)cc1Nc1ncccc1-c1ncnc2[nH]cnc12]

Synthesis of Example 20 followed the same procedure as Synthetic Method B except used N1-(3-(9H-purin-6-yl)pyridin-2-yl)-6-methylbenzene-1,3-diamine and 1-(4-fluorophenyl)-5-(methylsulfonyl)-1H-pyrazole-3-carboxylic acid and TFA for deprotection resulting in 56 mg, 48% yield of the title compound as an orange solid. ¹H NMR (500 MHz, DMSO-d₆) δ 12.27 (s, 1H), 10.32 (s, 1H), 9.77 (s, 1H), 9.07 (s, 1H), 8.71 (s, 1H), 8.65 (s, 1H), 8.32 (d, J=3.4 Hz, 1H), 7.76 (dd, J=8.7, 4.8 Hz, 2H), 7.67 (s, 1H), 7.48-7.42 (m, 3H), 7.22 (d, J=8.2 Hz, 1H), 7.02 (dd, J=7.9, 4.7 Hz, 1H), 3.33 (s, 3H), 2.41 (s, 3H); MS (ES⁺): m/z=584.4 [M+H]⁺; LCMS: t_{R}=1.81 min [Analytical Method A-6].

### Synthesis of 1-(4-fluorophenyl)-N-(3-hydroxy-5-(4-morpholinothieno[3,2-d]pyrimidin-2-yl)phenyl)-5-(methylsulfonyl)-1H-pyrazole-3-carboxamide [Example 21]

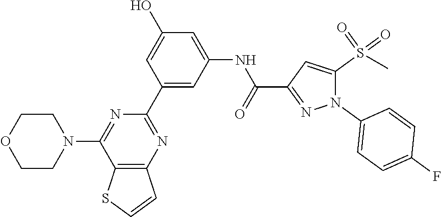 [SMILES: CS(=O)(=O)c1cc(C(=O)Nc2cc(O)cc(-c3nc(N4CCOCC4)c4sccc4n3)c2)nn1-c1ccc(F)cc1]

Synthesis of Example 21 followed same procedure as Synthetic Method A except used 3-amino-5-(4-morpholinothieno[3,2-d]pyrimidin-2-yl)phenol and 1-(4-fluorophenyl)-5-(methylsulfonyl)-1H-pyrazole-3-carboxylic acid. The crude material was purified via trituration with 4:1 DCM:MeOH to afford 0.091 g, 0.146 mmol, 56.52% yield of the title compound as a white solid. ¹H NMR (600 MHz, DMSO) δ 10.43 (s, 1H), 9.57 (s, 1H), 8.26 (d, J=5.5 Hz, 1H), 8.22 (s, 1H), 7.81-7.74 (m, 2H), 7.72 (s, 1H), 7.66-7.62 (m, 1H), 7.54 (s, 1H), 7.50 (d, J=5.5 Hz, 1H), 7.49-7.44 (m, 2H), 4.06-3.97 (m, 4H), 3.85-3.78 (m, 4H), 3.34 (s, 3H); MS (ES⁺) m/z calc'd for [M+H]⁺ [C₂H₂FN₆O₅S2+H]⁺: 595.64 found 595.2, LCMS: t_{R}=6.77 min [Analytical Method: 05991008_BB1.lcm]

### Synthesis of 1-(4-fluorophenyl)-N-(3-hydroxy-5-(4-morpholinothieno[3,2-d]pyrimidin-2-yl)phenyl)-5-(methylsulfinyl)-1H-pyrazole-3-carboxamide [Example 22]

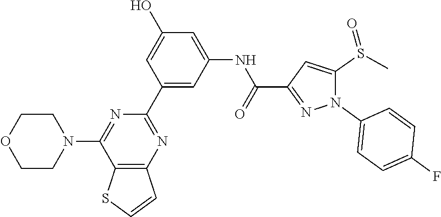 [SMILES: CS(=O)c1cc(C(=O)Nc2cc(O)cc(-c3nc(N4CCOCC4)c4sccc4n3)c2)nn1-c1ccc(F)cc1]

Synthesis of Example 22 followed the same procedure as Synthetic Method A except used 3-amino-5-(4-morpholinothieno[3,2-d]pyrimidin-2-yl)phenol and 1-(4-fluorophenyl)-5-(methylsulfinyl)-1H-pyrazole-3-carboxylic acid. The crude material was purified via trituration (3:1 Et₂O:EtOAc, then 4:1 DCM:MeOH) to afford 0.097 g, 0.161 mmol, 60.62% yield of the title compound as a an off-white solid. 1H NMR (600 MHz, DMSO) δ 10.36 (s, 1H), 8.26 (d, J=5.5 Hz, 1H), 8.23 (s, 1H), 7.85-7.81 (m, 2H), 7.73 (s, 1H), 7.64 (s, 1H), 7.56 (s, 1H), 7.55-7.48 (m, 3H), 4.10-3.96 (m, 4H), 3.87-3.75 (m, 4H), 3.09 (s, 3H); MS (ES⁺) m/z calc'd for [M+H]⁺[C²⁷H²³FN⁶O⁴S²+H]⁺: 579.64 found 579.2, LCMS: t_{R}=2.37 min [Analytical Method: 05990510_AA0.lcm]

### Synthesis of N-(3-((4-(2-aminopyridin-3-yl)pyrimidin-2-yl)amino)-4-methylphenyl)-1-(4-fluorophenyl)-5-(methylsulfinyl)-1H-pyrazole-3-carboxamide [Example 23]

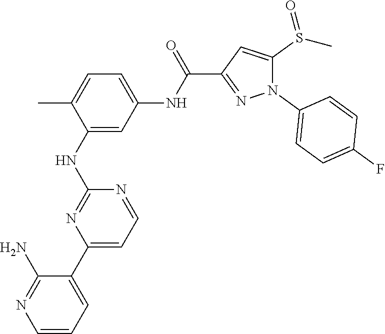 [SMILES: Cc1ccc(NC(=O)c2cc(S(C)=O)n(-c3ccc(F)cc3)n2)cc1Nc1nccc(-c2cccnc2N)n1]

The synthesis of Example 23 followed the same procedure as Synthetic Method A except used 1-(4-fluorophenyl)-5-(methylsulfinyl)-1H-pyrazole-3-carboxylic acid and N1-(4-(2-aminopyridin-3-yl)pyrimidin-2-yl)-6-methylbenzene-1,3-diamine. The crude was triturated with 3:1 Et₂O:EtOAc to afford 0.10 g, 0.182 mmol, 67.51% yield of the title compound as a pale yellow solid. 1H NMR (600 MHz, DMSO) δ 10.26 (s, 1H), 9.13 (s, 1H), 8.37 (d, J=5.4 Hz, 1H), 8.14 (dd, J=7.9, 1.7 Hz, 2H), 8.07 (dd, J=4.6, 1.7 Hz, 2H), 7.91 (d, J=2.0 Hz, 1H), 7.83-7.77 (m, 2H), 7.68 (s, 1H), 7.57 (dd, J=8.3, 2.1 Hz, 1H), 7.54-7.44 (m, 4H), 7.28 (d, J=5.6 Hz, 1H), 7.22 (d, J=8.4 Hz, 1H), 6.63 (dd, J=7.8, 4.7 Hz, 1H), 3.07 (s, 3H), 2.20 (s, 3H); MS (ES⁺) m/z calc'd for [M+H]⁺ [C₂₇H₂₃FN₈O₂S+H]⁺: 543.595 found 543.1, LCMS: t_{R}=3.78 min [Analytical Method: 05991008_AA1.lcm]

### Synthesis of N-(2-fluoro-5-((3-methyl-4-oxo-3,4-dihydroquinazolin-6-yl)oxy)phenyl)-1-(4-fluorophenyl)-5-(methylsulfinyl)-1H-pyrazole-3-carboxamide [Example 24]

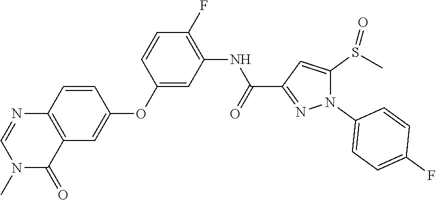 [SMILES: Cn1cnc2ccc(Oc3ccc(F)c(NC(=O)c4cc(S(C)=O)n(-c5ccc(F)cc5)n4)c3)cc2c1=O]

The synthesis of Example 24 followed the same procedure as Synthetic Method A except used 6-(3-amino-4-fluorophenoxy)-3-methylquinazolin-4(3H)-one and 1-(4-fluorophenyl)-5-(methylsulfinyl)-1H-pyrazole-3-carboxylic acid. The crude was further triturated with MeOH to afford 0.022 g, 0.0407 mmol, 14.50% yield of the title compound as a pale pink solid. 1H NMR (600 MHz, DMSO) δ 10.09 (s, 1H), 8.33 (s, 1H), 7.81-7.77 (m, 2H), 7.75 (d, J=8.8 Hz, 1H), 7.69 (s, 1H), 7.59 (dd, J=8.9, 2.9 Hz, 1H), 7.55 (dd, J=6.3, 3.0 Hz, 1H), 7.53 (d, J=2.9 Hz, 1H), 7.52-7.47 (m, 2H), 7.43-7.37 (m, 1H), 7.04 (dt, J=8.9, 3.4 Hz, 1H), 3.48 (s, 3H), 3.06 (s, 3H); MS (ES⁺) m/z calc'd for [M+H]⁺[C₂₆H₉F₂N₅O₄S+H]⁺: 536.52 found 536.2, LCMS: t_{R}=2.94 min [Analytical Method: 05990510_AA0.lcm]

### Synthesis of N-(2-fluoro-5-((3-methyl-4-oxo-3,4-dihydroquinazolin-6-yl)oxy)phenyl)-1-(4-fluorophenyl)-5-(methylsulfonyl)-1H-pyrazole-3-carboxamide [Example 25]

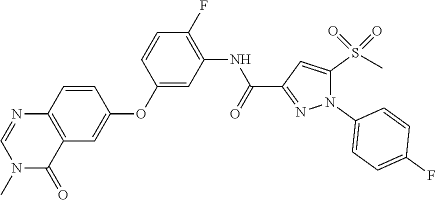 [SMILES: Cn1cnc2ccc(Oc3ccc(F)c(NC(=O)c4cc(S(C)(=O)=O)n(-c5ccc(F)cc5)n4)c3)cc2c1=O]

Synthesis of Example 25 followed the same procedure as Synthetic Method A except used 6-(3-amino-4-fluorophenoxy)-3-methylquinazolin-4(3H)-one and 1-(4-fluorophenyl)-5-(methylsulfonyl)-1H-pyrazole-3-carboxylic acid. The crude material was purified via trituration with MeOH to afford 0.069 g, 0.123 mmol, 44.03% yield of the title compound as an off-white solid. 1H NMR (600 MHz, DMSO) δ 10.22 (s, 1H), 8.33 (s, 1H), 7.78-7.73 (m, 3H), 7.64 (s, 1H), 7.59 (dd, J=8.8, 2.9 Hz, 1H), 7.52 (d, J=2.9 Hz, 1H), 7.49-7.43 (m, 3H), 7.42-7.36 (m, 1H), 7.05 (dt, J=8.9, 3.4 Hz, 1H), 3.48 (s, 3H), 3.32 (s, 3H); MS (ES⁺) m/z calc'd for [M+H]⁺ [C₂₆H₁₉F₂N₅O₅S]⁺ H]⁺: 552.52 found 552.2, LCMS: t_{R}=3.24 min [Analytical Method: 05990510_AA0.lcm]

### Synthesis of N-(3-((2-aminopyrimidin-5-yl)ethynyl)-2,4-difluorophenyl)-1-(4-fluorophenyl)-5-(methylsulfinyl)-1H-pyrazole-3-carboxamide [Example 26]

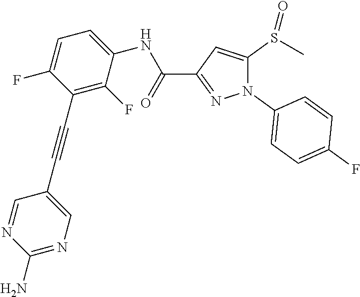 [SMILES: CS(=O)c1cc(C(=O)Nc2ccc(F)c(C#Cc3cnc(N)nc3)c2F)nn1-c1ccc(F)cc1]

Synthesis of Example 26 followed the same procedure used in Synthetic Method A except used 5-((3-amino-2,6-difluorophenyl)ethynyl)pyrimidin-2-amine and 1-(4-fluorophenyl)-5-(methylsulfinyl)-1H-pyrazole-3-carboxylic acid. The crude material was purified via trituration with 2:1 Et₂O:EtOAc to afford 0.092 g, 0.176 mmol, 51.49% yield of the title compound as a pale yellow solid. ¹H NMR (600 MHz, DMSO) δ 10.30 (s, 1H), 8.47 (s, 2H), 7.82-7.79 (m, 2H), 7.69 (s, 1H), 7.66-7.59 (m, 1H), 7.54-7.46 (m, 2H), 7.30-7.24 (m, 3H), 3.08 (s, 3H); MS (ES⁺) m/z calc'd for [M+H]⁺ [C₂₃H₁₅F₃N₆O₂S+H]⁺: 497.46 found 497.2, LCMS: t_{R}=2.75 min [Analytical Method: 05990510_AA0.lcm].

### Synthesis of 1-(4-fluorophenyl)-N-(4-methyl-3-((3-methyl-4-oxo-3,4-dihydroquinazolin-6-yl)oxy)phenyl)-5-(methylsulfinyl)-1H-pyrazole-3-carboxamide [Example 27]

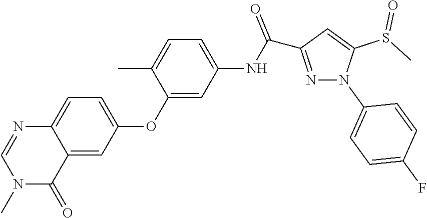 [SMILES: Cc1ccc(NC(=O)c2cc(S(C)=O)n(-c3ccc(F)cc3)n2)cc1Oc1ccc2ncn(C)c(=O)c2c1]

Synthesis of Example 27 followed the same procedure used in Synthetic Method A except used 1-(4-fluorophenyl)-5-(methylsulfinyl)-1H-pyrazole-3-carboxylic acid and 6-(5-amino-2-methyl-phenoxy)-3-methyl-quinazolin-4-one. The crude was recrystallized from MeOH:Et₂O to afford 0.11 g, 0.197 mmol, 80.59% yield of the title compound. 1H NMR (600 MHz, DMSO) δ 10.41 (s, 1H), 8.32 (s, 1H), 7.81-7.76 (m, 2H), 7.75 (d, J=8.8 Hz, 1H), 7.67 (dd, J=8.3, 2.1 Hz, 1H), 7.65 (s, 1H), 7.58 (dd, J=2.7, 1.3 Hz, 1H), 7.50-7.46 (m, 2H), 7.38 (d, J=2.9 Hz, 1H), 7.35 (d, J=8.4 Hz, 1H), 3.47 (s, 3H), 3.06 (s, 3H), 2.15 (s, 3H); MS (ES⁺) m/z calc'd for [M+H]⁺ [C₂₇H₂₂FN₅O₄S+H]⁺: 532.56 found 532.1, LCMS: t_{R}=3.11 min [Analytical Method: 05990510_AA0.lcm]

### Synthesis of N-(3-(5-chloro-1H-pyrrolo[2,3-b]pyridine-3-carbonyl)-2,4-difluorophenyl)-1-(4-fluorophenyl)-5-(methylsulfinyl)-1H-pyrazole-3-carboxamide [Example 28]

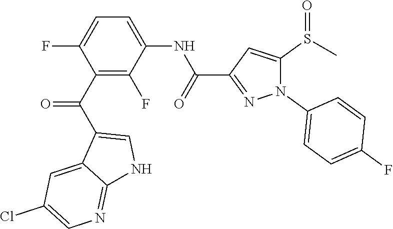 [SMILES: CS(=O)c1cc(C(=O)Nc2ccc(F)c(C(=O)c3c[nH]c4ncc(Cl)cc34)c2F)nn1-c1ccc(F)cc1]

Synthesis of Example 28 followed the same procedure used in the Synthetic Method A except used (3-amino-2,6-difluorophenyl)(5-chloro-1H-pyrrolo[2,3-b]pyridin-3-yl)methanone and 1-(4-fluorophenyl)-5-(methylsulfinyl)-1H-pyrazole-3-carboxylic acid. The crude was purified with 90 EtOAc:Hex to afford 49 mg, 0.0794 mmol, 34.47% yield of the title compound. 1H NMR (600 MHz, DMSO) δ 13.15 (d, J=3.3 Hz, 1H), 10.28 (s, 1H), 8.53-8.42 (m, 2H), 8.24 (d, J=3.2 Hz, 1H), 7.93-7.79 (m, 3H), 7.71 (s, 2H), 7.53-7.43 (m, 3H), 7.33 (t, J=8.8 Hz, 1H), 3.08 (s, 3H); MS (ES⁺) m/z calc'd for [M+H]⁺[C₂₅H₁₅ClF₃N₅O₃S+H]⁺: 558.93 found 558.0, LCMS: t_{R}=3.28 min [Analytical Method: 05990510_AA0.lcm]

### Synthesis of 1-(4-fluorophenyl)-N-(3-(imidazo[1,2-b]pyridazin-3-ylethynyl)-4-isopropylphenyl)-5-(methylsulfonyl)-1H-pyrazole-3-carboxamide [Example 29]

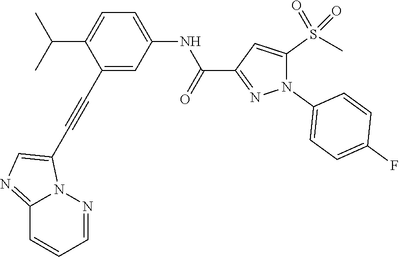 [SMILES: CC(C)c1ccc(NC(=O)c2cc(S(C)(=O)=O)n(-c3ccc(F)cc3)n2)cc1C#Cc1cnc2cccnn12]

Synthesis of Example 29 followed the same procedure used in the Synthetic Method A except used 1-(4-fluorophenyl)-5-(methylsulfonyl)-1H-pyrazole-3-carboxylic acid and 3-(2-imidazo[1,2-b]pyridazin-3-ylethynyl)-4-isopropyl-aniline. The crude was purified with 90 EtOAc:Hex to afford 21 mg, 0.0383 mmol, 14.68% yield of the title compound as a brown solid. 1H NMR (500 MHz, dmso) δ 10.53 (s, 1H), 8.70 (d, J=4.4 Hz, 1H), 8.08 (d, J=2.3 Hz, 1H), 7.83-7.74 (m, 5H), 7.66 (s, 1H), 7.48-7.35 (m, 6H), 3.54-3.47 (m, 1H), 3.33 (s, 4H), 1.28 (d, J=6.9 Hz, 5H); MS (ES⁺) m/z calc'd for [M+H]⁺ [C₂₈H₂₃FN₆O₃S+H]⁺: 543.58 found 543.2, LCMS: t_{R}=3.66 min [Analytical Method: 05990510_AA0.lcm]

### Synthesis of 1-(4-fluorophenyl)-N-(3-(imidazo[1,2-b]pyridazin-3-ylethynyl)-4-isopropylphenyl)-5-(methylsulfinyl)-1H-pyrazole-3-carboxamide [Example 30]

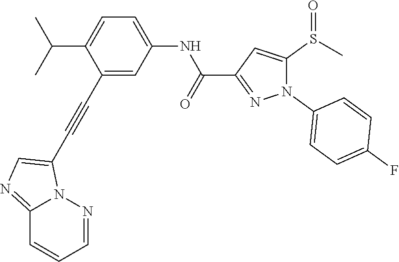 [SMILES: CC(C)c1ccc(NC(=O)c2cc(S(C)=O)n(-c3ccc(F)cc3)n2)cc1C#Cc1cnc2cccnn12]

Synthesis of Example 30 followed the same procedure used in Synthetic Method A except used 1-(4-fluorophenyl)-5-(methylsulfinyl)-1H-pyrazole-3-carboxylic acid and 3-(2-imidazo[1,2-b]pyridazin-3-ylethynyl)-4-isopropyl-aniline. The crude was purified with 5% MeOH:CH₂Cl₂ and this residue was recrystallized with DCM:Et₂O:Hex to afford 97 mg, 0.182 mmol, 75.51% yield of the title compound as colorless solid. 1H NMR (500 MHz, dmso) δ 10.44 (s, 1H), 8.69 (dd, J=4.4, 1.4 Hz, 1H), 8.25-8.19 (m, 2H), 8.09 (d, J=2.3 Hz, 1H), 7.84-7.78 (m, 3H), 7.69 (s, 1H), 7.50 (t, J=8.6 Hz, 2H), 7.41-7.34 (m, 2H), 3.51 (p, J=6.9 Hz, 1H), 3.07 (s, 3H), 1.28 (d, J=6.9 Hz, 6H); MS (ES⁺) m/z calc'd for [M+H]⁺[C₂₈H₂₃FN₆O₂S+H]⁺: 527.58 found 527.2, LCMS: t_{R}=6.14 min [Analytical Method:05991008_AA1.lcm]

### Synthesis of 1-(4-fluorophenyl)-N-(4-methyl-3-(1-methyl-2-oxo-2,3-dihydro-1H-benzo[d]imidazol-5-yl)phenyl)-5-(methylsulfinyl)-1H-pyrazole-3-carboxamide [Example 31]

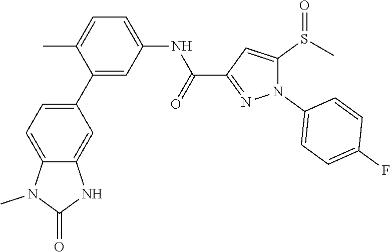 [SMILES: Cc1ccc(NC(=O)c2cc(S(C)=O)n(-c3ccc(F)cc3)n2)cc1-c1ccc2c(c1)[nH]c(=O)n2C]

Synthesis of Example 31 followed the same procedure used in Synthetic Method A except used 1-(4-fluorophenyl)-5-(methylsulfinyl)-1H-pyrazole-3-carboxylic acid and 5-(5-amino-2-methylphenyl)-1-methyl-1,3-dihydro-2H-benzo[d]imidazol-2-one. The residue was recrystallized with DCM:Et₂O:Hex to afford 84 mg, 0.166 mmol, 66.88% yield of the title compound as light beige solid. 1H NMR (500 MHz, dmso) δ 10.87 (s, 1H), 10.29 (s, 1H), 7.83-7.75 (m, 2H), 7.70 (d, J=8.3 Hz, 2H), 7.66 (d, J=2.1 Hz, 1H), 7.48 (t, J=8.7 Hz, 2H), 7.24 (d, J=8.1 Hz, 1H), 7.14 (d, J=8.0 Hz, 1H), 6.99 (dd, J=8.1, 1.7 Hz, 1H), 6.90 (d, J=1.8 Hz, 1H), 3.06 (s, 3H), 2.20 (s, 3H); MS (ES⁺) m/z calc'd for [M+H]⁺[C₂₆H₂₂FN₅O₃S+H]⁺: 504.55 found 504.1, LCMS: t_{R}=5.17 min [Analytical Method: 05991008_AA1.lcm]

### Synthesis of N-(3-((2-aminopyrimidin-5-yl)ethynyl)-2,4-difluorophenyl)-1-(4-fluorophenyl)-5-(methylsulfonyl)-1H-pyrazole-3-carboxamide [Example 32]

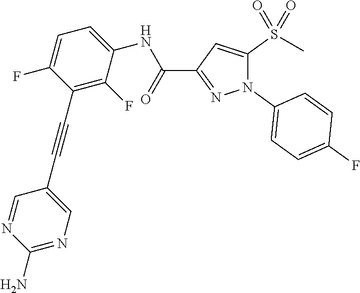 [SMILES: CS(=O)(=O)c1cc(C(=O)Nc2ccc(F)c(C#Cc3cnc(N)nc3)c2F)nn1-c1ccc(F)cc1]

Synthesis of Example 32 followed the same procedure used in Synthetic Method A except used 5-((3-amino-2,6-difluorophenyl)ethynyl)pyrimidin-2-amine and 1-(4-fluorophenyl)-5-(methylsulfonyl)-1H-pyrazole-3-carboxylic acid. The crude material was purified via trituration with 2:1 Et₂O:EtOAc to afford 0.075 g, 0.146 mmol, 58.96% yield of the title compound as a pale yellow solid. ¹H NMR (600 MHz, DMSO) δ 10.42 (br s, 1H), 8.45 (s, 2H), 7.78-7.72 (m, 2H), 7.64 (br s, 1H), 7.54 (br s, 1H), 7.49-7.41 (m, 2H), 7.26 (s, 2H), 7.15 (br s, 1H), 3.33 (s, 3H); MS (ES⁺) m/z calc'd for [M+H]⁺ [C₂₃H₁₅F₃N₆O₃S+H]⁺: 513.46 found 513.1, LCMS: t_{R}=3.28 min [Analytical Method: 05990510_AA0.lcm]

### Synthesis of 1-(4-fluorophenyl)-N-(4-methyl-3-((3-methyl-4-oxo-3,4-dihydroquinazolin-6-yl)oxy)phenyl)-5-(methylsulfonyl)-1H-pyrazole-3-carboxamide [Example 33]

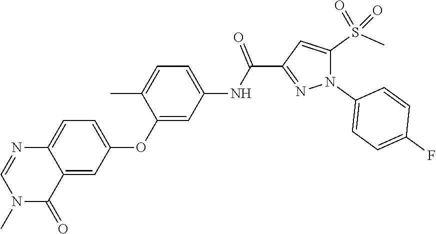 [SMILES: Cc1ccc(NC(=O)c2cc(S(C)(=O)=O)n(-c3ccc(F)cc3)n2)cc1Oc1ccc2ncn(C)c(=O)c2c1]

Synthesis of Example 33 followed the same procedure used in Synthetic Method A except used 1-(4-fluorophenyl)-5-(methylsulfonyl)-1H-pyrazole-3-carboxylic acid and 6-(5-amino-2-methyl-phenoxy)-3-methyl-quinazolin-4-one. The crude was recrystallized with MeOH:Et₂O to afford 93 mg, 0.160 mmol, 64.40% yield of the title compound. 1H NMR (600 MHz, DMSO) δ 10.49 (s, 1H), 8.32 (s, 1H), 7.74 (dd, J=8.9, 4.4 Hz, 3H), 7.66 (dd, J=8.3, 2.1 Hz, 1H), 7.61 (s, 1H), 7.59-7.53 (m, 2H), 7.48-7.40 (m, 2H), 7.40-7.32 (m, 2H), 3.47 (s, 3H), 2.15 (s, 3H); MS (ES⁺) m/z calc'd for [M+H]⁺[C₂₇H₂₂FN₅O₅S+H]⁺: 548.56 found 548.2, LCMS: t_{R}=3.39 min [Analytical Method: 05990510_AA0.lcm]

### Synthesis of 1-(4-fluorophenyl)-N-(4-methyl-3-(1-methyl-2-oxo-2,3-dihydro-1H-benzo[d]imidazol-5-yl)phenyl)-5-(methylsulfonyl)-1H-pyrazole-3-carboxamide [Example 34]

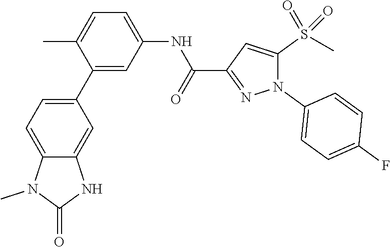 [SMILES: Cc1ccc(NC(=O)c2cc(S(C)(=O)=O)n(-c3ccc(F)cc3)n2)cc1-c1ccc2c(c1)[nH]c(=O)n2C]

Synthesis of Example 33 followed the same procedure used in Synthetic Method A except used 5-(5-amino-2-methylphenyl)-1-methyl-1,3-dihydro-2H-benzo[d]imidazol-2-one and 1-(4-fluorophenyl)-5-(methylsulfonyl)-1H-pyrazole-3-carboxylic acid. The crude was purified with 5% MeOH:CH₂Cl₂ and the resulting residue was recrystallized with DCM:Et₂O:Hex to afford 0.11 g, 0.205 mmol, 73.78% yield of the title compound. ¹H NMR (600 MHz, DMSO) δ 10.88 (s, 1H), 10.39 (s, 1H), 7.79-7.74 (m, 2H), 7.69 (dd, J=5.7, 2.5 Hz, 2H), 7.64 (s, 1H), 7.45 (t, J=8.7 Hz, 2H), 7.25 (d, J=9.0 Hz, 1H), 7.15 (d, J=8.0 Hz, 1H), 7.00 (dd, J=7.9, 1.6 Hz, 1H), 6.91 (d, J=1.6 Hz, 1H), 3.33 (s, 3H), 3.32 (s, 4H), 2.21 (s, 3H); MS (ES⁺) m/z calc'd for [M+H]⁺ [C₂₅H₁₅ClF₃N₅O₄S+H]⁺: 520.55 found 520.1, LCMS: t_{R}=3.26 min [Analytical Method: 05990510_AA0.lcm]

### Synthesis of N-(3-(5-chloro-1H-pyrrolo[2,3-b]pyridine-3-carbonyl)-2,4-difluorophenyl)-1-(4-fluorophenyl)-5-(methylsulfonyl)-1H-pyrazole-3-carboxamide [Example 35]

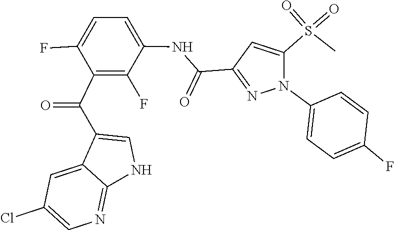 [SMILES: CS(=O)(=O)c1cc(C(=O)Nc2ccc(F)c(C(=O)c3c[nH]c4ncc(Cl)cc34)c2F)nn1-c1ccc(F)cc1]

Synthesis of Example 35 followed the same procedure used in Synthetic Method A except used 1-(4-fluorophenyl)-5-(methylsulfonyl)-1H-pyrazole-3-carboxylic acid and (3-amino-2,6-difluoro-phenyl)-(5-chloro-1H-pyrrolo[2,3-b]pyridin-3-yl)methanone. The crude was purified with 90% EtOAc:Hex to afford 0.052 g, 0.0767 mmol, 35.13% yield of the title compound. ¹H NMR (600 MHz, DMSO) δ 10.40 (s, 1H), 8.47 (s, 1H), 8.45 (d, J=2.4 Hz, 1H), 8.22 (s, 1H), 7.80-7.75 (m, 3H), 7.65 (s, 1H), 7.50-7.40 (m, 3H), 7.32 (s, 1H), 3.34 (s, 4H); MS (ES⁺) m/z calc'd for [M+H]⁺ [C₂₆H₂₂FN₅O₄S+H]⁺: 574.93 found 574.2, LCMS: I_{R}=5.87 min [Analytical Method: 05991008_BB0.lcm]

### Synthesis of N-(3-((4-(2-acetamido-4-methylthiazol-5-yl)pyrimidin-2-yl)amino)-4-methylphenyl)-1-(4-fluorophenyl)-5-(methylsulfonyl)-1H-pyrazole-3-carboxamide [Example 36]

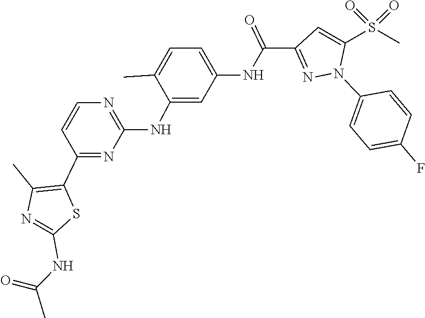 [SMILES: CC(=O)Nc1nc(C)c(-c2ccnc(Nc3cc(NC(=O)c4cc(S(C)(=O)=O)n(-c5ccc(F)cc5)n4)ccc3C)n2)s1]

Synthesis of Example 36 followed the same procedure used in Synthetic Method A except used 1-(4-fluorophenyl)-5-(methylsulfonyl)-1H-pyrazole-3-carboxylic acid and N-[5-[2-(5-amino-2-methyl-anilino)pyrimidin-4-yl]-4-methyl-thiazol-2-yl]acetamide. The crude was triturated resulting in 103 mg, 58.8% yield of the title compound as a brown solid. ¹H NMR (600 MHz, DMSO-d₆) δ 12.21 (s, 1H), 10.32 (s, 1H), 8.86 (s, 1H), 8.33 (d, J=5.3 Hz, 1H), 7.91 (s, 1H), 7.75 (dd, J=8.6, 4.7 Hz, 2H), 7.64 (s, 1H), 7.52 (d, J=8.4 Hz, 1H), 7.44 (t, J=8.6 Hz, 2H), 7.18 (d, J=8.3 Hz, 1H), 6.95 (d, J=5.3 Hz, 1H), 3.32 (s, 3H), 2.51 (s, 3H), 2.18 (s, 3H), 2.13 (s, 3H); MS (ES⁺) m/z calc'd for [M+H]⁺ [C₂₅H₂₅FN₈O₄S₂+H]⁺: 621.68 found 621.2, LCMS: t_{R}=3.04 min[Analytical Method: 05991008_AA1.lcm]

### Synthesis of N-(3-((3-(9H-purin-6-yl)pyridin-2-yl)amino)-4-methylphenyl)-1-(4-fluorophenyl)-5-(methylsulfinyl)-1H-pyrazole-3-carboxamide [Example 37]

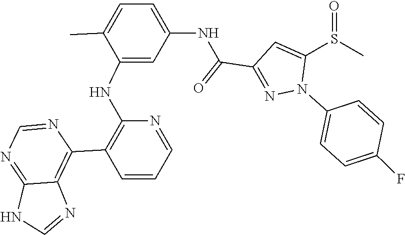 [SMILES: Cc1ccc(NC(=O)c2cc(S(C)=O)n(-c3ccc(F)cc3)n2)cc1Nc1ncccc1-c1ncnc2[nH]cnc12]

N1-(3-(9H-purin-6-yl)pyridin-2-yl)-6-methylbenzene-1,3-diamine (150 mg, 0.47 mmol, 1.00 eq), 1-(4-fluorophenyl)-5-(methylsulfinyl)-1H-pyrazole-3-carboxylic acid (142 mg, 0.57 mmol, 1.20 eq) and BOP (415 mg, 0.93 mmol, 2.00 eq) were dissolved in 3 ml DMF (3 ml, 0.10 M). DIPEA (0.25 ml, 1.4 mmol, 3.0 eq) was added and stirred for 30 minutes. The reaction was quenched with saturated aqueous sodium bicarbonate. The precipitate was filtered off and washed with water. The precipitate was triturated with ether/EtOAc and DCM/MeOH, filtered. The solid was washed with 1M LiCl then water, dried to afford N-(3-((3-(9H-purin-6-yl)pyridin-2-yl)amino)-4-methylphenyl)-1-(4-fluorophenyl)-5-(methylsulfinyl)-1H-pyrazole-3-carboxamide (151 mg, 56% yield) as a beige solid. ¹H NMR (500 MHz, DMSO-d₆) δ 13.84 (s, 1H), 12.28 (s, 1H), 9.81 (s, 2H), 9.07 (s, 1H), 8.71 (s, 1H), 8.58 (s, 1H), 8.34 (d, J=4.7 Hz, 1H), 8.24-8.18 (m, 2H), 7.45 (d, J=8.3 Hz, 1H), 7.34 (t, J=8.7, 8.7 Hz, 2H), 7.23 (d, J=8.2 Hz, 1H), 7.02 (dd, J=8.0, 4.6 Hz, 1H), 5.36 (q, J=8.7, 8.6, 8.6 Hz, 2H), 2.41 (s, 3H); MS (ES⁺) m/z=605.5 [M+H]⁺; LCMS: t_{R}=2.33 min [Analytical Method A-6].

### Synthesis of N-(2-(6-chloroimidazo[1,2-b]pyridazin-3-yl)benzofuran-5-yl)-1-(4-fluorophenyl)-5-(methylsulfinyl)-1H-pyrazole-3-carboxamide [Example 38]

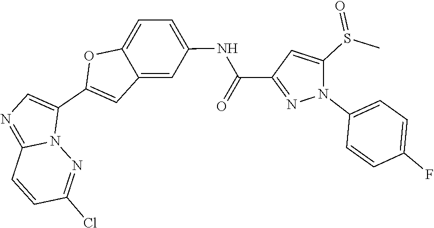 [SMILES: CS(=O)c1cc(C(=O)Nc2ccc3oc(-c4cnc5ccc(Cl)nn45)cc3c2)nn1-c1ccc(F)cc1]

Synthesis of Example 38 followed the same procedure used in Synthetic Method A except used 2-(6-chloroimidazo[1,2-b]pyridazin-3-yl)benzofuran-5-amine and 1-(4-fluorophenyl)-5-(methylsulfinyl)-1H-pyrazole-3-carboxylic acid. The crude material was purified first via trituration with MeOH then with column chromatography (silica gel; dry load; 25-90% ACN in toluene) to afford 0.025 g, 0.0412 mmol, 14.65% yield of the title compound as an orange solid. ¹H NMR (600 MHz, DMSO) δ 10.45 (s, 1H), 8.84 (s, 1H), 8.27 (d, J=9.5 Hz, 1H), 8.25 (d, J=2.0 Hz, 1H), 7.82 (dd, J=8.9, 4.8 Hz, 2H), 7.72-7.69 (m, 2H), 7.64 (d, J=8.9 Hz, 1H), 7.51 (t, J=8.7 Hz, 2H), 7.46 (d, J=9.5 Hz, 1H), 7.45 (s, 1H), 3.09 (s, 3H); MS (ES⁺) m/z calc'd for [M+H]⁺[C₂₅H₁₆ClFN₆O₃S+H]⁺: 535.95 found 535.1, LCMS: t_{R}=3.33 min [Analytical Method:05990510_AA0.lcm]

### Synthesis of N-(3-((4-aminothieno[2,3-d]pyrimidin-5-yl)ethynyl)phenyl)-1-(4-fluorophenyl)-5-(methylsulfonyl)-1H-pyrazole-3-carboxamide [Example 39]

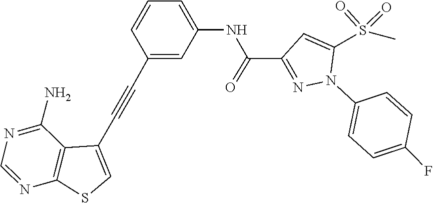 [SMILES: CS(=O)(=O)c1cc(C(=O)Nc2cccc(C#Cc3csc4ncnc(N)c34)c2)nn1-c1ccc(F)cc1]

Synthesis of Example 39 followed the same procedure used in Synthetic Method A except used 5-((3-aminophenyl)ethynyl)thieno[2,3-d]pyrimidin-4-amine and 1-(4-fluorophenyl)-5-(methylsulfonyl)-1H-pyrazole-3-carboxylic acid to afford 43 mg, 26% yield of the title compound as a beige solid. ¹H NMR (500 MHz, DMSO-d₆) δ 10.62 (s, 1H), 8.35 (s, 1H), 8.12 (s, 1H), 8.03 (s, 1H), 7.90-7.85 (m, 1H), 7.79-7.73 (m, 2H), 7.67 (d, J=1.6 Hz, 1H), 7.45 (td, J=8.4, 8.1, 2.4 Hz, 3H), 7.40-7.35 (m, 1H), 3.33 (s, 3H); MS (ES⁺): m/z=534.2 [M+H]⁺; LCMS: t_{R}=2.14 min [Analytical Method A-6].

### Synthesis of N-(3-((4-aminothieno[2,3-d]pyrimidin-5-yl)ethynyl)phenyl)-1-(4-fluorophenyl)-5-(methylsulfinyl)-1H-pyrazole-3-carboxamide [Example 40]

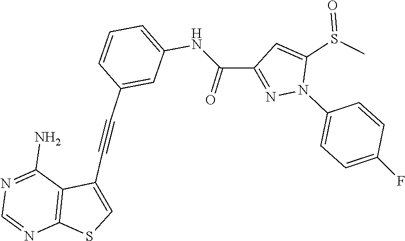 [SMILES: CS(=O)c1cc(C(=O)Nc2cccc(C#Cc3csc4ncnc(N)c34)c2)nn1-c1ccc(F)cc1]

Synthesis of Example 40 followed the same procedure used in Synthetic Method A except used 5-((3-aminophenyl)ethynyl)thieno[2,3-d]pyrimidin-4-amine and 1-(4-fluorophenyl)-5-(methylsulfinyl)-1H-pyrazole-3-carboxylic acid resulting in 223 mg, 75% yield of the title compound as a beige solid. ¹H NMR (500 MHz, DMSO-d₆) δ 10.53 (s, 1H), 8.35 (s, 1H), 8.15-8.10 (m, 1H), 8.03 (s, 1H), 7.92-7.86 (m, 1H), 7.84-7.76 (m, 2H), 7.70 (s, 1H), 7.53-7.46 (m, 2H), 7.44 (d, J=8.0 Hz, 1H), 7.37 (d, J=7.6 Hz, 1H), 3.07 (s, 3H); MS (ES⁺): m/z=517.0 [M+H]⁺; LCMS: t_{R}=1.96 min [Analytical Method A-6].

### Synthesis of N-(3-(2-acetamidohmidazo[1,2-a]pyridin-6-yl)-4-methylphenyl)-1-(4-fluorophenyl)-5-(methylsulfinyl)-1H-pyrazole-3-carboxamide [Example 41]

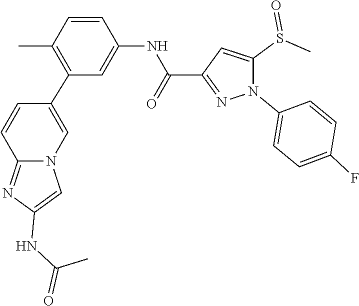 [SMILES: CC(=O)Nc1cn2cc(-c3cc(NC(=O)c4cc(S(C)=O)n(-c5ccc(F)cc5)n4)ccc3C)ccc2n1]

Synthesis of Example 41 followed the same procedure as Synthetic Method A except used N-(6-(5-amino-2-methylphenyl)imidazo[1,2-a]pyridin-2-yl)acetamide and 1-(4-fluorophenyl)-5-(methylsulfinyl)-1H-pyrazole-3-carboxylic acid resulting in 43 mg, 26% yield of the title compound as a beige solid. ¹H NMR (500 MHz, DMSO-d₆) δ 10.62 (s, 1H), 8.35 (s, 1H), 8.12 (s, 1H), 8.03 (s, 1H), 7.90-7.85 (m, 1H), 7.79-7.73 (m, 2H), 7.67 (d, J=1.6 Hz, 1H), 7.45 (td, J=8.4, 8.1, 2.4 Hz, 3H), 7.40-7.35 (m, 1H), 3.33 (s, 3H); MS (ES⁺) m/z calc'd for [M+H]⁺[C₂H₂₃FN₆O₃S+H]⁺: 531.57 found 531.4, LCMS: t_{R}=1.74 min [Analytical Method A-6].

### Synthesis of N-(3-((4-(2-acetamido-4-methylthiazol-5-yl)pyrimidin-2-yl)amino)-4-methylphenyl)-1-(4-fluorophenyl)-5-(methylsulfinyl)-1H-pyrazole-3-carboxamide [Example 42]

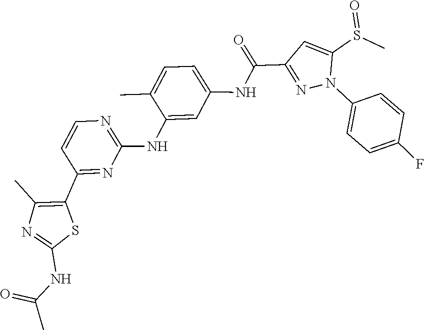 [SMILES: CC(=O)Nc1nc(C)c(-c2ccnc(Nc3cc(NC(=O)c4cc(S(C)=O)n(-c5ccc(F)cc5)n4)ccc3C)n2)s1]

Synthesis of Example 42 followed the same procedure used in Synthetic Method A except used N-[5-[2-(5-amino-2-methyl-anilino)pyrimidin-4-yl]-4-methyl-thiazol-2-yl]acetamide and 1-(4-fluorophenyl)-5-(methylsulfinyl)-1H-pyrazole-3-carboxylic acid. The crude was triturated resulting in 81.8 mg, 48% yield of the title compound as a light tan solid. ¹H NMR (600 MHz, DMSO-d₆) δ 12.20 (s, 1H), 10.23 (s, 1H), 8.87 (s, 1H), 8.33 (d, J=5.1 Hz, 1H), 7.91 (d, J=2.2 Hz, 1H), 7.80 (dd, J=8.8, 4.7 Hz, 2H), 7.67 (s, 1H), 7.59-7.44 (m, 3H), 7.18 (d, J=8.3 Hz, 1H), 6.95 (d, J=5.2 Hz, 1H), 3.07 (s, 3H), 2.52 (s, 3H), 2.19 (s, 3H), 2.13 (s, 3H); MS (ES⁺) m/z calc'd for [M+H]⁺ [C₂₈H₂₅FN₈O₃S₂+H]⁺: 605.68 found 605.1, LCMS: t_{R}=2.79 min [Analytical Method: 05990510_AA0.lcm]

### Synthesis of N-(3-(2-acetamidohmidazo[1,2-a]pyridin-6-yl)-4-methylphenyl)-1-(4-fluorophenyl)-5-(methylsulfonyl)-1H-pyrazole-3-carboxamide [Example 43]

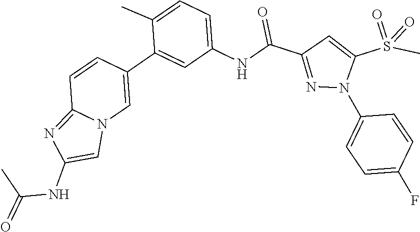 [SMILES: CC(=O)Nc1cn2cc(-c3cc(NC(=O)c4cc(S(C)(=O)=O)n(-c5ccc(F)cc5)n4)ccc3C)ccc2n1]

Synthesis of Example 43 followed the same procedure used in Synthetic Method A except used N-(6-(5-amino-2-methylphenyl)imidazo[1,2-a]pyridin-2-yl)acetamide and 1-(4-fluorophenyl)-5-(methylsulfonyl)-1H-pyrazole-3-carboxylic acid resulting in 150 mg, 76% yield of the title compound as a beige solid. ¹H NMR (500 MHz, DMSO-d₆) δ 10.69 (s, 1H), 10.43 (s, 1H), 8.56 (s, 1H), 8.12 (s, 1H), 7.75 (t, J=4.5, 4.5 Hz, 4H), 7.63 (s, 1H), 7.49-7.40 (m, 3H), 7.29 (d, J=8.9 Hz, 1H), 7.21 (d, J=9.2 Hz, 1H), 3.36 (s, 3H), 2.24 (s, 3H), 2.07 (s, 3H); MS (ES⁺) m/z calc'd for [M+H]⁺[C₂₇H₂₃FN₆O₄S+H]⁺: 547.57 found 547.3, LCMS: t_{R}=1.89 min [Analytical Method A-6].

### Synthesis of N-(2-fluoro-4-methyl-5-(2-(methylamino)pyrido[3,2-d]pyrimidin-6-yl)phenyl)-1-(4-fluorophenyl)-5-(methylsulfonyl)-1H-pyrazole-3-carboxamide [Example 44]

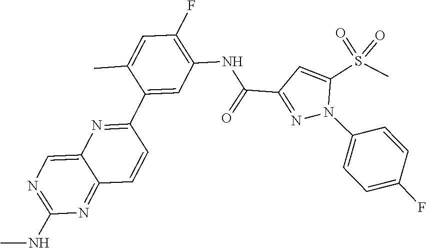 [SMILES: CNc1ncc2nc(-c3cc(NC(=O)c4cc(S(C)(=O)=O)n(-c5ccc(F)cc5)n4)c(F)cc3C)ccc2n1]

Synthesis of Example 44 followed the same procedure used in Synthetic Method A except used 1-(4-fluorophenyl)-5-(methylsulfonyl)-1H-pyrazole-3-carboxylic acid and 6-(5-amino-4-fluoro-2-methylphenyl)-N-methylpyrido[3,2-d]pyrimidin-2-amine. The crude material was then triturated with 3:1 Et₂O:EtOAc to afford 0.072 g, 0.129 mmol, 57.91% yield of the title compound as an off white solid. ¹H NMR (600 MHz, DMSO) δ 10.25 (s, 1H), 9.13 (s, 1H), 7.98 (d, J=8.3 Hz, 1H), 7.84 (d, J=8.8 Hz, 1H), 7.77 (dd, J=8.9, 4.8 Hz, 2H), 7.73 (d, J=7.9 Hz, 1H), 7.69 (br s, 1H), 7.64 (s, 1H), 7.45 (t, J=8.7 Hz, 2H), 7.31 (d, J=11.4 Hz, 1H), 3.34 (s, 3H), 2.93 (d, J=4.7 Hz, 3H), 2.39 (s, 3H); MS (ES⁺) m/z calc'd for [M+H]⁺ [C₂₆H₂₁F₂N₇O₃S+H]⁺: 550.55 found 550.3, LCMS: t_{R}=5.83 min [Analytical method: 05991008_BB1HT.lcm]

### Synthesis of N-(2-fluoro-4-methyl-5-(2-(methylamino)pyrido[3,2-d]pyrimidin-6-yl)phenyl)-1-(4-fluorophenyl)-5-(methylsulfinyl)-1H-pyrazole-3-carboxamide [Example 45]

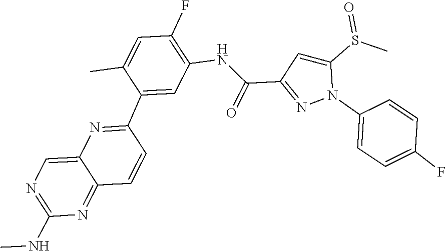 [SMILES: CNc1ncc2nc(-c3cc(NC(=O)c4cc(S(C)=O)n(-c5ccc(F)cc5)n4)c(F)cc3C)ccc2n1]

Synthesis of Example 45 followed the same procedure used in Synthetic Method A except used 1-(4-fluorophenyl)-5-(methylsulfinyl)-1H-pyrazole-3-carboxylic acid and 6-(5-amino-4-fluoro-2-methylphenyl)-N-methylpyrido[3,2-d]pyrimidin-2-amine. The crude material was triturated with 3:1 Et₂O:EtOAc to afford 0.071 g, 0.133 mmol, 58.85% yield of the title compound as a pale yellow solid. 1H NMR (600 MHz, DMSO) δ 10.14 (s, 1H), 9.14 (br s, 1H), 7.98 (d, J=6.9 Hz, 1H), 7.85 (d, J=8.8 Hz, 1H), 7.83-7.78 (m, 3H), 7.69 (br s, 2H), 7.50 (t, J=8.7 Hz, 2H), 7.32 (d, J=11.4 Hz, 1H), 3.07 (s, 3H), 2.94 (d, J=4.7 Hz, 3H), 2.39 (s, 3H); MS (ES⁺) m/z calc'd for [M+H]⁺ [C₂₆H₂₁F₂N₇O₂S+H]⁺: 541.56 found 534.2, LCMS: t_{R}=5.36 min [Analytical Method: 05991008_BB1HT.lcm]

### Synthesis of N-(2-fluoro-5-((2-(methylamino)thiazolo[5,4-b]pyridin-5-yl)oxy)phenyl)-1-(4-fluorophenyl)-5-(methylsulfinyl)-1H-pyrazole-3-carboxamide [Example 46]

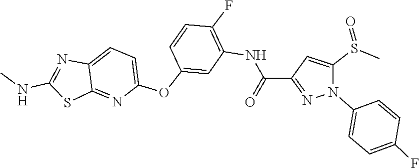 [SMILES: CNc1nc2ccc(Oc3ccc(F)c(NC(=O)c4cc(S(C)=O)n(-c5ccc(F)cc5)n4)c3)nc2s1]

Synthesis of Example 46 followed the same procedure used in Synthetic Method A except used 5-(3-amino-4-fluorophenoxy)-N-methylthiazolo[5,4-b]pyridin-2-amine and 1-(4-fluorophenyl)-5-(methylsulfinyl)-1H-pyrazole-3-carboxylic acid. The crude material was purified by chromatography over silica gel, ISCO, CombiFlash, 4 g cartridge eluting with 20-80% EtOAc in hexanes to afford 21 mg, 10% yield of the title compound as an off-white solid. ¹H NMR (500 MHz, DMSO-d₆) δ 8.28 (d, J=8.7 Hz, 1H), 7.83-7.75 (m, 3H), 7.49 (t, J=8.7, 8.7 Hz, 2H), 7.10 (d, J=8.7 Hz, 1H), 7.02 (dd, J=11.2, 8.7 Hz, 1H), 6.54 (dd, J=7.7, 2.9 Hz, 1H), 6.29 (dt, J=8.7, 3.2, 3.2 Hz, 1H), 5.31 (s, 2H), 4.03 (s, 3H), 3.09 (s, 3H); MS (ES⁺) m/z calc'd for [M+H]⁺[C₂₄H₁₈F₂N₆O₃S₂+H]⁺: 541.56 found 541.3, LCMS: t_{R}=2.36 min [Analytical Method A-6]

### Synthesis of N-(2-fluoro-5-((2-(methylamino)thiazolo[5,4-b]pyridin-5-yl)oxy)phenyl)-1-(4-fluorophenyl)-5-(methylsulfonyl)-1H-pyrazole-3-carboxamide [Example 47]

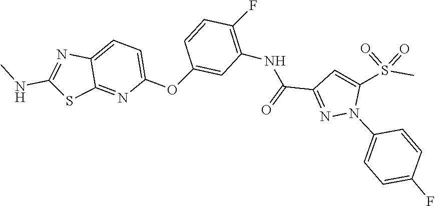 [SMILES: CNc1nc2ccc(Oc3ccc(F)c(NC(=O)c4cc(S(C)(=O)=O)n(-c5ccc(F)cc5)n4)c3)nc2s1]

Synthesis of Example 47 followed the same procedure used in Synthetic Method A except used 5-(3-amino-4-fluorophenoxy)-N-methylthiazolo[5,4-b]pyridin-2-amine and 1-(4-fluorophenyl)-5-(methylsulfonyl)-1H-pyrazole-3-carboxylic acid. The crude material was purified by chromatography over silica gel, ISCO, CombiFlash, 4 g cartridge eluting with 0-50% EtOAc in Hexane to afford the 9.1 mg, 5% yield of the title compound as an off-white solid. ¹H NMR (500 MHz, DMSO-d₆) δ 8.28 (d, J=8.7 Hz, 1H), 7.80-7.72 (m, 3H), 7.52-7.37 (m, 2H), 7.10 (d, J=8.7 Hz, 1H), 7.02 (dd, J=11.2, 8.7 Hz, 1H), 6.54 (dd, J=7.7, 2.9 Hz, 1H), 6.29 (dt, J=8.7, 3.3, 3.3 Hz, 1H), 5.30 (s, 2H), 3.98 (s, 3H), 3.36 (s, 3H); MS (ES⁺) m/z calc'd for [M+H]⁺[C₂₄H₁₈F₂N₆O₄S₂+H]⁺: 557.56 found 556.9 found, LCMS: t_{R}=2.59 min [Analytical Method A-6].

### Synthesis of 1-(4-fluorophenyl)-N-(4-methyl-3-((4-(pyridin-3-yl)thiazol-2-yl)amino)phenyl)-5-(methylsulfinyl)-1H-pyrazole-3-carboxamide [Example 48]

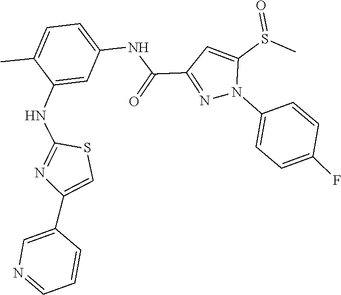 [SMILES: Cc1ccc(NC(=O)c2cc(S(C)=O)n(-c3ccc(F)cc3)n2)cc1Nc1nc(-c2cccnc2)cs1]

Synthesis of Example 48 followed the same procedure used in Synthetic Method A except used 1-(4-fluorophenyl)-5-(methylsulfinyl)-1H-pyrazole-3-carboxylic acid and 4-methyl-N3-[4-(3-pyridyl)thiazol-2-yl]benzene-1,3-diamine; dihydrochloride. The crude filter cake was triturated with MeOH afforded 0.10 g, 0.191 mmol, 50.94% yield of the title compound as an off-white solid. ¹H NMR (600 MHz, DMSO) δ 10.32 (s, 1H), 9.45 (s, 1H), 9.14 (d, J=1.7 Hz, 1H), 8.62 (d, J=2.0 Hz, 1H), 8.49 (dd, J=4.7, 1.6 Hz, 1H), 8.32-8.29 (m, 1H), 7.81 (dd, J=9.0, 4.8 Hz, 2H), 7.68 (s, 1H), 7.50 (t, J=8.7 Hz, 2H), 7.47 (s, 1H), 7.44-7.41 (m, 1H), 7.40 (dd, J=8.2, 2.1 Hz, 1H), 7.19 (d, J=8.4 Hz, 1H), 3.08 (s, 3H), 2.27 (s, 3H); MS (ES⁺) m/z calc'd for [M+H]⁺ [C₂₆H₂₁FN₆O₂S₂+H]⁺: 533.61 found 533.1, LCMS: t_{R}=5.30 min [Analytical Method: 05991008_AA0.lcm]

### Synthesis of N-(3-((4-(2-aminopyridin-3-yl)pyrimidin-2-yl)amino)-4-fluorophenyl)-1-(4-fluorophenyl)-5-(methylsulfinyl)-1H-pyrazole-3-carboxamide [Example 49]

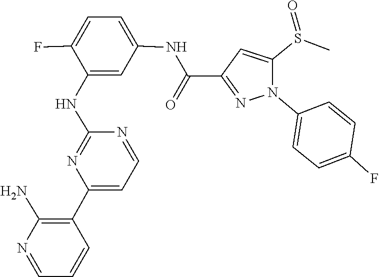 [SMILES: CS(=O)c1cc(C(=O)Nc2ccc(F)c(Nc3nccc(-c4cccnc4N)n3)c2)nn1-c1ccc(F)cc1]

Synthesis of Example 49 followed the same procedure used in Synthetic Method A except used 1-(4-fluorophenyl)-5-(methylsulfinyl)-1H-pyrazole-3-carboxylic acid and N-[3-[4-(2-amino-3-pyridyl)pyrimidin-2-yl]-4-fluoro-benzene-1,3-diamine. The precipitated solid was collected via vacuum filtration and rinsed with water (×2) followed by Et₂O to afford 0.11 g, 0.203 mmol, 83.64% yield of the title compound as a pale orange solid. ¹H NMR (600 MHz, DMSO) δ 10.42 (s, 1H), 9.53 (s, 1H), 8.43 (d, J=5.5 Hz, 1H), 8.25 (dd, J=7.3, 2.3 Hz, 1H), 8.16-8.14 (m, 1H), 8.09 (dd, J=4.6, 1.7 Hz, 1H), 7.80 (dd, J=8.9, 4.8 Hz, 2H), 7.70 (s, 1H), 7.64-7.55 (m, 3H), 7.50 (t, J=8.7 Hz, 2H), 7.37 (d, J=5.6 Hz, 1H), 7.26-7.22 (m, 1H), 6.64 (dd, J=7.8, 4.7 Hz, 1H), 3.07 (s, 3H); MS (ES⁺) m/z calc'd for [M+H]⁺[C₂₆H₂₀F₂N₈O₂S+H]⁺: 547.55 found 547.1, LCMS: t_{R}=4.56 min [Analytical Method: 05991008_AA0.lcm].

### Synthesis of N-(3-(1-(4-fluorophenyl)-5-(methylsulfinyl)-1H-pyrazole-3-carboxamido)phenyl)-6-(1H-pyrazol-5-yl)picolinamide [Example 50]

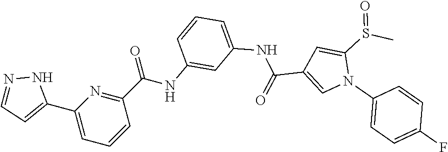 [SMILES: CS(=O)c1cc(C(=O)Nc2cccc(NC(=O)c3cccc(-c4ccn[nH]4)n3)c2)cn1-c1ccc(F)cc1]

Synthesis of Example 50 followed the same procedure used in Synthetic Method A except used N-(3-aminophenyl)-6-(1H-pyrazol-5-yl)pyridine-2-carboxamide; hydrochloride and 1-(4-fluorophenyl)-5-(methylsulfinyl)-1H-pyrazole-3-carboxylic acid The crude was purified by chromatography on silica gel, ISCO CombiFlash, 12 g cartridge [eluting with a step gradient 0% MeOH in DCM to 5% MeOH in DCM] resulting in 71.9 mg, 39.9% yield of the title compound as an off-white solid. ¹H NMR (600 MHz, DMSO-d₆) δ 14.21-14.04 (m, 1H), 10.79 (s, 1H), 10.44 (s, 1H), 8.44 (s, 1H), 8.10 (d, J=8.1 Hz, 3H), 7.87-7.80 (m, 2H), 7.73 (s, 1H), 7.70-7.58 (m, 3H), 7.52 (t, J=8.7 Hz, 2H), 7.46-7.37 (m, 1H), 7.09 (s, 1H), 3.10 (s, 3H); MS (ES⁺) m/z calc'd for [M+H]⁺ [C₂₆H₂₀FN₇O₃S+H]⁺: 530.55 found 530.2, LCMS: t_{R}=5.91 min [Analytical Method:05991008_AA0.lcm]

### Synthesis of N-(4-((6,7-dimethoxyquinolin-4-yl)oxy)-3-fluorophenyl)-1-(4-fluorophenyl)-5-(methylsulfinyl)-1H-pyrazole-3-carboxamide [Example 51]

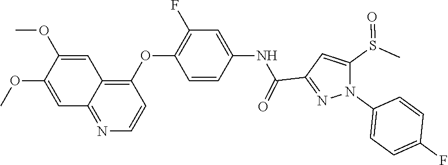 [SMILES: COc1cc2nccc(Oc3ccc(NC(=O)c4cc(S(C)=O)n(-c5ccc(F)cc5)n4)cc3F)c2cc1OC]

Synthesis of Example 51 followed the same procedure used in Synthetic Method A except used 1-(4-fluorophenyl)-5-(methylsulfinyl)-1H-pyrazole-3-carboxylic acid and 4-((6,7-dimethoxyquinolin-4-yl)oxy)-3-fluoroaniline. The solid was purified with 5% MeOH:DCM to afford 33 mg, 0.0584 mmol, 19.02% yield of the title compound. ¹H NMR (600 MHz, DMSO) δ 10.75 (s, 1H), 8.50 (d, J=5.2 Hz, 1H), 8.07 (dd, J=13.1, 2.5 Hz, 1H), 7.84-7.79 (m, 3H), 7.74 (s, 1H), 7.55-7.47 (m, 4H), 7.42 (s, 1H), 6.49 (d, J=5.3 Hz, 1H), 3.96 (s, 6H), 3.10 (s, 3H); MS (ES⁺) m/z calc'd for [M+H]⁺ [C₂₈H₂₂F₂N₄O₅S+H]⁺: 565.56 found 565.1, LCMS: t_{R}=5.17 min [Analytical Method:05991008_AA0.lcm]

### Synthesis of N-(2-fluoro-5-((7-oxo-5,6,7,8-tetrahydro-1,8-naphthyridin-4-yl)oxy)benzyl)-1-(4-fluorophenyl)-5-(methylsulfinyl)-1H-pyrazole-3-carboxamide [Example 52]

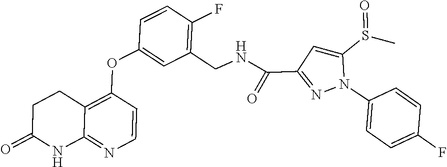 [SMILES: CS(=O)c1cc(C(=O)NCc2cc(Oc3ccnc4c3CCC(=O)N4)ccc2F)nn1-c1ccc(F)cc1]

Synthesis of Example 52 followed the same procedure used in Synthetic Method A except used 5-(3-(aminomethyl)-4-fluorophenoxy)-3,4-dihydro-1,8-naphthyridin-2(1H)-one and 1-(4-fluorophenyl)-5-(methylsulfinyl)-1H-pyrazole-3-carboxylic acid. The crude material was purified by HPLC to afford 96 mg, 51.40% yield of the title compound as a white solid. ¹H NMR (500 MHz, DMSO-d₆) δ 10.45 (s, 1H), 9.07 (t, J=6.0 Hz, 1H), 7.95 (d, J=5.8 Hz, 1H), 7.72 (ddd, J=8.8, 4.8, 1.3 Hz, 2H), 7.53 (d, J=1.2 Hz, 1H), 7.51-7.42 (m, 2H), 7.27 (t, J=8.9 Hz, 1H), 7.08 (t, J=6.2 Hz, 2H), 6.28 (dd, J=5.9, 1.2 Hz, 1H), 4.49 (t, J=5.3 Hz, 2H), 3.28 (s, 2H), 3.02 (d, J=1.2 Hz, 3H), 2.85 (t, J=7.8 Hz, 2H); MS (ES⁺) m/z=538.6 [M+H]⁺; LCMS: t_{R}=1.76 min [Analytical Method A-6].

### Synthesis of N-(2-fluoro-5-((7-oxo-5,6,7,8-tetrahydro-1,8-naphthyridin-4-yl)oxy)benzyl)-1-(4-fluorophenyl)-5-(methylsulfonyl)-1H-pyrazole-3-carboxamide [Example 53]

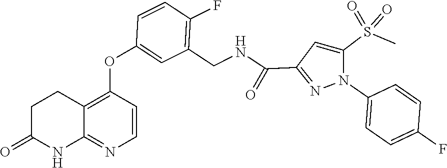 [SMILES: CS(=O)(=O)c1cc(C(=O)NCc2cc(Oc3ccnc4c3CCC(=O)N4)ccc2F)nn1-c1ccc(F)cc1]

Synthesis of Example 53 followed the same procedure used in Synthetic Method A except used 5-(3-(aminomethyl)-4-fluorophenoxy)-3,4-dihydro-1,8-naphthyridin-2(1H)-one and 1-(4-fluorophenyl)-5-(methylsulfonyl)-1H-pyrazole-3-carboxylic acid. The crude material was purified by HPLC to afford 69.8 mg, 33% yield of the title compound as a white solid. ¹H NMR (500 MHz, DMSO-d₆) δ 10.47 (s, 1H), 9.15 (t, J=6.0, 6.0 Hz, 1H), 7.95 (d, J=5.7 Hz, 1H), 7.69 (ddt, J=7.0, 4.8, 2.9, 2.9 Hz, 2H), 7.49 (s, 1H), 7.46-7.36 (m, 2H), 7.33-7.22 (m, 1H), 7.08 (dd, J=7.1, 4.2 Hz, 2H), 6.27 (d, J=5.8 Hz, 1H), 4.48 (d, J=6.0 Hz, 2H), 3.29 (s, 3H), 2.86 (t, J=7.7, 7.7 Hz, 2H); MS(ES⁺) m/z calc'd for [M+H]⁺[C₂₆H₂₁F₂N₅O₅S+H]⁺: 554.1 found 554.4, LCMS t_{R}=1.91 min [Analytical Method A-6].

### Synthesis of N-(4-((6,7-dimethoxyquinolin-4-yl)oxy)-2-fluorophenyl)-1-(4-fluorophenyl)-5-(methylsulfinyl)-1H-pyrazole-3-carboxamide [Example 54]

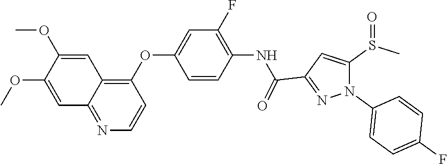 [SMILES: COc1cc2nccc(Oc3ccc(NC(=O)c4cc(S(C)=O)n(-c5ccc(F)cc5)n4)c(F)c3)c2cc1OC]

Synthesis of Example 54 followed the same procedure used in Synthetic Method A except used 1-(4-fluorophenyl)-5-(methylsulfinyl)-1H-pyrazole-3-carboxylic acid and 4-[(6,7-Dimethoxy-4-quinolinyl)oxy]-2-fluoroaniline. The crude solid was purified with 5% MeOH:CH₂Cl₂ to afford 0.13 g, 0.221 mmol, 71.69% yield of the title compound. ¹H NMR (500 MHz, DMSO) δ 10.19 (s, 1H), 8.56 (d, J=5.3 Hz, 1H), 7.84-7.75 (m, 3H), 7.72 (s, 1H), 7.55-7.48 (m, 3H), 7.43 (s, 1H), 7.40 (dd, J=11.0, 2.7 Hz, 1H), 7.18-7.14 (m, 1H), 6.66 (d, J=5.3 Hz, 1H), 3.97 (s, 3H), 3.94 (s, 3H), 3.09 (s, 3H); MS (ES⁺) m/z calc'd for [M+H]⁺ [C₂₈H₂₂F₂N₄O₅S+H]⁺: 565.56 found 565.2, LCMS: t_{R}=5.11 min [Analytical Method: 05991008_AA0.lcm]

### Synthesis of 1-(4-fluorophenyl)-N-(4-methyl-3-((3-methyl-4-oxo-3,4-dihydropyrido[3,2-d]pyrimidin-6-yl)oxy)phenyl)-5-(methylsulfinyl)-1H-pyrazole-3-carboxamide [Example 55]

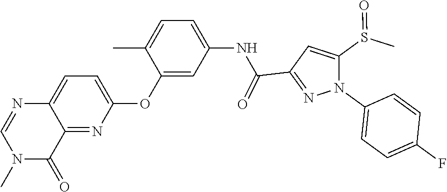 [SMILES: Cc1ccc(NC(=O)c2cc(S(C)=O)n(-c3ccc(F)cc3)n2)cc1Oc1ccc2ncn(C)c(=O)c2n1]

Synthesis of Example 55 followed the same procedure used in Synthetic Method A except used 6-(5-amino-2-methylphenoxy)-3-methylpyrido[3,2-d]pyrimidin-4(3H)-one and 11-(4-fluorophenyl)-5-(methylsulfinyl)-1H-pyrazole-3-carboxylic acid. The crude was triturated resulting in 149 mg, 75.8% yield of the title compound as a light tan solid. ¹H NMR (600 MHz, DMSO-d₆) δ 10.41 (s, 1H), 8.39 (s, 1H), 8.17 (d, J=8.9 Hz, 1H), 7.82-7.76 (m, 2H), 7.70-7.63 (m, 3H), 7.53-7.45 (m, 3H), 7.31 (d, J=8.9 Hz, 1H), 3.46 (s, 3H), 3.06 (s, 3H), 2.07 (s, 3H); MS (ES⁺) m/z calc'd for [M+H]⁺ [C₂₆H₂₁FN₆O₄S+H]⁺: 533.55 found 533.2, LCMS: t_{R}=5.61 min [Analytical Method: 05991008_AA0.lcm]

### Synthesis of 1-(4-fluorophenyl)-N-(6-methyl-5-(4-morpholinothieno[3,2-d]pyrimidin-2-yl)pyridin-3-yl)-5-(methylsulfonyl)-1H-pyrazole-3-carboxamide [Example 56]

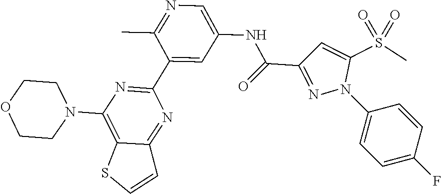 [SMILES: Cc1ncc(NC(=O)c2cc(S(C)(=O)=O)n(-c3ccc(F)cc3)n2)cc1-c1nc(N2CCOCC2)c2sccc2n1]

Synthesis of Example 56 followed the same procedure used in Synthetic Method A except used 1-(4-fluorophenyl)-5-(methylsulfonyl)-1H-pyrazole-3-carboxylic acid and 6-methyl-5-(4-morpholinothieno[3,2-d]pyrimidin-2-yl)pyridin-3-amine. The crude material was then purified via trituration with 4:1 Et₂O:EtOAc to afford 0.10 g, 0.162 mmol, 66.39% yield of the title compound as an off-white solid. ¹H NMR (600 MHz, DMSO) δ 10.74 (s, 1H), 8.94 (d, J=2.5 Hz, 1H), 8.66 (d, J=2.5 Hz, 1H), 8.30 (d, J=5.5 Hz, 1H), 7.78 (dd, J=8.9, 4.8 Hz, 2H), 7.68 (s, 1H), 7.54 (d, J=5.5 Hz, 1H), 7.46 (t, J=8.7 Hz, 2H), 4.01-3.96 (m, 4H), 3.81-3.76 (m, 4H), 3.34 (s, 3H), 2.71 (s, 3H); MS (ES⁺) m/z calc'd for [M+H]⁺[C₂₇H₂₄FN₇O₄S₂+H]⁺: 594.65 found 594.2, LCMS: t_{R}=5.61 min [Analytical Method: 05991008_AA0.lcm]

### Synthesis of 5-amino-3-(5-chloro-1H-pyrrolo[2,3-b]pyridine-3-carbonyl)-2,4-difluorophenyl 1-(4-fluorophenyl)-5-(methylsulfonyl)-1H-pyrazole-3-carboxylate [Example 57]

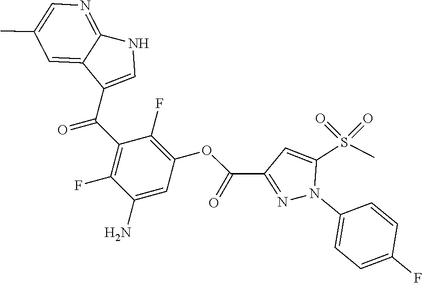 [SMILES: Cc1cnc2[nH]cc(C(=O)c3c(F)c(N)cc(OC(=O)c4cc(S(C)(=O)=O)n(-c5ccc(F)cc5)n4)c3F)c2c1]

Synthesis of Example 57 followed the same procedure used in Synthetic Method A except used 1-(4-fluorophenyl)-5-(methylsulfonyl)-1H-pyrazole-3-carboxylic acid and (3-amino-2,6-difluoro-5-hydroxy-phenyl)-(5-chloro-1H-pyrrolo[2,3-b]pyridin-3-yl)methanone. The crude material was purified by column chromatography (silica gel; dry load; 5-50% ACN in toluene resulted in 0.028 g, 0.045 mmol, 28.5% of the tile compound as an off-white solid. ¹H NMR (600 MHz, DMSO) δ 13.12 (s, 1H), 8.44 (s, 2H), 8.24 (s, 1H), 7.84 (s, 1H), 7.80-7.70 (m, 2H), 7.45 (t, J=8.7 Hz, 2H), 6.98-6.85 (m, 1H), 5.53 (s, 2H), 3.37 (s, 3H); MS (ES⁺) m/z calc'd for [M+H]⁺ [C₂₅H₁₅ClF₃N₅O₅S+H]⁺: 590.93 found 590.0, LCMS: t_{R}=7.07 min [Analytical Method: 05991008_AA0.lcm]

### Synthesis of N-(3-fluoro-4-methyl-5-(4-morpholinothieno[3,2-d]pyrimidin-2-yl)phenyl)-1-(4-fluorophenyl)-5-(methylsulfonyl)-1H-pyrazole-3-carboxamide [Example 58]

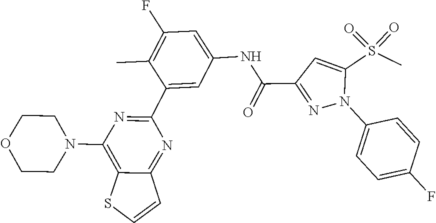 [SMILES: Cc1c(F)cc(NC(=O)c2cc(S(C)(=O)=O)n(-c3ccc(F)cc3)n2)cc1-c1nc(N2CCOCC2)c2sccc2n1]

Synthesis of Example 58 followed the same procedure used in Synthetic Method A except used 1-(4-fluorophenyl)-5-(methylsulfonyl)-1H-pyrazole-3-carboxylic acid and 3-fluoro-4-methyl-5-(4-morpholinothieno[3,2-d]pyrimidin-2-yl)aniline. The crude material was purified via trituration with 4:1 Et₂O:EtOAc to afford 0.11 g, 0.171 mmol, 83.97% yield of the tile compound as an off-white solid. ¹H NMR (600 MHz, DMSO) δ 10.67 (s, 1H), 8.29 (d, J=5.5 Hz, 1H), 8.07 (s, 1H), 7.84 (dd, 1H), 7.76 (dd, J=8.9, 4.8 Hz, 2H), 7.68 (s, 1H), 7.52 (d, J=5.5 Hz, 1H), 7.45 (t, J=8.7 Hz, 2H), 4.00-3.94 (m, 4H), 3.81-3.75 (m, 4H), 3.34 (s, 3H), 2.37 (d, J=1.9 Hz, 3H); MS (ES⁺) m/z calc'd for [M+H]⁺[C₂₈H₂₄F₂N₆O₄S₂+H]⁺: 611.65 found 611.1, LCMS: t_{R}=6.78 min [Analytical Method: 05991008_AA0.lcm]

### Synthesis of N-(3-fluoro-4-((2-(5-(((2-methoxyethyl)amino)methyl)pyridin-2-yl)thieno[3,2-b]pyridin-7-yl)oxy)phenyl)-1-(4-fluorophenyl)-5-(methylsulfonyl)-1H-pyrazole-3-carboxamide [Example 59]

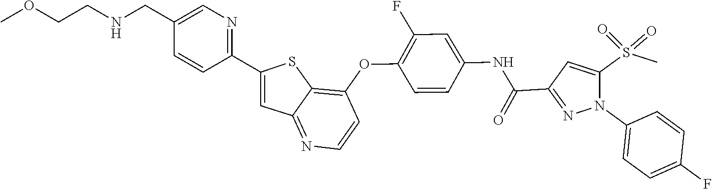 [SMILES: COCCNCc1ccc(-c2cc3nccc(Oc4ccc(NC(=O)c5cc(S(C)(=O)=O)n(-c6ccc(F)cc6)n5)cc4F)c3s2)nc1]

Synthesis of Example 59 followed the same procedure used in Synthetic Method A except used 1-(4-fluorophenyl)-5-(methylsulfonyl)-1H-pyrazole-3-carboxylic acid and 3-fluoro-4-[2-[5-[(2-methoxyethylamino)methyl]-2-pyridyl]thieno[3,2-b]pyridin-7-yl]oxy-aniline. The crude was purified with 75% EtOAc:Hex via chromatography to afford 0.058 g, 0.0832 mmol, 31.98% yield of the title compound as colorless solid. ¹H NMR (500 MHz, DMSO) δ 8.62 (d, J=2.2 Hz, 1H), 8.51 (d, J=5.4 Hz, 1H), 8.24 (t, J=9.7 Hz, 1H), 7.90-7.84 (m, 1H), 7.72 (dd, J=8.9, 4.8 Hz, 1H), 7.65 (dd, J=8.7, 4.8 Hz, 1H), 7.52 (s, 1H), 7.44 (t, J=8.8 Hz, 1H), 7.37 (t, J=8.7 Hz, 1H), 7.13 (t, J=8.9 Hz, 1H), 6.61 (dd, J=5.3, 0.9 Hz, 1H), 6.55 (dd, J=13.2, 2.6 Hz, 1H), 6.48-6.45 (m, 1H), 5.54 (s, 2H), 5.09 (s, 1H), 4.82 (s, 1H), 3.91 (t, J=5.5 Hz, 1H), 3.60 (d, J=5.6 Hz, 1H), 3.57 (t, J=5.5 Hz, 2H), 3.35 (s, 3H), 3.25 (s, 1H), 3.16 (s, 2H); MS (ES⁺) m/z calc'd for [M+H]⁺ [C₃₃H₂₈F₂N₆O₅S₂+H]⁺: 691.74 found 691.1, LCMS: t_{R}=6.33 min [Analytical Method: 05991008_AA0.lcm]

### Synthesis of 1-(4-fluorophenyl)-N-(3-hydroxy-4-methyl-5-(1-methyl-5-morpholino-6-oxo-1,6-dihydropyridin-3-yl)phenyl)-5-(methylsulfonyl)-1H-pyrazole-3-carboxamide [Example 60]

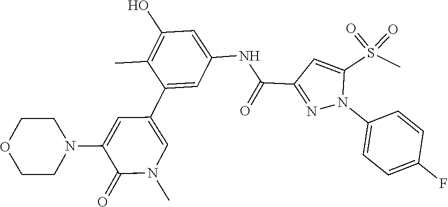 [SMILES: Cc1c(O)cc(NC(=O)c2cc(S(C)(=O)=O)n(-c3ccc(F)cc3)n2)cc1-c1cc(N2CCOCC2)c(=O)n(C)c1]

Synthesis of Example 60 followed the same procedure used in Synthetic Method A except used 5-(5-amino-3-hydroxy-2-methyl-phenyl)-1-methyl-3-morpholino-pyridin-2-one and 1-(4-fluorophenyl)-5-(methylsulfonyl)-1H-pyrazole-3-carboxylic acid. The crude was purified by column chromatography (silica gel; solid load; 30-55% ACN in toluene) followed by methanol trituration then afforded 0.029 g, 0.0486 mmol, 25.96% yield of the title compound as a white solid. ¹H NMR (600 MHz, DMSO) δ 10.21 (s, 1H), 9.53 (s, 1H), 7.75 (dd, J=9.0, 4.8 Hz, 2H), 7.64 (s, 1H), 7.51 (d, J=2.0 Hz, 1H), 7.45 (t, J=8.7 Hz, 2H), 7.33 (d, J=2.2 Hz, 1H), 7.05 (d, J=2.0 Hz, 1H), 6.64 (d, J=2.2 Hz, 1H), 3.72-3.69 (m, 4H), 3.48 (s, 3H), 3.33 (s, 3H), 3.11-3.06 (m, 4H), 2.04 (s, 3H); MS (ES⁺) m/z calc'd for [M+H]⁺[C₂₈H₂₈FN₅O₆S+H]⁺: 582.62 found 582.2, LCMS: t_{R}=5.52 min [Analytical Method: 05991008_AA0.lcm]

### Synthesis of N-(3-(5-chloro-1H-pyrrolo[2,3-b]pyridine-3-carbonyl)-2,4-difluoro-5-hydroxyphenyl)-1-(4-fluorophenyl)-5-(methylthio)-1H-pyrazole-3-carboxamide [Example 61]

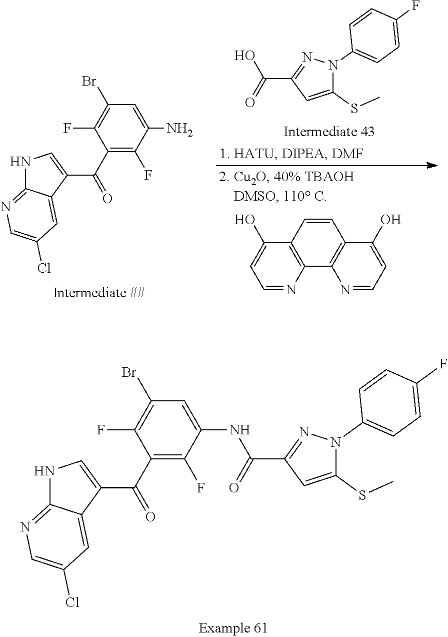 [SMILES: Nc1cc(Br)c(F)c(C(=O)c2c[nH]c3ncc(Cl)cc23)c1F ; Oc1ccnc2c1ccc1c(O)ccnc12 ; CSc1cc(C(=O)O)nn1-c1ccc(F)cc1 ; CSc1cc(C(=O)Nc2cc(Br)c(F)c(C(=O)c3c[nH]c4ncc(Cl)cc34)c2F)nn1-c1ccc(F)cc1]

### N-[5-bromo-3-(5-chloro-1H-pyrrolo[2,3-b]pyridine-3-carbonyl)-2,4-difluoro-phenyl]-5-methylsulfanyl-1-phenyl-pyrazole-3-carboxamide

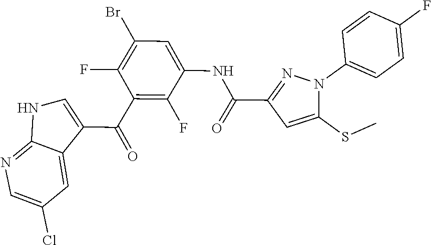 [SMILES: CSc1cc(C(=O)Nc2cc(Br)c(F)c(C(=O)c3c[nH]c4ncc(Cl)cc34)c2F)nn1-c1ccc(F)cc1]

HATU (0.31 g, 0.823 mmol, 1.05 eq) was added to an oven-dried vial containing a solution of (3-amino-5-bromo-2,6-difluoro-phenyl)-(5-chloro-1H-pyrrolo[2,3-b]pyridin-3-yl)methanone (0.30 g, 0.784 mmol, 1.00 eq), 1-(4-fluorophenyl)-5-(methylthio)-1H-pyrazole-3-carboxylic acid (0.30 g, 1.18 mmol, 1.50 eq), and DIPEA (0.24 mL, 1.37 mmol, 1.75 eq) in DMF (7.838 mL, 0.1000 M) at rt. The following day, after LC/MS revealed remaining starting material, additional 1-(4-fluorophenyl)-3-(methylthio)-1H-pyrazole-5-carboxylic acid (0.5 equiv, 0.099 g), DIPEA (0.5 equiv., 0.068 mL) and HATU (0.5 equiv., 0.149 g) were added and the reaction mixture was heated to 45° C. and stirred overnight. The following day, after LC/MS revealed remaining starting material, additional HATU (0.2 equiv., 0.060 g) was added and the reaction mixture was once more stirred overnight at 45° C. The reaction was cooled to rt and quenched with half sat. sodium bicarbonate. The resulting precipitate was collected via vacuum filtration, rinsing with water (×2) and Et₂O to afford crude N-[5-bromo-3-(5-chloro-1H-pyrrolo[2,3-b]pyridine-3-carbonyl)-2,4-difluoro-phenyl]-1-(4-fluorophenyl)-5-methylsulfanyl-pyrazole-3-carboxamide (0.38 g, 0.452 mmol) as a solid that was used in the subsequent reaction without further purification. 1H NMR (500 MHz, DMSO-d₆) δ 10.10 (s, 1H), 8.50 (d, J=2.1 Hz, 1H), 8.45 (d, J=2.4 Hz, 1H), 8.37 (s, 1H), 8.20 (t, J=7.6 Hz, 1H), 7.69 (dd, J=9.0, 4.9 Hz, 2H), 7.44 (t, J=8.8 Hz, 2H), 7.04 (s, 1H), 2.52 (s, 3H). MS(ES⁺) m/z calc'd for [M+H]⁺ [C₂₅H₁₅BiClF₂N₅O₂S+H]⁺ 619.9: found 619.8, LCMS t_{R}=8.97 min [Analytical Method: 05991008_AA0.lcm].

### N-(3-(5-chloro-1H-pyrrolo[2,3-b]pyridine-3-carbonyl)-2,4-difluoro-5-hydroxyphenyl)-1-(4-fluorophenyl)-5-(methylthio)-1H-pyrazole-3-carboxamide [Example 61]

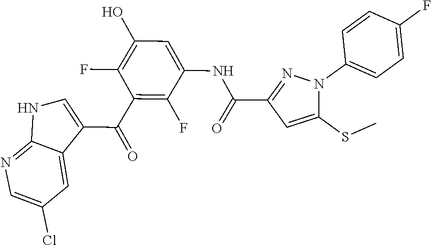 [SMILES: CSc1cc(C(=O)Nc2cc(O)c(F)c(C(=O)c3c[nH]c4ncc(Cl)cc34)c2F)nn1-c1ccc(F)cc1]

A mixture of N-(5-bromo-3-(5-chloro-1H-pyrrolo[2,3-b]pyridine-3-carbonyl)-2,4-difluorophenyl)-1-(4-fluorophenyl)-5-(methylthio)-1H-pyrazole-3-carboxamide (0.080 g, 0.129 mmol, 1.00 eq), Cu₂O (0.0074 g, 0.0515 mmol, 0.400 eq), 4,7-dihydroxy-1,10-phenanthroline (0.022 g, 0.103 mmol, 0.800 eq), and TBAOH (0.17 mL, 0.258 mmol, 2.00 eq) in DMSO (0.13 M, 1 mL) was purged with nitrogen for several minutes before lowering into an oil bath at 115° C. The following day, additional TBAOH (0.340 mL, 4 eq) was added, the headspace was purged with nitrogen and the reaction mixture lowered back into an oil bath at 115° C. to once more stir overnight. The reaction mixture was cooled to rt, quenched with 2M HCl dropwise, diluted with water, pH adjusted to ˜5 with sodium bicarbonate (aq., sat.), and the resulting solid collected via vacuum filtration. Column chromatography (SiO₂; solid load; 5-25% ACN in Toluene) followed by MeOH trituration of concentrated fractions then provided N-[3-(5-chloro-1H-pyrrolo[2,3-b]pyridine-3-carbonyl)-2,4-difluoro-5-hydroxy-phenyl]-1-(4-fluorophenyl)-5-methylsulfanyl-pyrazole-3-carboxamide (0.0090 g, 0.0158 mmol, 12.29% yield) as a white solid. ¹H NMR (600 MHz, DMSO) δ 13.11 (s, 1H), 10.26 (s, 1H), 9.81 (s, 1H), 8.47 (s, 1H), 8.44 (d, J=2.4 Hz, 1H), 8.23 (s, 1H), 7.68 (dd, J=9.0, 4.9 Hz, 2H), 7.57-7.52 (m, 1H), 7.43 (t, J=8.8 Hz, 2H), 7.03 (s, 1H), 2.52 (s, 3H); MS (ES⁺) m/z calc'd for [M+H]⁺ [C₂₅H₁₅ClF₃N₅O₃S+H]⁺: 558.93 found 558.1, LCMS: t_{R}=7.65 min [Analytical Method: 05991008_AA0.lcm]

### Synthesis of 1-(4-fluorophenyl)-N-(5-hydroxy-2′-methyl-5′-morpholino-[1,1′-biphenyl]-3-yl)-5-(methylsulfonyl)-1H-pyrazole-3-carboxamide [Example 62]

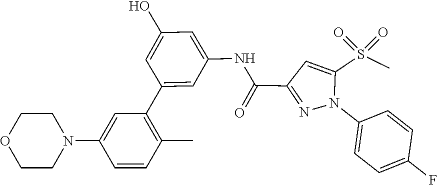 [SMILES: Cc1ccc(N2CCOCC2)cc1-c1cc(O)cc(NC(=O)c2cc(S(C)(=O)=O)n(-c3ccc(F)cc3)n2)c1]

Synthesis of Example 62 followed the same procedure used in Synthetic Method A except used 3-amino-5-(2-methyl-5-morpholino-phenyl)phenol and 1-(4-fluorophenyl)-5-(methylsulfonyl)-1H-pyrazole-3-carboxylic acid. The crude was purified by column chromatography (silica gel; dry load; 5-30% ACN in toluene) afforded 0.041 g, 0.0738 mmol, 28.36% yield of the title compound as a white solid. ¹H NMR (600 MHz, DMSO) δ 10.32 (s, 1H), 9.52 (s, 1H), 7.75 (dd, J=9.0, 4.8 Hz, 2H), 7.66 (s, 1H), 7.45 (t, J=8.7 Hz, 2H), 7.39 (t, J=2.0 Hz, 1H), 7.15 (t, J=1.5 Hz, 1H), 7.12 (d, J=8.5 Hz, 1H), 6.85 (dd, J=8.4, 2.7 Hz, 1H), 6.71 (d, J=2.6 Hz, 1H), 6.46 (dd, 1H), 3.74-3.70 (m, 4H), 3.33 (s, 3H), 3.09-3.04 (m, 4H), 2.14 (s, 3H); MS (ES⁺) m/z calc'd for [M+H]⁺ [C₂₈H₂₇FN₄O₅S+H]⁺: 551.60 found 551.2, LCMS: t_{R}=6.65 min [Analytical Method: 05991008_BB1.lcm]

### Synthesis of 1-(4-fluorophenyl)-5-(methylsulfinyl)-N-(p-tolyl)-1H-pyrazole-3-carboxamide [Example 63]

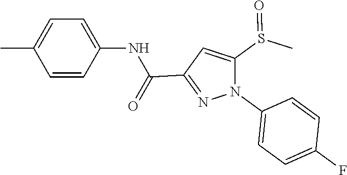 [SMILES: Cc1ccc(NC(=O)c2cc(S(C)=O)n(-c3ccc(F)cc3)n2)cc1]

To a suspension of 4-methylaniline (30 mg, 0.280 mmol, 1.00 eq) and 1-(4-fluorophenyl)-5-(methylsulfinyl)-1H-pyrazole-3-carboxylic acid (83 mg, 0.308 mmol, 1.10 eq) in 3 ml of DCM was added a solution of EDCI (107 mg, 0.560 mmol, 2.00 eq) in 2 ml of DCM and N,N-diisopropylethylamine (0.11 mL, 0.616 mmol, 2.20 eq), the resulting reaction solution was stirred at room temperature, with LCMS monitoring. The reaction was stirred for 24 hours and found to be about 70% complete. The reaction was quenched with aq. NaHCO₃, diluted with DCM, washed successively with aq. NaHCO₃, water and brine, dried with anhydrous Na₂SO₄. Filtered and concentrated and the residue was purified by flash chromatography over SiO₂, ISCO, CombiFlash, 4 g cartridge (dry load, 30-80% ethyl acetate in hexane) to provide solid which was taken to acetonitrile-water (1:1) and lyophilized overnight to afford 39 mg, 0.106 mmol, 37.84% yield of the title compound as a light-yellow solid. ¹H NMR (500 MHz, DMSO-d₆) δ 10.23 (s, 1H), 7.83-7.75 (m, 2H), 7.71-7.64 (m, 3H), 7.53-7.45 (m, 2H), 7.18-7.10 (m, 2H), 3.06 (s, 3H), 2.27 (s, 3H). MS(ES⁺) m/z calc'd for [M+H]⁺ 258.1: found 257.7, LCMS t_{R}=2.35 min [Analytical Method B].

### Synthesis of N-(2-fluoro-5-((3-methyl-4-thioxo-3,4-dihydroquinazolin-6-yl)oxy)phenyl)-1-(4-fluorophenyl)-5-(methylsulfinyl)-1H-pyrazole-3-carboxamide [Example 64]

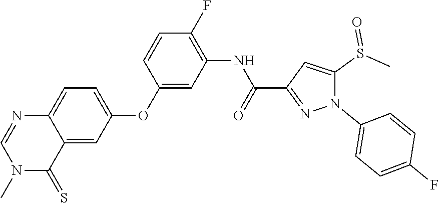 [SMILES: Cn1cnc2ccc(Oc3ccc(F)c(NC(=O)c4cc(S(C)=O)n(-c5ccc(F)cc5)n4)c3)cc2c1=S]

Synthesis of Example 64 followed the same procedure used in Synthetic Method A except used 6-(3-amino-4-fluoro-phenoxy)-3-methyl-quinazoline-4-thione and 1-(4-fluorophenyl)-5-(methylsulfinyl)-1H-pyrazole-3-carboxylic acid. The crude was purified by flash chromatography over silica gel, ISCO, CombiFlash, 4 g cartridge (dry load, 0-10% MeOH/DCM as eluent) and the residue was suspended in DCM (5 mL) and to this was added diethyl ether (5 mL). Suspension was stirred at rt for 15 mins before it was filtered. Solids were washed with diethyl ether suspended in ACN and water (1:10) and dried under lyophilization to afford 17 mg, 0.0293 mmol, 29.41% yield of the title compound as off white solid. ¹H NMR (500 MHz, DMSO-d₆) δ 10.09 (s, 1H), 8.67 (s, 1H), 8.13 (d, J=2.9 Hz, 1H), 7.85-7.75 (m, 3H), 7.68-7.63 (m, 2H), 7.60 (dt, J=6.3, 3.8 Hz, 1H), 7.53-7.44 (m, 2H), 7.38 (t, J=9.5 Hz, 1H), 7.02 (dd, J=8.3, 4.1 Hz, 1H), 3.87 (s, 3H), 3.05 (s, 3H). MS(ESI⁺) m/z calc'd for [M+H]⁺[C₂₆H₁₉F₂N₅O₃S₂+H]⁺: 552.1 found: 552.1, t_{R}=2.68 mins. [Analytical Method B].

### Synthesis of 1-(4-fluorophenyl)-N-(4-methyl-3-((3-methyl-4-oxo-3,4-dihydropyrido[3,2-d]pyrimidin-6-yl)amino)phenyl)-5-(methylsulfinyl)-1H-pyrazole-3-carboxamide [Example 65]

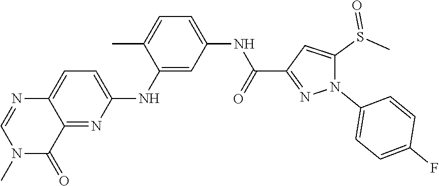 [SMILES: Cc1ccc(NC(=O)c2cc(S(C)=O)n(-c3ccc(F)cc3)n2)cc1Nc1ccc2ncn(C)c(=O)c2n1]

Synthesis of Example 65 followed the same procedure used in Synthetic Method A except used 6-(5-amino-2-methyl-anilino)-3-methyl-pyrido[3,2-d]pyrimidin-4 and 1-(4-fluorophenyl)-5-(methylsulfinyl)-1H-pyrazole-3-carboxylic acid. The crude was suspended in water (4 mL) and acetone (2 mL) and the suspension was stirred at rt for 10 mins before it was filtered. Solids were washed with diethyl ether and dried under reduced pressure to afford 0.027 g, 0.0490 mmol, 45.96% yield of the title compound. ¹H NMR (500 MHz, DMSO-d₆) δ 10.26 (s, 1H), 8.95 (s, 1H), 8.19 (s, 1H), 7.92 (d, J=2.3 Hz, 1H), 7.83-7.75 (m, 3H), 7.66 (s, 1H), 7.53 (dd, J=8.2, 2.3 Hz, 1H), 7.48 (t, J=8.8 Hz, 2H), 7.22 (d, J=8.4 Hz, 1H), 7.13 (d, J=8.9 Hz, 1H), 3.46 (s, 3H), 3.05 (s, 3H), 2.19 (s, 3H). MS(ESI⁺) m/z calc'd for [M+H]⁺[C₂₆H₂₂FN₇O₃S+H]⁺: 532.1 found: 532.0, t_{R}=2.05 mins. [Analytical Method B].

### Synthesis of 1-(4-fluorophenyl)-N-(4-methyl-3-((3-methyl-4-thioxo-3,4-dihydroquinazolin-6-yl)amino)phenyl)-5-(methylsulfinyl)-1H-pyrazole-3-carboxamide [Example 66]

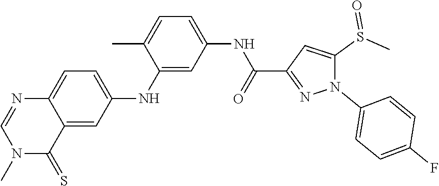 [SMILES: Cc1ccc(NC(=O)c2cc(S(C)=O)n(-c3ccc(F)cc3)n2)cc1Nc1ccc2ncn(C)c(=S)c2c1]

Synthesis of Example 66 followed the same procedure used in Synthetic Method A except used 6-(5-amino-2-methyl-anilino)-3-methyl-quinazoline-4-thione and 1-(4-fluorophenyl)-5-(methylsulfinyl)-1H-pyrazole-3-carboxylic acid. The crude was suspended in water (4 mL) and acetone (2 mL) and the suspension was stirred at rt for 10 mins before it was filtered. Solids were washed with diethyl ether and dried under reduced pressure to afford 37 mg, 0.0635 mmol, 75.27% yield of the title compound as yellow solid. ¹H NMR (500 MHz, DMSO-d₆) δ 10.27 (s, 1H), 8.49 (s, 1H), 8.23 (s, 1H), 7.96 (d, J=2.7 Hz, 1H), 7.81 (d, J=2.3 Hz, 1H), 7.80-7.75 (m, 2H), 7.64 (s, 1H), 7.60 (d, J=8.8 Hz, 1H), 7.51-7.44 (m, 4H), 7.23 (d, J=8.8 Hz, 1H), 3.86 (s, 3H), 3.05 (s, 3H), 2.17 (s, 3H). MS(ESI⁺) m/z calc'd for [M+H]⁺[C₂₇H₂₃FN₆O₂S₂+H]⁺: 547.1 found: 547.3, t_{R}=2.54 mins. [Analytical Method B].

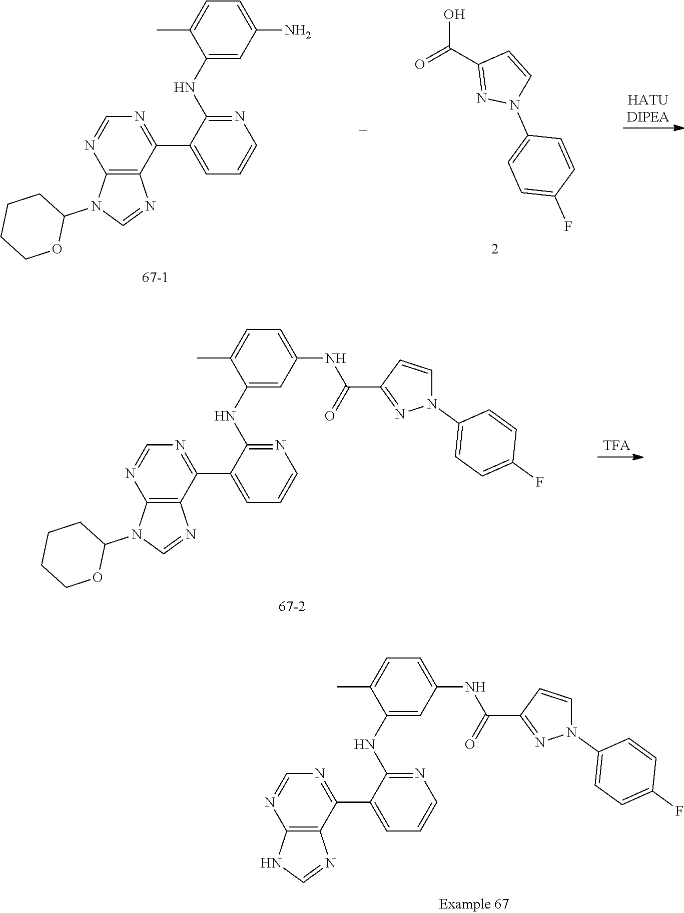 [SMILES: Cc1ccc(N)cc1Nc1ncccc1-c1ncnc2c1ncn2C1CCCCO1 ; O=C(O)c1ccn(-c2ccc(F)cc2)n1 ; Cc1ccc(NC(=O)c2ccn(-c3ccc(F)cc3)n2)cc1Nc1ncccc1-c1ncnc2c1ncn2C1CCCCO1 ; Cc1ccc(NC(=O)c2ccn(-c3ccc(F)cc3)n2)cc1Nc1ncccc1-c1ncnc2[nH]cnc12]

### Synthesis of N-(3-((3-(9H-purin-6-yl)pyridin-2-yl)amino)-4-methylphenyl)-1-(4-fluorophenyl)-1H-pyrazole-3-carboxamide [Example 67]

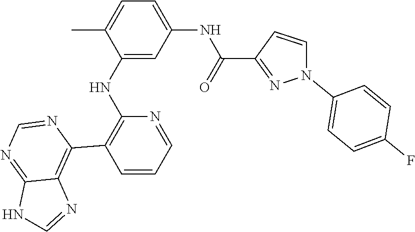 [SMILES: Cc1ccc(NC(=O)c2ccn(-c3ccc(F)cc3)n2)cc1Nc1ncccc1-c1ncnc2[nH]cnc12]

Synthesis of Example 67 followed the same procedure used in Synthetic Method B except used 1-(4-fluorophenyl)-N-[4-methyl-3-[[3-(9-tetrahydropyran-2-ylpurin-6-yl)-2-pyridyl]amino]-phenyl]pyrazole-3-carboxamide afford N-(3-((3-(9H-purin-6-yl)pyridin-2-yl)amino)-4-methylphenyl)-1-(4-fluorophenyl)-1H-pyrazole-3-carboxamide 49 mg, 0.0944 mmol, 87.52% yield of the title compound as a ginger yellow solid. ¹H NMR (500 MHz, DMSO-d₆) δ 12.29 (s, 1H), 10.02 (s, 1H), 9.80-9.74 (m, 1H), 9.04 (s, 1H), 8.69-8.64 (m, 2H), 8.60 (d, J=2.6 Hz, 1H), 8.34 (dd, J=4.6, 2.0 Hz, 1H), 8.10-8.02 (m, 2H), 7.46 (dd, J=8.2, 2.3 Hz, 1H), 7.45-7.37 (m, 2H), 7.22 (d, J=8.5 Hz, 1H), 7.06-6.98 (m, 2H), 2.42 (s, 3H). MS(ES⁺) m/z calc'd for [M+H]⁺: 506.2, found: 506.2 t_{R}=2.13 min. [Analytical Method: B].

### 1-(4-fluorophenyl)-N-(4-methyl-3-((3-(9-(tetrahydro-2H-pyran-2-yl)-9H-purin-6-yl)pyridin-2-yl)amino)phenyl)-1H-pyrazole-3-carboxamide [67-2]

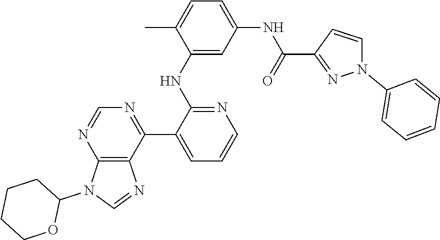 [SMILES: Cc1ccc(NC(=O)c2ccn(-c3ccccc3)n2)cc1Nc1ncccc1-c1ncnc2c1ncn2C1CCCCO1]

To a stirred solution of 1-(4-fluorophenyl)-1H-pyrazole-3-carboxylic acid (28 mg, 0.137 mmol, 1.10 eq) in 0.5 ml DMF at rt was added a solution of HATU (66 mg, 0.174 mmol, 1.40 eq) in 0.5 ml of DMF, and diisopropylethylamine (0.061 mL, 0.349 mmol, 2.00 eq). The resulting solution was stirred for 10 minutes, then solution of 4-methyl-N-[3-[3-(9-tetrahydropyran-2-ylpurin-6-yl)-2-pyridyl]benzene-1,3-diamine (50 mg, 0.125 mmol, 1.00 eq) in 1 ml DMF and N,N-Diisopropylethylamine (0.039 mL, 0.224 mmol, 1.80 eq) was added. The resulting reaction mixture was stirred at rt for 1 h, then quenched with aq. NaHCO₃, stirred for 30 minutes, filtered, the solid was washed successively with water, 1M LiCl, water and ether to afford 1-(4-fluorophenyl)-N-(4-methyl-3-((3-(9-(tetrahydro-2H-pyran-2-yl)-9H-purin-6-yl)pyridin-2-yl)amino)phenyl)-1H-pyrazole-3-carboxamide (65 mg, 0.108 mmol, 86.61% yield) as a bright yellow solid. ¹H NMR (500 MHz, CDCl₃) δ 12.11 (s, 1H), 9.89-9.60 (m, 1H), 9.10-8.71 (m, 2H), 8.66-8.29 (m, 3H), 8.03-7.52 (m, 3H), 7.42-6.85 (m, 5H), 6.04-5.81 (m, 1H), 4.40-4.12 (m, 1H), 3.96-3.75 (m, 1H), 2.48 (d, J=3.9 Hz, 3H), 2.33-1.97 (m, 3H), 1.97-1.47 (m, 5H). MS(ES⁺) m/z calc'd for [M+H]⁺: 590.4, found: 590.3, t_{R}=5.17 mins. [Method: A-12].

### Synthesis of 1-(4-fluorophenyl)-N-(4-(4-methyl-2-(pyridin-3-yl)thiazol-5-yl)pyridin-2-yl)-5-(methylsulfonyl)-1H-pyrazole-3-carboxamide [Example 68]

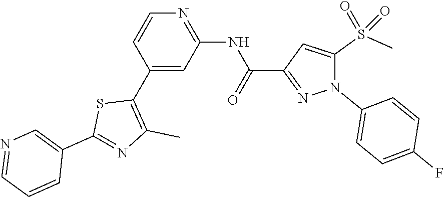 [SMILES: Cc1nc(-c2cccnc2)sc1-c1ccnc(NC(=O)c2cc(S(C)(=O)=O)n(-c3ccc(F)cc3)n2)c1]

Synthesis of Example 68 followed the same procedure used in Synthetic Method A except used 4-[4-methyl-2-(3-pyridyl)thiazol-5-yl]pyridin-2-amine and 1-(4-fluorophenyl)-5-(methylsulfonyl)-1H-pyrazole-3-carboxylic acid. The crude after filtration was purified by column chromatography on silica gel; dry load; 20-70% EtOAc in hexanes afforded 0.0060 g, 0.0110 mmol, 21.00% yield of the title compound as an orange solid. ¹H NMR (600 MHz, DMSO) δ 10.56 (s, 1H), 8.67 (s, 1H), 8.49 (d, J=3.2 Hz, 1H), 8.41 (s, 1H), 8.16 (d, J=8.0 Hz, 1H), 8.00 (t, J=7.8 Hz, 1H), 7.83-7.82 (m, 1H), 7.81-7.77 (m, 2H), 7.56-7.52 (m, 1H), 7.47 (t, J=8.7 Hz, 2H), 7.42 (d, J=5.0 Hz, 1H), 3.36 (s, 3H), 2.65 (s, 3H); MS (ES⁺) m/z calc'd for [M+H]⁺ [C₂₅H₁₉FN₆O₃S₂+H]⁺: 535.59 found 535.2, LCMS: t_{R}=3.60 min [Analytical Method:05990510_AA1.lcm]

### Synthesis of N-(4-chloro-3-(5,8-dimethyl-2-(methylamino)-7-oxo-7,8-dihydropyrido[2,3-d]pyrimidin-6-yl)phenyl)-1-(4-fluorophenyl)-1H-pyrazole-3-carboxamide [Example 69]

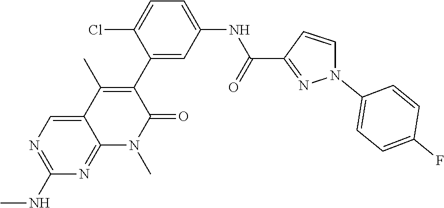 [SMILES: CNc1ncc2c(C)c(-c3cc(NC(=O)c4ccn(-c5ccc(F)cc5)n4)ccc3Cl)c(=O)n(C)c2n1]

Synthesis of Example 69 followed the same procedure used in Synthetic Method A except used 6-(5-amino-2-chlorophenyl)-5,8-dimethyl-2-(methylamino)pyrido[2,3-d]pyrimidin-7(8H)-one and 1-(4-fluorophenyl)-1H-pyrazole-3-carboxylic acid. The crude after filtration was purified by column chromatography on silica gel; dry load; 0-70% EtOAc in n-heptane afforded 0.0037 g, 0.0110 mmol, 45.78% yield of the title compound as a pale brown solid. ¹H NMR (400 MHz, DMSO-d₆): δ 10.33 (bs, 1H), 8.87-8.73 (m, 1H), 8.63 (d, J=2.6 Hz, 1H), 8.09-8.02 (m, 2H), 7.92 (dd, J=2.5, 8.8 Hz, 1H), 7.83-7.77 (m, 1H), 7.74 (d, J=2.5 Hz, 1H), 7.54 (d, J=8.8 Hz, 1H), 7.46-7.38 (m, 2H), 7.03 (d, J=2.5 Hz, 1H), 3.64-3.49 (m, 3H), 2.93 (d, J=4.5 Hz, 3H), 2.16 (s, 3H); MS (ES)⁺ m/z calc'd for [M+H]⁺ [C₂₆H₂₁ClFN₇02+H]⁺: 518.14, found 518.00, LCMS: t_{R}=3.56 min [Method C]:

### Synthesis of N-(5-bromo-3-(5-chloro-1H-pyrrolo[2,3-b]pyridine-3-carbonyl)-2,4-difluorophenyl)-1-(4-fluorophenyl)-5-(methylsulfonyl)-1H-pyrazole-3-carboxamide [Example 70]

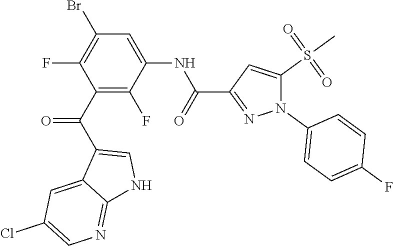 [SMILES: CS(=O)(=O)c1cc(C(=O)Nc2cc(Br)c(F)c(C(=O)c3c[nH]c4ncc(Cl)cc34)c2F)nn1-c1ccc(F)cc1]

Synthesis of Example 70 followed the same procedure used in Synthetic Method A except used (3-amino-5-bromo-2,6-difluorophenyl)(5-chloro-1H-pyrrolo[2,3-b]pyridin-3-yl)methanone and 1-(4-fluorophenyl)-5-(methylsulfonyl)-1H-pyrazole-3-carboxylic acid. The crude after filtration was purified by Preparative HPLC (Method B) resulting in 0.007 g, 14.8% yield of the title compound as a pale brown solid. ¹H NMR (400 MHz, DMSO-d6) δ=13.27-13.15 (m, 1H), 10.57-10.47 (m, 1H), 8.56-8.42 (m, 2H), 8.42-8.33 (m, 1H), 8.19-8.07 (m, 1H), 7.83-7.72 (m, 2H), 7.69-7.61 (m, 1H), 7.53-7.36 (m, 2H), 2.56-2.53 (m, 3H); MS (ES⁺) m/z calc'd for [M+H]⁺[C₂₅H₁₄BrClF₃N₅O₄S+H]⁺: 651.96, found: 653.95, t_{R}=4.50 min [Analytical Method: Method C].

### Synthesis of N-(4-((6,7-dimethoxyquinolin-4-yl)oxy)-2-fluorophenyl)-1-(4-fluorophenyl)-5-(methylsulfonyl)-1H-pyrazole-3-carboxamide [Example 71]

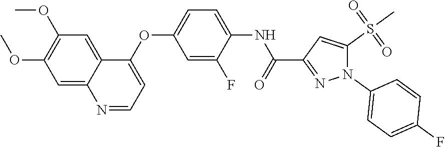 [SMILES: COc1cc2nccc(Oc3ccc(NC(=O)c4cc(S(C)(=O)=O)n(-c5ccc(F)cc5)n4)c(F)c3)c2cc1OC]

To a stirred solution of 4-((6,7-dimethoxyquinolin-4-yl)oxy)-2-fluoroaniline (30 mg, 0.104 mmol, 1.0 eq), 1-(4-fluorophenyl)-5-(methylsulfonyl)-1H-pyrazole-3-carboxylic acid (40.69 mg, 0.143 mmol, 1.5 eq) in DMF (1 mL, 0.012 mol) was added HATU (72.58 mg, 0.190 mmol, 2 eq) and followed by addition of DIPEA (36.93 mg, 0.28 mmol, 3 eq) and reaction mixture was irradiated at 80° C. in a Mw vessel for 30 min. Reaction completion was monitored by LCMS. The crude LCMS showed 37.50% desired product formation. Reaction mixture was quenched with ice cold water (10 ml) and sat NaHCO₃ (10 mL), the obtained solid which was filtered and washed with ACN (2 mL) and pentane (2 mL) to get solid compound which was dried under high vacuum to get title compound (12.67 mg, 22.51%) as an off-white solid. ¹H NMR (400 MHz, DMSO-d₆): δ 10.61 (s, 1H), 8.48 (d, J=5.1 Hz, 1H), 7.95 (d, J=9.0 Hz, 2H), 7.77 (dd, J=8.9, 4.9 Hz, 2H), 7.68 (s, 1H), 7.51 (s, 1H), 7.46 (t, J=8.8 Hz, 2H), 7.40 (s, 1H), 7.27 (d, J=9.0 Hz, 2H), 6.48 (d, J=5.3 Hz, 1H), 3.95 (s, 3H), 3.94 (s, 3H), 3.35 (s, 3H); MS (ES) calc'd for [M+H]⁺[C₂₈H₂₂F₂N₄O₆S+H]⁺: 581.56, found: 581.20, LCMS: t: 2.92 min.[Analytical method: D]

### Synthesis of N-(4-((6,7-dimethoxyquinolin-4-yl)oxy)phenyl)-1-(4-fluorophenyl)-5-(methylsulfonyl)-1H-pyrazole-3-carboxamide [Example 72]

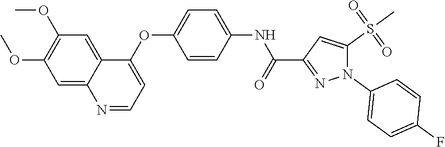 [SMILES: COc1cc2nccc(Oc3ccc(NC(=O)c4cc(S(C)(=O)=O)n(-c5ccc(F)cc5)n4)cc3)c2cc1OC]

Synthesis of Example 2 followed the same procedure used in Synthetic Method A except used 4-((6,7-dimethoxyquinolin-4-yl)oxy)aniline and 1-(4-fluorophenyl)-5-(methylsulfonyl)-1H-pyrazole-3-carboxylic acid. The solid was collected by filtration and dried under vacuum to get the title compound (50.00 mg, 87.19%) as light brown solid. ¹H NMR (400 MHz, DMSO-d₆): δ 10.61 (s, 1H), 8.48 (d, J=5.1 Hz, 1H), 7.95 (d, J=9.0 Hz, 2H), 7.77 (dd, J=8.9, 4.9 Hz, 2H), 7.68 (s, 1H), 7.51 (s, 1H), 7.46 (t, J=8.8 Hz, 2H), 7.40 (s, 1H), 7.27 (d, J=9.0 Hz, 2H), 6.48 (d, J=5.3 Hz, 1H), 3.95 (s, 3H), 3.94 (s, 3H), 3.35 (s, 3H); LCMS: MS(ES) calc'd for [M+H]⁺[C₂₈H₂₃FN₄O₆S+H]⁺: 563.54, found: 563.10, LCMS: t_{R}: 1.40 min. [Analytical Method: D].

### Synthesis of N-(4-((6,7-dimethoxyquinolin-4-yl)oxy)-3-fluorophenyl)-1-(4-fluorophenyl)-5-(methylsulfonyl)-1H-pyrazole-3-carboxamide [Example 73]

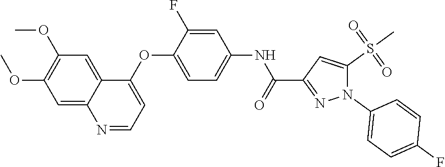 [SMILES: COc1cc2nccc(Oc3ccc(NC(=O)c4cc(S(C)(=O)=O)n(-c5ccc(F)cc5)n4)cc3F)c2cc1OC]

Synthesis of Example 3 followed the same procedure used in Synthetic Method A except used 4-((6,7-dimethoxyquinolin-4-yl)oxy)-3-fluoroaniline and 1-(4-fluorophenyl)-5-(methylsulfonyl)-1H-pyrazole-3-carboxylic acid. The solid was collected by filtration and dried under vacuum to get the title compound (31.03 mg, 54.44%) as brown solid. ¹H NMR (400 MHz, DMSO-d₆): δ 10.81 (br s, 1H), 8.50 (br d, J=4.8 Hz, 1H), 8.05 (br d, J=12.6 Hz, 1H), 7.78 (br s, 3H), 7.69 (br s, 1H), 7.54 (br s, 1H), 7.51-7.38 (m, 4H), 6.50 (br d, J=4.6 Hz, 1H), 3.96 (br s, 6H), 3.35 (br s, 3H); MS (ES) calc'd for [M+H]⁺ [C₂₈H₂₂F₂N₄O₆S+H]⁺: 581.56, found: 581.10, LCMS: t: 1.44 min. [Analytical Method: D].

### Synthesis of 1-(4-fluorophenyl)-N-(4-methyl-3-(8-methyl-2-(methylamino)-7-oxo-7,8-dihydropyrido[2,3-d]pyrimidin-6-yl)phenyl)-1H-pyrazole-3-carboxamide [Example 74]

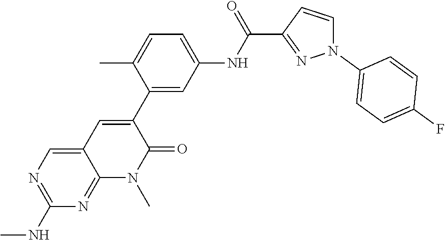 [SMILES: CNc1ncc2cc(-c3cc(NC(=O)c4ccn(-c5ccc(F)cc5)n4)ccc3C)c(=O)n(C)c2n1]

To a stirred solution of 6-(5-amino-2-methylphenyl)-8-methyl-2-(methylamino)pyrido[2,3-d]pyrimidin-7(8H)-one (30 mg, 0.101 mmol, 1.0 eq), 1-(4-fluorophenyl)-1H-pyrazole-3-carboxylic acid (31 mg, 0.152 mmol, 1.50 eq) in DMF (1 mL, 0.012 mol) was added HATU (57.93 mg, 0.150 mmol, 1.5 eq) and followed by addition of DIPEA (65.64 mg, 0.50 mmol, 5 eq) and reaction mixture was heated to 80-90° C. for 8 h. Reaction completion was monitored by LCMS. The crude LCMS showed desired product formation (31.29%). Reaction mixture was quenched with ice cold water (10 ml) and sat NaHCO₃ (10 ml) to obtained solid which was filtered and washed with ACN (2 mL) and pentane (2 mL) to get solid compound which was dried under high vacuum to get title compound (19.52 mg, 36.06%) as light brown solid. ¹H NMR (400 MHz, DMSO-d₆): δ 10.09 (s, 1H), 8.62 (d, J=2.5 Hz, 2H), 8.10-8.01 (m, 2H), 7.86-7.79 (m, 1H), 7.75 (dd, J=8.3, 2.2 Hz, 1H), 7.73 (s, 1H), 7.67 (d, J=2.3 Hz, 1H), 7.42 (t, J=8.8 Hz, 2H), 7.23 (d, J=8.4 Hz, 1H), 7.01 (d, J=2.6 Hz, 1H), 3.71-3.52 (m, 3H), 2.93 (br d, J=4.1 Hz, 3H), 2.12 (s, 3H); MS (ES) calc'd for [M+H]⁺ [C₂₆H₂₂FN₇O₂+H]⁺: 484.50, found: 484.05, LCMS: t_{R}: 1.85 min. [Analytical Method: D].

### Synthesis of 1-(4-fluorophenyl)-N-(4-methyl-3-((4-(pyridin-3-yl)pyrimidin-2-yl)amino)phenyl)-1H-pyrazole-3-carboxamide [Example 75]

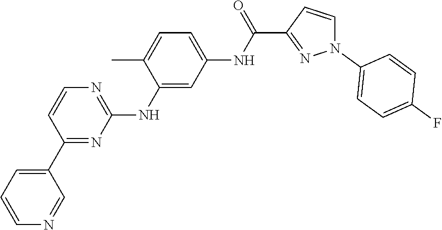 [SMILES: Cc1ccc(NC(=O)c2ccn(-c3ccc(F)cc3)n2)cc1Nc1nccc(-c2cccnc2)n1]

Synthesis of Example 4 followed the same procedure used in Synthetic Method B except used 2-(5-Amino-2-methylanilino)-4-(3-pyridyl)pyrimidine and 1-(4-fluorophenyl)-1H-pyrazole-3 carboxylic acid. The solid was collected by filtration and dried under vacuum to get the title compound (47.07 mg, 87.80%) as brown solid. ¹H NMR (400 MHz, DMSO-d₆): δ 10.08 (s, 1H), 9.27 (d, J=1.6 Hz, 1H), 8.97 (s, 1H), 8.68 (dd, J=4.8, 1.6 Hz, 1H), 8.61 (d, J=2.5 Hz, 1H), 8.52 (s, 1H), 8.47 (dt, J=8.1, 1.9 Hz, 1H), 8.11 (d, J=2.0 Hz, 1H), 8.06 (dd, J=4.8, 9.1 Hz, 2H), 7.57-7.49 (m, 2H), 7.46-7.37 (m, 3H), 7.22 (d, J=8.3 Hz, 1H), 7.02 (d, J=2.6 Hz, 1H), 2.23 (s, 3H); MS (ES)calc'd for [M+H]⁺[C₂₆H₂₀FN₇O+H]⁺: 466.48, found: 466.0, LCMS: t_{R}: 1.76 min. [Analytical Method: D].

### Synthesis of N-(4-chloro-3-(8-methyl-2-(methylamino)-7-oxo-7,8-dihydropyrido[2,3-d]pyrimidin-6-yl)phenyl)-1-(4-fluorophenyl)-5-(methylsulfonyl)-1H-pyrazole-3-carboxamide [Example 76]

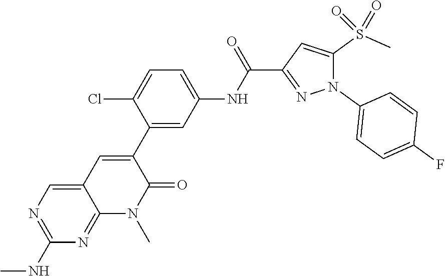 [SMILES: CNc1ncc2cc(-c3cc(NC(=O)c4cc(S(C)(=O)=O)n(-c5ccc(F)cc5)n4)ccc3Cl)c(=O)n(C)c2n1]

Synthesis of Example 6 followed the same procedure used in Synthetic Method B except used 6-(5-amino-2-chloro-phenyl)-8-methyl-2-(methylamino)pyrido[2,3-d]pyrimidin-7-one and 1-(4-fluorophenyl)-5-methylsulfonyl-pyrazole-3-carboxylic acid and DIPEA (3 eq). The solid was collected by filtration and dried under vacuum to get the title compound (6.77 mg, 12.18%) as an off white solid. ¹H NMR (400 MHz, DMSO-d₆): δ 10.61 (s, 1H), 8.76-8.57 (m, 1H), 7.93-7.73 (m, 6H), 7.65 (s, 1H), 7.50 (d, J=8.8 Hz, 1H), 7.45 (t, J=8.7 Hz, 2H), 3.66-3.51 (m, 3H), 3.33 (s, 3H), 2.93 (br d, J=4.0 Hz, 3H); MS (ES)⁺ calc'd for [M+H]⁺ [C₂₆H₂₁ClFN₇O₄S+H]⁺: 583.01 found: 582.05, LCMS: t: 1.78 min. [Analytical Method: D].

### Synthesis of N-(4-((6,7-dimethoxyquinolin-4-yl)oxy)-2-fluorophenyl)-1-(4-fluorophenyl)-5-(methylthio)-1H-pyrazole-3-carboxamide [Example 77]

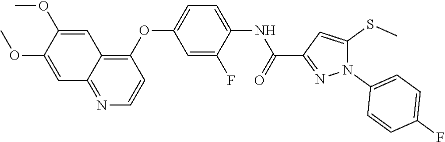 [SMILES: COc1cc2nccc(Oc3ccc(NC(=O)c4cc(SC)n(-c5ccc(F)cc5)n4)c(F)c3)c2cc1OC]

Synthesis of Example 7 followed the same procedure used in Synthetic Method of Example 6 except used 4-[(6,7-Dimethoxy-4-quinolinyl)oxy]-2-fluoroaniline and 1-(4-fluorophenyl)-5-1-(4-fluorophenyl)-5-methylsulfanyl-pyrazole-3-carboxylic acid. The solid was collected by filtration and dried under vacuum to get the title compound (9.87 mg, 18.80%) as off white solid. ¹H NMR (400 MHz, DMSO-d₆): δ 9.93 (s, 1H), 8.54 (d, J=5.3 Hz, 1H), 7.81 (t, J=8.8 Hz, 1H), 7.76-7.68 (m, 2H), 7.50-7.41 (m, 4H), 7.37 (dd, J=11.1, 2.5 Hz, 1H), 7.15 (dd, J=8.8, 1.8 Hz, 1H), 7.05 (s, 1H), 6.63 (d, J=5.3 Hz, 1H), 3.96 (s, 3H), 3.94 (s, 3H), 2.54 (s, 3H); MS (ES) calc'd for [M+H]⁺ [C₂₈H₂₂F₂N₄O₄S+H]⁺: 549.56, found: 549.05, LCMS: t_{R}: 1.57 min. [Analytical Method: F].

### Synthesis of N-(4-((6,7-dimethoxyquinolin-4-yl)oxy)-2-fluorophenyl)-1-(4-fluorophenyl)-1H-pyrazole-3-carboxamide [Example 78]

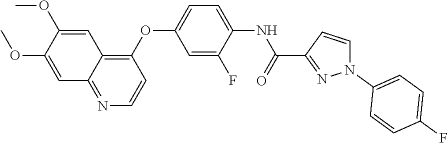 [SMILES: COc1cc2nccc(Oc3ccc(NC(=O)c4ccn(-c5ccc(F)cc5)n4)c(F)c3)c2cc1OC]

Synthesis of Example 8 followed the same procedure used in Synthetic Method B except used 4-[(6,7-Dimethoxy-4-quinolinyl)oxy]-2-fluoroaniline and 1-(4-fluorophenyl)-1H-pyrazole-3-carboxylic acid. The solid was collected by filtration and dried under vacuum to get the title compound to get the title compound (24.34 mg, 0.0461 mmol, 48.32% yield) as an off-white solid. ¹H NMR (400 MHz, DMSO-d₆): δ 9.98 (br s, 1H), 8.65 (d, J=2.3 Hz, 1H), 8.54 (d, J=5.1 Hz, 1H), 8.04 (br dd, J=8.8, 4.6 Hz, 2H), 7.85 (br t, J=8.8 Hz, 1H), 7.51-7.35 (m, 5H), 7.16 (br d, J=7.8 Hz, 1H), 7.06 (br d, J=2.3 Hz, 1H), 6.64 (d, J=5.1 Hz, 1H), 3.96 (s, 3H), 3.94 (s, 3H); MS (ES) calc'd for [M+H]⁺[C₂₇H₂₀F₂N₄O₄+H]⁺: 503.47, found: 503.45, LCMS: t_{R}: 1.52 min. [Analytical Method: D].

### Synthesis of 1-(4-fluorophenyl)-N-(3-(imidazo[1,2-b]pyridazin-3-ylethynyl)-4-methylphenyl)-1H-pyrazole-3-carboxamide [Example 79]

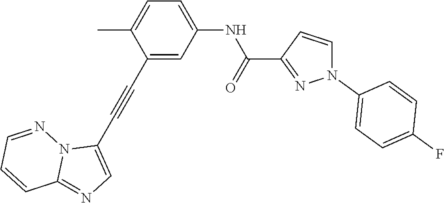 [SMILES: Cc1ccc(NC(=O)c2ccn(-c3ccc(F)cc3)n2)cc1C#Cc1cnc2cccnn12]

Synthesis of Example 6 followed the same procedure used in General Synthetic Method A except used 3-(imidazo[1,2-b]pyridazin-3-ylethynyl)-4-methylaniline and 1-(4-fluorophenyl)-1H-pyrazole-3-carboxylic acid. The solid was collected by filtration and dried to afford 15.74 mg of 1-(4-fluorophenyl)-N-(3-(imidazo[1,2-b]pyridazin-3-ylethynyl)-4-methylphenyl)-1H-pyrazole-3-carboxamide [Example 79]. ¹H NMR (400 MHz, DMSO) δ 10.23 (s, 1H), 8.72 (dd, J=4.5, 1.6 Hz, 1H), 8.64 (d, J=2.6 Hz, 1H), 8.29-8.20 (m, 2H), 8.14-8.02 (m, 3H), 7.80 (dd, J=8.3, 2.3 Hz, 1H), 7.50-7.32 (m, 4H), 7.04 (d, J=2.6 Hz, 1H), 2.49 (s, 3H); MS(ES⁺) m/z calc'd for [M+H]⁺ [C25H17FN6O]*436.45, found: 437.1, LCMS: t_{R}=3.82 min, Method C: Column: X-Select CSH C18, (50 mm*3.0 mm, 2.5p) Mobile Phase A: 0.05% Formic Acid in Water:Acetonitrile (95:5) Mobile Phase B: 0.05% Formic Acid in Acetonitrile Flow rate: 1.2 mL/min. Column temperature: 50° C.

### General Synthetic Method C:

### Synthesis of N-(2-fluoro-5-((3-methyl-4-oxo-3,4-dihydroquinazolin-6-yl)oxy)phenyl)-1-(4-fluorophenyl)-1H-pyrazole-3-carboxamide [Example 80]: Method C

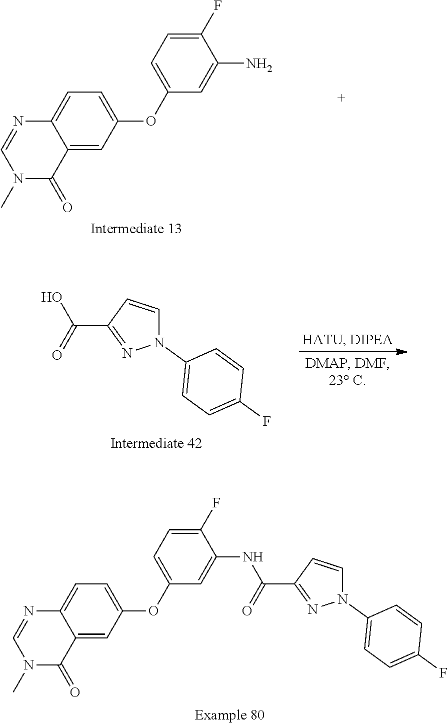 [SMILES: Cn1cnc2ccc(Oc3ccc(F)c(N)c3)cc2c1=O ; O=C(O)c1ccn(-c2ccc(F)cc2)n1 ; Cn1cnc2ccc(Oc3ccc(F)c(NC(=O)c4ccn(-c5ccc(F)cc5)n4)c3)cc2c1=O]

A solution of 6-(3-amino-4-fluorophenoxy)-3-methylquinazolin-4(3H)-one, 1-(4-fluorophenyl)-1H-pyrazole-3-carboxylic acid (0.030 g, 0.10 mmol, 1.00 eq) in anhydrous DMF (1 mL, 0.1771 M) was charged with diisopropylethylamine (0.09 mL, 0.52 mmol, 5.00 eq), DMAP (0.002 g, 0.016 mmol, 0.15 eq.) and a solution of 1-[Bis (Dimethylamino)methylene]-1H-1,2,3-triazolo[4,5-b]pyridinium 3-oxide hexafluorophosphate (0.060 g, 0.15 mmol, 1.5 eq) in anhydrous DMF (1 mL, 0.1771 M) and stirred at room temperature for 2-192 h. Progress of reaction was monitored by TLC. After the completion of reaction on TLC, quenched with ice cold water (10 mL), and stirred for 30 min at room temperature. The solid was filtered through a Buchner funnel and the filter cake was washed with 1N HCl (5 mL), saturated Aq. NaHCO₃ (2×5 mL), water (2×5 mL), diethyl ether (2×5 mL). Crude compound was purified by silica 100-200 mesh size in MeOH: DCM. Resulting in 6.87 mg of the title compound [Example 81]. ¹H NMR (400 MHz, DMSO) δ 9.88 (s, 1H), 8.63 (d, J=2.6 Hz, 1H), 8.33 (s, 1H), 8.06-7.98 (m, 2H), 7.75 (d, J=8.8 Hz, 1H), 7.65 (dd, J=6.4, 3.0 Hz, 1H), 7.60 (dd, J=8.8, 2.9 Hz, 1H), 7.54 (d, J=2.9 Hz, 1H), 7.47-7.36 (m, 3H), 7.07-6.98 (m, 2H), 3.48 (s, 3H); MS(ES⁺) m/z calc'd for [M+H]⁺[C₂₅H₁₇F₂N₅O₃]⁺473.44, found: 474.0, LCMS: t_{R}=3.71 min [Method C] Method: Column: X-Select CSH C18, (50 mm*3.0 mm, 2.5p) Mobile Phase A: 0.05% Formic Acid in Water:Acetonitrile (95:5) Mobile Phase B: 0.05% Formic Acid in Acetonitrile Flow rate: 1.2 mL/min. Column temperature: 50° C.

### Synthesis of N-(3-((4-aminothieno[2,3-d]pyrimidin-5-yl)ethynyl)phenyl)-1-(4-fluorophenyl)-1H-pyrazole-3-carboxamide [Example 81]

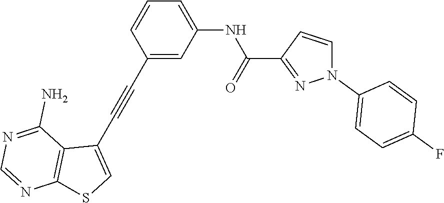 [SMILES: Nc1ncnc2scc(C#Cc3cccc(NC(=O)c4ccn(-c5ccc(F)cc5)n4)c3)c12]

Synthesis of Example 6 followed the same procedure used in General Synthetic Method C except used 5-((3-aminophenyl)ethynyl)-4a,7a-dihydrothieno[2,3-d]pyrimidin-4-amine and 1-(4-fluorophenyl)-1H-pyrazole-3-carboxylic acid. The solid was collected by filtration and dried to afford 27.87 mg of the title compound [Example 81]. ¹H NMR (400 MHz, DMSO) δ 10.32 (s, 1H), 8.64 (d, J=2.5 Hz, 1H), 8.39 (s, 1H), 8.15 (t, J=1.9 Hz, 1H), 8.12-8.03 (m, 3H), 7.93 (ddd, J=8.2, 2.2, 1.1 Hz, 1H), 7.54-7.35 (m, 5H), 7.05 (d, J=2.5 Hz, 1H); MS(ES⁺) m/z calc'd for [M+H]⁺ [C22H18N6OS]*414.49, found: 415.2, LCMS: t_{R}=3.39 min, Method A: Column: X-Select CSH C18, (50 mm*3.0 mm, 2.5p) Mobile Phase A: 0.05% Formic Acid in Water:Acetonitrile (95:5) Mobile Phase B: 0.05% Formic Acid in Acetonitrile Flow rate: 1.2 mL/min. Column temperature: 50° C.

### Synthesis of N-(2,4-dichloro-3-(8-methyl-2-(methylamino)-7-oxo-7,8-dihydropyrido[2,3-d]pyrimidin-6-yl)phenyl)-1-(4-fluorophenyl)-1H-pyrazole-3-carboxamide [Example 82]

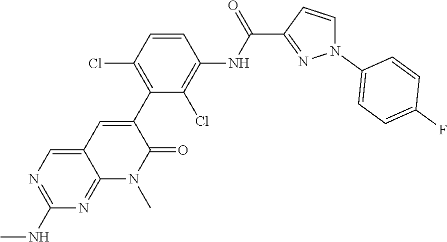 [SMILES: CNc1ncc2cc(-c3c(Cl)ccc(NC(=O)c4ccn(-c5ccc(F)cc5)n4)c3Cl)c(=O)n(C)c2n1]

### Synthesis of Example 7 Followed the Same Procedure Used in Synthetic Method B Except Used

6-(3-amino-2,6-dichloro-phenyl)-8-methyl-2-(methylamino)pyrido[2,3-d]pyrimidin-7-one and 1-(4-fluorophenyl)-1H-pyrazole-3-carboxylic acid. The crude compound was purified by preparative HPLC purification Method A. The preparative fractions were lyophilized to get solid compound which was dried under high vacuum to get the title compound (2.19 mg, 0.004 mmol, 4.71% yield) as an off-white solid. MS (ES⁺) calc'd for [M+H]⁺ [C₂₅H₁₈Cl₂FN₇O₂+H]⁺:539.36, found: 538.08, LCMS: t_{R}: 2.10 min. [Analytical Method: D].

### Synthesis of N-(3-((1H-pyrrolo[2,3-b]pyridin-4-yl)oxy)-4-methylphenyl)-1-(4-fluorophenyl)-1H-pyrazole-3-carboxamide [Example 83]

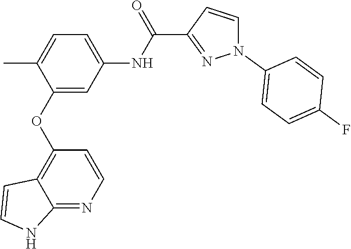 [SMILES: Cc1ccc(NC(=O)c2ccn(-c3ccc(F)cc3)n2)cc1Oc1ccnc2[nH]ccc12]

### Synthesis of Example 13 Followed the Same Procedure Used in Synthetic Method E Except Used

6-(5-amino-4-fluoro-2-methyl-phenyl)-N,7-dimethylpyrido[2,3-d]pyrimidin-2-amine and 1-(oxetan-3-yl)pyrazole-3-carboxylic acid and TCFH (3 eq). The solid was collected by filtration and dried under vacuum to get the title compound (21.3 mg, 0.0490 mmol, 39.05% yield). ¹H NMR (400 MHz, DMSO-d₆): δ 11.72 (br s, 1H), 10.20 (br s, 1H), 8.60 (br s, 1H), 8.16-7.99 (m, 3H), 7.81-7.59 (m, 2H), 7.47-7.27 (m, 4H), 6.99 (br s, 1H), 6.36 (br s, 1H), 6.22 (br s, 1H), 2.13 (br s, 3H); MS(ES⁺)calc'd for [M+H]⁺[C₂₅H₁₉N₃O₃+H]⁺:428.40, found:428.40, LCMS: t_{R}: 1.76 min. [Analytical Method: D].

### Synthesis of N-(3-(1-(4-fluorophenyl)-1H-pyrazole-3-carboxamido)phenyl)-6-(1H-pyrazol-5-yl)picolinamide [Example 84]

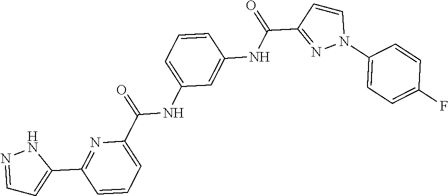 [SMILES: O=C(Nc1cccc(NC(=O)c2ccn(-c3ccc(F)cc3)n2)c1)c1cccc(-c2ccn[nH]2)n1]

Synthesis of Example 16 followed the same procedure used in Synthetic Method D except used N-(3-aminophenyl)-6-(1H-pyrazol-5-yl)pyridine-2-carboxamide; hydrochloride and 1-(4-fluorophenyl)-1H-pyrazole-3-carboxylic acid. The solid was collected by filtration and dried under vacuum to get the title compound (6.68 mg, 14.95% yield) as an off-white solid. ¹H NMR (400 MHz, DMSO-d₆): δ 14.46-12.83 (m, 1H), 10.84-10.44 (m, 1H), 10.30-10.06 (m, 1H), 8.64 (d, J=2.5 Hz, 1H), 8.44-8.36 (m, 1H), 8.18-8.01 (m, 5H), 7.69-7.60 (m, 3H), 7.49-7.38 (m, 3H), 7.09 (t, J=2.0 Hz, 1H), 7.06 (d, J=2.5 Hz, 1H); MS (ES⁺) calc'd for [M+H]⁺ [C₂₅H₁₈FN₇O₂+H]⁺: 468.45, found: 468.00, LCMS: t: 1.52 min. [Analytical Method: D]; Column: X-Select CSH (3.0*50) mm 2.5 u].

### Synthesis of N-(2-fluoro-4-methyl-5-(7-methyl-2-(methylamino)pyrido[2,3-d]pyrimidin-6-yl)phenyl)-1-(4-fluorophenyl)-1H-pyrazole-3-carboxamide [Example 85]

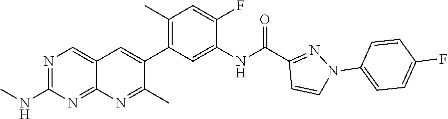 [SMILES: CNc1ncc2cc(-c3cc(NC(=O)c4ccn(-c5ccc(F)cc5)n4)c(F)cc3C)c(C)nc2n1]

### Synthesis of Example 21 Followed the Same Procedure Used in Synthetic Method B Except Used

6-(5-amino-4-fluoro-2-methyl-phenyl)-N,7-dimethylpyrido[2,3-d]pyrimidin-2-amine and 1-(4-fluorophenyl)-1H-pyrazole-3-carboxylic acid. The crude compound was purified by flash column chromatography eluting in (5% MeOH in DCM) to get title compound (8.13 mg, 0.0156 mmol, 15.47% yield) as off white solid. ¹H NMR (400 MHz, DMSO-d₆): δ 9.90 (s, 1H), 9.09 (br s, 1H), 8.63 (d, J=2.5 Hz, 1H), 8.03 (dd, J=9.0, 4.6 Hz, 2H), 7.97 (s, 1H), 7.75-7.63 (m, 1H), 7.59 (d, J=7.9 Hz, 1H), 7.43 (t, J=8.8 Hz, 2H), 7.35 (d, J=11.5 Hz, 1H), 7.01 (d, J=2.5 Hz, 1H), 2.93 (br d, J=4.5 Hz, 3H), 2.35 (s, 3H), 2.06 (s, 3H); MS (ES⁺) calc'd for [M+H]⁺ [C₂₆H₂₁F₂N₇O+H]⁺:486.51, found: 486.10, LCMS: t_{R}:1.74 min. [Analytical Method: D].

### Synthesis of N-(3-((4-(2-acetamido-4-methylthiazol-5-yl)pyrimidin-2-yl)amino)-4-methylphenyl)-1-(4-fluorophenyl)-1H-pyrazole-3-carboxamide [Example 86]

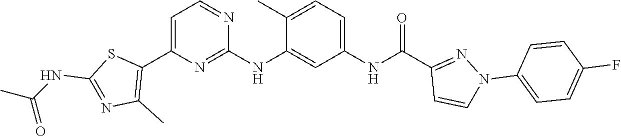 [SMILES: CC(=O)Nc1nc(C)c(-c2ccnc(Nc3cc(NC(=O)c4ccn(-c5ccc(F)cc5)n4)ccc3C)n2)s1]

### Synthesis of Example 24 Followed the Same Procedure Used in Synthetic Method B Except Used

N-[5-[2-(5-amino-2-methyl-anilino)pyrimidin-4-yl]-4-methyl-thiazol-2-yl]acetamide and 1-1-(4-fluorophenyl)-1H-pyrazole-3-carboxylic acid. The crude compound was purified by preparative HPLC purification Method A. The preparative fractions were lyophilized to get solid compound which was dried under high vacuum to get the title compound (4.0 mg, 0.0073 mmol, 8.70% yield) as an off-white solid. ¹H NMR (400 MHz, DMSO-d₆): δ 12.19 (s, 1H), 10.01 (s, 1H), 8.86 (s, 1H), 8.61 (d, J=2.5 Hz, 1H), 8.34 (d, J=5.4 Hz, 1H), 8.05 (dd, J=4.8, 9.3 Hz, 2H), 7.90 (d, J=2.0 Hz, 1H), 7.57 (dd, J=2.1, 8.3 Hz, 1H), 7.41 (t, J=8.8 Hz, 2H), 7.19 (d, J=8.5 Hz, 1H), 7.00 (d, J=2.5 Hz, 1H), 6.95 (d, J=5.4 Hz, 1H), 2.52 (s, 3H), 2.19 (s, 3H), 2.11 (s, 3H); MS(ES⁺) calc'd for[M+H]⁺[C₂₇H₂₃FN₈O₂S+H]⁺:543.59, found: 543.55, LCMS: t_{R}: 1.81 min. [Analytical Method: D].

### Synthesis of N-(6-fluoro-5-((5-methyl-1H-pyrrolo[2,3-b]pyridin-3-yl)methyl)pyridin-2-yl)-1-(4-fluorophenyl)-5-(methylsulfonyl)-1H-pyrazole-3-carboxamide [Example 87]

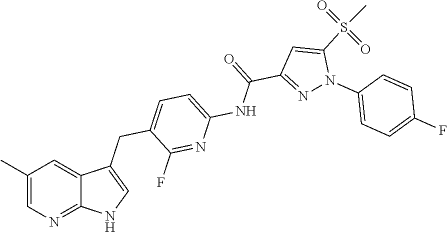 [SMILES: Cc1cnc2[nH]cc(Cc3ccc(NC(=O)c4cc(S(C)(=O)=O)n(-c5ccc(F)cc5)n4)nc3F)c2c1]

To a stirred solution of 1-(4-fluorophenyl)-5-methylsulfonyl-pyrazole-3-carboxylic acid (48 mg, 0.168 mmol, 1.50 eq), N,N,N′,N′-tetramethylchloroformamidinium hexafluorophosphate (47 mg, 0.168 mmol, 1.50 eq) in ACN (0.5000 mL, 0.1684 M) was added N-methylimidazole (0.027 mL, 0.337 mmol, 3.00 eq), followed by addition of tert-butyl 3-[(6-amino-2-fluoro-3-pyridyl)methyl]-5-methyl-pyrrolo[2,3-b]pyridine-1-carboxylate (40 mg, 0.112 mmol, 1.00 eq) at rt. The reaction was stirred at 70° C. for 16 h. The Progress of reaction was monitored by LCMS. After completion of the reaction, the reaction mixture was cooled to 0° C., 4 M HCl in 1,4-dioxane (1 mL) was added and the reaction mixture was stirred at rt for 5 h. After completion of the reaction, the pH of the reaction mixture was neutralized with solid NaHCO₃, diluted ice-cold water and the precipitated solid was filtered, dried under vacuum. The solid was triturated with DCM: n-pentane (1: 4, 5 mL), and dried in vacuo to get the title compound (27.88 mg, 47.05% yield) as an off-white solid. ¹H NMR (400 MHz, DMSO-d₆): δ 11.42 (br s, 1H), 10.61 (br s, 1H), 8.07 (br s, 1H), 7.97 (br d, J=7.0 Hz, 1H), 7.87 (br t, J=8.4 Hz, 1H), 7.80-7.74 (m, 4H), 7.45 (br t, J=7.8 Hz, 2H), 7.28 (br s, 1H), 4.02 (br s, 2H), 3.34 (br s, 3H), 2.35 (br s, 3H); MS (ES⁺) calc'd for [M+H]+[C₂₅H₂₀F₂N₆O₃S+H]⁺: 523.53, found: 523.5, LCMS: t: 1.77 min. [Analytical Method: D]

### Hinge Binding Intermediates

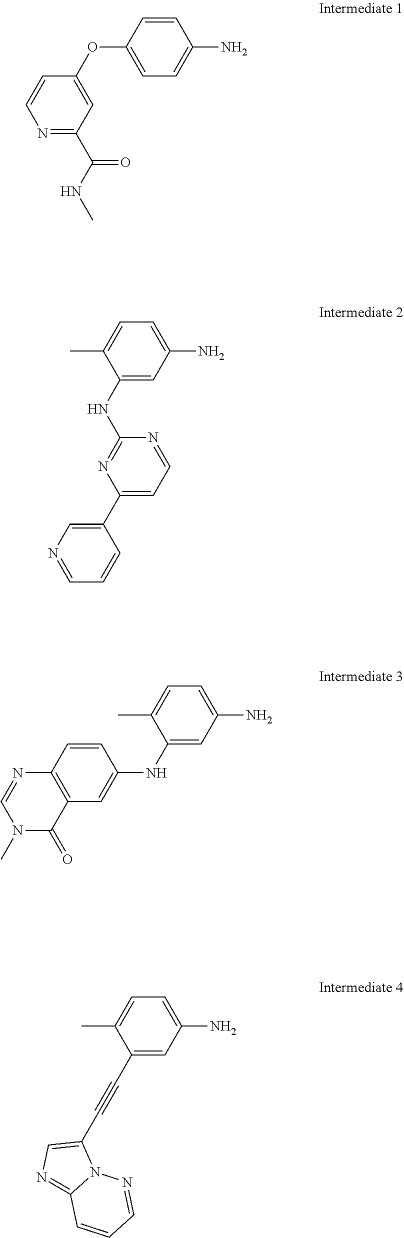 [SMILES: CNC(=O)c1cc(Oc2ccc(N)cc2)ccn1 ; Cc1ccc(N)cc1Nc1nccc(-c2cccnc2)n1 ; Cc1ccc(N)cc1Nc1ccc2ncn(C)c(=O)c2c1 ; Cc1ccc(N)cc1C#Cc1cnc2cccnn12]

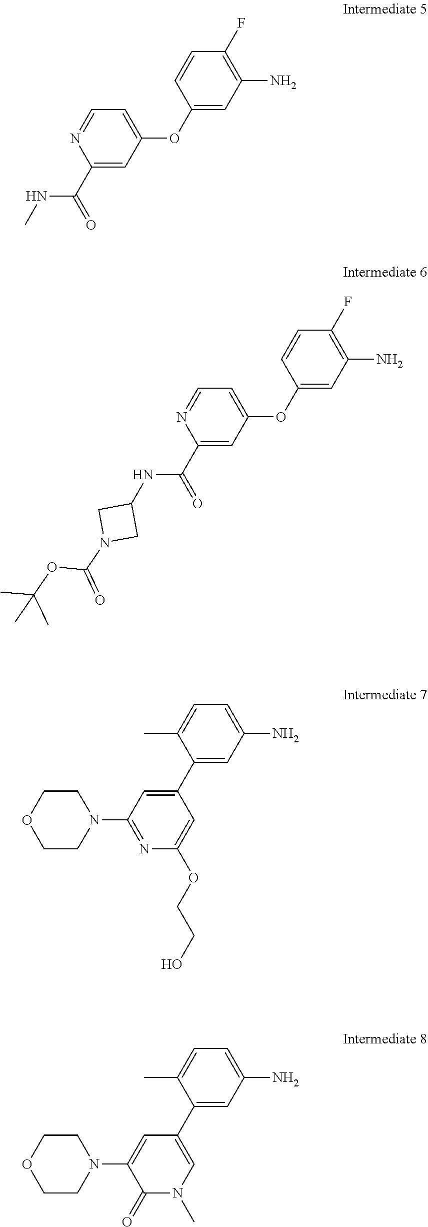 [SMILES: CNC(=O)c1cc(Oc2ccc(F)c(N)c2)ccn1 ; CC(C)(C)OC(=O)N1CC(NC(=O)c2cc(Oc3ccc(F)c(N)c3)ccn2)C1 ; Cc1ccc(N)cc1-c1cc(OCCO)nc(N2CCOCC2)c1 ; Cc1ccc(N)cc1-c1cc(N2CCOCC2)c(=O)n(C)c1]

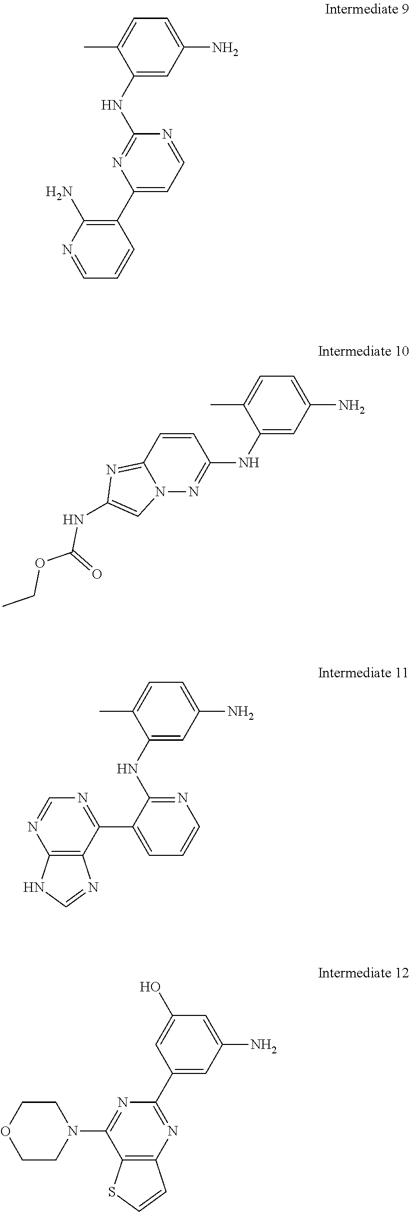 [SMILES: Cc1ccc(N)cc1Nc1nccc(-c2cccnc2N)n1 ; CCOC(=O)Nc1cn2nc(Nc3cc(N)ccc3C)ccc2n1 ; Cc1ccc(N)cc1Nc1ncccc1-c1ncnc2[nH]cnc12 ; Nc1cc(O)cc(-c2nc(N3CCOCC3)c3sccc3n2)c1]

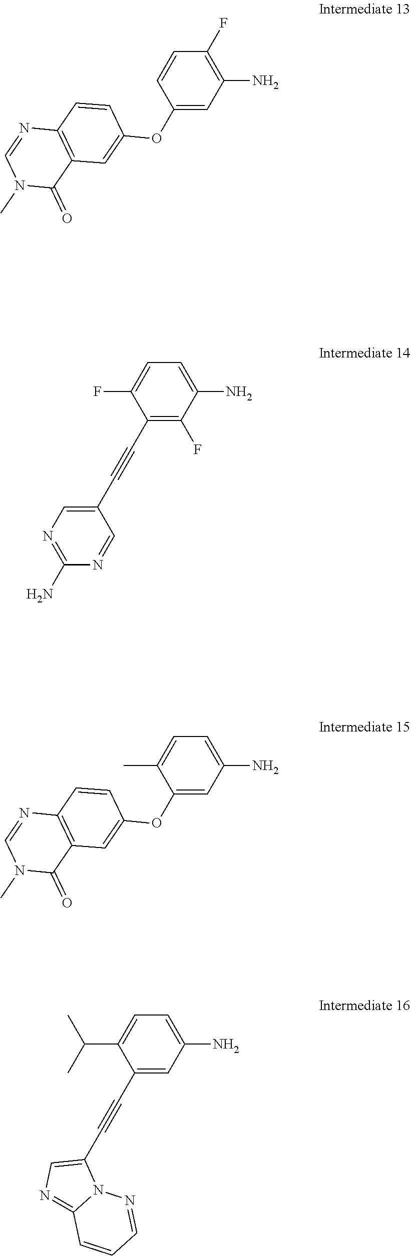 [SMILES: Cn1cnc2ccc(Oc3ccc(F)c(N)c3)cc2c1=O ; Nc1ncc(C#Cc2c(F)ccc(N)c2F)cn1 ; Cc1ccc(N)cc1Oc1ccc2ncn(C)c(=O)c2c1 ; CC(C)c1ccc(N)cc1C#Cc1cnc2cccnn12]

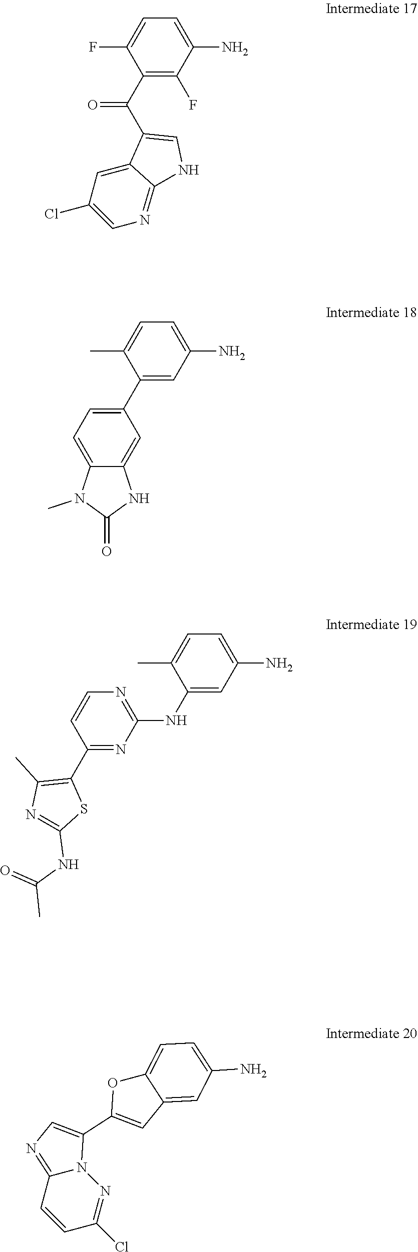 [SMILES: Nc1ccc(F)c(C(=O)c2c[nH]c3ncc(Cl)cc23)c1F ; Cc1ccc(N)cc1-c1ccc2c(c1)[nH]c(=O)n2C ; CC(=O)Nc1nc(C)c(-c2ccnc(Nc3cc(N)ccc3C)n2)s1 ; Nc1ccc2oc(-c3cnc4ccc(Cl)nn34)cc2c1]

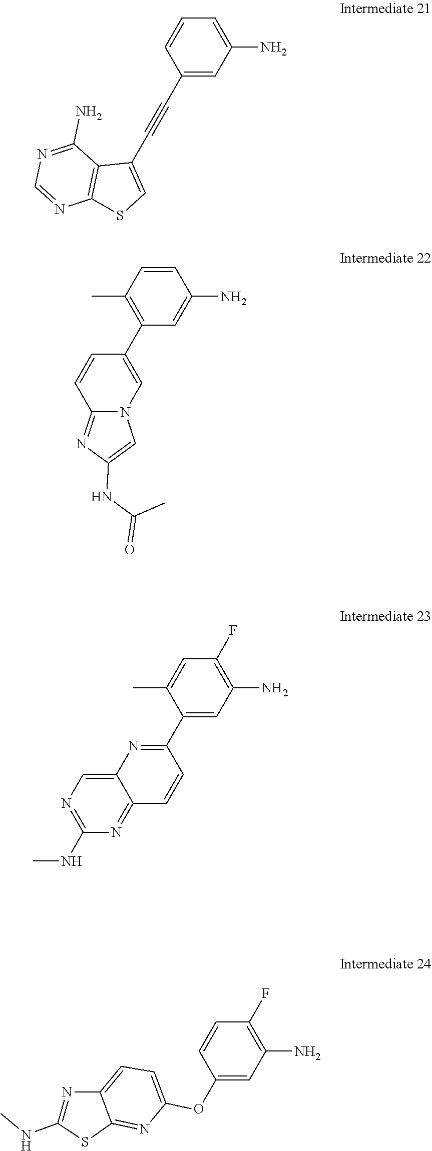 [SMILES: Nc1cccc(C#Cc2csc3ncnc(N)c23)c1 ; CC(=O)Nc1cn2cc(-c3cc(N)ccc3C)ccc2n1 ; CNc1ncc2nc(-c3cc(N)c(F)cc3C)ccc2n1 ; CNc1nc2ccc(Oc3ccc(F)c(N)c3)nc2s1]

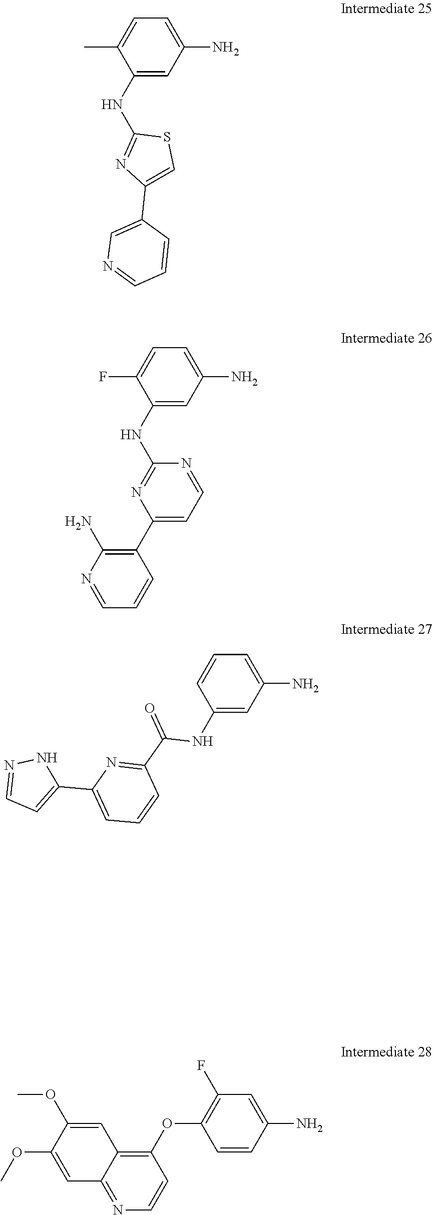 [SMILES: Cc1ccc(N)cc1Nc1nc(-c2cccnc2)cs1 ; Nc1ccc(F)c(Nc2nccc(-c3cccnc3N)n2)c1 ; Nc1cccc(NC(=O)c2cccc(-c3ccn[nH]3)n2)c1 ; COc1cc2nccc(Oc3ccc(N)cc3F)c2cc1OC]

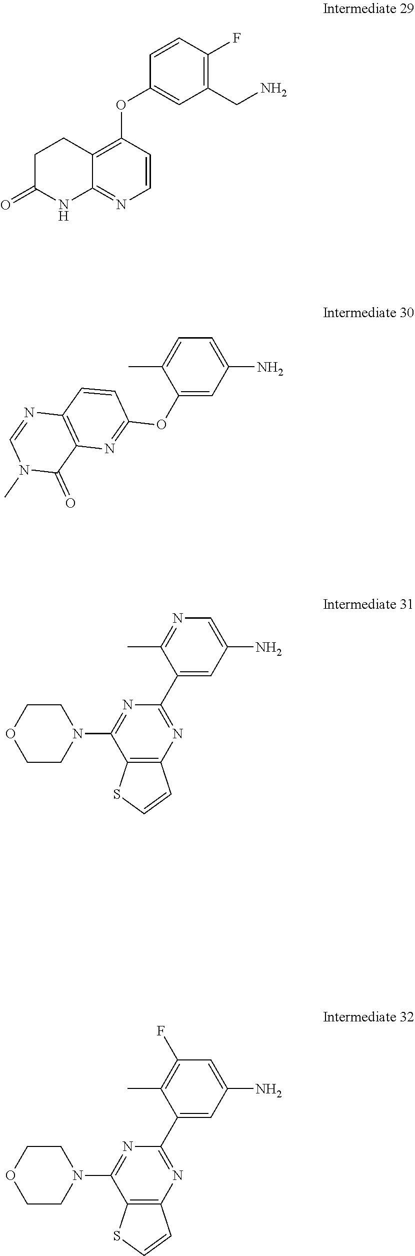 [SMILES: NCc1cc(Oc2ccnc3c2CCC(=O)N3)ccc1F ; Cc1ccc(N)cc1Oc1ccc2ncn(C)c(=O)c2n1 ; Cc1ncc(N)cc1-c1nc(N2CCOCC2)c2sccc2n1 ; Cc1c(F)cc(N)cc1-c1nc(N2CCOCC2)c2sccc2n1]

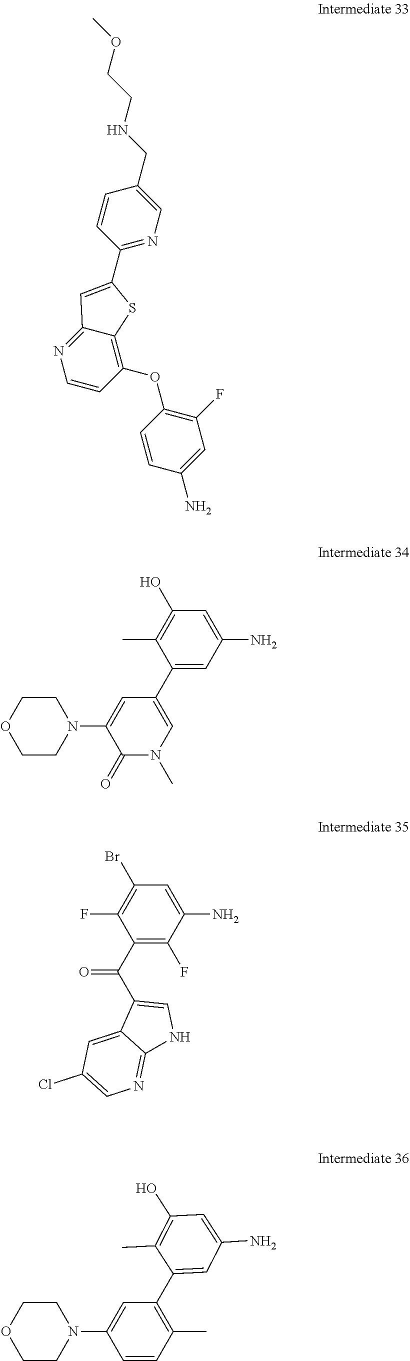 [SMILES: COCCNCc1ccc(-c2cc3nccc(Oc4ccc(N)cc4F)c3s2)nc1 ; Cc1c(O)cc(N)cc1-c1cc(N2CCOCC2)c(=O)n(C)c1 ; Nc1cc(Br)c(F)c(C(=O)c2c[nH]c3ncc(Cl)cc23)c1F ; Cc1ccc(N2CCOCC2)cc1-c1cc(N)cc(O)c1C]

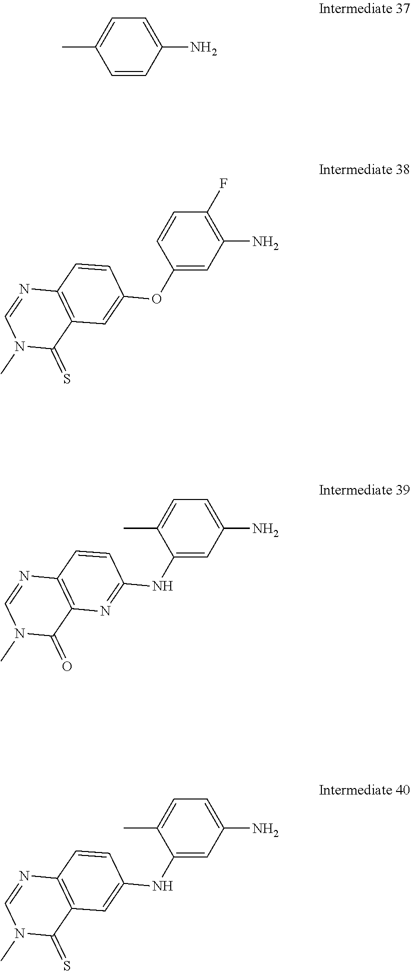

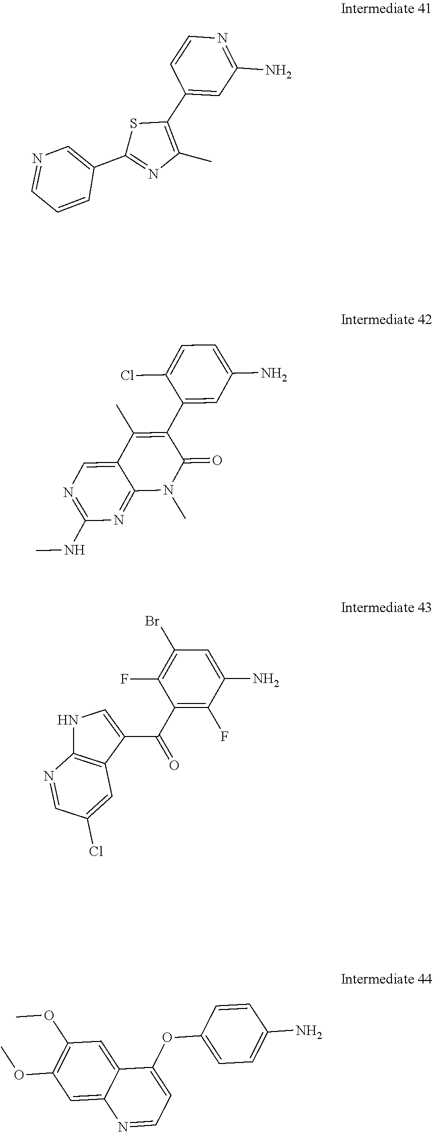 [SMILES: Cc1nc(-c2cccnc2)sc1-c1ccnc(N)c1 ; CNc1ncc2c(C)c(-c3cc(N)ccc3Cl)c(=O)n(C)c2n1 ; Nc1cc(Br)c(F)c(C(=O)c2c[nH]c3ncc(Cl)cc23)c1F ; COc1cc2nccc(Oc3ccc(N)cc3)c2cc1OC]

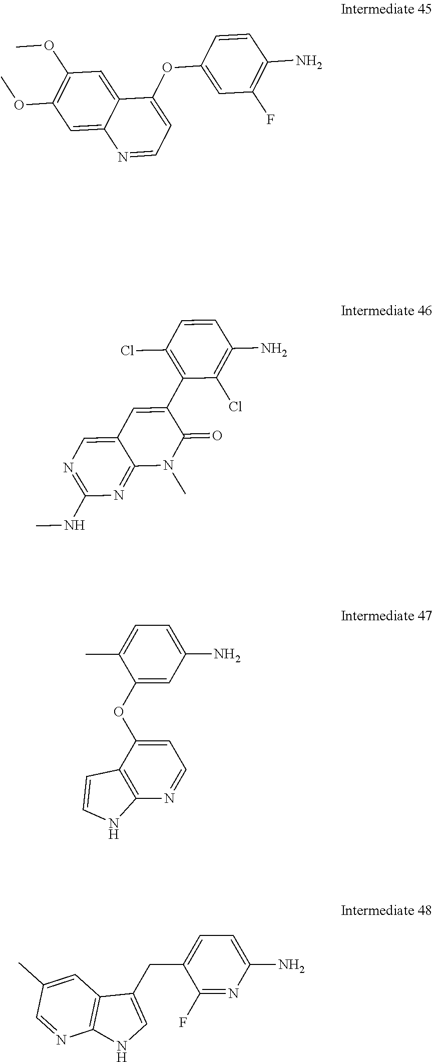 [SMILES: COc1cc2nccc(Oc3ccc(N)c(F)c3)c2cc1OC ; CNc1ncc2cc(-c3c(Cl)ccc(N)c3Cl)c(=O)n(C)c2n1 ; Cc1ccc(N)cc1Oc1ccnc2[nH]ccc12 ; Cc1cnc2[nH]cc(Cc3ccc(N)nc3F)c2c1]

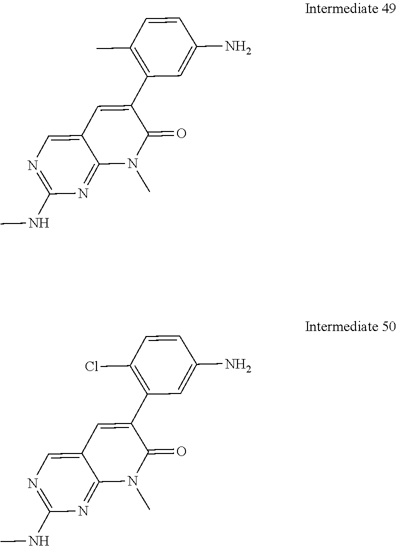 [SMILES: CNc1ncc2cc(-c3cc(N)ccc3C)c(=O)n(C)c2n1 ; CNc1ncc2cc(-c3cc(N)ccc3Cl)c(=O)n(C)c2n1]

### Synthesis of Hinge Intermediates:

### Synthesis of 4-(4-aminophenoxy)-N-methylpicolinamide [Intermediate 1]

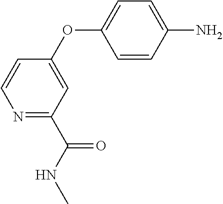 [SMILES: CNC(=O)c1cc(Oc2ccc(N)cc2)ccn1]

Available from Commercial Sources

### Synthesis of 6-methyl-N1-(4-(pyridin-3-yl)pyrimidin-2-yl)benzene-1,3-diamine [Intermediate 2]

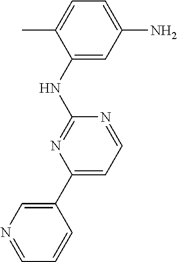 [SMILES: Cc1ccc(N)cc1Nc1nccc(-c2cccnc2)n1]

Available from Commercial Sources

### Synthesis of 6-((5-amino-2-methylphenyl)amino)-3-methylquinazolin-4(3H)-one [Intermediate 3]

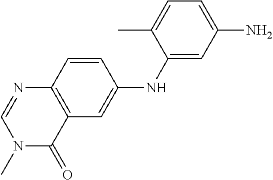 [SMILES: Cc1ccc(N)cc1Nc1ccc2ncn(C)c(=O)c2c1]

Intermediate 3 is known in the literature and the synthesis can be performed according to the reference below:
Reference: WO2006/24834A1

### Synthesis of 3-(imidazo[1,2-b]pyridazin-3-ylethynyl)-4-methylaniline [Intermediate 4]

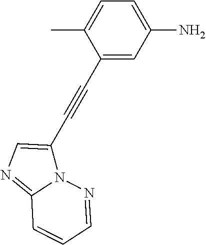 [SMILES: Cc1ccc(N)cc1C#Cc1cnc2cccnn12]

Intermediate 4 is known in the literature and the synthesis can be performed according to the reference below:
Reference: KR2020/88945, 2020, A

### Synthesis of 4-(3-amino-4-fluorophenoxy)-N-methylpicolinamide [Intermediate 5]

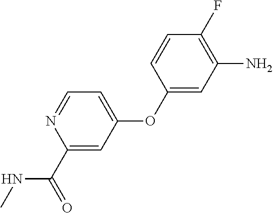 [SMILES: CNC(=O)c1cc(Oc2ccc(F)c(N)c2)ccn1]

Available from Commercial Sources

### Synthesis of tert-butyl 3-(4-(3-amino-4-fluorophenoxy)picolinamido)azetidine-1-carboxylate [Intermediate 6]

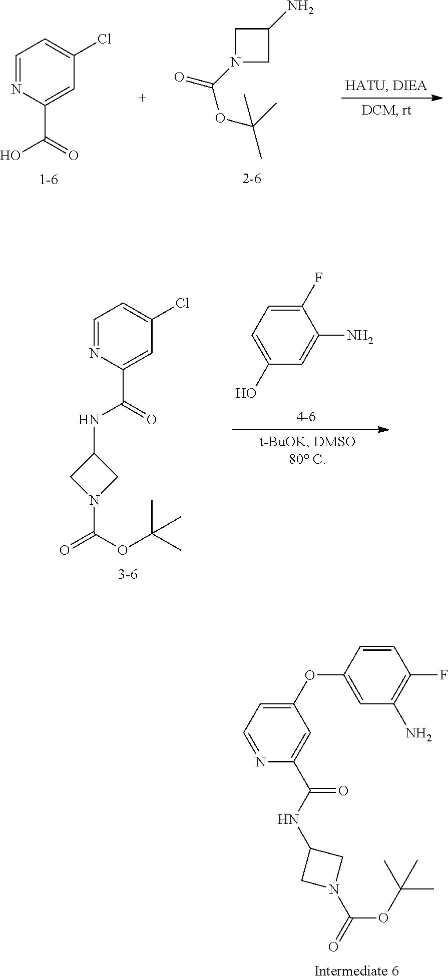 [SMILES: O=C(O)c1cc(Cl)ccn1 ; CC(C)(C)OC(=O)N1CC(N)C1 ; CC(C)(C)OC(=O)N1CC(NC(=O)c2cc(Cl)ccn2)C1 ; Nc1cc(O)ccc1F ; CC(C)(C)OC(=O)N1CC(NC(=O)c2cc(Oc3ccc(F)c(N)c3)ccn2)C1]

### tert-butyl 3-(4-chloropicolinamido)azetidine-1-carboxylate [3-6]

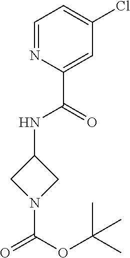 [SMILES: CC(C)(C)OC(=O)N1CC(NC(=O)c2cc(Cl)ccn2)C1]

To a solution of 4-chloropicolinic acid (2 g, 12.69 mmol) in DCM (127 mL) was tert-butyl 3-aminoazetidine-1-carboxylate (2.62 g, 15.22 mmol) and HATU (5.78 g, 15.22 mmol). To the above mixture DIEA (4.43 mL, 25.38 mmol) was added drop wise at rt and stirring continued further for 16 h at rt. At the end of this period mixture was diluted with DCM (100 mL) and washed with Sat. NaHCO₃ solution (2×50 mL) followed by water (50 mL). Organic layer was collected and dried (Na₂SO₄), filtered and solvent evaporated to dryness. The crude was chromatographed over SiO₂ using gradient of EtOAc in DCM. Yield: 4.0 g (quantitative). ¹H NMR (500 MHz, DMSO) δ 9.51 (d, J=7.6 Hz, 1H), 8.65 (d, J=5.2 Hz, 1H), 8.06-7.96 (m, 1H), 7.78 (dd, J=5.3, 2.2 Hz, 1H), 4.69 (qt, J=7.9, 5.8 Hz, 1H), 4.17-3.87 (m, 4H), 1.39 (s, 10H).

### tert-butyl 3-(4-(3-amino-4-fluorophenoxy)picolinamido)azetidine-1-carboxylate [Intermediate 6]

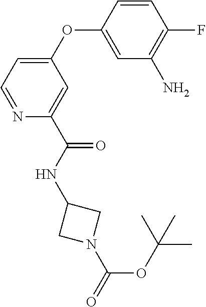 [SMILES: CC(C)(C)OC(=O)N1CC(NC(=O)c2cc(Oc3ccc(F)c(N)c3)ccn2)C1]

To a solution of 3-amino-4-fluorophenol (2.01 g, 15.22 mmol) in DMSO (10 ml) was added t-BuOK (1.73 g, 15.40 mmol) at rt and stirring continued further for 2 h at rt. To the above mixture a solution of tert-butyl 3-(4-chloropicolinamido)azetidine-1-carboxylate (4.00 g, 12.83 mmol) in DMSO (7.8 mL) was added at rt and after complete addition, the mixture was heated at 80 C for 20 h. At the end of this period the reaction mixture was cooled to rt and water was added and extracted with EtOAc (2×125 mL). The organic layer was washed with water (100 mL) and brine (2×50 mL). The organic layer was collected and dried (Na₂SO₄), filtered and the solvent was evaporated and the crude was chromatographed over SiO₂ using gradient of EtOAc in Hexanes resulting in 3.8 g, 77% yield of the title compound. ¹H NMR (600 MHz, DMSO) δ 9.42 (d, J=7.6 Hz, 1H), 8.53 (d, J=5.6 Hz, 1H), 7.39 (d, J=2.6 Hz, 1H), 7.17 (dd, J=5.6, 2.6 Hz, 1H), 7.09 (dd, J=11.2, 8.6 Hz, 1H), 6.53 (dd, J=7.6, 2.9 Hz, 1H), 6.31 (dt, J=8.6, 3.2 Hz, 1H), 5.46 (s, 2H), 4.65 (qt, J=7.9, 5.9 Hz, 1H), 4.17-3.75 (m, 4H), 1.39 (s, 9H).

### Synthesis of 2-((4-(5-amino-2-methylphenyl)-6-morpholinopyridin-2-yl)oxy)ethan-1-ol [Intermediate 7]

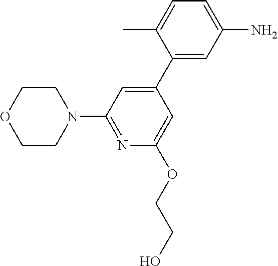 [SMILES: Cc1ccc(N)cc1-c1cc(OCCO)nc(N2CCOCC2)c1]

Intermediate 7 is known in the literature and the synthesis can be performed according to the reference below:
Reference: Journal of Medicinal Chemistry, 2020, vol. 63, #5, p. 2013-2027

### Synthesis of 5-(5-amino-2-methylphenyl)-3-morpholinopyridin-2(1H)-one [Intermediate 8]

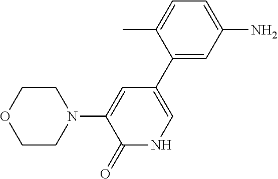 [SMILES: Cc1ccc(N)cc1-c1c[nH]c(=O)c(N2CCOCC2)c1]

Intermediate 8 is known in the literature and the synthesis can be performed according to the reference below:
Reference: Journal of Medicinal Chemistry, 2020, vol. 63, #5, p. 2013-2027

### Synthesis of N1-(4-(2-aminopyridin-3-yl)pyrimidin-2-yl)-6-methylbenzene-1,3-diamine [Intermediate 9]

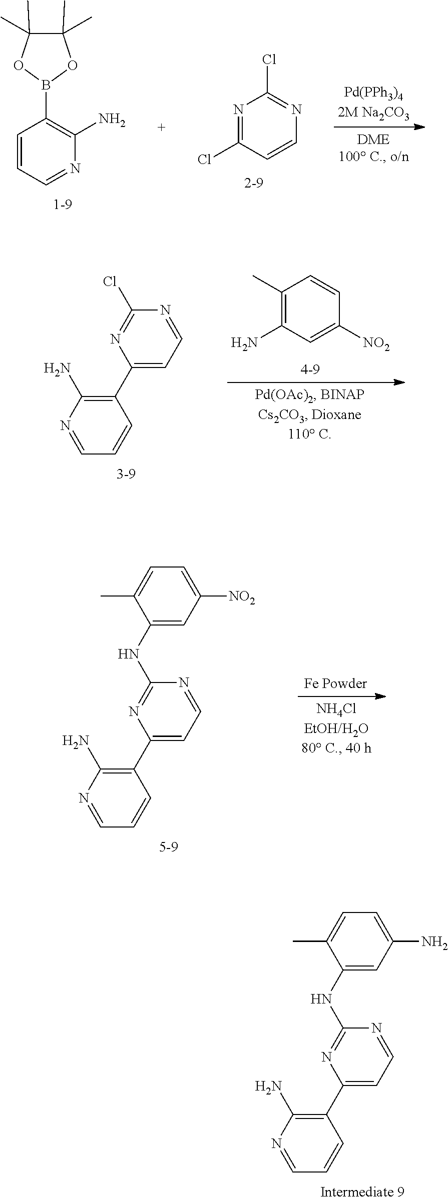 [SMILES: CC1(C)OB(c2cccnc2N)OC1(C)C ; Clc1ccnc(Cl)n1 ; Nc1ncccc1-c1ccnc(Cl)n1 ; Cc1ccc([N+](=O)[O-])cc1N ; Cc1ccc([N+](=O)[O-])cc1Nc1nccc(-c2cccnc2N)n1 ; Cc1ccc(N)cc1Nc1nccc(-c2cccnc2N)n1]

### N3-[4-(2-amino-3-pyridyl)pyrimidin-2-yl]-4-methyl-benzene-1,3-diamine [Intermediate 9]

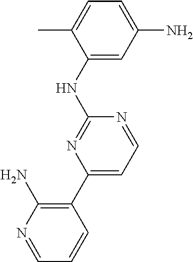 [SMILES: Cc1ccc(N)cc1Nc1nccc(-c2cccnc2N)n1]

To a mixture of 4-(2-amino-3-pyridyl)-N-(2-methyl-5-nitro-phenyl)pyrimidin-2-amine (0.55 g, 1.71 mmol, 1.00 eq), ammonium chloride (3.0 equiv.), and Fe powder (3.5 equiv.) in EtOH (95%) (8.25 mL, 0.067 M), and water (0.95 mL, 0.067 M) was stirred at 80° C. for 18 h. Reaction was monitored by TLC and LCMS. Then ammonium chloride (1.0 equiv.), Fe powder (1.1 equiv.), and water (0.95 mL, 0.067 M) was stirred at 80° C. for 24 h. Then ammonium chloride (1.0 equiv.), Fe powder (1.1 equiv.), and water (0.95 mL, 0.067 M) was stirred at 80° C. for 24 h. Reaction mixture was cooled to rt and filtered through celite bed, washed with 10% MeOH-DCM (50 mL). The filtrate was collected and dried. Crude was purified by column chromatography over silica gel, ISCO, CombiFlash, 12 g cartridge (dry load, 20% MeOH-DCM with 1% triethylamine in MeOH as eluent) to afford as yellow solid. Then the HCl salt was partitioned between 10% MeOH-DCM (10 mL) and satd. NaHCO₃ (10 mL) and stirred for 30 min. aqueous layer was extracted with 10% MeOH-DCM (2×5 mL). The combined organic layers were dried, concentrated to provide 0.22 g, 32.9% of the tile compound as a yellow solid. ¹H NMR (500 MHz, DMSO-d₆) δ 8.68 (s, 1H), 8.27 (d, J=5.4 Hz, 1H), 8.07 (dd, J=7.9, 1.8 Hz, 1H), 8.03 (dd, J=4.7, 1.8 Hz, 1H), 7.46 (s, 2H), 7.14 (d, J=5.5 Hz, 1H), 6.91 (d, J=8.3 Hz, 1H), 6.69-6.53 (m, 1H), 6.44 (d, J=2.6 Hz, 1H), 6.39 (dd, J=8.3, 2.6 Hz, 1H), 4.90 (s, 2H), 2.04 (s, 3H). MS(ESI⁺) m/z calc'd for [M+H]⁺ C₁₆H₁₆N₆+H]⁺: 293.1 found: 293.2, LCMS t_{R}=0.92 min [Analytical Method B].

### 4-(2-Amino-3-pyridyl)-N-(2-methyl-5-nitro-phenyl)pyrimidin-2-amine [5-9]

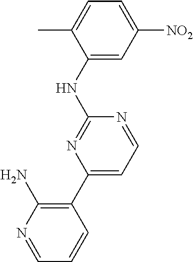 [SMILES: Cc1ccc([N+](=O)[O-])cc1Nc1nccc(-c2cccnc2N)n1]

Nitrogen gas was bubbled through a mixture of 3-(2-chloropyrimidin-4-yl)pyridin-2-amine (1.50 g, 7.26 mmol, 1.00 eq), 2-Methyl-5-nitroaniline (3.30 g, 21.7 mmol, 3.00 eq), BINAP (0.45 g, 0.726 mmol, 0.100 eq), Pd(OAc)₂ (0.16 g, 0.726 mmol, 0.100 eq), and Cs₂CO₃ (4.73 g, 14.5 mmol, 2.00 eq) in 1,4-Dioxane (73 mL, 0.1000 M) for 20 min. Reaction was heated to 80° C. and stirred for 2 h. Reaction was cooled to room temperature, diluted with ethyl acetate (100 mL) and filtered through celite. Volatiles were removed under reduced pressure and loaded onto silica gel. Crude material was purified by CombiFlash chromatography (dry load; silica gel; 20-50% ethyl acetate in hexanes) to afford 4-(2-amino-3-pyridyl)-N-(2-methyl-5-nitro-phenyl)pyrimidin-2-amine (0.75 g, 2.33 mmol, 32.00% yield) as a pale yellow solid. ¹H NMR (500 MHz, DMSO-d₆) δ 9.39 (s, 1H), 8.51 (d, 1H), 8.15 (m, 3H), 8.05 (m, 3H), 7.57 (Br, 2H), 7.48 (d, 1H), 6.66 (dd, 1H), 2.42 (s, 3H). MS (ES⁺) m/z calc'd for [M+H]⁺ [C₁₆H₁₄N₆O₂+H]⁺: 323.1 found 323.4, LCMS t_{R}=2.92 min [Analytical Method A-6].

### 3-(2-Chloropyrimidin-4-yl)pyridin-2-amine [3-9]:

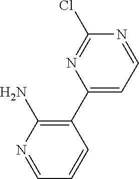 [SMILES: Nc1ncccc1-c1ccnc(Cl)n1]

Nitrogen gas was bubbled through a mixture of 2-Aminopyridine-3-boronic acid pinacol ester (0.44 g, 2.00 mmol, 1.00 eq), 2,4-Dichloropyrimidine (0.45 g, 3.00 mmol, 1.50 eq) and Pd(PPh₃)₄ (0.46 g, 0.400 mmol, 0.200 eq) in 4:1 DME:2M Na₂CO₃ (0.25 M, 8 mL) for 15 min. Pressure vessel was capped under nitrogen atmosphere and lowered down to oil bath at 105-110° C. and stirred overnight. Reaction mixture was filtered through celite, washed with ethyl acetate, and loaded onto silica gel. Crude material was purified by combiflash chromatography (dry load; SiO₂; 10-50% ethyl acetate in Hexanes) to afford 3-(2-chloropyrimidin-4-yl)pyridin-2-amine (0.30 g, 1.34 mmol, 66.79% yield). ¹H NMR (500 MHz, CDCl₃) δ 8.62 (d, 1H), 8.25 (dd, 1H), 7.98 (dd, 1H), 7.62 (d, 1H), 6.92 (Br, 1H), 6.72 (dd, 1H). MS (ES⁺) m/z calc'd for [M+H]⁺ [C₉H₇ClN₄+H]⁺: 206.0, Mass (ES⁺) found: 207.1 [M+H]⁺, LCMS t_{R}=2.52 min [Analytical Method A-6].

### Synthesis of ethyl (6-((5-amino-2-methylphenyl)amino)imidazo[1,2-b]pyridazin-2-yl)carbamate-2,2,2-trifluoroacetaldehyde [Intermediate 10]:

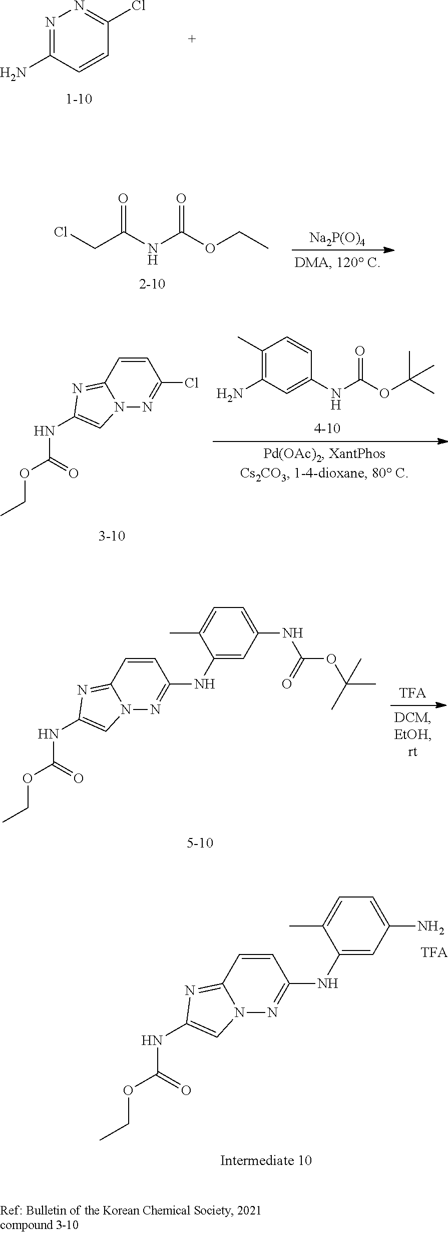

### ethyl (6-((5-((tert-butoxycarbonyl)amino)-2-methylphenyl)amino)imidazo[1,2-b]pyridazin-2-yl)carbamate [5-10]:

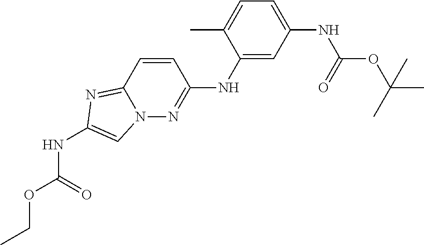 [SMILES: CCOC(=O)Nc1cn2nc(Nc3cc(NC(=O)OC(C)(C)C)ccc3C)ccc2n1]

Pd(OAc)₂ 975 mg, 0.033 mmol) was added to a mixture of ethyl (6-chloroimidazo[1,2-b]pyridazine-2-yl) (784 mg, 0.325 mmol), tert-butyl (3-amino-4-methylphenyl)carbamate (869 mg, 0.390 mmol), Xantphos (238 mg, 0.049 mmol) and Cs₂CO₃ (1484 mg, 0.455 mmol) in 1,4-dioxane (30 mL) and the resulting mixture stirred at 80 C for 12 h. Following cooling, the aqueous phase was extracted twice with EtOAc (2×30 mL) and the combined organic phases were washed with water (100 mL) and brine (100 mL), then filtered and concentrated. The residue was purified by flash chromatography on silica gel eluting with DCM/MeOH 99/1 gradually increasing to 90/10 to yield crude ethyl (6-((5-((tert-butoxycarbonyl)amino)-2-methylphenyl)amino)imidazo[1,2-b]pyridazin-2-yl)carbamate (580 mg) which was further purified by trituration with DCM/hexanes to give 562 mg, 40% yield of the title compound as a yellow/green solid. ¹H-NMR (400 MHz, CDCl₃): δ 7.75 (bs, 1H), 7.46 (bs, 1H), 7.40 (d, 1H, J=9.6 Hz), 7.00 (bs, 2H), 6.63 (d, 1H, J=9.2 Hz), 4.11 (q, 2H, J=7.2 Hz), 3.78 (s, 3H), 2.09 (s, 3H), 1.37 (s, 9H), 1.20 (t, 3H, J=7.2 Hz); MS (ES⁺): m/z 427.34 [M+H]⁺

### ethyl (6-((5-amino-2-methylphenyl)amino)imidazo[1,2-b]pyridazin-2-yl)carbamate-2,2,2-trifluoroacetaldehyde [Intermediate 10]

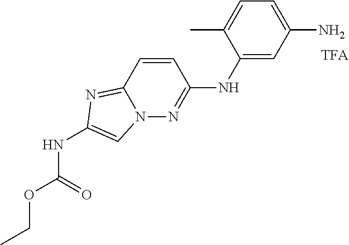 [SMILES: CCOC(=O)Nc1cn2nc(Nc3cc(N)ccc3C)ccc2n1]

A solution of ethyl (6-((5-((tert-butoxycarbonyl)amino)-2-methylphenyl)amino)imidazo[1,2-b]pyridazin-2-yl)carbamate (0.50 g, 1.17 mmol, 1.00 eq) in a mixture of DCM (8.3333 mL, 0.0469 M) and ethanol (8.3333 mL, 0.0469 M) was charged with trifluoroacetic acid (2.0 mL, 26.1 mmol, 22.3 eq) and stirred at rt for 16 h. The reaction mixture was concentrated in vacuo and dried further on the high-vac pump overnight. This resulted in 0.516 g, 100% yield of the title compound as an off-white solid ¹H NMR (600 MHz, DMSO) δ 10.38 (s, 1H), 10.12 (s, 1H), 8.61 (s, 1H), 8.05 (d, J=2.3 Hz, 1H), 7.81 (d, J=9.6 Hz, 1H), 7.75 (s, 1H), 7.35-7.31 (m, 1H), 7.17 (d, J=9.7 Hz, 1H), 6.99 (dd, J=8.0, 2.3 Hz, 1H), 4.15 (q, J=7.1 Hz, 2H), 2.31 (s, 3H), 1.24 (t, J=7.1 Hz, 3H); MS (ES⁺) m/z calc'd for [M+H]⁺[C18H₁₈N₆O₂+H]⁺: 327.36 found 327.0, LCMS: t_{R}=3.40 min [Analytical Method: 05991008_AA1.lcm].

### Synthesis of N1-(3-(9H-purin-6-yl)pyridin-2-yl)-6-methylbenzene-1,3-diamine [Intermediate 11]

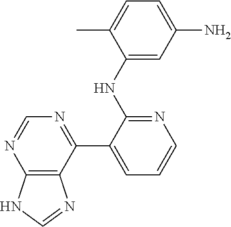 [SMILES: Cc1ccc(N)cc1Nc1ncccc1-c1ncnc2[nH]cnc12]

Intermediate 11 is known in the literature and the synthesis can be performed according to the reference below:
Reference: CN-103102349-B

### Synthesis of 3-amino-5-(4-morpholinothieno[3,2-d]pyrimidin-2-yl)phenol [Intermediate 12]

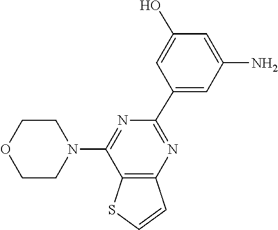 [SMILES: Nc1cc(O)cc(-c2nc(N3CCOCC3)c3sccc3n2)c1]

Intermediate 12 is known in the literature and the synthesis can be performed according to the reference below:
Reference: Chemical Biology and Drug Design, 2019, vol. 94, #6, p. 2013-2022

### Synthesis of 6-(3-amino-4-fluorophenoxy)-3-methylquinazolin-4(3H)-one [Intermediate 13]

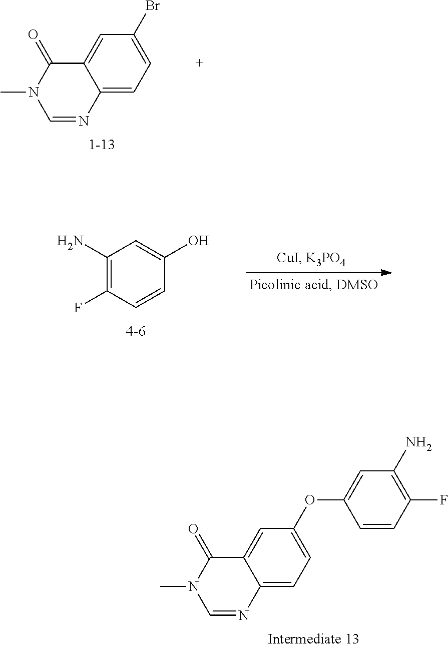 [SMILES: Cn1cnc2ccc(Br)cc2c1=O ; Nc1cc(O)ccc1F ; Cn1cnc2ccc(Oc3ccc(F)c(N)c3)cc2c1=O]

An oven dried round-bottom flask was charged with 6-bromo-3-methylquinazolin-4(3H)-one (35.00 g, 146 mmol), 3-amino-4-fluorophenol (37.20 g, 293 mmol), K₃PO₄ (62.10 g, 293 mmol), CuI (2.79 g, 14.6 mmol) and picolinic acid (3.60 g, 29.2 mmol). To this mixture was added DMSO (500 mL), and the flask was purged with nitrogen gas for 20 min. The reaction was then heated at 100° C. for 12 h. The reaction was cooled to room temperature, and crushed ice (500 ml) was added. The precipitate obtained was filtered and washed with EtOAc (500 ml), then suspended in a mixture of 20% methanol in DCM solution (1500 ml) and filtered to remove the residual copper iodide. The filtrate was evaporated to get the title compound 6-(3-amino-4-fluoro-phenoxy)-3-methyl-quinazolin-4-one (31.00 g, 72.22%) as a greyish brown solid. ¹H NMR (500 MHz, DMSO-d₆) δ 8.29 (s, 1H), 7.69 (d, J=8.8 Hz, 1H), 7.50 (dd, J=8.8, 2.9 Hz, 1H), 7.44 (d, J=2.9 Hz, 1H), 7.02 (dd, J=11.2, 8.7 Hz, 1H), 6.46 (dd, J=7.7, 2.9 Hz, 1H), 6.22 (dt, J=8.6, 3.2, 3.2 Hz, 1H), 5.35 (s, 2H), 3.46 (s, 3H). MS (ES⁺) m/z calcd for [M+H]⁺ [C₁₅H₁₂FN₃O₂+H]⁺: 285.1 found 286.0, LCMS t_{R}=1.60 min [Analytical Method: B]

### Synthesis of 5-((3-amino-2,6-difluorophenyl)ethynyl)pyrimidin-2-amine [Intermediate 14]

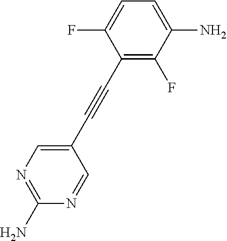 [SMILES: Nc1ncc(C#Cc2c(F)ccc(N)c2F)cn1]

Intermediate 14 is known in the literature and the synthesis can be performed according to the reference below:
Reference: US2019/169166, 2019, A1

### Synthesis of 6-(5-amino-2-methylphenoxy)-3-methylquinazolin-4(3H)-one [Intermediate 15]

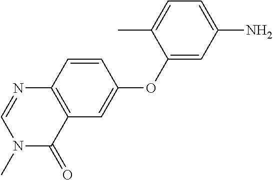 [SMILES: Cc1ccc(N)cc1Oc1ccc2ncn(C)c(=O)c2c1]

Intermediate is known in the literature and the synthesis can be performed according to the reference below:
Reference: WO2006/24834A1

### Synthesis of 3-(imidazo[1,2-b]pyridazin-3-ylethynyl)-4-isopropylaniline [Intermediate 16]

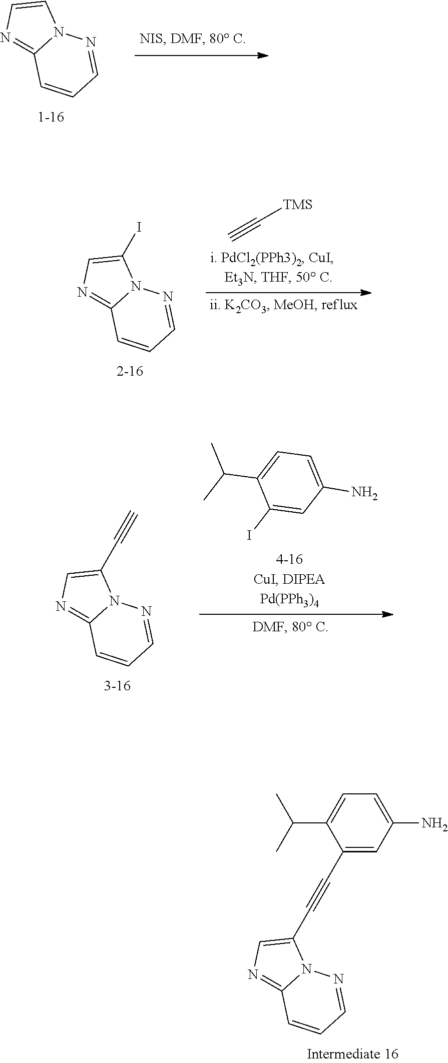

### 3-iodoimidazo[1,2-b]pyridazine [2-16]

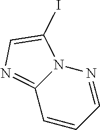

The headspace of a mixture of imidazo[1,2-b]pyridazine (5.57 g, 46.8 mmol, 1.00 eq) and NIS (12.62 g, 56.1 mmol, 1.20 eq) in DMF (0.53 M, 88 mL) was purged with nitrogen prior to being lowered into an oil bath and stirred at 80° C. overnight. The reaction mixture was cooled to rt, diluted with DCM, and transferred to a separatory funnel where it was washed with H₂O (3×), LiCl (aq., 1M), H₂O, and then brine before being dried over sodium sulfate. Concentration in vacuo afforded 8.32 g, 33.8 mmol, 72.34% yield of the title compound as a brown solid. ¹H NMR (500 MHz, DMSO-d₆) δ 8.65 (dd, J=4.4, 1.5 Hz, 1H), 8.12 (dd, J=9.2, 1.5 Hz, 1H), 7.91 (s, 1H), 7.29 (dd, J=9.2, 4.4 Hz, 1H); MS (ES⁺) m/z calc'd for [M+H]⁺[C₆H₄IN₃+H]⁺: 245.9 found 245.9, LCMS: t_{R}=3.52 min [Analytical Method: 05991008_AA1.lcm]

### 3-ethynylimidazo[1,2-b]pyridazine [3-16]

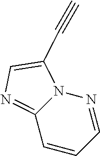

A flame dried pressure vessel containing ethynyl(trimethyl)silane (4.1 mL, 28.8 mmol, 3.50 eq), 3-iodoimidazo[1,2-b]pyridazine (2.01 g, 8.22 mmol, 1.00 eq), Pd(PPh₃)₂Cl₂ (0.29 g, 0.411 mmol, 0.0500 eq), and Et₃N (2.5 mL, 18.1 mmol, 2.20 eq) in THF (0.2M, 41 mL) was purged with nitrogen at rt for 10 minutes prior to the addition of CuI (0.047 g, 0.247 mmol, 0.0300 eq). The resulting mixture was lowered into an oil bath at 50° C. and stirred overnight. The reaction was cooled to rt, additional ethynyl(trimethyl)silane (1.5 equiv., 1.76 mL) added, headspace purged with nitrogen, and lowered back into oil bath to stir overnight again at 50° C. The reaction mixture was concentrated in vacuo. The resulting residue was dissolved in MeOH (100 mL), K₂CO₃ (0.2 equiv., 0.227 g) was added, and the resulting reaction mixture was stirred at rt overnight. Began heating the mixture to 50° C. the following morning. After 2.66 hours additional K₂CO₃ (0.8 equiv., 0.909 g) was added and the reaction mixture was heated further to reflux. After 2.5 hours the reaction was cooled to rt, concentrated in vacuo, and the resulting residue taken up in EtOAc and water prior to filtering through celite (rinsing with EtOAc). Aqueous was extracted further with EtOAc and combined organics were rinsed with brine and dried over sodium sulfate before concentrating in vacuo to afford 0.99 g, 6.90 mmol crude title compound as a solid which was used directly in the subsequent step. ¹H NMR (600 MHz, DMSO-d₆) δ 8.65 (dd, J=4.4, 1.5 Hz, 1H), 8.21 (dd, J=9.2, 1.5 Hz, 1H), 8.11 (s, 1H), 7.35 (dd, J=9.2, 4.4 Hz, 1H), 4.95 (s, 1H); MS (ES⁺) m/z calc'd for [M+H]⁺ [C₈H₅N₃+H]⁺: 144.1 found 144.2, LCMS: t_{R}=3.00 min [Analytical Method: 05991008_AA1.lcm]

### 3-(imidazo[1,2-b]pyridazin-3-ylethynyl)-4-isopropylaniline [Intermediate 16]

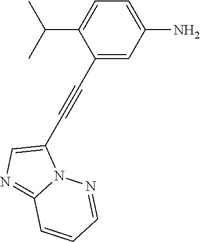 [SMILES: CC(C)c1ccc(N)cc1C#Cc1cnc2cccnn12]

To a solution of 3-ethynylimidazo[1,2-b]pyridazine (1.76 g, 12.3 mmol, 1.00 eq) in DMF (12.299 mL, 1 M) was added 3-iodo-4-isopropyl-aniline (3.99 g, 15.3 mmol, 1.24 eq), tetrakis(triphenylphosphine)palladium(0) (0.93 g, 0.803 mmol, 0.0653 eq), copper(I) iodide (0.38 g, 1.98 mmol, 0.161 eq), and diisopropylethylamine (6.5 mL, 36.9 mmol, 3.00 eq). The solution was stirred at 80° C. for 15 hours. The reaction solution was quenched with water (100 mL) and extracted with EtOAc (50 mL×3), The combined organic layers were washed with saturated brine, dried over MgSO4, filtered, and concentrated in vacuo. The Crude was purified by flash column chromatography over silica gel, ISCO, CombiFlash, 24 g cartridge (eluting with 0-15% MeOH:DCM to afford 2.63 g, 9.42 mmol, 76.63% yield of the title compound as yellow solid. ¹H NMR (500 MHz, DMSO-d₆) δ 8.73 (s, 2H), 8.41 (d, J=18.4 Hz, 1H), 7.87-7.70 (m, 1H), 7.70-7.31 (m, 4H), 7.05 (d, J=8.2 Hz, 1H), 6.65 (d, J=59.2 Hz, 2H), 1.34 (t, J=7.1 Hz, 1H), 1.19 (d, J=6.8 Hz, 6H). MS (ES⁺) m/z calcd. for [M+H]⁺ [C₁₇H₁₆N₄+H]⁺: 276.14 found 277.0 LCMS t_{R}=4.50 min [Buff A—0.1% FA/H2O, Buff B—0.1% FA/AcN].

### Synthesis of (3-amino-2,6-difluorophenyl)(5-chloro-1H-pyrrolo[2,3-b]pyridin-3-yl)methanone [Intermediate 17]

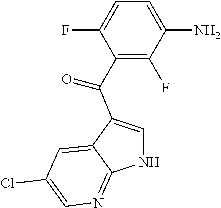 [SMILES: Nc1ccc(F)c(C(=O)c2c[nH]c3ncc(Cl)cc23)c1F]

Intermediate 17 is known in the literature and the synthesis can be performed according to the reference below:
Reference: WO2007/2325A1

### Synthesis of 5-(5-amino-2-methylphenyl)-1-methyl-1,3-dihydro-2H-benzo[d]imidazol-2-one [Intermediate 18]

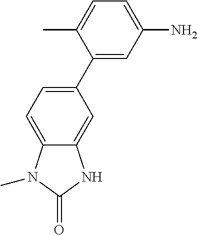 [SMILES: Cc1ccc(N)cc1-c1ccc2c(c1)[nH]c(=O)n2C]

Intermediate 18 is known in the literature and the synthesis can be performed according to the reference below:
Reference: Journal of Medicinal Chemistry, 2020, vol. 63, #5, p. 2013-2027

### Synthesis of N-(5-(2-((5-amino-2-methylphenyl)amino)pyrimidin-4-yl)-4-methylthiazol-2-yl)acetamide [Intermediate 19]

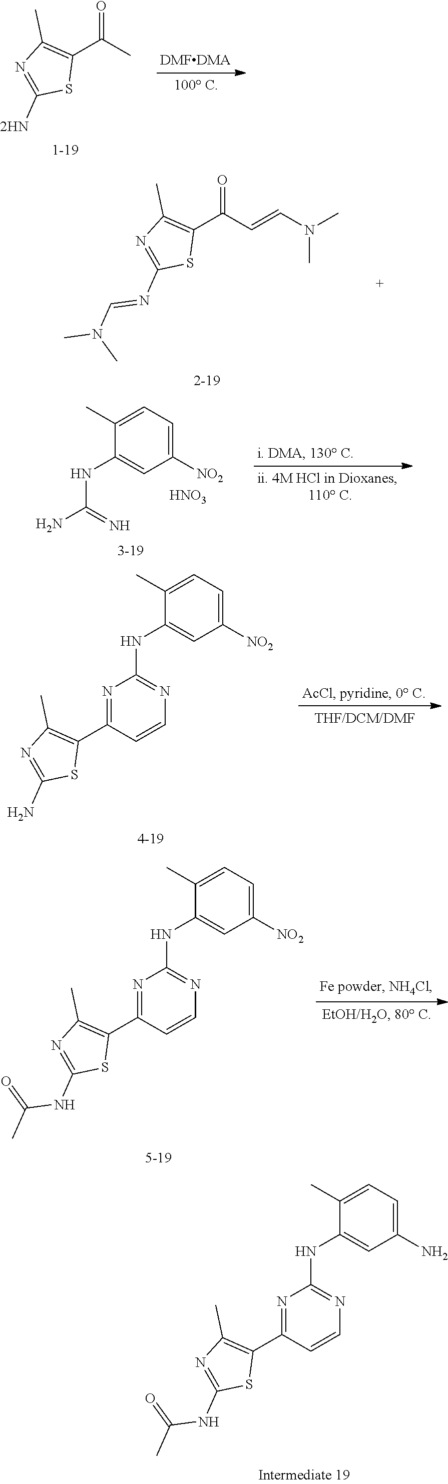

### (E)-N′-(5-((E)-3-(dimethylamino)acryloyl)-4-methylthiazol-2-yl)-N,N-dimethylformimidamide [2-19]

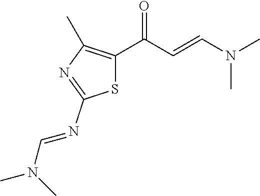

A solution of DMFDMA (40 mL, 80.0 mmol, 1.00 eq) in 2-Amino-4-methyl-5-acetylthiazole (12.50 g, 80.0 mmol, 1.00 eq) was heated to 100° C. for 16 h. After cooling to rt the reaction mixture is concentrated in vacuo and the crude was triturated from ethyl acetate and filtered through a fritted Buchner funnel resulting in 10.5 g the title compound as a light orange solid. The mother liquor was concentrated in vacuo and the crude solid was recrystallized from EtOAc which resulted in an additional 0.675 g. The total yield was 11.2 g, 52.5% yield of the title compounds as a light orange solid. ¹H NMR (600 MHz, DMSO-d₆) δ 8.35 (s, 1H), 7.56 (d, J=12.1 Hz, 1H), 5.27 (d, J=12.1 Hz, 1H), 3.13 (s, 4H), 2.98 (s, 3H), 2.83 (s, 3H), 2.48 (s, 3H). MS (ES⁺) m/z calc'd for [M+H]⁺ [Cl₂H₁₈N₄OS+H]⁺: 267.36 found 267.20, LCMS: t_{R}=3.75 min [Analytical Method: 05991008_AA1.lcm]

### 4-methyl-5-[2-(2-methyl-5-nitro-anilino)pyrimidin-4-yl]thiazol-2-amine [4-19]

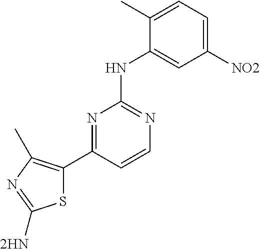

A mixture of 1-(2-methyl-5-nitro-phenyl)guanidine; nitric acid (3.05 g, 11.3 mmol, 1.00 eq) and N′-[5-[(E)-3-(dimethylamino)prop-2-enoyl]-4-methyl-thiazol-2-yl]-N,N-dimethyl-formamidine (3.00 g, 11.3 mmol, 1.00 eq) in DMA (28.157 mL, 0.4000 M) was stirred at 130° C. for 16 hours. The reaction mixture was cooled to rt and then quenched with NaHCO₃ (half sat., aq.). The resulting precipitate was collected via vacuum filtration. Without further drying, 4M HCl in Dioxanes (23 mL) was added to the 3.2 g of crude “wet” material after transferring to a pressure flask. This was heated to 110° C. while stirring overnight. The reaction mixture was cooled to rt before diluting with Et₂O. The top layer was decanted off and the remaining material loaded onto silica gel. Column chromatography (silica gel; dry load; ACN/toluene then MeOH/DCM) provided 0.95 g, 2.71 mmol, 24.09% yield of the title compound as a solid. ¹H NMR (600 MHz, DMSO-d₆) δ 8.49 (d, J=2.4 Hz, 1H), 8.47 (d, J=5.5 Hz, 1H), 7.95 (dd, J=8.4, 2.4 Hz, 1H), 7.54 (d, J=8.5 Hz, 1H), 7.08 (d, J=5.5 Hz, 1H), 2.50 (s, 3H), 2.40 (s, 3H); MS (ES⁺) m/z calc'd for [M+H]⁺ [C₁₅H₁₄N₆O₂S+H]⁺:343.1 found 343.1, LCMS: t_{R}=4.77 min [Analytical Method: 05991008_AA1.lcm]

### N-[4-methyl-5-[2-(2-methyl-5-nitro-anilino)pyrimidin-4-yl]thiazol-2-yl]acetamide [5-19]

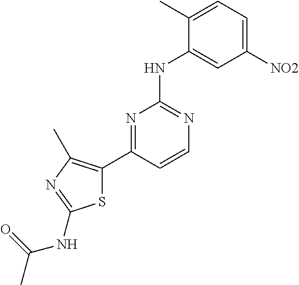 [SMILES: CC(=O)Nc1nc(C)c(-c2ccnc(Nc3cc([N+](=O)[O-])ccc3C)n2)s1]

Acetyl Chloride (0.098 mL, 1.37 mmol, 1.00 eq) was added dropwise to a mixture of 4-methyl-5-[2-(2-methyl-5-nitro-anilino)pyrimidin-4-yl]thiazol-2-amine (0.47 g, 1.37 mmol, 1.00 eq) and pyridine (0.33 mL, 4.10 mmol, 3.00 eq) in DCM (0.75 M, 1.8 mL), THF (0.5 M, 2.7 mL), and DMF (1.3 M, 1.1 mL) at 0° C. After 20 minutes of stirring at 0° C. the reaction mixture was quenched with MeOH and loaded directly onto silica gel. Column chromatography (silica gel; dry load; 30-75% EtOAc in Hex) afforded 0.16 g, 0.403 mmol, 29.48% yield of the title compound as a solid. MS (ES⁺) m/z calc'd for [M+H]⁺ [C₁₇H₁₆N₆O₃S+H]⁺: 385.1 found 385.0, LCMS: t_{R}=6.13 min [Analytical Method: 05991008_AA1.lcm]

### N-[5-[2-(5-amino-2-methyl-anilino)pyrimidin-4-yl]-4-methyl-thiazol-2-yl]acetamide [Intermediate 19]

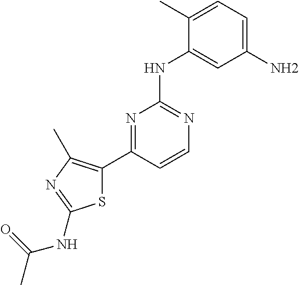 [SMILES: CC(=O)Nc1nc(C)c(-c2ccnc(Nc3cc(N)ccc3C)n2)s1]

The headspace for a mixture of N-[4-methyl-5-[2-(2-methyl-5-nitro-anilino)pyrimidin-4-yl]thiazol-2-yl]acetamide (0.50 g, 1.30 mmol, 1.00 eq), ammonium chloride (0.21 g, 3.90 mmol, 3.00 eq), and Fe powder (0.25 g, 4.55 mmol, 3.50 eq) in 95% EtOH (0.1 M, 13 mL) and H₂O (0.65 M, 2.6 mL) was purged with nitrogen prior to lowering into an oil bath 80° C. for 3 hours. The reaction mixture was then cooled to rt and loaded directly onto silica gel. Column chromatography (silica gel; dry load; 0-10% MeOH in DCM) provided 0.40 g, 1.08 mmol, 83.12% yield of the title compound as a pale yellow solid. ¹H NMR (500 MHz, DMSO-d₆) δ 12.21 (s, 1H), 8.49 (s, 1H), 8.31 (d, J=5.3 Hz, 1H), 6.91 (d, J=5.3 Hz, 1H), 6.84 (d, J=8.1 Hz, 1H), 6.76 (d, J=2.3 Hz, 1H), 6.31 (dd, J=8.0, 2.3 Hz, 1H), 4.75 (s, 2H), 2.53 (s, 3H), 2.15 (s, 3H), 2.04 (s, 3H); MS (ES⁺) m/z calc'd for [M+H]⁺ [C₁₇H₁₈N₆OS+H]⁺: 355.1 found 355.1, LCMS: t_{R}=3.64 min [Analytical Method: 05991008_AA1.lcm]

### Synthesis of 2-(6-chloroimidazo[1,2-b]pyridazin-3-yl)benzofuran-5-amine [Intermediate 20]

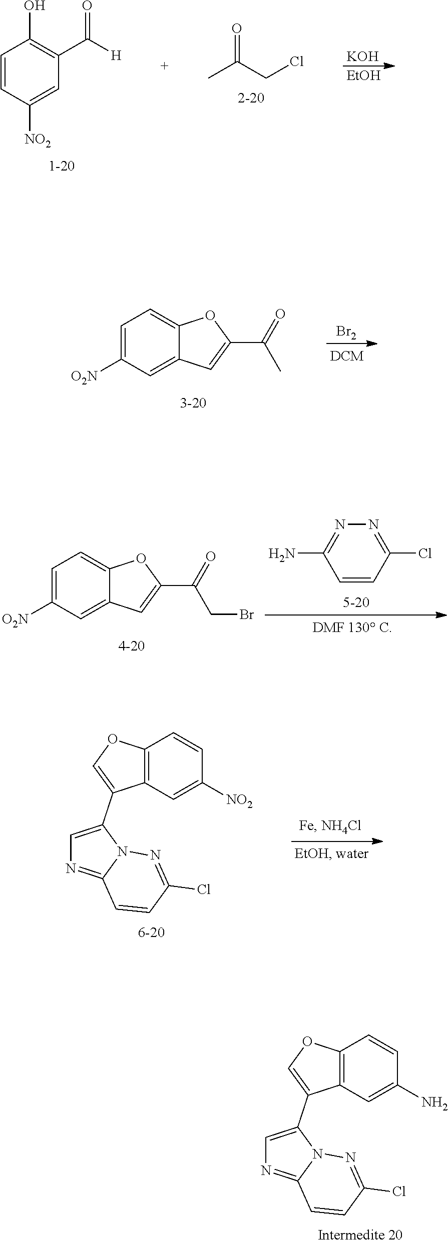

### 3-(6-chloroimidazo[1,2-b]pyridazin-3-yl)benzofuran-5-amine [Intermediate 20]

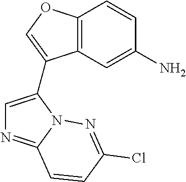 [SMILES: Nc1ccc2occ(-c3cnc4ccc(Cl)nn34)c2c1]

To a mixture of 6-chloro-3-(5-nitrobenzofuran-3-yl)imidazo[1,2-b]pyridazine (12 g, 38.4 mmol) in ethanol (210 mL) and water (105 mL) was added ammonium chloride (4.11 g, 76.9 mmol) and Fe (10.73 g, 192.2 mmol). The resulting reaction mixture was stirred at 80° C. for 6 h or until the reaction was complete. Reaction mixture was filtered and concentrated to remove ethanol. Saturated aqueous sodium bicarbonate solution was added and extracted with dichloromethane (3×100 mL). Combined organic layer was dried over anhydrous sodium sulfate, filtered and concentrated to obtain crude 3-(6-chloroimidazo[1,2-b]pyridazin-3-yl)benzofuran-5-amine (1.7 g, 15.6 yield) which was used in the next step without further purification. Note: This crude material had suspected contamination of 3-Amino-6-chloropyridazine from previous steps. LCMS: RT=1.76 min; m/z=284.04, found=285.3 [M+H]⁺

### 6-chloro-3-(5-nitrobenzofuran-3-yl)imidazo[1,2-b]pyridazine [6-20]

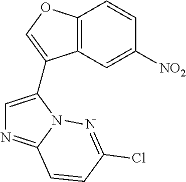 [SMILES: O=[N+]([O-])c1ccc2occ(-c3cnc4ccc(Cl)nn34)c2c1]

3-Amino-6-chloropyridazine (5.7 g, 43.8 mmol) was added to a solution of 2-bromo-1-(5-nitrobenzofuran-2-yl)ethanone (12.32 g, 43.4 mmol) in DMF (350 ml) and the resulting reaction mixture was stirred at 130° C. for 16 h. Reaction mixture was cooled in an ice/water bath and diluted with ice/water to obtain solid precipitates which were filtered, washed with water and dried under high vacuum to obtain 6-chloro-3-(5-nitrobenzofuran-3-yl)imidazo[1,2-b]pyridazine (12.1 g, 89% yield).

### 2-bromo-1-(5-nitrobenzofuran-2-yl)ethanone[4-20]

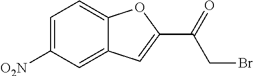 [SMILES: O=C(CBr)c1cc2cc([N+](=O)[O-])ccc2o1]

1-(5-nitrobenzofuran-2-yl)ethanone (317 mg, 1.5 mmol) from step 1 was dissolved in dichloromethane (15 mL) and cooled under an ice/water bath. A solution of bromine (0.079 mL, 1.5 mmol) in dichloromethane (5 mL) was added drop wise at 0° C. Resulting reaction mixture was further stirred at 0° C. for 30 min and then gradually warmed up to room temperature. After 30 min, reaction mixture was quenched by the addition of saturated aqueous sodium bicarbonate solution (15 mL). Organic layer was separated and the aqueous layer was back extracted with dichloromethane (3×10 mL). Combined organic layer was washed with brine, dried over anhydrous sodium sulfate, filtered and concentrated in vacuo to give crude product 2-bromo-1-(5-nitrobenzofuran-2-yl)ethanone (400 mg, 91% yield) which was used in the next step without any further purification.

### 1-(5-Nitrobenzofuran-2-yl)ethanone [3-20]

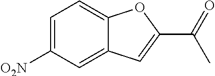

A solution of 2-hydroxy-5-nitrobenzaldehyde (500 mg, 3 mmol) and potassium hydroxide (167 mg, 3 mmol) in ethanol (8 mL) was heated at 75° C. for 5 min. Solution was gradually brought to approximately 0° C. under an ice/water bath. Chloroacetone (0.3 mL, 3.5 mmol) was added drop wise. Reaction mixture was slowly warmed up to 75° C. and stirred at this temperature for 20 h. Reaction mixture was gradually brought to room temperature and concentrated in vacuo. Water (10 mL) was added and the resulting crude mixture was extracted with ethyl acetate (3×15 mL). Combined organic layer was washed with brine (20 mL), dried over anhydrous sodium sulfate, filtered and concentrated under reduced pressure. Residue was dissolved in minimum amount of ethanol by heating and subsequently cooled down to obtain precipitates. Solids were filtered off, washed with ethanol and dried under high vacuum to obtain desired product 1-(5-nitrobenzofuran-2-yl)ethanone which was used in the next step without any further purification (317 mg, 52% yield).

### Synthesis of 5-((3-aminophenyl)ethynyl)thieno[2,3-d]pyrimidin-4-amine [Intermediate 21]

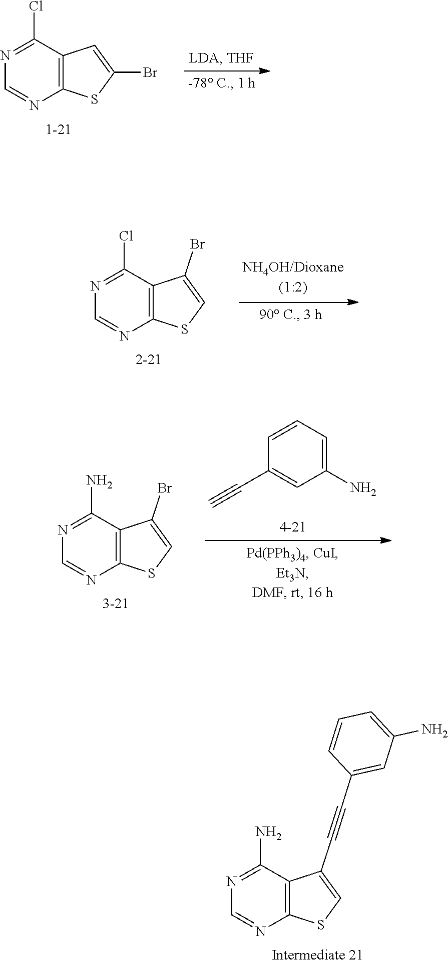

### 5-Bromo-4-chlorothieno[2,3-d]pyrimidine [2-21]

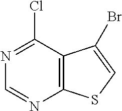 [SMILES: Clc1ncnc2scc(Br)c12]

To a stirred solution of 6-bromo-4-chlorothieno[2,3-d]pyrimidine (50.0 g, 200.4 mmol) in anhydrous THF (2000 ml) at −78° C. was added LDA (2M, 100 mL, 200.0 mmol) dropwise. The reaction mixture was stirred at this temperature for 1 h, after which time was added H₂O: THF (62.5 ml: 250 mL). The reaction mixture was warmed to 0° C. and poured into water (1000 mL), extracted with DCM (2×500 mL). The combined organic layers were dried over Na₂SO₄ and concentrated. The crude product was triturated with hexanes to afford 5-bromo-4-chlorothieno[2,3-d]pyrimidine (32 g, 128.24 mmol, 64.1%) as light yellow solid.

¹H NMR (500 MHz, CDCl₃) δ 8.88 (s, 1H), 7.67 (s, 1H), MS(ESI⁺) m/z calcd for [M+H]⁺[C₆H₂BrClN₂S+H]⁺: 248.9 found: non ionizable, t_{R}=2.82 mins. [Analytical Method: A-6]

### 5-Bromothieno[2,3-d]pyrimidin-4-amine [3-21]

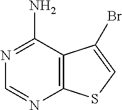 [SMILES: Nc1ncnc2scc(Br)c12]

To a stirred Suspension of 5-bromo-4-chlorothieno[2,3-d]pyrimidine (20 g, 80.15 mmol) in 1, 4 dioxane (300 ml) was added ammonium hydroxide (150 ml). The suspension was heated to 90° C. and stirred for 3 h in an autoclave reactor. Crushed ice was added, and the solids were filtered, washed with H₂O and dried under reduced pressure to afford 5-bromothieno[2,3-d]pyrimidin-4-amine (14.8 g, 64.9 mmol, 81% yield) as a brown solid. ¹H NMR (500 MHz, DMSO-d₆) δ 8.31 (s, 1H), 7.76 (d, J=4.0 Hz, 1H), 7.46 (bs, 2H). MS(ESI⁺) m/z calcd for [M+H]⁺[C₆H₄BrN₃S+H]⁺: 229.9 found: 230.0, t_{R}=1.46 mins. [Analytical Method: A-6]

### 5-((3-aminophenyl)ethynyl)thieno[2,3-d]pyrimidin-4-amine [Intermediate 21]

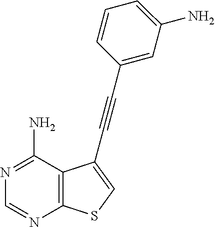 [SMILES: Nc1cccc(C#Cc2csc3ncnc(N)c23)c1]

A stirred solution of 5-bromothieno[2,3-d]pyrimidin-4-amine (20 g, 86.92 mmol, 1.00 equiv) in anhydrous DMF (1000 ml) was bubbled with argon for 15 minutes. To this was added Et₃N (24.4 ml, 173.85 mmol) followed by 3-ethynylbenzenamine (12.21 g, 104.31 mmol) while degassing was continued for another 15 min. CuI (1.65 g, 8.69 mmol) and Pd(PPh₃)₄ (11.1 g, 9.56 mmol) were then added to the mixture. The resulting brown suspension was stirred at room temperature for 16 h. The reaction mixture was filtered directly onto crushed ice through Celite, the solids were filtered, washed with H₂O (200 ml), followed by hexanes, and dried to afford a crude product which was purified by column chromatography over silica gel, ISCO, CombiFlash, 120 g cartridge (1-20% ammoniacal MeOH/DCM as eluent). Fractions containing the desired compound were combined, concentrated and triturated with Et₂O to afford 5.6 g, 21.05 mmol, 24.2% yield of the title compound as light brown solid. ¹H NMR (500 MHz, DMSO-d₆) δ 8.34 (s, 1H), 7.94 (s, 1H), 7.08 (t, J=7.8 Hz, 1H), 6.75 (t, J=1.9 Hz, 1H), 6.72 (dt, J=7.5, 1.2 Hz, 1H), 6.65 (ddd, J=8.1, 2.3, 1.0 Hz, 1H), 5.31 (s, 2H). MS(ESI+) m/z calcd for [M+H]⁺[C₁₄H₁₀N₄S+H]⁺: 267.07 found: 267.1, t_{R}=1.13 mins. [Analytical Method: B]

### N-(6-(5-amino-2-methylphenyl)imidazo[1,2-a]pyridin-2-yl)acetamide [Intermediate 22]

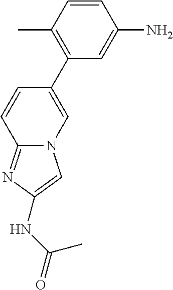 [SMILES: CC(=O)Nc1cn2cc(-c3cc(N)ccc3C)ccc2n1]

Intermediate 22 is known in the literature and the synthesis can be performed according to the reference below:
Reference: Bioorganic and Medicinal Chemistry Letters, 2017, vol. 27, #23, p. 5221-5224

### Synthesis of 6-(5-amino-4-fluoro-2-methylphenyl)-N-methylpyrido[3,2-d]pyrimidin-2-amine [Intermediate 23]

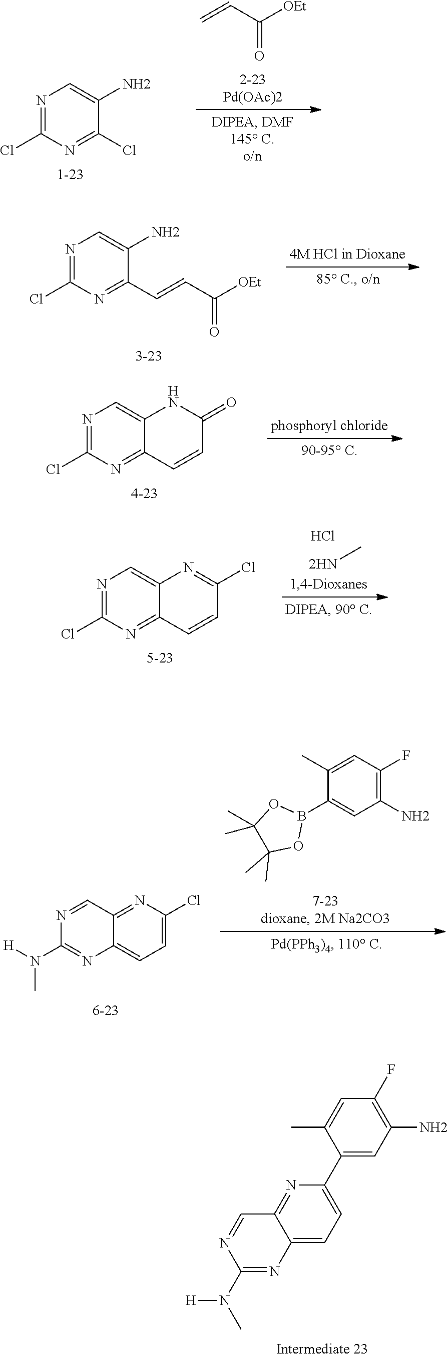

### 2-chloropyrido[3,2-d]pyrimidin-6(5H)-one [4-23]

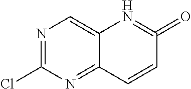

To a stirred solution of 2,4-dichloropyrimidin mmol). The mixture was heated to 145° C. in a flask fitted with a condenser and stirred for 4 h. After cooling to room temperature, the reaction mixture was diluted with EtOAc, washed with H₂O, and filtered through Celite. The filtrate was evaporated to provide crude ethyl (E)-3-(5-amino-2-chloro-pyrimidin-4-yl)acrylate (33 g) which was dissolved in 4.0 M HCl in dioxane, and the mixture was heated to 90° C. and stirred for 2 days. After cooling to room temperature, the reaction mixture was evaporated, and the residue was triturated with saturated NaHCO₃ solution and filtered. The cake was triturated with H₂O and filtered. The product thus obtained was dried under in vacuo to provide the title compound as a brown solid (23 g, 52% over 2 steps). ¹H NMR (500 MHz, DMSO-d₆) δ 8.72 (s, 1H), 7.83 (d, J=9.8 Hz, 1H), 7.01 (d, J=9.8 Hz, 1H). MS (ES⁺) m/z calcd. for [2M+H]⁺ [2(C₇H₄ClN₃0)+H]⁺: 182.1 found 363.1, LCMS t_{R}=1.63 min [Analytical Method A].

### 2,6-Dichloropyrido[3,2-d]pyrimidine [5-23]

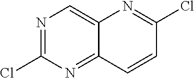 [SMILES: Clc1ccc2nc(Cl)ncc2n1]

A stirred solution of 2-chloropyrido[3,2-d]pyrimidin-6(5H)-one (28.0 g, 44.1 mmol) in phosphoryl chloride (150 mL) was heated to 95° C. and stirred overnight. After cooling to room temperature, the reaction mixture was concentrated in vacuo. The resulting residue was taken up in EtOAc (500 mL) and then washed with saturated NaHCO₃ solution, brine, dried over Na₂SO₄, and concentrated in vacuo to afford 12.3 g, 42% of crude title compound as a dark brown solid which was used in the subsequent reaction without further purification. ¹H NMR (500 MHz, DMSO-d₆) δ 9.61 (d, J=0.7 Hz, 1H), 8.49 (dd, J=8.9, 0.8 Hz, 1H), 8.14 (d, J=8.9 Hz, 1H).

### 6-Chloro-N-methylpyrido[3,2-d]pyrimidin-2-amine [6-23]

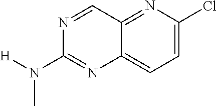

DIPEA (47.0 mL, 271 mmol) was added to a mixture of 2,6-dichloropyrido[3,2-d]pyrimidine 5 (12.3 g, 61.5 mmol) and methylamine hydrochloride 6 (9.1 g, 135 mmol) in 1,4-dioxane (250 ml). The mixture was heated to 90° C. and stirred overnight. The reaction mixture was concentrated to provide crude material which was purified by flash column chromatography over silica gel, ISCO, CombiFlash, 220 g cartridge (eluting with 15-40% ethyl acetate in hexane) to give 6-chloro-N-methylpyrido[3,2-d]pyrimidin-2-amine 7 as a yellow solid (11.5 g, 95%). ¹H NMR (500 MHz, DMSO-d₆) δ 69.05 (s, 1H), 7.94 (d, J=9.1 Hz, 1H), 7.79 (s, 1H), 7.71 (d, J=8.9 Hz, 1H), 2.89 (d, J=4.8 Hz, 3H). MS (ES⁺) m/z calcd. for [M+H]⁺[C₈H₇ClN₄+H]⁺: 195.0 found 195.4, LCMS t_{R}=1.51 min [Method A].

### 2-Fluoro-4-methyl-5-(4,4,5,5-tetramethyl-1,3,2-dioxaborolan-2-yl)aniline [7-23]

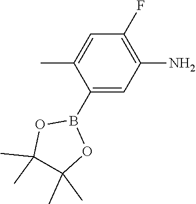 [SMILES: Cc1cc(F)c(N)cc1B1OC(C)(C)C(C)(C)O1]

To a stirred solution of 5-bromo-2-fluoro-4-methylaniline (19.7 g, 96.5 mmol), bis(pinacolato)diboron (27 g, 106 mmol), and potassium acetate (28.4 g, 289.6 mmol) in dioxane (250 mL) was added [1,1′-bis(diphenylphosphino)ferrocene]-dichloropalladium(II)-dichloromethane complex (3.53 g, 4.83 mmol). The reaction mixture was heated to 100° C. and stirred overnight. The reaction mixture was filtered while still hot and concentrated in vacuo. The crude material was purified by flash column chromatography over silica gel, ISCO, CombiFlash, 40 g cartridge (eluting with 0-20% ethyl acetate in hexane) to give 3.24 g, 85% yield of the title compound as an off-white solid. ¹H NMR (500 MHz, DMSO-d₆) δ 7.13 (d, J=10.4 Hz, 1H), 6.77 (d, J=12.7 Hz, 1H), 4.84 (s, 2H), 2.30 (s, 3H), 1.26 (s, 12H). MS (ES⁺) m/z calcd. for [M+H]⁺[C₁₃H₁₉BFNO₂+H]⁺: 252.2 found 252.0 LCMS t_{R}=3.74 min [Analytical Method A].

### 6-(5-Amino-4-fluoro-2-methylphenyl)-N-methylpyrido[3,2-d]pyrimidin-2-amine [Intermediate 23]

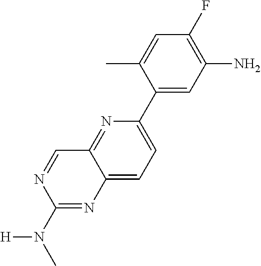 [SMILES: CNc1ncc2nc(-c3cc(N)c(F)cc3C)ccc2n1]

A mixture of 6-chloro-N-methylpyrido[3,2-d]pyrimidin-2-amine (7.98 g, 41.0 mmol), 2-fluoro-4-methyl-5-(4,4,5,5-tetramethyl-1,3,2-dioxaborolan-2-yl)aniline (20.5 g, 82.0 mmol) and Pd(PPh₃)₄ (1.42 g, 1.23 mmol) in 1,4-dioxane (250 ml) was treated with 2M Na₂CO₃ (41 ml). The mixture was heated to 110° C. and stirred overnight. The reaction mixture was cooled to room temperature and diluted with EtOAc. The organic layer washed with H₂O, brine, dried over anhydrous Na₂SO₄, filtered, and concentrated in vacuo. The crude was purified by flash column chromatography over silica gel, ISCO, CombiFlash, 220 g cartridge (eluting with 40-70% ethyl acetate in hexane) to give 5.9 g, 51% yield of the title compound as a yellow solid. ¹H NMR (500 MHz, DMSO-d₆) δ 9.10 (s, 1H), 7.91 (d, J=8.8 Hz, 1H), 7.74 (dd, J=8.8, 1.2 Hz, 1H), 7.61 (s, 1H), 6.95 (d, J=12.4 Hz, 1H), 6.91 (d, J=9.4 Hz, 1H), 5.03 (s, 2H), 2.92 (d, J=4.8 Hz, 3H), 2.19 (s, 3H). MS (ES⁺) m/z calcd. for [M+H]⁺ [C₁₅H₁₄FN₅+H₂O]⁺: 302.1 found 302.2 LCMS t_{R}=1.11 min [Analytical Method B].

### Synthesis of 5-(3-amino-4-fluorophenoxy)-N-methylthiazolo[5,4-b]pyridin-2-amine [Intermediate 24]

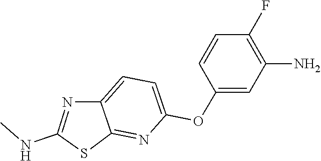 [SMILES: CNc1nc2ccc(Oc3ccc(F)c(N)c3)nc2s1]

Intermediate 24 is known in the literature and the synthesis can be performed according to the reference below:
Reference: Bioorganic and Medicinal Chemistry, 2012, vol. 20, #15, p. 4680-4692

### Synthesis of 6-methyl-N1-(4-(pyridin-3-yl)thiazol-2-yl)benzene-1,3-diamine [Intermediate 25]

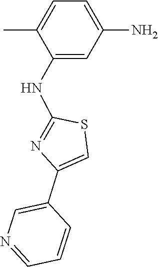 [SMILES: Cc1ccc(N)cc1Nc1nc(-c2cccnc2)cs1]

Available from Commercial Sources

### Synthesis of N¹-(4-(2-aminopyridin-3-yl)pyrimidin-2-yl)-6-fluorobenzene-1,3-diamine [Intermediate 26]

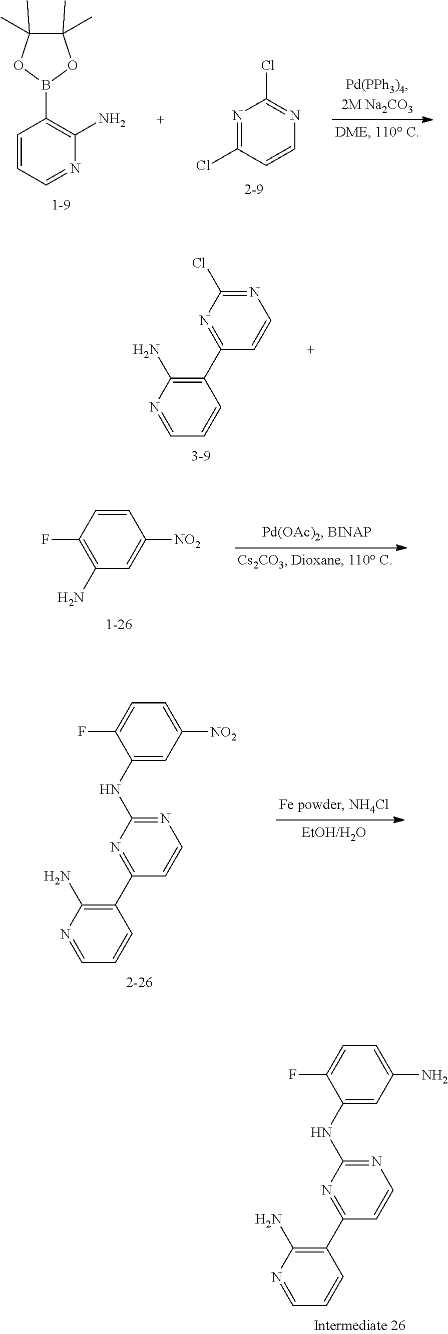 [SMILES: CC1(C)OB(c2cccnc2N)OC1(C)C ; Clc1ccnc(Cl)n1 ; Nc1ncccc1-c1ccnc(Cl)n1 ; Nc1cc([N+](=O)[O-])ccc1F ; Nc1ncccc1-c1ccnc(Nc2cc([N+](=O)[O-])ccc2F)n1 ; Nc1ccc(F)c(Nc2nccc(-c3cccnc3N)n2)c1]

### 4-(2-amino-3-pyridyl)-N-(2-fluoro-5-nitro-phenyl)pyrimidin-2-amine [2-26]

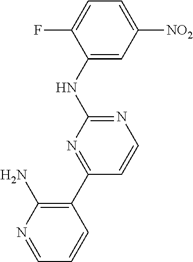 [SMILES: Nc1ncccc1-c1ccnc(Nc2cc([N+](=O)[O-])ccc2F)n1]

Nitrogen gas was bubbled through a mixture of 3-(2-chloropyrimidin-4-yl)pyridin-2-amine (0.56 g, 2.73 mmol, 1.00 eq), 2-fluoro-5-nitro-aniline (1.28 g, 8.19 mmol, 3.00 eq), BINAP (0.17 g, 0.273 mmol, 0.100 eq), Pd(OAc)2 (0.061 g, 0.273 mmol, 0.100 eq), and Cesium Carbonate (1.78 g, 5.46 mmol, 2.00 eq) in 1,4-Dioxane (27.295 mL, 0.1000 M) for 20 minutes prior to lowering into an oil bath at 75-80° C. After 1.5 hours, the reaction was diluted with EtOAc and concentrated directly onto silica gel. The crude material was purified via column chromatography (dry load on silica gel; 20-50% ACN in toluene) to afford 0.56 g, 1.69 mmol, 61.93% yield of the title compound as a solid. MS(ESI⁺) m/z calc'd for [C₁₅H₁₁FN₆O₂+H]⁺: 327.1 found: 327.1, t_{R}=4.27 min. [Analytical Method 05991008_AA0.lcm]

### N3-[4-(2-amino-3-pyridyl)pyrimidin-2-yl]-4-fluoro-benzene-1,3-diamine [Intermediate 26]

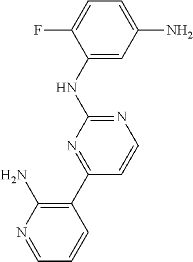 [SMILES: Nc1ccc(F)c(Nc2nccc(-c3cccnc3N)n2)c1]

A mixture of 4-(2-amino-3-pyridyl)-N-(2-fluoro-5-nitro-phenyl)pyrimidin-2-amine (0.56 g, 1.73 mmol, 1.00 eq), Ammonium Chloride (0.28 g, 5.19 mmol, 3.00 eq), and Fe powder (0.34 g, 6.05 mmol, 3.50 eq) in 95% EtOH (0.13 M, 13.8 mL) and H₂O (0.65 M, 2.7 mL) was stirred at 80° C. for 2.5 h. The reaction mixture was then cooled to rt and loaded directly onto silica gel. Column chromatography (silica gel; dry load; 0-10% MeOH in DCM) then provided 0.38 g, 1.21 mmol, 69.82% yield of the title compound as a yellow solid. ¹H NMR (600 MHz, DMSO-d₆) δ 9.19 (s, 1H), 8.39 (d, J=5.4 Hz, 1H), 8.12 (dd, J=7.9, 1.7 Hz, 1H), 8.08 (dd, J=4.7, 1.8 Hz, 1H), 7.56 (s, 2H), 7.30 (d, J=5.6 Hz, 1H), 6.94 (dd, J=6.8, 2.4 Hz, 1H), 6.88 (dd, J=10.8, 8.7 Hz, 1H), 6.63 (dd, J=7.8, 4.7 Hz, 1H), 6.33-6.28 (m, 1H), 4.93 (s, 2H); MS(ESI⁺) m/z calc'd for [C₁₅H₁₃FN₆+H]⁺: 297.1 found: 297.1, t_{R}=2.33/2.39 min. [Method 05991008_AA0.lcm]

### Synthesis of N-(3-aminophenyl)-6-(1H-pyrazol-5-yl)picolinamide [Intermediate 27]

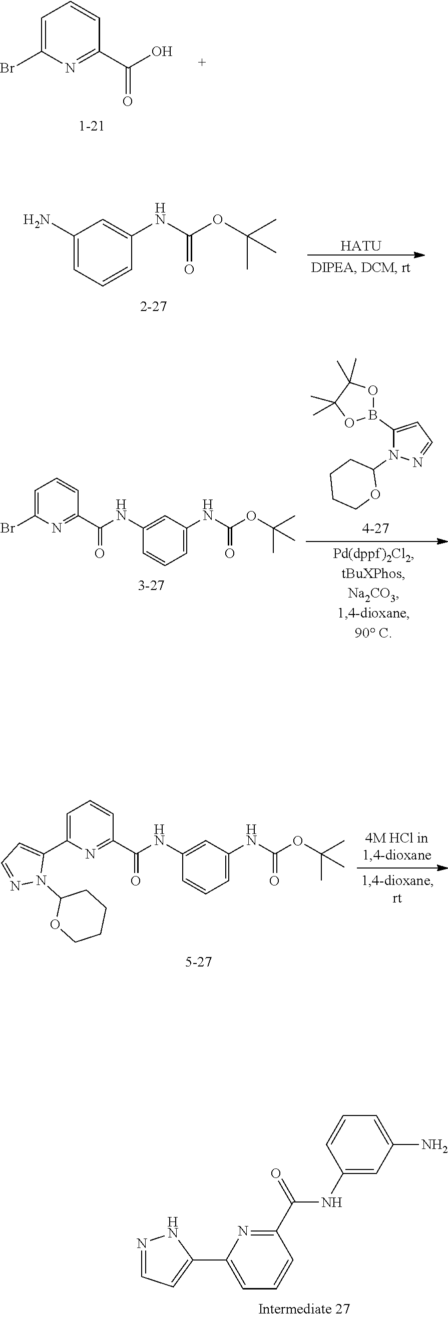

### tert-butyl (3-(6-bromopicolinamido)phenyl)carbamate [3-27]

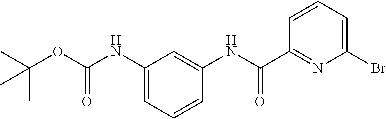 [SMILES: CC(C)(C)OC(=O)Nc1cccc(NC(=O)c2cccc(Br)n2)c1]

A solution of 6-Bromopyridine-2-carboxylic acid (2.00 g, 9.90 mmol, 1.00 eq) and (3-Amino-phenyl)-carbamic acid tert-butyl ester (2.27 g, 10.9 mmol, 1.10 eq) in anhydrous DCM (80 mL, 0.0792 M) was charged with diisopropylethylamine (8.8 mL, 49.5 mmol, 5.00 eq) and a solution of 1-[bis(dimethylamino)methylene]-1H-1,2,3-triazolo[4,5-b]pyridinium 3-oxide hexafluorophosphate (7.53 g, 19.8 mmol, 2.00 eq) in anhydrous DCM (45 mL, 0.0792 M) and stirred at rt for 2 h. The reaction mixture was charged with H₂O (75 mL) and separated. The aqueous was extracted with DCM (3×75 mL) and the combined organic layers were washed with brine (1×75 mL), dried over anhydrous Na₂SO₄, filtered and concentrated in vacuo. The crude solid was triturated with EtOAc (40 mL) and filtered through a fritted funnel and dried over high vac overnight resulting in 2.09 g, 50% yield of the title compound as a white solid. ¹H NMR (600 MHz, DMSO-d₆) δ 10.30 (s, 1H), 9.39 (s, 1H), 8.12 (dd, J=7.5, 0.9 Hz, 1H), 8.05 (t, J=2.0 Hz, 1H), 8.00 (t, J=7.8 Hz, 1H), 7.92 (dd, J=8.0, 0.9 Hz, 1H), 7.44 (ddd, J=8.1, 2.1, 1.0 Hz, 1H), 7.22 (t, J=8.1 Hz, 1H), 7.15 (ddd, J=8.2, 2.1, 1.0 Hz, 1H), 1.49 (s, 9H).; MS (ES⁺) m/z calc'd for [M−H]⁺ [C₁₇H₁₈BrN₃O₃+H]⁺: 392.25 found 392.0, LCMS: t_{R}=7.71 min [Analytical Method:05991008_BB0.lcm]

1

### tert-butyl (3-(6-(1-(tetrahydro-2H-pyran-2-yl)-1H-pyrazol-5-yl)picolinamido)phenyl)carbamate [5-27]

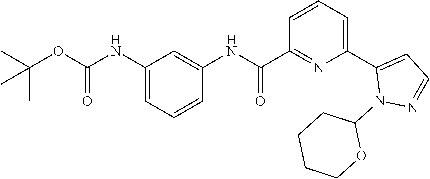 [SMILES: CC(C)(C)OC(=O)Nc1cccc(NC(=O)c2cccc(-c3ccnn3C3CCCCO3)n2)c1]

A solution of tert-butyl N-[3-[(6-bromopyridine-2-carbonyl)amino]phenyl]carbamate (1.00 g, 2.55 mmol, 1.00 eq) and 1-(2-Tetrahydropyranyl)-1H-pyrazole-5-boronic acid pinacol ester (0.78 g, 2.80 mmol, 1.10 eq) in 1,4-Dioxane (70 mL, 0.0364 M) was charged with Sodium carbonate, anhydrous, granular (7.6 mL, 15.3 mmol, 6.00 eq) and the reaction mixture was degassed by evacuating reaction mixture and charging with N₂ gas 3 times. The reaction mixture was charged with [1,1′-Bis(diphenylphosphino)ferrocene]dichloropalladium(II) (0.22 g, 0.298 mmol, 0.117 eq) and 2-di-tert-Butylphosphino-2?,4?,6?-triisopropylbiphenyl (0.19 g, 0.454 mmol, 0.178 eq) and the reaction mixture was degassed again then heated to 90° C. for 1 h. The reaction mixture was charged with H₂O (75 mL) and separated. The aqueous was extracted with DCM (3×75 mL) and the combined organic layers were washed with brine (1×75 mL), dried over anhydrous Na₂SO₄, filtered and concentrated in vacuo. The crude solid was triturated with EtOAc (40 mL) and filtered through a fritted funnel and dried over high vac overnight resulting in 2.09 g, 50% yield of the title compound as a white solid. ¹H NMR (600 MHz, DMSO-d₆) δ 10.37 (s, 1H), 9.43 (s, 1H), 8.18 (t, J=7.8 Hz, 1H), 8.12 (dd, J=7.7, 1.1 Hz, 1H), 8.09 (t, J=2.1 Hz, 1H), 8.02 (dd, J=7.8, 1.1 Hz, 1H), 7.67 (d, J=1.8 Hz, 1H), 7.53-7.49 (m, 1H), 7.26 (t, J=8.1 Hz, 1H), 7.13 (ddd, J=8.1, 2.1, 1.0 Hz, 1H), 6.99 (d, J=1.8 Hz, 1H), 6.30 (dd, J=8.4, 3.0 Hz, 1H), 3.93 (s, 1H), 3.90-3.83 (m, 1H), 3.56 (ddd, J=11.3, 8.0, 4.9 Hz, 1H), 2.41-2.31 (m, 1H), 2.14-2.02 (m, 2H), 1.67 (dq, J=10.5, 6.4, 5.6 Hz, 1H), 1.54 (t, J=5.6 Hz, 2H), 1.49 (s, 9H), 1.08 (s, 9H); MS (ES⁺) m/z calc'd for [M−H]⁺ [C₂₅H₂₉N₅O₄+H]⁺: 464.53 found 464.2, LCMS: t_{R}=8.03 min [Analytical Method:05991008_BB0.lcm]

### N-(3-aminophenyl)-6-(1H-pyrazol-5-yl)picolinamide [Intermediate 27]

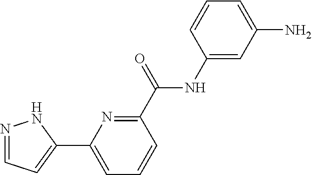 [SMILES: Nc1cccc(NC(=O)c2cccc(-c3ccn[nH]3)n2)c1]

A solution of tert-butyl N-[3-[[6-(2-tetrahydropyran-2-ylpyrazol-3-yl)pyridine-2-carbonyl]amino]phenyl]carbamate (0.50 g, 1.08 mmol, 1.00 eq) in 1,4-Dioxane (4.25 mL, 0.2538 M) was charged with a solution of hydrogen chloride, 4.0 m in dioxane (2.7 mL, 10.8 mmol, 10.0 eq) and stirred under nitrogen atmosphere. [Note: after 15 min the reaction mixture became cloudy and a precipitate began to form]. The reaction was allowed to stir for a further 3 h and concentrated in vacuo. The solid was triturated from diethyl ether and filtered through a fritted funnel and dried on a high vac overnight resulting in 315 mg, 92% yield of the title compound as an off-white solid. ¹H NMR (600 MHz, DMSO-d₆) δ 10.88 (s, 1H), 8.16 (dd, J=7.5, 1.5 Hz, 2H), 8.13 (t, J=7.6 Hz, 1H), 8.09 (dd, J=7.4, 1.4 Hz, 1H), 7.88-7.83 (m, 1H), 7.80-7.73 (m, 1H), 7.54 (t, J=8.0 Hz, 1H), 7.24-7.16 (m, 2H); MS (ES⁺) m/z calc'd for [M−H]⁺ [C₁₅H₁₃N₅O+H]⁺: 280.30 found 280.1, LCMS: t_{R}=3.63 min [Analytical Method: 05991008_AA0.lcm]

### 4-((6,7-dimethoxyquinolin-4-yl)oxy)-3-fluoroaniline [Intermediate 28]

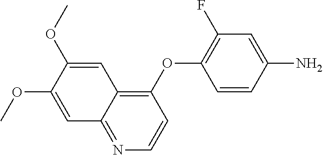 [SMILES: COc1cc2nccc(Oc3ccc(N)cc3F)c2cc1OC]

Available from Commercial Sources

### Synthesis of 5-(3-(aminomethyl)-4-fluorophenoxy)-3,4-dihydro-1,8-naphthyridin-2(1H)-one [Intermediate 29]

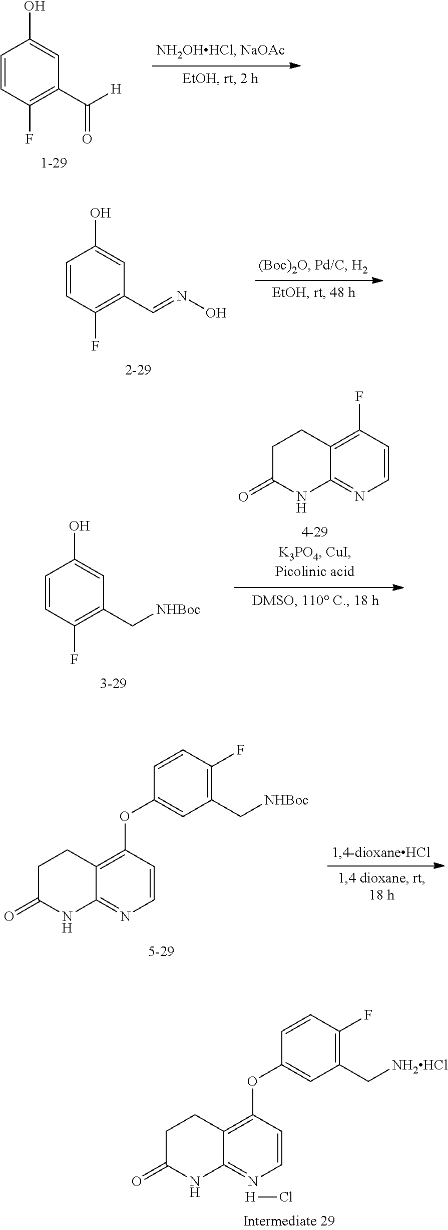

### (1E)-2-Fluoro-5-hydroxy-benzaldehyde oxime [2-29]

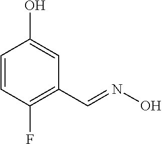 [SMILES: O/N=C/c1cc(O)ccc1F]

A stirred suspension of 2-fluoro-5-hydroxy-benzaldehyde (7.0 g, 49.96 mmol), hydroxylaminehydrochloride (6.94 g, 99.92 mmol) and sodium acetate (10.25 g, 124.9 mmol) in EtOH (280 mL) was stirred at room temperature for 2 h. The reaction mixture was concentrated under reduced pressure and the residue was partitioned between H₂O and EtOAc (200 mL each). The aqueous layer was extracted with EtOAc (2×100 ml). and the combined organic layers were washed with H₂O, dried over Na₂SO₄, and concentrated under reduced pressure to afford 7.5 g, 47.4 mmol, 95% yield of the title compound as light-yellow solid. ¹H NMR (500 MHz, DMSO-d₆) δ 11.50 (d, J=0.9 Hz, 1H), 9.52 (d, J=0.9 Hz, 1H), 8.11 (s, 1H), 7.13-6.98 (m, 2H), 6.85-6.72 (m, 1H). MS(ESI⁺) m/z calcd for [M+H]⁺[C₇H₆FNO₂+H]⁺: 156.0 found: 156.4, t_{R}=2.58 mins. [Analytical Method: A-6] tert-butyl N-[(2-fluoro-5-hydroxy-phenyl)methyl]carbamate [3-29]

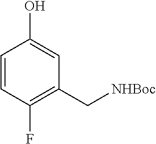 [SMILES: CC(C)(C)OC(=O)NCc1cc(O)ccc1F]

A suspension of (1E)-2-fluoro-5-hydroxy-benzaldehyde oxime (15.00 g, 96.7 mmol), Pd/C (10%, 3.00 g) and (Boc)₂O (31.66 g, 145 mmol) in EtOH (500 mL) was stirred at room temperature under a H₂ atmosphere for 24 h. TLC (25% EtOAc/hexanes) showed the formation of the desired product along with unreacted oxime. The H₂ balloon was refilled, and the reaction mixture was stirred for another 24 h, after which time the mixture was filtered through a pad of Celite, which was then rinsed with EtOAc (2×200 mL). The filtrate was concentrated, and the crude was purified by column chromatography over silica gel, ISCO, CombiFlash, 120 g cartridge (dry load, 5-15% EtOAc/hexanes as eluent) to afford 11.20 g, 44.1 mmol, 45.61% yield of the title compound as white solid. ¹H NMR (500 MHz, DMSO-d₆) δ 9.29 (s, 1H), 7.32 (d, J=8.1 Hz, 1H), 6.91 (t, J=9.3, Hz, 1H), 6.73-6.53 (m, 2H), 4.07 (d, J=6.1 Hz, 2H), 1.39 (s, 9H). MS(ESI⁺) m/z calcd for [M+H]⁺[2× Cl₂H₁₆FNO₃+H]⁺: 483.2 found: 483.4, t_{R}=3.89 mins. [Analytical Method: A-6]

### tert-butyl N-[[2-fluoro-5-[(7-oxo-6,8-dihydro-5H-1,8-naphthyridin-4-yl)oxy]phenyl]methyl]carbamate [5-29]

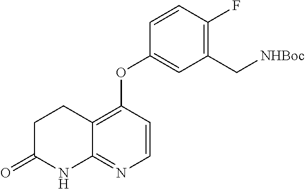 [SMILES: CC(C)(C)OC(=O)NCc1cc(Oc2ccnc3c2CCC(=O)N3)ccc1F]

In a sealed tube, a solution of 5-fluoro-3,4-dihydro-1H-1,8-naphthyridin-2-one (2.50 g, 15.0 mmol), tert-butyl N-[(2-fluoro-5-hydroxy-phenyl)methyl]carbamate (7.26 g, 30.1 mmol) and potassium phosphate (6.39 g, 30.1 mmol) in DMSO (45 mL) was degassed with nitrogen for 10 mins. To this were added picolinic acid (0.37 g, 3.01 mmol) and copper iodide (0.29 g, 1.50 mmol). The sealed tube was capped, and the resulting suspension was heated for 18 h at 110° C. The reaction mixture was cooled to room temperature, and ice-cold H₂O was added. The resulting solid was filtered, washed with H₂O, and dried. The crude solid was purified by column chromatography over silica gel, ISCO, CombiFlash, 40 g cartridge (dry load, 20-100% EtOAc/hexanes as eluent) to afford (3.6 g, 9.11 mmol, 60.5% yield of the title compound as off-white solid. ¹H NMR (500 MHz, DMSO-d₆) δ 10.49 (s, 1H), 7.96 (d, J=5.7 Hz, 1H), 7.39 (d, J=5.9 Hz, 1H), 7.25 (t, J=9.2, Hz, 1H), 7.08 (d, J=9.0 Hz, 1H), 6.99 (d, J=6.0 Hz, 1H), 6.29 (d, J=5.7 Hz, 1H), 4.15 (d, J=6.1 Hz, 2H), 2.88 (t, J=7.7, Hz, 2H), 2.53-2.50 (m, 2H), 1.34 (s, 9H). MS(ESI⁺) m/z calcd for [M+H]⁺[C₂₀H₂₂FN₃O₄+H]⁺: 388.1 found: 388.1, t_{R}=3.95 mins. [Analytical Method: A-6]

### 5-[3-(Aminomethyl)-4-fluoro-phenoxy]-3,4-dihydro-1H-1,8-naphthyridin-2-one; dihydrochloride [Intermediate 29]

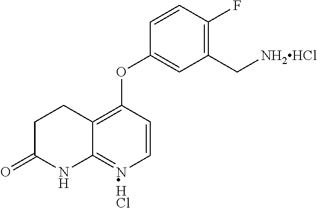

To a stirred solution of tert-butyl N-[[2-fluoro-5-[(7-oxo-6,8-dihydro-5H-1,8-naphthyridin-4-yl)oxy]phenyl]methyl]carbamate (3.00 g, 7.74 mmol) in 1,4-dioxane (30 mL) at room temperature was added HCl in dioxane (4 M, 30 ml). The reaction mixture was stirred at room temperature for 16 h, after which the volatiles were removed, the solid was triturated twice with Et₂O (100 mL) and the Et₂O was decanted. The residue was dried under reduced pressure to afford 2.70 g, 7.12 mmol, 92% yield of the title compound as an off-white solid. ¹H NMR (500 MHz, DMSO-d₆) δ 10.85 (s, 1H), 8.70 (s, 3H), 8.10 (d, J=6.2 Hz, 1H), 7.49 (dd, J=6.1, 3.0 Hz, 1H), 7.39 (t, J=9.1 Hz, 1H), 7.26 (ddd, J=8.9, 4.2, 3.0 Hz, 1H), 6.49 (d, J=6.2 Hz, 1H), 4.05 (q, J=5.8 Hz, 2H), 2.95 (t, J=7.7 Hz, 2H), 2.59 (dd, J=8.3, 7.1 Hz, 2H). MS(ESI⁺) m/z calcd for [M+H]⁺[C₁₅H₁₄FN₃O₂+H]⁺: 288.1 found: 288.1, t_{R}=0.80 mins. [Analytical Method: B]

### Synthesis of 6-(5-amino-2-methylphenoxy)-3-methylpyrido[3,2-d]pyrimidin-4(3H)-one [Intermediate 30]; and 6-((5-amino-2-methylphenyl)amino)-3-methylpyrido[3,2-d]pyrimidin-4(3H)-one [Example 39]

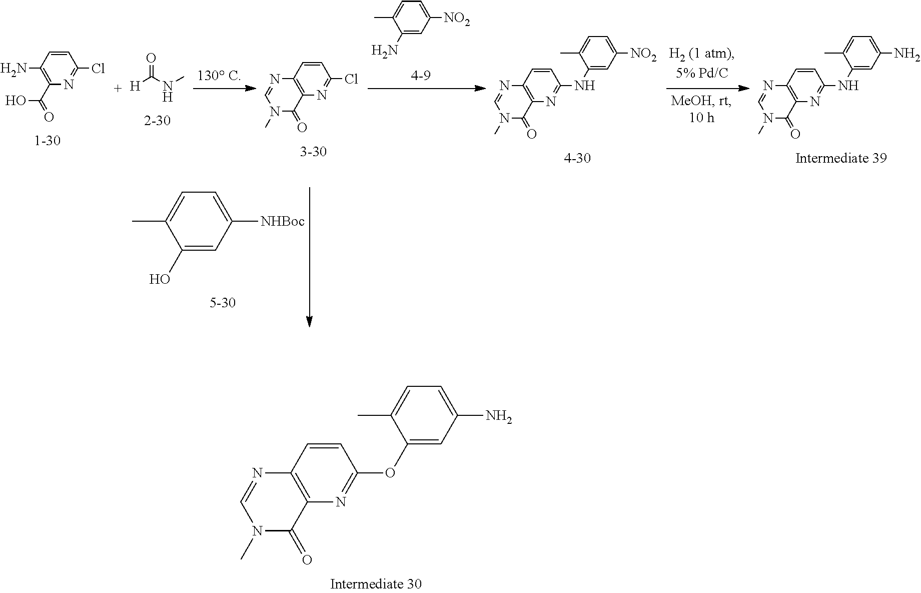

### 6-chloro-3-methylpyrido[3,2-d]pyrimidin-4(3H)-one [3-30]

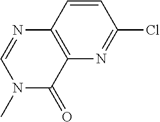

A solution of 3-amino-6-chloro-pyridine-2-carboxylic acid (10.00 g, 57.9 mmol, 1.00 eq) in N-methylformamide (142 mL, 1778 mmol, 30.7 eq) was heated to 130° C. for 16 h. The reaction mixture was partitioned between EtOAc and H₂O. The aqueous was extracted with EtOAc (3×10 mL) and the combined organic fractions were washed with 1M LiCl (2×10 mL), H₂O (1×10 mL), brine (1×10 mL), dried over any Na₂SO₄, filtered and concentrated in vacuo. The solid was triturated with 20% EtOAc in hexanes and filtered through a fritted funnel. The solid was washed with additional 20% EtOAc in hexanes (3×50 mL) and dried overnight on a high vac pump resulting in 3.51 g, 30.9% yield of the title compound as an orange solid. ¹H NMR (500 MHz, DMSO) δ 8.50 (s, OH), 8.16 (d, J=8.6 Hz, OH), 7.89 (d, J=8.6 Hz, OH), 3.53 (s, 1H); MS(ESI⁺) m/z calc'd for [M+H]⁺[C₈H₆ClN₃0+H]⁺: 196.61 found: 196.0, 197.0, t_{R}=2.67 mins. [Analytical Method 05991008_AA0.lcm].

### 3-methyl-6-((2-methyl-5-nitrophenyl)amino)pyrido[3,2-d]pyrimidin-4(3H)-one [4-30]

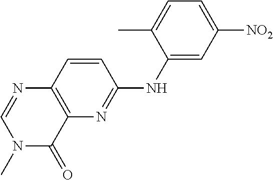 [SMILES: Cc1ccc([N+](=O)[O-])cc1Nc1ccc2ncn(C)c(=O)c2n1]

A solution of 6-chloro-3-methyl-pyrido[3,2-d]pyrimidin-4-one (1.71 g, 8.74 mmol, 1.00 eq), 2-Methyl-5-nitroaniline (3.99 g, 26.2 mmol, 3.00 eq), Palladium(II) acetate (0.20 g, 0.874 mmol, 0.100 eq), (S)-BINAP (0.54 g, 0.874 mmol, 0.100 eq), and cesium carbonate (5.70 g, 17.5 mmol, 2.00 eq) in 1,4-Dioxane (85 mL, 0.1028 M) was degassed by evacuating and charging the reaction flask with N₂ gas (3×) and heated to 80° C. for 3 h. From TLC, the starting material quinazoline-one was completed consumed. The reaction mixture was partitioned between DCM and water and separated. The aqueous was extracted with DCM (4×25 mL) and the combined organic fractions were dried over anhydrous Na₂SO₄, filtered, and concentrated in vacuo resulting in a crude solid. The solid was triturated in hot EtOAc and filtered through a fritted funnel resulting in 2.46 g, 82.9% yield of the title compound as a dark orange solid. ¹H NMR (500 MHz, DMSO-d₆) δ 9.23 (d, J=2.4 Hz, 1H), 8.95 (s, 1H), 8.28 (s, 1H), 7.93 (d, J=8.9 Hz, 1H), 7.84 (dd, J=8.4, 2.4 Hz, 1H), 7.51 (dd, J=13.8, 8.6 Hz, 2H), 3.50 (s, 3H), 2.43 (s, 3H); MS (ES⁺) m/z calc'd for [M+H]⁺ [C₁₅H₁₃N₅O₃+H]⁺: 312.30 found 312.1, LCMS: t_{R}=4.84 min [Analytical Method: 05991008_AA0.lcm]

### 6-(5-amino-2-methyl-anilino)-3-methyl-pyrido[3,2-d]pyrimidin-4-one [Intermediate 39]

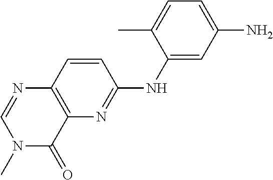 [SMILES: Cc1ccc(N)cc1Nc1ccc2ncn(C)c(=O)c2n1]

A suspension of 3-methyl-6-(2-methyl-5-nitro-anilino)pyrido[3,2-d]pyrimidin-4-one (2.20 g, 7.07 mmol, 1.00 eq) in Methanol (80 mL, 0.0707 M) and Ethyl acetate (20 mL, 0.0707 M) was added 5% Pd—C(0.99 g, 9.30 mmol, 1.32 eq) and stirred under hydrogen atmosphere (1 atm) using a balloon at rt for 10 h. Reaction mixture was filtered through wet celite bed and washed with MeOH (100 mL) and the filtrate was concentrated under reduced pressure. Crude product was washed with MeOH (2×10 mL) and filtered through fritted funnel, washed with ether (2×25 mL). The solid was dried under high vacuum for 70 h to afford 6-(5-amino-2-methyl-anilino)-3-methyl-pyrido[3,2-d]pyrimidin-4-one (1.12 g, 3.93 mmol, 55.63% yield) as tan solid. 1H NMR (500 MHz, DMSO-d₆) δ 8.70 (s, 1H), 8.17 (s, 1H), 7.75 (d, J=9.0 Hz, 1H), 6.98 (d, J=9.0 Hz, 1H), 6.88 (d, J=8.1 Hz, 1H), 6.68 (d, J=2.3 Hz, 1H), 6.34 (dd, J=8.1, 2.3 Hz, 1H), 4.84 (s, 2H), 3.47 (s, 3H), 2.02 (s, 3H). MS(ESI+) m/z calc'd for [M+H−HCl]⁺[C₁₅H₁₅N₅O+H]⁺: 282.13 found: 282.4, t_{R}=1.19 mins. [Method B].

### 6-(5-amino-2-methylphenoxy)-3-methylpyrido[3,2-d]pyrimidin-4(3H)-one [Intermediate 30]

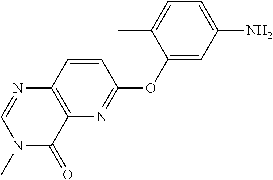 [SMILES: Cc1ccc(N)cc1Oc1ccc2ncn(C)c(=O)c2n1]

A solution of 6-chloro-3-methyl-pyrido[3,2-d]pyrimidin-4-one (0.10 g, 0.511 mmol, 1.00 eq), tert-butyl N-(3-hydroxy-4-methyl-phenyl)carbamate (0.23 g, 1.02 mmol, 2.00 eq) and Cesium carbonate (0.33 g, 1.02 mmol, 2.00 eq) in anhydrous DMF (2 mL, 0.2556 M) was heated to 90° C. for 16 h. The reaction mixture was partitioned between EtOAc and H₂0. The aqueous was extracted with EtOAc (3×10 mL) and the combined organic fractions were washed with 1M LiCl (2×10 mL), H₂O (1×10 mL), brine (1×10 mL), dried over anhydrous Na₂SO₄, filtered and concentrated in vacuo. The crude was purified by chromatography on silica gel, ISCO CombiFlash, 24 g cartridge [eluting with a 40 min, step gradient of 0% MeOH in DCM→8% MeOH in DCM] resulting in 54 mg, 37.4% yield of the title compound as a dark yellow solid. ¹H NMR (500 MHz, DMSO-d₆) δ 8.37 (s, 1H), 8.11 (d, J=8.8 Hz, 1H), 7.33 (d, J=8.8 Hz, 1H), 6.95 (dd, J=8.0, 0.8 Hz, 1H), 6.41 (dd, J=8.1, 2.3 Hz, 1H), 6.28 (d, J=2.3 Hz, 1H), 5.02 (s, 2H), 3.47 (s, 3H), 1.91 (s, 3H); MS (ES⁺) m/z calc'd for [M+H]⁺ [C₂₇H₂₄FN₉O₃S₂+H]⁺: 283.3 found 283.1, LCMS: t_{R}=3.21 min [05991008_AA0.lcm]

### Synthesis of 6-methyl-5-(4-morpholinothieno[3,2-d]pyrimidin-2-yl)pyridin-3-amine [Intermediate 31]

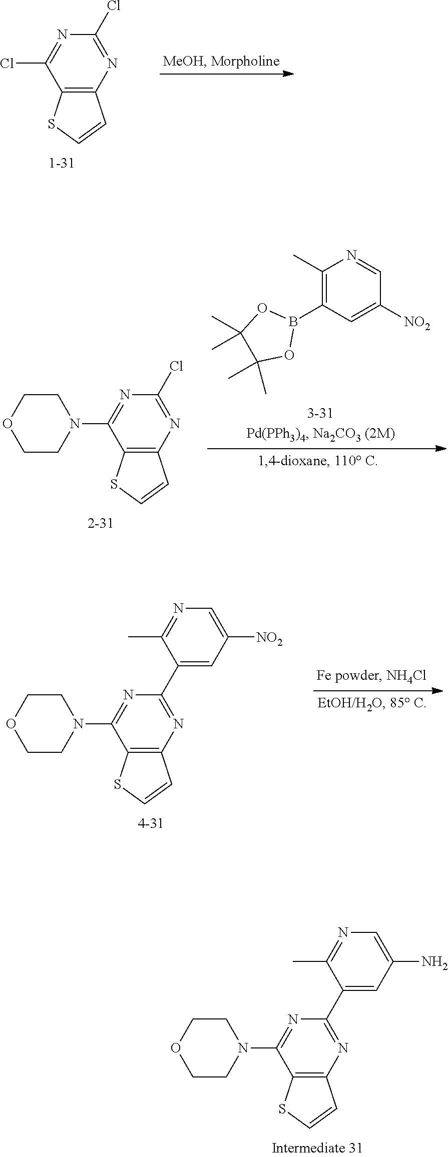

### 4-[2-(2-methyl-5-nitro-3-pyridyl)thieno[3,2-d]pyrimidin-4-yl]morpholine [4-31]

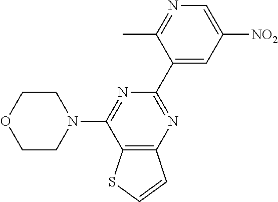 [SMILES: Cc1ncc([N+](=O)[O-])cc1-c1nc(N2CCOCC2)c2sccc2n1]

Nitrogen was bubbled through a mixture of 2-methyl-5-nitro-3-(4,4,5,5-tetramethyl-1,3,2-dioxaborolan-2-yl)pyridine (0.51 g, 1.93 mmol, 1.00 eq), 4-(2-chlorothieno[3,2-d]pyrimidin-4-yl)morpholine (0.49 g, 1.93 mmol, 1.00 eq), and Pd(PPh₃)₄ (0.045 g, 0.0387 mmol, 0.0200 eq) in 4:1 Dioxane:2M Na₂CO₃ (0.15 M, 13 mL) at rt for 10 minutes prior to capping the pressure vessel and lowering into an oil bath at 100° C. After 2 hours the reaction was cooled to rt and extracted with water. Organics were then concentrated in vacuo to afford 4-[2-(2-methyl-5-nitro-3-pyridyl)thieno[3,2-d]pyrimidin-4-yl]morpholine (0.68 g, 1.89 mmol) as a solid which was used directly in the next step without further purification.

### 6-methyl-5-(4-morpholinothieno[3,2-d]pyrimidin-2-yl)pyridin-3-amine [Intermediate 31]

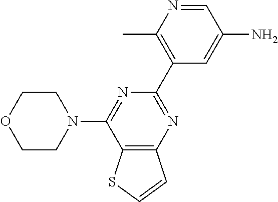 [SMILES: Cc1ncc(N)cc1-c1nc(N2CCOCC2)c2sccc2n1]

A mixture of 4-[2-(2-methyl-5-nitro-3-pyridyl)thieno[3,2-d]pyrimidin-4-yl]morpholine (0.68 g, 1.89 mmol, 1.00 eq), NH₄C₁ (0.30 g, 5.67 mmol, 3.00 eq), and Fe powder (0.37 g, 6.61 mmol, 3.50 eq) in EtOH (95%, 0.05 M, 38 mL) and H₂O (0.5 M, 3.8 mL) was lowered into an oil bath at 80-85° C. After 1.25 hours the reaction mixture was cooled to rt and loaded directly onto silica gel. Column chromatography (SiO₂; 0-15% MeOH in DCM) then afforded 0.46 g, 1.39 mmol, 72% yield over 2 steps of the title compound as a brown solid. ¹H NMR (600 MHz, DMSO-d₆) δ 8.27 (d, J=5.5 Hz, 1H), 7.89 (d, J=2.8 Hz, 1H), 7.50 (d, J=5.5 Hz, 1H), 7.43 (d, J=2.7 Hz, 1H), 5.17 (s, 2H), 3.97-3.94 (m, 4H), 3.81-3.76 (m, 4H), 2.54 (s, 3H); MS(ESI⁺) m/z calc'd for [C₁₆H₁₇N₅OS+H]⁺: 328.1 found: 328.1, t_{R}=3.50 min. [Analytical Method 05991008_AA0.lcm]

### 2-methyl-5-nitro-3-(4,4,5,5-tetramethyl-1,3,2-dioxaborolan-2-yl)pyridine [3-31]

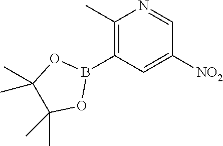 [SMILES: Cc1ncc([N+](=O)[O-])cc1B1OC(C)(C)C(C)(C)O1]

Pd(dppf)Cl₂ with DCM (0.20 g, 0.242 mmol, 0.0500 eq) was added to a mixture of 3-bromo-2-methyl-5-nitro-pyridine (1.05 g, 4.84 mmol, 1.00 eq), bis(pinacolato)diboron (1.47 g, 5.81 mmol, 1.20 eq), and KOAc (1.42 g, 14.5 mmol, 3.00 eq) in 1,4-dioxanes (0.3 M, 16 mL) after purging with nitrogen at rt for 20 minutes. The resulting reaction mixture was lowered into an oil bath at 100° C. and stirred for 3 hours. After cooling to rt the reaction mixture was loaded directly onto silica gel. Column chromatography (SiO₂; 0-20% EtOAc in Hex) then afforded 2-methyl-5-nitro-3-(4,4,5,5-tetramethyl-1,3,2-dioxaborolan-2-yl)pyridine (1.16 g, 4.36 mmol, 90.19% yield) as an off-white solid. ¹H NMR (600 MHz, DMSO-d₆) δ 9.29 (d, J=2.8 Hz, 1H), 8.55 (d, J=2.8 Hz, 1H), 2.78 (s, 3H), 1.35 (s, 12H); MS(ESI⁺) m/z calc'd for [Cl₂H₁₇BN₂O₄+H]⁺: 265.09 found: 265.0, t_{R}=2.67 min. [Method 05991008_AA0.lcm]

### Synthesis of 3-fluoro-4-methyl-5-(4-morpholinothieno[3,2-d]pyrimidin-2-yl)aniline [Intermediate 32]

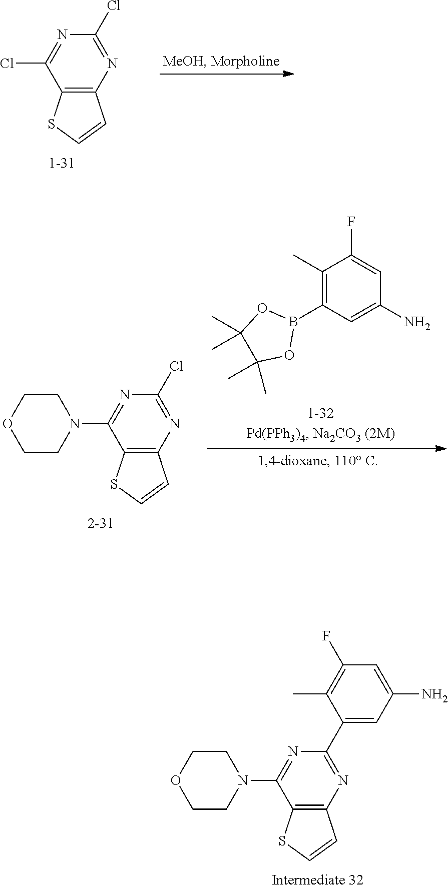

### 3-fluoro-4-methyl-5-(4-morpholinothieno[3,2-d]pyrimidin-2-yl)aniline [Intermediate 32]

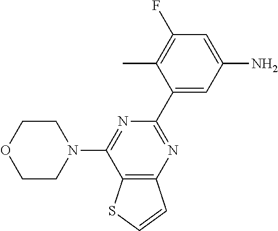 [SMILES: Cc1c(F)cc(N)cc1-c1nc(N2CCOCC2)c2sccc2n1]

Nitrogen was bubbled through a mixture of 3-fluoro-4-methyl-5-(4,4,5,5-tetramethyl-1,3,2-dioxaborolan-2-yl)aniline (0.19 g, 0.737 mmol, 1.00 eq), 4-(2-chlorothieno[3,2-d]pyrimidin-4-yl)morpholine (0.19 g, 0.737 mmol, 1.00 eq), and Pd(PPh₃)₄ (0.017 g, 0.0147 mmol, 0.0200 eq) in 4:1 Dioxane:2M Na₂CO₃ (0.15 M, 4.9 mL) at rt for 18 minutes prior to capping the pressure vessel and lowering into an oil bath at 100° C. to stir overnight. The reaction mixture was cooled to rt and loaded directly onto silica gel using EtOAc to transfer. Column chromatography (silica gel; 20-75% EtOAc in Hex) then afforded 0.22 g, 0.611 mmol, 82.91% yield of the title compound as a yellow solid. ¹H NMR (600 MHz, DMSO-d₆) δ 8.26 (d, J=5.5 Hz, 1H), 7.48 (d, J=5.5 Hz, 1H), 6.86 (d, J=2.2 Hz, 1H), 6.40 (dd, J=12.2, 2.3 Hz, 1H), 5.24 (s, 2H), 3.96-3.93 (m, 4H), 3.79-3.75 (m, 4H), 2.19 (d, J=2.0 Hz, 3H); MS(ESI⁺) m/z calc'd for [C₁₇H₁₇FN₄OS+H]⁺: 345.1 found: 345.1, t_{R}=4.29 min. [Analytical Method 05991008_AA0.lcm]

### 3-fluoro-4-methyl-5-(4,4,5,5-tetramethyl-1,3,2-dioxaborolan-2-yl)aniline [1-32]

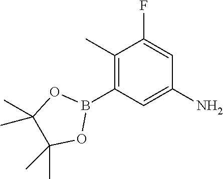 [SMILES: Cc1c(F)cc(N)cc1B1OC(C)(C)C(C)(C)O1]

Pd(dppf)Cl₂ with DCM (0.14 g, 0.167 mmol, 0.0500 eq) was added to a mixture of 3-bromo-5-fluoro-4-methyl-aniline (0.68 g, 3.33 mmol, 1.00 eq), Bis(pinacolato)diboron (0.93 g, 3.67 mmol, 1.10 eq), and KOAc (0.98 g, 10.0 mmol, 3.00 eq) in 1,4-dioxanes (0.3 M, 11.1 mL) after purging with nitrogen at rt for 20 minutes. The resulting reaction mixture was lowered into an oil bath at 105° C. and stirred for 2.75 hours. After cooling to rt the reaction mixture was loaded directly onto silica gel. Column chromatography (silica gel; 0-20% EtOAc in Hex) then afforded 0.59 g, 2.33 mmol, 69.91% yield of the title compound as a solid. ¹H NMR (600 MHz, DMSO-d₆) δ 6.74 (d, J=2.3 Hz, 1H), 6.38 (dd, J=12.5, 2.3 Hz, 1H), 5.13 (s, 2H), 2.18 (d, J=2.1 Hz, 3H), 1.28 (s, 12H)

### Synthesis of 3-fluoro-4-((2-(5-(((2-methoxyethyl)amino)methyl)pyridin-2-yl)thieno[3,2-b]pyridin-7-yl)oxy)aniline [Intermediate 33]

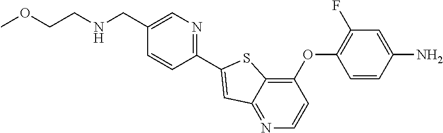 [SMILES: COCCNCc1ccc(-c2cc3nccc(Oc4ccc(N)cc4F)c3s2)nc1]

Intermediate 33 is known in the literature and the synthesis can be performed according to the reference below:
Reference: WO2009/26720A1

### Synthesis of 5-(5-amino-3-hydroxy-2-methylphenyl)-1-methyl-3-morpholinopyridin-2(1H)-one [Intermediate 34]

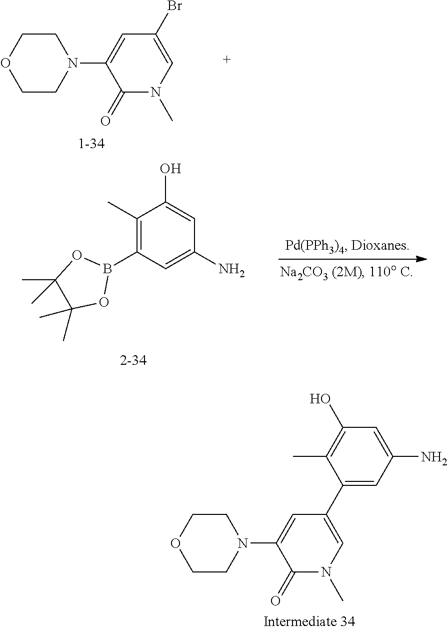 [SMILES: Cn1cc(Br)cc(N2CCOCC2)c1=O ; Cc1c(O)cc(N)cc1B1OC(C)(C)C(C)(C)O1 ; Cc1c(O)cc(N)cc1-c1cc(N2CCOCC2)c(=O)n(C)c1]

### 5-(5-amino-3-hydroxy-2-methyl-phenyl)-1-methyl-3-morpholino-pyridin-2-one [Intermediate 34]

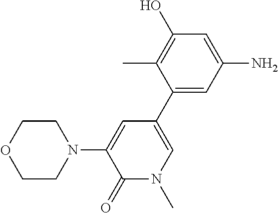 [SMILES: Cc1c(O)cc(N)cc1-c1cc(N2CCOCC2)c(=O)n(C)c1]

Nitrogen was bubbled through a mixture of 5-amino-2-methyl-3-(4,4,5,5-tetramethyl-1,3,2-dioxaborolan-2-yl)phenol (0.26 g, 1.04 mmol, 1.00 eq), 5-bromo-1-methyl-3-morpholino-pyridin-2-one (0.28 g, 1.04 mmol, 1.00 eq) [From intermediate 12 synthesis], and Pd(PPh3)4 (0.060 g, 0.0520 mmol, 0.0500 eq) in 4:1 Dioxane:2M Na₂CO₃ (0.15 M, 6.9 mL) at rt for 20 minutes prior to lowering into an oil bath at 110° C. to stir overnight. The reaction mixture was cooled to rt and concentrated onto silica gel. Column chromatography (silica; dry load; 20-80% ACN in Toluene) then afforded 0.13 g, 0.394 mmol, 12% yield over 2 steps of the title compound as a yellow-brown solid. 1H NMR (500 MHz, DMSO-d₆) δ 8.88 (s, 1H), 7.23 (d, J=2.2 Hz, 1H), 6.58 (d, J=2.2 Hz, 1H), 6.09 (d, J=2.1 Hz, 1H), 5.92 (d, J=2.2 Hz, 1H), 4.78 (s, 2H), 3.76-3.66 (m, 4H), 3.46 (s, 3H), 3.07 (d, J=4.1 Hz, 4H), 1.89 (s, 3H); MS (ES⁺) m/z calcd. for [M+H]⁺ [C₁₇H₂₁N₃03+H]⁺: 316.1 found 316.1 LCMS t_{R}=2.71 min [Analytical Method 05991008_AA0.lcm].

### 5-amino-2-methyl-3-(4,4,5,5-tetramethyl-1,3,2-dioxaborolan-2-yl)phenol [2-34]

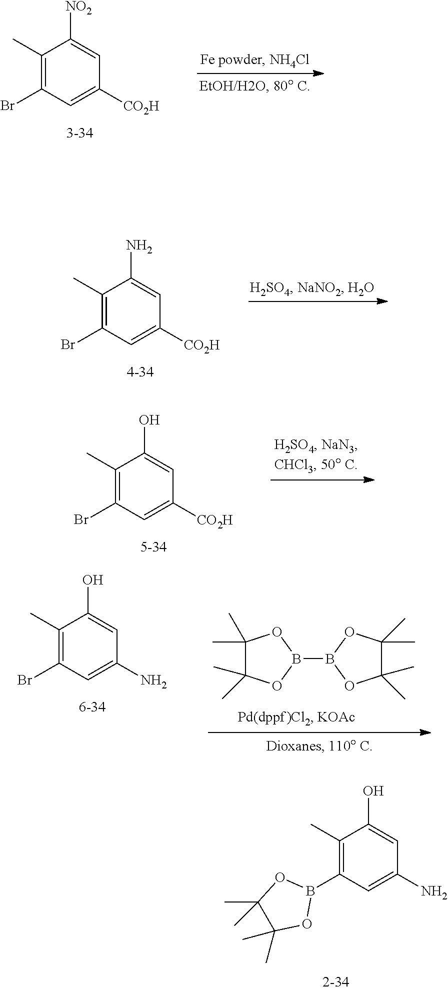

### 3-amino-5-bromo-4-methyl-benzoic acid [4-34]

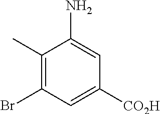

A mixture of 3-bromo-4-methyl-5-nitro-benzoic acid (2.70 g, 10.4 mmol, 1.00 eq), NH₄Cl (1.25 g, 23.3 mmol, 2.25 eq), and Fe powder (1.48 g, 26.4 mmol, 2.55 eq) in 95% EtOH (0.14 M, 74 mL) and water (0.65 M, 16 mL) was lowered into an oil bath at 80-85° C. After one hour the reaction mixture was cooled to rt and loaded onto silica gel. Column chromatography (silica gel; 0-10% MeOH in DCM) afforded 2.30 g, 10.0 mmol, 96.39% yield of the title compound as an orange solid. 1H NMR (600 MHz, DMSO-d₆) δ 12.81 (s, 1H), 7.26 (s, 1H), 7.22 (s, 1H), 5.47 (s, 2H), 2.19 (s, 3H).

### 3-bromo-5-hydroxy-4-methyl-benzoic acid [5-34]

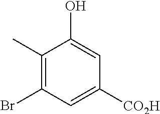

3-amino-5-bromo-4-methyl-benzoic acid (0.13 g, 0.565 mmol, 1.00 eq) in 30% v/v H2SO4 (aq., 0.22 M, 2.57 mL) was lowered stirred at 95° C. for 10 minutes. After cooling to 0° C., NaNO2 (0.041 g, 0.593 mmol, 1.05 eq) in water (0.57 mL) was added dropwise. The reaction mixture was slowly allowed to warm to rt, poured into 20% v/v H₂SO₄ (aq., 0.11 M, 5.14 mL), and then heated to 135° C. After 1.3 hours the reaction was cooled to rt, poured onto ice, and extracted with EtOAc. The combined organics were rinsed with brine, dried over sodium sulfate, and concentrated to afford 3-bromo-5-hydroxy-4-methyl-benzoic acid (0.12 g, 0.411 mmol, 72.76% yield) as a solid which was used in subsequent reactions without further purification. 1H NMR (600 MHz, DMSO-d₆) δ 13.03 (s, 1H), 10.26 (s, 1H), 7.53 (d, J=1.5 Hz, 1H), 7.39 (d, J=1.4 Hz, 1H), 2.24 (s, 3H); MS (ES⁺) m/z calcd. for [M+H]⁺ [C₈H₇BrO₃+H]⁺: 231.05 found 231.05 LCMS t_{R}=5.43 min [Analytical Method 05991008_AA0.lcm].

### 5-amino-3-bromo-2-methyl-phenol [6-34:]

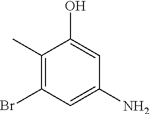

H2SO4 (0.43 M, 15 mL) was added to 3-bromo-5-hydroxy-4-methyl-benzoic acid (1.49 g, 6.44 mmol, 1.00 eq), and NaN3 (0.63 g, 9.67 mmol, 1.50 eq) in chloroform (0.32 M, 20 mL) and the resulting mixture stirred at 50° C. overnight. The reaction was cooled to rt, poured over ice, and extracted with EtOAc. The pH of the aqueous layer was then adjusted to ˜7 via the portion wise addition of sodium hydroxide pellets and then extracted with EtOAc. The second round of extractions were combined, rinsed with brine, dried over sodium sulfate, and concentrated in vacuo to provide 0.66 g, 0.871 mmol, 13.52% yield of the title compound as a dark residue used in the next reaction without further purification. 1H NMR (500 MHz, DMSO-d₆) δ 11.94 (s, 1H), 9.27 (s, 1H), 6.28 (d, J=2.1 Hz, 1H), 6.08 (d, J=2.1 Hz, 1H), 2.01 (s, 3H); MS (ES⁺) m/z calcd. for [M+H]⁺[C₇H₈BrNO+H]⁺: 201.9 found 202.0 LCMS t_{R}=3.88 min [Analytical Method 05991008_AA0.lcm].

### 5-amino-2-methyl-3-(4,4,5,5-tetramethyl-1,3,2-dioxaborolan-2-yl)phenol [2-34]

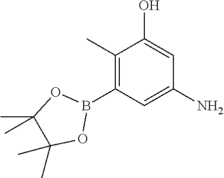 [SMILES: Cc1c(O)cc(N)cc1B1OC(C)(C)C(C)(C)O1]

Pd(dppf)Cl₂ with DCM (0.13 g, 0.163 mmol, 0.0500 eq) was added to a mixture of 5-amino-3-bromo-2-methyl-phenol (0.66 g, 3.25 mmol, 1.00 eq), Bis(pinacolato)diboron (0.91 g, 3.58 mmol, 1.10 eq), and KOAc (0.96 g, 9.76 mmol, 3.00 eq) in 1,4-dioxanes (0.3 M, 4.5 mL) after purging with nitrogen at rt for 20 minutes. The resulting reaction mixture was lowered into an oil bath at 105-110° C. and stirred overnight. After cooling to rt the reaction mixture was diluted with EtOAc and loaded directly onto silica gel. Column chromatography (silica; 30-75% EtOAc in Hex) then afforded 5-amino-2-methyl-3-(4,4,5,5-tetramethyl-1,3,2-dioxaborolan-2-yl)phenol (0.26 g, 0.780 mmol) as a semi-solid composing of a 3:1 mixture of desired product to de-brominated starting material (75% NMR purity). NMR peaks for desired product: ¹H NMR (600 MHz, DMSO-d₆) δ 8.67 (s, 1H), 6.39 (d, J=2.3 Hz, 1H), 6.16 (d, J=2.3 Hz, 1H), 4.67 (br s, 2H), 1.91 (s, 3H), 1.26 (s, 12H).

### Synthesis of (3-amino-5-bromo-2,6-difluoro-phenyl)-(5-chloro-1H-pyrrolo[2,3-b]pyridin-3-yl)methanone [Intermediate 35]

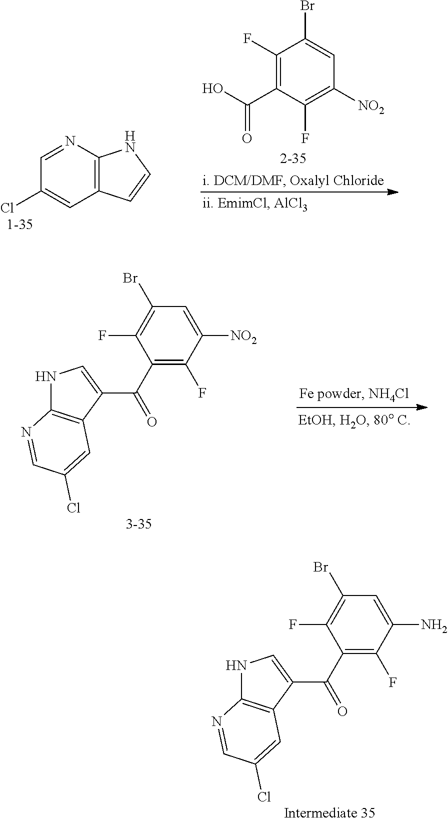 [SMILES: O=C(O)c1c(F)c(Br)cc([N+](=O)[O-])c1F ; Clc1cnc2[nH]ccc2c1 ; O=C(c1c(F)c(Br)cc([N+](=O)[O-])c1F)c1c[nH]c2ncc(Cl)cc12 ; Nc1cc(Br)c(F)c(C(=O)c2c[nH]c3ncc(Cl)cc23)c1F]

### (3-bromo-2,6-difluoro-5-nitro-phenyl)-(5-chloro-1H-pyrrolo[2,3-b]pyridin-3-yl)methanone [3-35]

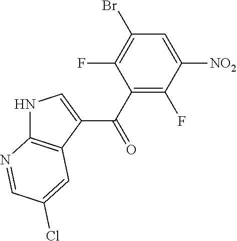 [SMILES: O=C(c1c(F)c(Br)cc([N+](=O)[O-])c1F)c1c[nH]c2ncc(Cl)cc12]

To a flame-dried flask charged with 3-bromo-2,6-difluoro-5-nitro-benzoic acid (0.75 g, 2.66 mmol, 1.50 eq) in DCM (0.2 M, 8.9 mL) and DMF (0.075 equiv., 0.01 mL) was added Oxalyl Chloride (0.35 mL, 4.00 mmol, 2.25 eq) at rt. After stirring for 1 hour, the reaction mixture was concentrated in vacuo. Separately, AlCl₃ (1.42 g, 10.7 mmol, 6.00 eq) was added to 1-Ethyl-3-methylimidazolium chloride (0.52 g, 3.55 mmol, 2.00 eq) in a flame-dried flask and stirred vigorously at rt. Once this mixture had cooled to rt, 5-chloro-1H-pyrrolo[2,3-b]pyridine (0.27 g, 1.78 mmol, 1.00 eq) was added. This was then poured into the reaction flask containing the acyl chloride residue and the resulting mixture was stirred vigorously overnight at rt. Ice was added to quench the reaction which was further diluted with water and sodium bicarbonate (sat., aq.) until neutral pH. Aqueous was extracted with EtOAc (×5). Combined organics were washed with sodium bicarbonate (aq., sat.), water, then brine and dried over sodium sulfate to provide 0.45 g, 1.01 mmol, 56.63% yield of the title compound as an orange solid which was used in the subsequent reaction without further purification. ¹H NMR (600 MHz, DMSO-d₆) δ 13.28 (s, 1H), 8.80 (t, J=7.4 Hz, 1H), 8.55 (s, 1H), 8.52 (d, J=2.4 Hz, 1H), 8.48 (d, J=2.4 Hz, 1H); MS(ESI⁺) m/z calc'd for [C₁₄H₅BrClF₂N₃O₃+H]⁺: 415.9 found: 415.9, t_{R}=7.67 min. [Analytical Method 05991008_AA0.lcm]

### (3-amino-5-bromo-2,6-difluoro-phenyl)-(5-chloro-1H-pyrrolo[2,3-b]pyridin-3-yl)methanone [Intermediate 35]

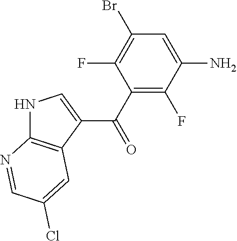 [SMILES: Nc1cc(Br)c(F)c(C(=O)c2c[nH]c3ncc(Cl)cc23)c1F]

The headspace of a mixture of (3-bromo-2,6-difluoro-5-nitro-phenyl)-(5-chloro-1H-pyrrolo[2,3-b]pyridin-3-yl)methanone (0.19 g, 0.461 mmol, 1.00 eq), Ammonium Chloride (0.074 g, 1.38 mmol, 3.00 eq), and Fe powder (0.090 g, 1.61 mmol, 3.50 eq) in 95% EtOH (0.075 M, 6.1 mL) and H₂O (0.5 M, 0.9 mL) was purged with nitrogen before lowering into an oil bath and stirring at 80° C. for 3.5 hours. The reaction mixture was then cooled to rt and loaded directly onto silica gel. Column chromatography (SiO₂; dry load; 0-10% MeOH in DCM) then provided (3-amino-5-bromo-2,6-difluoro-phenyl)-(5-chloro-1H-pyrrolo[2,3-b]pyridin-3-yl)methanone (0.15 g, 0.366 mmol, 79.48% yield) as a solid. 1H NMR (500 MHz, DMSO-d₆) δ 13.09 (br s, 1H), 8.45 (d, J=2.4 Hz, 1H), 8.43 (d, J=2.4 Hz, 1H), 8.30 (s, 1H), 7.15 (dd, J=9.1, 7.2 Hz, 1H), 5.49 (s, 2H); MS(ESI⁺) m/z calc'd for [M+H]⁺ [C₁₄H₇BiClF₂N₃O+H]⁺: 385.9 found: 385.9, t_{R}=6.68 min. [Analytical Method 05991008_AA0.lcm]

### 3-bromo-2,6-difluoro-5-nitro-benzoic acid [2-35]

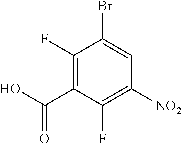 [SMILES: O=C(O)c1c(F)c(Br)cc([N+](=O)[O-])c1F]

To a slurry of 3-bromo-2,6-difluoro-benzoic acid (1.02 g, 4.28 mmol, 1.00 eq) in H₂SO₄ (1.25 M, 3.4 mL) was added 15.8 N HNO₃ (6.3 M, 0.68 mL) dropwise at 0° C. The reaction mixture was then allowed to warm to rt and subsequently heated to 50° C. to stir overnight. The reaction mixture was cooled to rt, poured over ice, and extracted with EtOAc. Combined organics were washed with brine, dried over sodium sulfate, and concentrated in vacuo to afford crude 3-bromo-2,6-difluoro-5-nitro-benzoic acid (1.09 g, 3.85 mmol, 89.86% yield) which was used in subsequent steps without further purification. 1H NMR (600 MHz, DMSO-d₆) δ 8.73 (t, J=7.4 Hz, 1H). MS(ESI⁺) m/z calc'd for [2M−H]⁺ [C₁₄H₄Br₂F₄N₂O₈−H]⁻: 560.8 found: 560.8, t_{R}=6.68 min. [Analytical Method 05991008_AA0.lcm]

### Synthesis of 5-amino-2′-methyl-5′-morpholino-[1,1′-biphenyl]-3-ol [Intermediate 36]

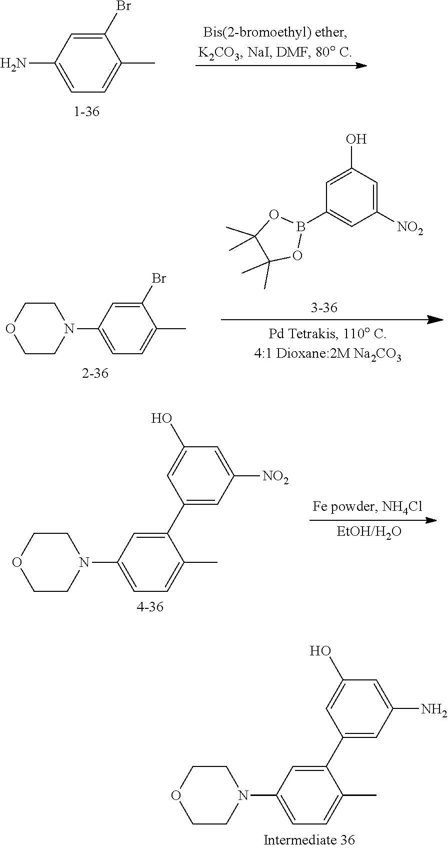

### 4-(3-bromo-4-methyl-phenyl)morpholine [2-36]

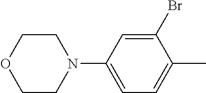

A mixture of 3-bromo-4-methylaniline (1.14 g, 6.11 mmol, 1.00 eq) and K₂CO₃ (0.93 g, 6.72 mmol, 1.10 eq) in DMF (6.1113 mL, 1 M) was purged with nitrogen for 10 minutes at rt before adding Bis(2-bromoethyl) ether (0.84 mL, 6.72 mmol, 1.10 eq). The reaction mixture was further purged for 10 minutes before adding NaI (0.046 g, 0.306 mmol, 0.0500 eq) and lowering into an oil bath at 80° C. After 3 days, the reaction was cooled to rt, quenched with ammonium chloride (aq., sat.), and extracted with EtOAc. Combined organics were washed with LiCl (1M, aq.), water, brine, and then dried over sodium sulfate before concentrating in vacuo onto silica gel. Column chromatography (silica gel; dry load; 0-10% EtOAc in Hex) afforded 1.21 g, 4.71 mmol, 77.11% yield of the title compound as an orange oil. ¹H NMR (600 MHz, CDCl₃) δ 7.11 (d, J=8.4 Hz, 1H), 7.08 (d, J=2.5 Hz, 1H), 6.77 (dd, J=8.4, 2.5 Hz, 1H), 3.87-3.77 (m, 4H), 3.13-3.05 (m, 4H), 2.31 (s, 3H); MS(ESI⁺) m/z calc'd for [C₁₁H₁₄BrNO+H]⁺: 256.0 found: 256.0, t_{R}=7.52 min. [Analytical Method 05991008_AA0.lcm]

### 3-(2-methyl-5-morpholino-phenyl)-5-nitro-phenol [4-36]

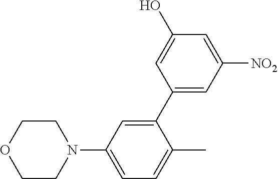 [SMILES: Cc1ccc(N2CCOCC2)cc1-c1cc(O)cc([N+](=O)[O-])c1]

A mixture of 3-nitro-5-(4,4,5,5-tetramethyl-1,3,2-dioxaborolan-2-yl)phenol (0.25 g, 0.958 mmol, 1.00 eq), 4-(3-bromo-4-methyl-phenyl)morpholine (0.26 g, 1.01 mmol, 1.05 eq), and Pd(PPh₃)₄ (0.022 g, 0.0192 mmol, 0.0200 eq) in 4:1 dioxane:2M Na₂CO₃ (0.15 M, 6.4 mL) was purged with nitrogen at rt for 10 minutes prior to capping the pressure vessel and lowering into an oil bath at 110° C. After 17 hours, the reaction was cooled to rt and concentrated directly onto silica gel. Column chromatography (silica gel; dry load; 5-30% EtOAc in Hexanes) afforded 0.35 g, 1.10 mmol, 114.54% yield of the title compound as an orange semi-solid. NMR revealed to be an ˜2:1 mixture of desired product to pinacol. Used in next reaction without further purification. ¹H NMR (600 MHz, CDCl₃, peaks for desired product only) δ 7.77-7.73 (m, 1H), 7.66 (t, J=2.2 Hz, 1H), 7.19 (d, J=8.4 Hz, 1H), 7.03 (s, 1H), 6.90 (dd, J=8.4, 2.7 Hz, 1H), 6.75 (d, J=2.6 Hz, 1H), 6.15 (s, 1H), 3.90-3.86 (m, 4H), 3.18-3.14 (m, 4H), 2.16 (s, 3H); MS(ESI⁺) m/z calc'd for [C₁₇H₁₈N₂O₄+H]⁺: 315.1 found: 315.1, t_{R}=3.93 min. [Analytical Method 05990510_AA1.lcm]

### 3-amino-5-(2-methyl-5-morpholino-phenyl)phenol [Intermediate 36]

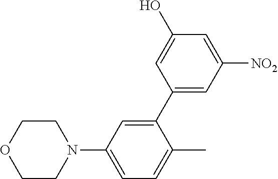 [SMILES: Cc1ccc(N2CCOCC2)cc1-c1cc(O)cc([N+](=O)[O-])c1]

A mixture of 3-(2-methyl-5-morpholino-phenyl)-5-nitro-phenol (0.34 g, 1.08 mmol, 1.00 eq), Ammonium Chloride (0.17 g, 3.25 mmol, 3.00 eq), and Fe powder (0.21 g, 3.80 mmol, 3.50 eq) in 95% EtOH (0.13 M, 8.0 mL) and H₂O (0.65 M, 1.7 mL) was stirred at 80° C. for 30 minutes. The reaction mixture was then cooled to rt and loaded directly onto silica gel. Column chromatography (silica gel; dry load; 0-10% MeOH in DCM) provided 0.28 g, 0.985 mmol, 90.77% yield of the title compound as a white solid. NMR revealed to be ˜2:1 mixture of desired to pinacol—likely carried over from previous reaction. Material was used in subsequent reaction without further purification. ¹H NMR (600 MHz, CDCl₃) δ 7.13 (d, J=8.3 Hz, 1H), 6.81 (dd, J=8.3, 2.7 Hz, 1H), 6.78 (d, J=2.6 Hz, 1H), 6.21 (d, J=1.5 Hz, 1H), 6.19-6.14 (m, 2H), 4.64 (s, 1H), 3.90-3.80 (m, 4H), 3.70 (s, 2H), 3.21-3.09 (m, 4H), 2.19 (s, 3H); MS(ESI⁺) m/z calc'd for [C₁₇H₂₀N₂O₂+H]⁺: 285.2 found: 285.2, t_{R}=4.70 min. [Analytical Method 05991008_BB1.lcm]

### Synthesis of 6-(3-amino-4-fluorophenoxy)-3-methylquinazoline-4(3H)-thione [Intermediate 38]

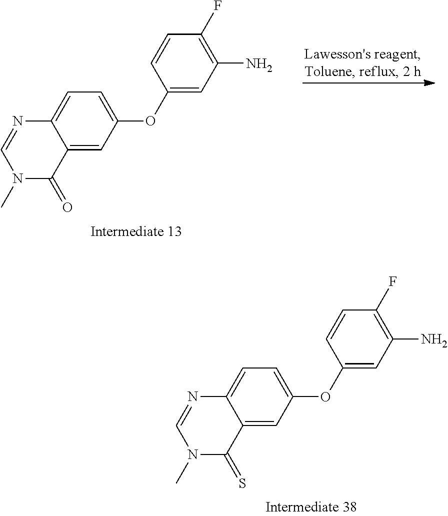 [SMILES: Cn1cnc2ccc(Oc3ccc(F)c(N)c3)cc2c1=O ; Cn1cnc2ccc(Oc3ccc(F)c(N)c3)cc2c1=S]

To a suspension of 6-(3-amino-4-fluorophenoxy)-3-methylquinazolin-4(3H)-one (1.50 g, 5.26 mmol, 1.00 eq) in toluene (55 mL, 0.0956 M) at rt was added Lawesson's reagent (3.19 g, 7.89 mmol, 1.50 eq). The suspension was heated to reflux for 2 h. Reaction mixture was concentrated under reduced pressure. Residue was diluted with water and ethyl acetate (50 mL each). The biphasic solution was stirred at rt for 5 mins. Two layers were separated; aqueous was extracted with ethyl acetate (2×50 mL). Combined organic part was washed with water (3×25 mL), followed by brine (25 mL) dried over sodium sulfate and concentrated under reduced pressure. Crude was purified by column chromatography over silica gel, ISCO, CombiFlash, 12 g cartridge (dry load, 0-10% MeOH/DCM as eluent). Pure fractions were combined and concentrated under reduced pressure to afford 0.68 g, 2.15 mmol, 40.83% yield of the title compound as yellow solid. ¹H NMR (500 MHz, DMSO-d₆) δ 8.64 (s, 1H), 8.06 (d, J=2.8 Hz, 1H), 7.77 (d, J=8.9 Hz, 1H), 7.58 (dd, J=8.9, 2.9 Hz, 1H), 7.04 (dd, J=11.2, 8.7 Hz, 1H), 6.58-6.39 (m, 1H), 6.24 (dt, J=8.7, 3.3 Hz, 1H), 3.86 (s, 3H), 3.80-3.77 (m, 2H). MS(ESI⁺) m/z calc'd for [M+H]⁺[C₁₅H₁₂FN₃OS+H]⁺: 302.0 found: 302.2, t_{R}=2.31 mins. [Analytical Method B].

### Synthesis of 6-((5-amino-2-methylphenyl)amino)-3-methylquinazoline-4(3H)-thione [Intermediate 40]

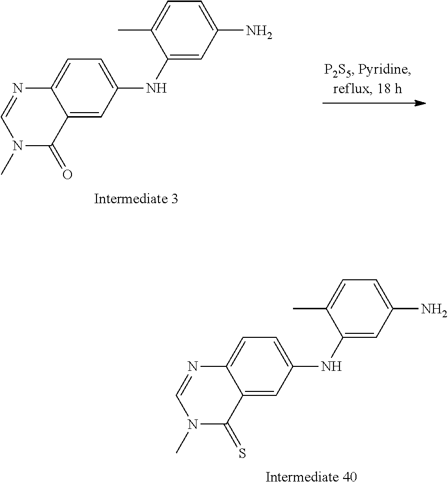 [SMILES: Cc1ccc(N)cc1Nc1ccc2ncn(C)c(=O)c2c1 ; Cc1ccc(N)cc1Nc1ccc2ncn(C)c(=S)c2c1]

To a suspension of phosphorus pentasulfide (0.59 g, 1.34 mmol, 1.50 eq) in pyridine (4 mL, 0.2230 M) at rt was added 6-((5-amino-2-methylphenyl)amino)-3-methylquinazolin-4(3H)-one (0.25 g, 0.892 mmol, 1.00 eq). The suspension was heated to 121° C. for 18 h. Reaction mixture was concentrated under reduced pressure. Residue was diluted with water and ethyl acetate (25 mL each). The biphasic solution was stirred at rt for 5 mins. Two layers were separated; aqueous was extracted with ethyl acetate (2×25 mL). Combined organic fractions were washed with water (3×25 mL), followed by brine (5 mL) dried over sodium sulfate, filtered, and concentrated under reduced pressure. Crude was purified by column chromatography over silica gel, ISCO, CombiFlash, 4 g cartridge (dry load, 0% to 10% MeOH/DCM as eluent). Pure fractions were combined and concentrated under reduced pressure to dryness before it was diluted with diethyl ether (15 mL). The suspension was stirred at rt for 10 mins and filtered. Solids were dried under reduced pressure to afford 0.070 g, 0.207 mmol, 23.20% yield of the title compound as yellow solid. ¹H NMR (500 MHz, DMSO-d₆) δ 8.46 (d, J=1.3 Hz, 1H), 7.97 (s, 1H), 7.86 (t, J=2.0 Hz, 1H), 7.55 (dd, J=8.8, 1.2 Hz, 1H), 7.37 (dt, J=8.8, 2.1 Hz, 1H), 6.90 (d, J=8.2 Hz, 1H), 6.48 (d, J=2.3 Hz, 1H), 6.31 (dd, J=8.1, 2.3 Hz, 1H), 4.94 (s, 2H), 3.86 (d, J=1.2 Hz, 3H), 2.00 (s, 3H). MS(ESI⁺) m/z calc'd for [M+H]⁺[C₁₆H₁₆N₄S+H]⁺: 297.1 found: 297.4, t_{R}=1.77 mins. [Analytical Method B].

### Synthesis of 4-(4-methyl-2-(pyridin-3-yl)thiazol-5-yl)pyridin-2-amine [Intermediate 41]

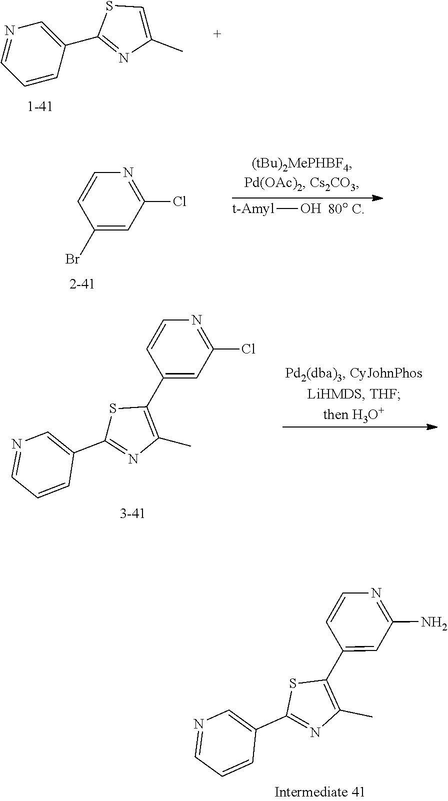

### 5-(2-chloro-4-pyridyl)-4-methyl-2-(3-pyridyl)thiazole [3-41]

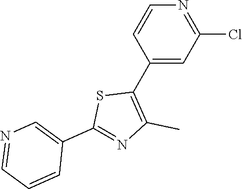 [SMILES: Cc1nc(-c2cccnc2)sc1-c1ccnc(Cl)c1]

4-methyl-2-(3-pyridyl)thiazole (0.35 g, 1.97 mmol, 1.00 eq) was added to a flame dried flask containing a mixture of 4-bromo-2-chloro-pyridine (0.33 mL, 2.96 mmol, 1.50 eq), cesium carbonate (0.97 g, 2.96 mmol, 1.50 eq), (tBu)₂MePHBF₄ (0.20 g, 0.790 mmol, 0.400 eq), and Pd(OAc)₂ (0.088 g, 0.395 mmol, 0.200 eq) in t-Amyl-OH (0.52 M, 3.8 mL) while purging with nitrogen for 25 minutes at rt. The reaction mixture was then lowered into an oil bath and stirred at 80° C. overnight. The following day, Pd(dppf)Cl₂ with DCM (0.081 g, 0.0987 mmol, 0.0500 eq) was added to the cooled reaction mixture which was subsequently lowered back into the oil bath to stir at 80° C. once more overnight. After cooling to rt, the reaction mixture was diluted with EtOAc and water before filtering through celite. The organic layer was isolated and rinsed with brine before concentrating directly onto silica gel. Column chromatography (silica gel; 0-30% EtOAc in hexanes) then provided 0.35 g, 1.22 mmol, 61.81% yield of the title compound as an off-white solid. ¹H NMR (600 MHz, DMSO-d₆) δ 8.66 (ddd, J=4.8, 1.6, 0.9 Hz, 1H), 8.51 (d, J=5.2 Hz, 1H), 8.15 (d, J=7.9 Hz, 1H), 8.00 (td, J=7.7, 1.7 Hz, 1H), 7.73 (d, J=1.0 Hz, 1H), 7.63 (dd, J=5.2, 1.6 Hz, 1H), 7.54 (ddd, J=7.5, 4.8, 1.1 Hz, 1H), 2.60 (s, 3H); MS(ESI⁺) m/z calc'd for [C₁₄H₁₀ClN₃S+H]⁺: 288.0 found: 288.0, t_{R}=4.11 min.[Analytical Method 05990510_AA1.lcm]

### 4-[4-methyl-2-(3-pyridyl)thiazol-5-yl]pyridin-2-amine [Intermediate 41]

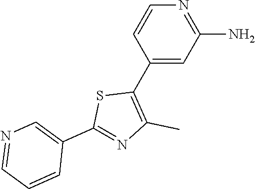 [SMILES: Cc1nc(-c2cccnc2)sc1-c1ccnc(N)c1]

A mixture of 5-(2-chloro-4-pyridyl)-4-methyl-2-(3-pyridyl)thiazole (0.10 g, 0.358 mmol, 1.00 eq), Pd₂(dba)₃ (0.049 g, 0.0537 mmol, 0.150 eq), and CyJohnPhos (0.040 g, 0.115 mmol, 0.320 eq) in THF (1.7896 mL, 0.2000 M), charged to a flame-dried flask, was purged with nitrogen for 20 minutes at rt. LiHMDS (0.54 mL, 0.537 mmol, 1.50 eq) was added and the resulting reaction mixture was lowered into an oil bath at 80° C. After cooling to rt 17 hours later, 2M HCl was added to quench. After 30 minutes of stirring, the mixture was diluted with water, neutralized with saturated sodium bicarbonate (aq.) and extracted with EtOAc. Combined organics were rinsed with brine and dried over sodium sulfate prior to concentrating onto silica gel. Column chromatography (silica gel; 0-10% 1:1:1 ACN:MeOH:H₂O in EtOAc) provided 0.017 g, 0.0594 mmol, 16.58% yield of the title compound as an orange solid. ¹H NMR (600 MHz, DMSO-d₆) δ 8.64 (d, J=4.7 Hz, 1H), 8.12 (d, J=7.9 Hz, 1H), 8.00-7.95 (m, 2H), 7.51 (ddd, J=7.5, 4.8, 1.0 Hz, 1H), 6.66 (dd, J=5.3, 1.6 Hz, 1H), 6.62 (s, 1H), 6.12 (s, 2H), 2.56 (s, 3H); MS(ESI⁺) m/z calc'd for [C₁₄H₁₂N₄S+H]⁺: 269.1 found: 269.0, t_{R}=1.54 min.[Analytical Method 05990510_AA1.1 cm]

### Synthesis of 6-(5-amino-2-chlorophenyl)-5,8-dimethyl-2-(methylamino) pyrido[2,3-d] pyrimidin-7(8H)-one [Intermediate 42]

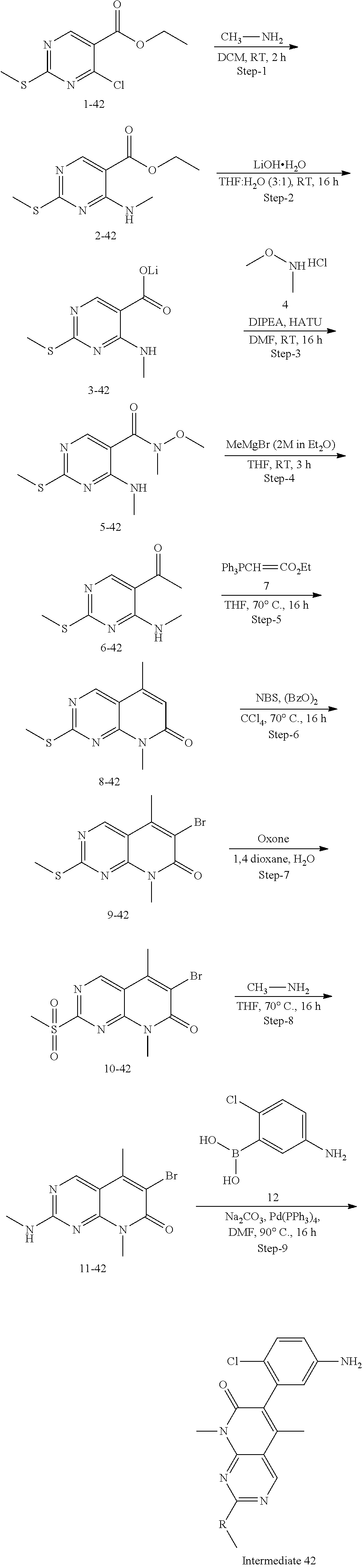

### Synthesis of ethyl 4-(methylamino)-2-methylsulfanyl-pyrimidine-5-carboxylate [2-42]

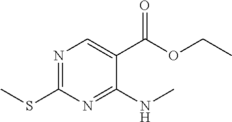

To a stirred solution of ethyl 4-chloro-2-(methylthio) pyrimidine-5-carboxylate (20 g, 85.9 mmol, 1 eq.) in DCM (250 mL), under nitrogen atmosphere was added methyl amine in 33% ethanol (37.4 mL, 278.4 mmol, 3.24 eq.) at 0° C. The resulting reaction mixture was slowly warm to room temperature and stirred for 2 h. The reaction mixture was diluted with water (100 mL) and extracted with DCM (2×200 mL). The combined organic layer was washed with brine solution (100 mL), dried over sodium sulfate, filtered, and concentrated under reduced pressure to afford 19 g, 96.28% yield of the title compound ethyl 4-(methylamino)-2-methylsulfanyl-pyrimidine-5-carboxylate as an off-white solid. The crude compound was used as in the next step without any further purification. 1H NMR (400 MHz, DMSO-d6): δ 8.52 (s, 1H), 8.25 (d, J=3.1 Hz, 1H), 4.30-4.25 (m, 2H), 2.98 (d, J=4.4 Hz, 3H), 2.49 (s, 3H), 1.30 (t, J=7.0 Hz, 3H). LCMS: MS (ES)+m/z calc'd for [M+H]⁺[C₉H₁₃N₃O₂S+H]⁺: 228.07, found 227.85, LCMS:t_{R}=1.92 min, [Method: C].

### Synthesis of lithium 4-(methylamino)-2-(methylthio) pyrimidine-5-carboxylate [3-42]

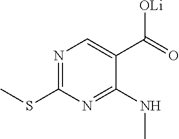

To a stirred solution of ethyl 4-(methylamino)-2-(methylthio) pyrimidine-5-carboxylate (7.8 g, 34.3 mmol, 1.0 eq.) in THF (150 mL) and water (50 mL), was added LiOH·H₂O (2.88 g, 68.6 mmol, 2.0 eq.) at room temperature. The reaction mixture was stirred at room temperature for 16 h. The reaction mixture was concentrated under reduced pressure to afford compound 6.8 g, 95% yield of the title compound as an off-white solid as a lithium salt which was used as such for next step without any further purification. ¹H NMR (400 MHz, DMSO-d₆): δ 10.36 (br s, 1H), 8.34 (s, 1H), 2.88 (s, 3H), 2.42 (s, 3H). LCMS: MS (ES⁺) m/z calc'd for [M+H]⁺[C₇H₈LiN₃O₂S+H]⁺: 206.05, found 200.15+Li⁺, LCMS: t_{R}=1.05 min, [Method: C].

### Synthesis of N-methoxy-N-methyl-4-(methylamino)-2-(methylthio)pyrimidine-5-carboxamide [5-42]

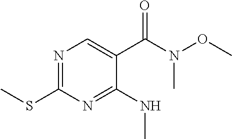

To a stirred solution of lithium 4-(methylamino)-2-(methylthio) pyrimidine-5-carboxylate (6.80 g, 33.1 mmol, 1.0 eq) in DMF (80 mL), was added compound (3.92 g, 39.8 mmol, 1.2 eq) and DIPEA (16 mL, 99.4 mmol, 3.0 eq) followed by HATU (18.9 g, 49.7 mmol, 1.5 eq) at room temperature under nitrogen atmosphere. The reaction mixture was stirred for 16 h. The reaction mixture was diluted with water (50 mL) and extracted with ethyl acetate (2×200 mL). The combined organic layer was washed with cold water (2×100 mL) and brine solution (50 mL), dried over sodium sulphate, filtered and concentrated under vacuum to afford 8 g of crude compound of the title compound as an off white solid. The crude compound was used as such for next step without any further purification. ¹H NMR (400 MHz, CDCl₃): δ 8.58 (s, 1H), 8.09 (br s, 1H), 3.63 (s, 3H), 3.06 (d, J=4.9 Hz, 3H), 2.81 (s, 3H), 2.58 (s, 3H); MS (ES)+m/z calc'd for [M+H]⁺[C₉H₁₄N₄O₂S+H]⁺: 243.08, found 242.90, LCMS: t_{R}=1.38 min [Method: C].

### Synthesis of 1-(4-(methylamino)-2-(methylthio)pyrimidin-5-yl)ethan-1-one [6-42]

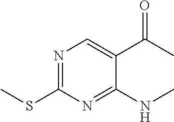

To a stirred solution of N-methoxy-N-methyl-4-(methylamino)-2-(methylthio) pyrimidine-5-carboxamide (2.0 g, 8.25 mmol, 1.0 eq) in THF (30 mL), methyl magnesium bromide (2.0 M in DEE) (12 mL, 24.8 mmol, 3.0 eq) was added drop wise at 0° C. under nitrogen atmosphere. The reaction mixture was stirred at room temperature for 3 h. The reaction mixture was quenched with sat. NH₄Cl solution (50 mL) and extracted with ethyl acetate (2×100 mL). The combined organic layer was washed with brine solution (50 mL), dried over sodium sulphate, filtered, and concentrated under reduced pressure to get crude compound. The crude compound was purified by comb flash using YMC-12 g cartridge, with 0-30% EA/heptane as an eluent to afford 1.2 g, 73% yield of the title compound as an off-white solid. ¹H NMR (400 MHz, CDCl₃): δ 9.37 (br s, 1H), 8.58 (s, 1H), 3.13 (d, J=5.0 Hz, 3H), 2.59 (s, 3H), 2.52 (s, 3H); MS (ES⁺) m/z calc'd for [M+H]⁺ [C₈H₁₁N₃OS+H]⁺: 198.06, found 197.85, LCMS: t_{R}=1.74 min [Method: C].

### Synthesis of 5,8-dimethyl-2-(methylthio) pyrido[2,3-d]pyrimidin-7(8H)-one [8-42]

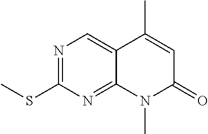

To a stirred solution of 1-(4-(methylamino)-2-(methylthio) pyrimidin-5-yl) ethan-1-one (1.0 g, 5.07 mmol, 1.0 eq.) in THF (10 mL), was added compound 6-42 (3.53 g, 10.1 mmol, 2.0 eq) at room temperature under nitrogen atmosphere. The reaction mixture was heated to 70° C. and stirred for 16 h. The reaction was quenched with water (50 mL) and extracted with ethyl acetate (2×50 mL). The combined organic layer was washed with brine solution (10 mL), dried over sodium sulphate, filtered, and concentrated under reduced pressure to get crude compound. The crude compound was purified by comb flash using YMC-12 g cartridge, eluting with 0-40% EA/heptane to afford compound 7 (0.7 g, 62% yield) as an off-white solid. ¹H NMR (400 MHz, CDCl₃): δ 8.70 (s, 1H), 6.48 (s, 1H), 3.72 (s, 3H), 2.64 (s, 3H), 2.44 (s, 3H); MS (ES)⁺ m/z calc'd for [M+H]⁺ [C₁₀H₁₁N₃OS+H]⁺: 222.06, found 221.85, LCMS: t_{R}=1.47 min [Method: C].

### Synthesis of 6-bromo-5,8-dimethyl-2-(methylthio)pyrido[2,3-d]pyrimidin-7(8H)-one [9-42]

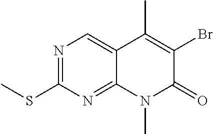 [SMILES: CSc1ncc2c(C)c(Br)c(=O)n(C)c2n1]

To a stirred solution of 5,8-dimethyl-2-(methylthio) pyrido[2,3-d] pyrimidin-7(8H)-one (0.7 g, 3.16 mmol, 1.0 eq) in carbon tetrachloride (20 mL), was added NBS (1.13 g, 6.33 mmol, 2.0 eq.) and (BzO)₂ (0.038 g, 0.158 mmol, 0.05 eq) at room temperature under nitrogen atmosphere. The reaction mixture was heated to 70° C. and stirred for 16 h. The reaction mixture was quenched with water (50 mL) and extracted with DCM (2×50 mL). The organic layer was washed with brine solution (50 mL), dried over sodium sulphate, filtered and concentrated under vacuum to get crude compound. The crude compound was purified by comb flash using YMC-12 g cartridge, eluting with 0-40% EA/heptane to afford compound 9 (0.3 g, 8.5% yield) as an off-white solid. ¹H NMR (400 MHz, DMSO-d₆): δ 9.07 (s, 1H), 3.66 (s, 3H), 2.63 (s, 3H), 2.61 (s, 3H); MS (ES)⁺ m/z calc'd for [M+H]⁺ [C₁₀H₁₀BrN₃OS+H]⁺: 299.97, found 300.06, LCMS: t_{R}=1.85 min [Method: C].

### Synthesis of 6-bromo-5,8-dimethyl-2-(methylsulfonyl)pyrido[2,3-d]pyrimidin-7(8H)-one [10-42]

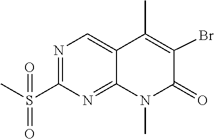 [SMILES: Cc1c(Br)c(=O)n(C)c2nc(S(C)(=O)=O)ncc12]

To a stirred solution of 6-bromo-5,8-dimethyl-2-(methylthio) pyrido[2,3-d]pyrimidin-7(8H)-one (0.2 g, 0.67 mmol, 1.0 eq.) in 1,4-dioxane (6 mL), was added Oxone (0.61 g, 2.0 mmol, 3.0 eq.) in water (2 mL) at room temperature under nitrogen atmosphere. The reaction mixture was stirred for 16 h. The reaction mixture was quenched with sat. NaHCO₃ solution (20 mL) and extracted with ethyl acetate (2×30 mL). The organic layer was washed with brine solution (30 mL), dried over sodium sulphate, filtered, and concentrated under reduced pressure to get crude compound. The crude compound was purified by comb flash using YMC-12 g cartridge, eluting with 0-50% EA/heptane to afford 0.110 g, 49.7% yield of the title compound as an off-white solid. ¹H NMR (400 MHz, DMSO-d₆): δ 9.48 (s, 1H), 3.72 (s, 3H), 3.49 (s, 3H), 2.72 (s, 3H); MS (ES⁺) m/z calc'd for [M+H]⁺ [C₁₀H₁₀BrN₃O₃S+H]⁺: 331.96, found 331.65, LCMS: t_{R}=1.33 min [Method: C].

### Synthesis of 6-bromo-5,8-dimethyl-2-(methylamino)pyrido[2,3-d]pyrimidin-7(8H)-one [11-42]

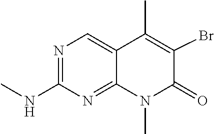 [SMILES: CNc1ncc2c(C)c(Br)c(=O)n(C)c2n1]

To a stirred solution of 6-bromo-5,8-dimethyl-2-(methylsulfonyl)pyrido[2,3-d]pyrimidin-7(8H)-one (0.110 g, 0.33 mmol, 1.0 eq) in THF (10 mL), was added MeNH₂ in 33% ethanol (2 mL) at room temperature under nitrogen atmosphere. The reaction mixture was heated 70° C. for 16 h. The reaction mixture was evaporated under reduced pressure to afford 0.1 g, 97% yield of the title compound as a pale-yellow solid. MS (ES)⁺ m/z calc'd for [M+H]⁺ [C₁₀H₁₀BrN₃O₃S+H]⁺: 283.01, found 282.80, LCMS: t_{R}=1.41 min [Method: C]

### Synthesis of 6-(5-amino-2-chlorophenyl)-5,8-dimethyl-2-(methylamino)pyrido[2,3-d]pyrimidin-7(8H)-one [Intermediate 42]

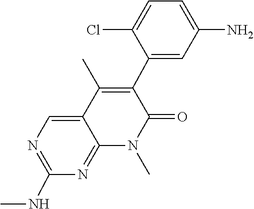 [SMILES: CNc1ncc2c(C)c(-c3cc(N)ccc3Cl)c(=O)n(C)c2n1]

To a stirred solution of 6-bromo-5,8-dimethyl-2-(methylamino)pyrido[2,3-d]pyrimidin-7-one (0.130 g, 0.459 mmol, 1.0 eq) and (5-amino-2-chloro-phenyl)boronic acid (0.157 g, 0.918 mmol, 2.0 eq) in DMF (5 mL), was added Na₂CO₃ (0.121 g, 1.14 mmol, 2.5 eq) at room temperature under nitrogen atmosphere. The reaction mixture was degassed with nitrogen atmosphere for 10 min and added Pd(PPh₃)₄ (0.053 g, 0.046 mmol, 0.1 eq). The resulting reaction mixture was heated to 90° C. for 16 h. The reaction mixture was diluted with water (20 mL) and extracted with Ethyl acetate (2×50 mL). The combined organic layer was washed with brine solution (20 mL), dried over sodium sulphate, filtered, and concentrated under vacuum to get crude compound. The crude compound was purified by combi flash using YMC-4 g cartridge, with 60-80% ethyl acetate in heptane to afford 50 mg, 31.95% yield of the title compound as a pale brown solid. ¹H NMR (400 MHz, DMSO-d₆): δ 8.81-8.68 (m, 1H), 7.79-7.59 (m, 1H), 7.12 (d, J=8.8 Hz, 1H), 6.58-6.51 (m, 1H), 6.40-6.33 (m, 1H), 5.24 (br s, 2H), 3.61-3.47 (m, 3H), 2.91 (br d, J=4.4 Hz, 3H), 2.11 (s, 3H); MS (ES)⁺ m/z calc'd for [M+H]⁺ [C₁₆H₁₆ClN₅O+H]⁺: 330.10, found 330.15, LCMS: t_{R}=2.27 min [Method-A] HPLC: 92.11% [Method: C].

### Synthesis of (3-amino-5-bromo-2,6-difluoro-phenyl)-(5-chloro-1H-pyrrolo[2,3-b]pyridin-3-yl)methanone [Intermediate 43]

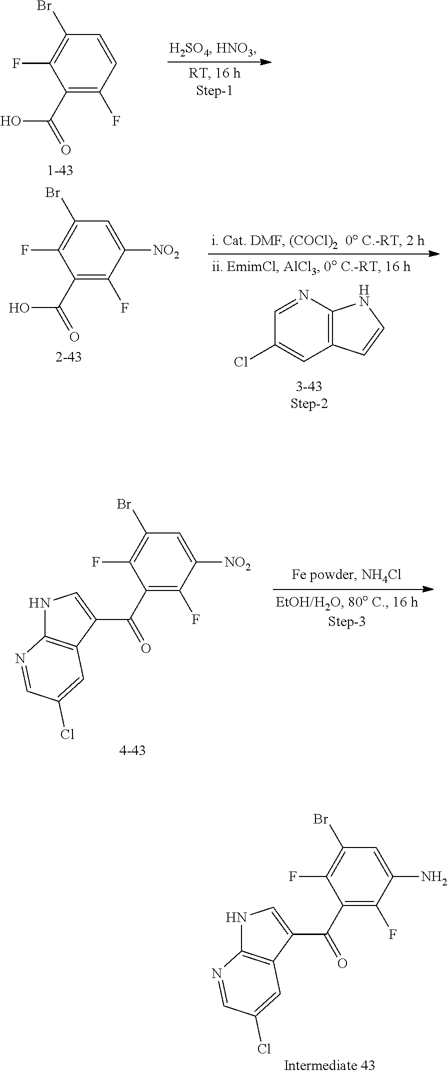

### Synthesis of 3-bromo-2,6-difluoro-5-nitro-benzoic acid [2-43]

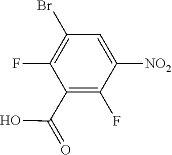 [SMILES: O=C(O)c1c(F)c(Br)cc([N+](=O)[O-])c1F]

To a stirred solution of 3-Bromo-2,6-difluorobenzoic acid (10.00 g. 42.2 mmol. 1.00 eq.) in sulfuric acid (34.02 g, 347 mmol, 8.22 eq.) was added nitric Acid (7.98 g. 127 mmol, 3.00 eq.) drop wise at 0° C. The reaction was slowly warm to room temperature and stirred for 16 h. The reaction mixture was turned to light brown from clear solution. The reaction was poured in ice cold water (100 ml) and the obtained white precipitate was filtered through Buchner funnel and washed with water (10 ml). The solid was dried by co-distillation with toluene (3×10 mL) to afford 10 g, 83.76% yield of the title compound as an off-white solid. 1H NMR (400 MHz, DMSO-d6): δ 8.75-8.71 (m, 1H).

### Synthesis of (3-bromo-2,6-difluoro-5-nitro-phenyl)-(5-chloro-1H-pyrrolo[2,3-b]pyridin-3-yl)methanone [4-43]

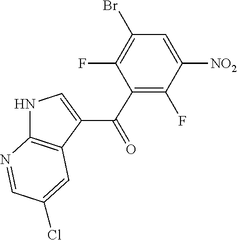 [SMILES: O=C(c1c(F)c(Br)cc([N+](=O)[O-])c1F)c1c[nH]c2ncc(Cl)cc12]

To a stirred solution of 3-bromo-2,6-difluoro-1-nitro-benzoic acid (5.00 g, 17.7 mmol, 1.00 eq.) in DCM (50 mL, 0.3546 M) was added DMF (0.14 mL, 1.77 mmol, 0.1000 eq.). The reaction was cooled to 0° C. and added oxalyl chloride (3.1 mL, 35.5 mmol, 2.00 eq.) dropwise. The reaction mixture was slowly warm to room temperature and stirred for 2 h. The solvent was evaporated under reduced pressure to obtain the crude acid chloride. To a stirred solution of 5-chloro-1H-pyrrolo[2,3-B]Pyridine (2.71 g, 17.7 mmol, 1.00 eq.) in DCM (30 mL) was added emim-chloride (5.20 g, 35.5 mmol, 2.00 eq.), aluminum chloride (14.19 g, 106 mmol, 6.00 eq.) and the above prepared acid chloride at 0° C. During the reaction, highly exothermic observed and the reaction mixture was turned to black color gummy liquid from clear solution. The reaction was allowed to room temperature and stirred for 16 h. The reaction mixture was basified using saturated solution of sodium bicarbonate (50 mL) and the product was extracted in Ethyl acetate (3×50 mL). The combined organic layer was dried over sodium sulphate and concentrated to give the crude product as yellow gummy solid. The crude was triturated with diethyl ether (2×25 mL) and pentane (15 mL) to afford 2.5 g, 67.69% yield of the titled compound as a yellow solid. 1H NMR (400 MHz, DMSO-d₆): δ 13.48-13.11 (m, 1H), 8.86-8.74 (m, 1H), 8.55-8.51 (m, 1H), 8.51-8.49 (m, 1H), 8.47-8.44 (m, 1H); MS (ES⁺) m/z calc'd for [M+H]⁺ [C₁₄H₅BrClF₂N₃O₃+H]⁺: 415.92, found: 418.05, LCMS: t_{R}=1.91 min [Method: C].

### Synthesis of (3-amino-5-bromo-2,6-difluoro-phenyl)-(5-chloro-1H-pyrrolo[2,3-b]pyridin-3-yl)methanone [Intermediate 43]

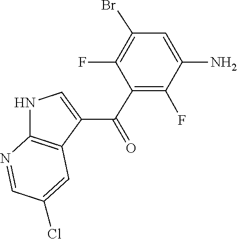 [SMILES: Nc1cc(Br)c(F)c(C(=O)c2c[nH]c3ncc(Cl)cc23)c1F]

To a stirred solution of (3-bromo-2,6-difluoro-5-nitro-phenyl)-(5-chloro-1H-pyrrolo[2,3-b]pyridin-3-yl)methanone (2.50 g, 6.00 mmol, 1.00 eq.) in EtOH (20 mL, 0.2401 M) was added iron (Powder) (1.68 g, 30.0 mmol, 5.00 eq.) and aq. ammonium chloride (1.61 g, 30.0 mmol, 5.00 eq.) in Water (5 mL, 0.2401 M). The reaction mixture was refluxed at 80° C. for 6 h. The reaction mixture was filtered through celite bed and washed with methanol (5 mL). The combined filtrate was concentrated under reduced pressure to give the crude. The crude was dissolved in ethyl acetate (50 mL) and washed with saturated solution of sodium bicarbonate solution (2×50 mL) and water (50 mL). The organic layer was dried over sodium sulphate and concentrated to give 1.0 g, 43% yield of the crude titled compound as yellow solid. ¹H NMR (400 MHz, DMSO-d₆): δ 13.11 (br s, 1H), 8.45 (br d, J=7.3 Hz, 2H), 8.31 (s, 1H), 7.19-7.11 (m, 1H), 5.51 (br s, 2H); MS (ES⁺) m/z calc'd for [M+H]⁺[C₁₄H₇BrClF₂N₃O+H]⁺: 385.94, found: 387.80, LCMS: t_{R}=1.86 min [Method: C].

### Synthesis of 4-((6,7-dimethoxyquinolin-4-yl)oxy)aniline [Intermediate 44]

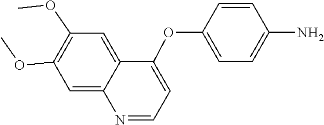 [SMILES: COc1cc2nccc(Oc3ccc(N)cc3)c2cc1OC]

Available from Commercial Sources

### Synthesis of 4-((6,7-dimethoxyquinolin-4-yl)oxy)-2-fluoroaniline [Intermediate 45]

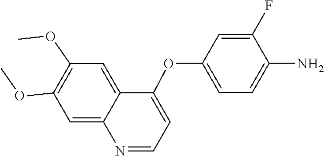 [SMILES: COc1cc2nccc(Oc3ccc(N)c(F)c3)c2cc1OC]

Available from Commercial Sources

### Synthesis of 6-(3-amino-2,6-dichloro-phenyl)-8-methyl-2-(methylamino) pyrido[2,3-d]pyrimidin-7-one [Intermediate 46]

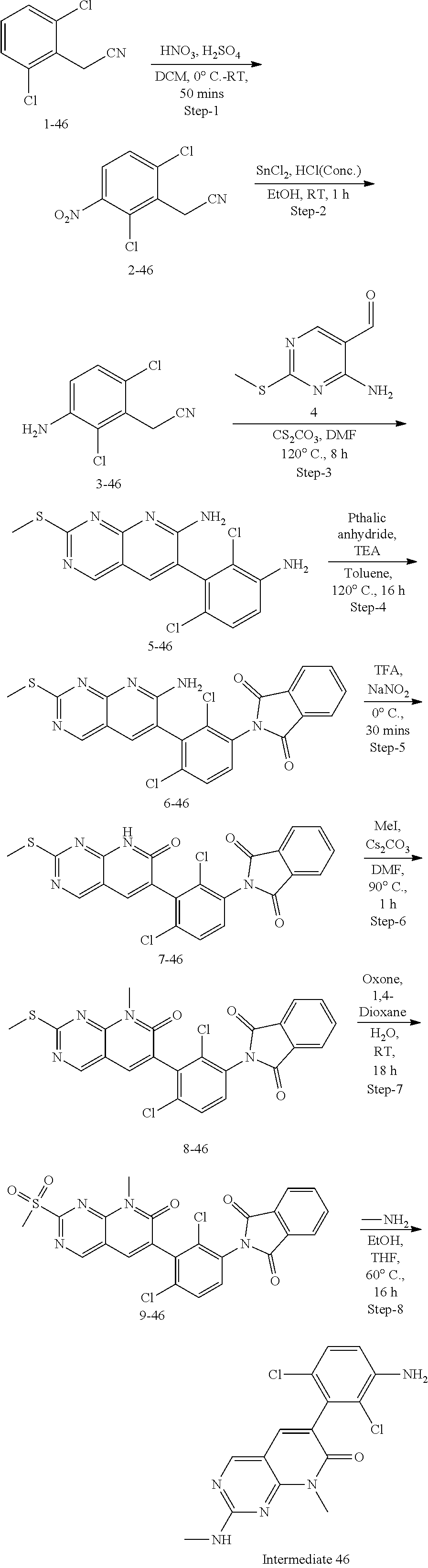

### Synthesis of 2-(2,6-dichloro-3-nitro-phenyl)acetonitrile [2-46]

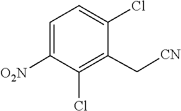 [SMILES: N#CCc1c(Cl)ccc([N+](=O)[O-])c1Cl]

To a solution of 2,6-dichloro-phenyl-acetonitrile (30 g, 161.26 mmol 1 eq.) in dichloromethane (100 mL) and H2SO4 (80 mL) was added a mixture of H2SO4 (28.5 mL) and HNO3 (11 mL) slowly at 0° C. The reaction mixture was stirred at same temp. for 20 minutes followed by stirring at room temperature for 30 min. The progress of the reaction was monitored by TLC. After the completion, the reactant mass was concentrated to remove the organic solvent. The solution was then poured into a beaker containing ice-water (500 mL) to give a crystalline precipitate, which was filtered and washed with water to afford 20 g, 48% yield of the title compound. 1H NMR (400 MHz, CDCl₃): δ 7.72 (d, J=8.8 Hz, 2H), 7.51 (d, J=8.8 Hz, 2H), 4.06 (s, 2H); MS (ES)+m/z calc'd for [M+H]⁺[C₈H₄Cl₂N₂O₂+H]⁺: 230.97 found 230.65, and t_{R}=1.814 min. LCMS: [Method: C].

### Step 2: Synthesis of 2-(3-amino-2,6-dichloro-phenyl) acetonitrile [3-46]

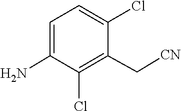 [SMILES: N#CCc1c(Cl)ccc(N)c1Cl]

To a stirred solution of ethyl 2-(2,6-dichloro-3-nitro-phenyl) acetonitrile (30 g, 129.85 mmol, 1 eq.) in Ethanol (1500 mL) was added Tin(II) chloride (81.243 g, 428.5 mmol, 3.3 eq.) in conc. HCl (300 mL) at 75° C. The reaction mixture was then stirred at room temperature for 1 h. The progress of reaction was monitored by TLC. After completion, the reaction mixture was neutralized with K₂CO₃ to a pH of 8 and was filtered through a pad of celite bed. The filtrate was concentrated under reduced pressure to remove the solvent and dried under vacuum to get 25 g, 88.1% yield of the title compound as off white solid. 1H NMR (400 MHz, DMSO-d6): δ 7.20 (d, J=8.9 Hz, 1H), 6.83 (d, J=8.9 Hz, 1H), 5.72 (s, 2H), 4.05 (s, 2H); MS (ES)⁺ m/z calc'd for [M+H]⁺ [C₈H₆Cl₂N₂+H]⁺: 201 found 200.85, and t_{R}=1.586 min. LCMS: [Method: C]

### Synthesis of 6-(3-amino-2,6-dichloro-phenyl)-2-methylsulfanyl-pyrido[2,3-d]pyrimidin-7-amine [5-46]

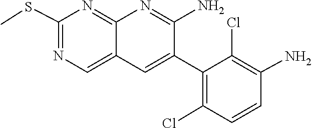 [SMILES: CSc1ncc2cc(-c3c(Cl)ccc(N)c3Cl)c(N)nc2n1]

To a stirred solution of 4-amino-2-(methylthio)pyrimidine-5-carbaldehyde (8.5 g, 50.236 mmol, 1 eq.) in DMF (100 mL) were added 2-(3-amino-2,6-dichloro-phenyl)acetonitrile (12.12 g, 60.284 mmol, 1.2 eq.) and cesium carbonate (49.131 g, 150.71 mmol, 3 eq.) at room temperature and then the reaction mixture was stirred at 120° C. for 8 h. After the completion, the reaction mixture was cooled to RT. The reaction mixture was diluted with water (250 mL) to obtain the precipitate. The precipitate was filtered and washed with water to afford 12 g, 60% yield of the title compound as a yellow solid. 1H NMR (400 MHz, DMSO-d6): δ 8.87 (s, 1H), 7.93 (s, 2H), 7.80 (s, 1H), 7.24 (d, J=8.8 Hz, 1H), 6.90 (d, J=8.8 Hz, 1H), 5.62 (s, 2H), 2.54 (s, 3H); MS (ES)+m/z calc'd for [M+H]⁺[C₁₄H₁₁Cl₂N₅S+H]⁺: 352.02 found 353.75, and t_{R}=1.447 min. LCMS: [Method: C].

### Step 4: Synthesis of 2-[3-(7-amino-2-methylsulfanyl-pyrido[2,3-d]pyrimidin-6-yl)-2,4-dichloro-phenyl]isoindoline-1,3-dione [6-46]

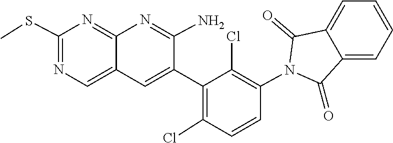 [SMILES: CSc1ncc2cc(-c3c(Cl)ccc(N4C(=O)c5ccccc5C4=O)c3Cl)c(N)nc2n1]

To a solution of 6-(3-amino-2,6-dichloro-phenyl)-2-methylsulfanyl-pyrido[2,3-d]pyrimidin-7-amine (15 g, 42.585 mmol, 1.00 eq.) in Toluene (800 mL) were added Phthalic anhydride (6.3076 g, 42.585 mmol, 1.00 eq.) and TEA (5.9849 mL, 42.585 mmol, 1.00 eq.) at RT under N₂-atm. The reaction was stirred at 120° C. for 16 h. The reaction was monitored by TLC. After the completion, the reaction was evaporated under vacuum to get crude. The crude was purified by combi flash using 120 g cartridge, eluting with 0-3% MeOH in DCM to afford 8 g, 38% yield of the title compound as yellow solid. 1H NMR (400 MHz, DMSO-d6): δ 8.90 (s, 1H), 8.05-8.01 (m, 2H), 7.97-7.94 (m, 3H), 7.85-7.77 (m, 2H), 7.39-7.10 (m, 2H), 2.55 (s, 3H); MS (ES)+m/z calc'd for [M+H]⁺[C₂₂H₁₃Cl₂N₅O+H]⁺: 482.02 found 481.90, and t_{R}=1.698 min. LCMS: [Method: C].

### Synthesis of 2-[2,4-dichloro-3-(2-methylsulfanyl-7-oxo-8H-pyrido[2,3-d]pyrimidin-6-yl)phenyl]isoindoline-1,3-dione [7-46]

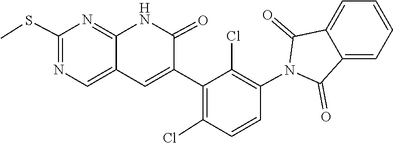 [SMILES: CSc1ncc2cc(-c3c(Cl)ccc(N4C(=O)c5ccccc5C4=O)c3Cl)c(=O)[nH]c2n1]

To a stirred solution of 2-[3-(7-amino-2-methylsulfanyl-pyrido[2,3-d]pyrimidin-6-yl)-2,4-dichloro-phenyl]isoindoline-1,3-dione (8 g, 16.586 mmol, 1.00 eq.) in TFA (73 mL) was added NaNO₂ (4.0055 g, 58.05 mmol, 3.5 eq.) at 0° C. under nitrogen atmosphere and the resultant reaction mixture was stirred at same temp. for another 30 mins. The reaction was monitored by TLC. After the completion of starting material, the reaction mixture was evaporated under reduced pressure and the crude was diluted with ethyl acetate and basified with Saturated K₂CO₃ solution. The organic layer was extracted with EtOAc and the combined organic layer was dried over Na₂SO₄ and evaporated under reduced pressure to afford the titled product 2-[2,4-dichloro-3-(2-methylsulfanyl-7-oxo-8H-pyrido[2,3-d]pyrimidin-6-yl)phenyl]isoindoline-1,3-dione. MS (ES)⁺ m/z calc'd for [M+H]⁺[C₂₂H₁₂Cl₂N₄O₃S+H]⁺: 483.01 found 482.90, and t_{R}=1.816 min. LCMS: [Method: C].

### Synthesis of 2-[2,4-dichloro-3-(8-methyl-2-methylsulfanyl-7-oxo-pyrido[2,3-d]pyrimidin-6-yl)phenyl]isoindoline-1,3-dione [8-46]

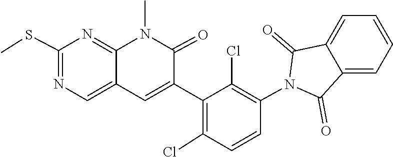 [SMILES: CSc1ncc2cc(-c3c(Cl)ccc(N4C(=O)c5ccccc5C4=O)c3Cl)c(=O)n(C)c2n1]

To a stirred solution of 2-[2,4-dichloro-3-(2-methylsulfanyl-7-oxo-8H-pyrido[2,3-d]pyrimidin-6-yl)phenyl]isoindoline-1,3-dione (7.5 g, 15.517 mmol, 1 eq.) in DMF (75 mL) were added Mel (1.437 mL, 23.276 mmol, 1.5 eq.), and Cs₂CO₃ (10.086 g. 31.035 mmol, 2 eq.). The Reaction mixture was then heated at 90° C. for 1 h. The progress of the reaction was monitored by TLC. After the completion, the reaction mixture was cooled to RT and diluted with water. The product precipitated, filtered and washed with water. The crude was further purified by chromatography by Sepaflash 40 g YMC cartridge with gradient 0-60% Ethyl acetate in n-heptane to yield 3.2 g. 27% yield of the title compound as an off-white solid. MS (ES)⁺ m/z calc'd for [M+H]⁺[C₂3H₁₄Cl₂N₄O₃S+H]⁺:497.02 found 496.90, t_{R}=2.085. [Method: C].

### Synthesis of 2-[2,4-dichloro-3-(8-methyl-2-methylsulfonyl-7-oxo-pyrido[2,3-d]pyrimidin-6-yl)phenyl]isoindoline-1,3-dione [9-46]

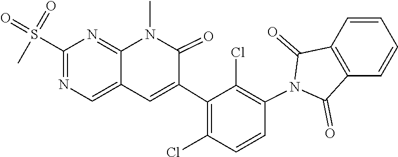 [SMILES: Cn1c(=O)c(-c2c(Cl)ccc(N3C(=O)c4ccccc4C3=O)c2Cl)cc2cnc(S(C)(=O)=O)nc21]

To a stirred solution of 2-[2,4-dichloro-3-(8-methyl-2-methylsulfanyl-7-oxo-pyrido[2,3-d]pyrimidin-6-yl)phenyl]isoindoline-1,3-dione (3 g, 6.032 mmol, 1 eq.) in 1,4-Dioxane (40 mL) was added Oxone (4.6295 g, 15.08 mmol, 2.5 eq.) in water (15 mL). The resultant reaction mixture was stirred at 25° C. for 18 h. The progress of reaction was monitored by TLC. After the completion, the reaction mixture was diluted with water (20 mL) and the product precipitated, filtered and washed with water to afford 2-[2,4-dichloro-3-(8-methyl-2-methylsulfonyl-7-oxo-pyrido[2,3-d] pyrimidin-6-yl) phenyl] isoindoline-1,3-dione (2.3 g mg, 72%) as a red solid. MS (ES)+m/z calc'd for [M+H]⁺[C₂₃H₁₄Cl₂N₄O₅S+H]: 529.01 found 529, t_{R}=1.786 mins. [Method: C].

### Synthesis of 6-(3-amino-2,6-dichloro-phenyl)-8-methyl-2-(methylamino) pyrido[2,3-d] pyrimidin-7-one [Intermediate 46]

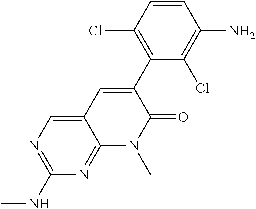 [SMILES: CNc1ncc2cc(-c3c(Cl)ccc(N)c3Cl)c(=O)n(C)c2n1]

A mixture of 2-[2,4-dichloro-3-(8-methyl-2-methylsulfonyl-7-oxo-pyrido[2,3-d]pyrimidin-6-yl)phenyl]isoindoline-1,3-dion (1.8 g, 3.4004 mmol, 1 eq.) and methylamine 33% in EtOH (20 mL) and THF (20 mL) was heated to 60° C. for 16 h. The reaction mixture was cooled to room temperature and the progress of the reaction was monitored by TLC. The reaction mixture was diluted with water (30 mL) and the obtained solid was filtered, washed with water, and dried under vacuum to afford 1.3 g, 98% yield of the title compound as a white solid. 1H NMR (400 MHz, DMSO-d₆): δ 8.62 (s, 1H), 7.63 (s, 1H), 7.53 (br d, J=4.4 Hz, 1H), 7.17 (br d, J=8.8 Hz, 1H), 6.86 (br d, J=8.3 Hz, 1H), 5.31 (br s, 1H), 3.60 (s, 3H), 2.96 (br d, J=3.9 Hz, 3H); MS (ES)+m/z calc'd for [M+H]⁺ [C₁₅H₁₃Cl₂N₅O+H]⁺: 350.06 found 349.85, t_{R}=1.572 mins. [Method: C].

### Synthesis of 4-methyl-3-(1H-pyrrolo[2,3-b]pyridin-4-yloxy)aniline [Example 47]

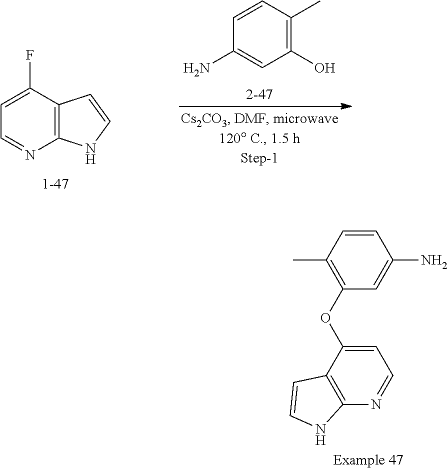

### Synthesis of 4-methyl-3-(1H-pyrrolo[2,3-b] pyridin-4-yloxy) aniline [Example 47]

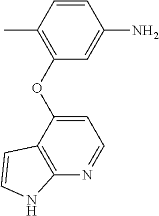 [SMILES: Cc1ccc(N)cc1Oc1ccnc2[nH]ccc12]

To a stirred solution of 4-Fluoro-7-Azaindole (3.50 g, 25.7 mmol, 1.00 eq.) in DMF (20 mL, 1.2855 M) was added 3-Hydroxy-4-methylaniline (4.75 g, 38.6 mmol, 1.50 eq.) and Cs₂CO₃ (25.07 g, 77.1 mmol, 3.00 eq.) at room temperature. The reaction mixture was heated to 120° C. for 1.5 h under microwave. The reaction mixture was treated with water (100 mL) and extracted with ethyl acetate (3×100 mL). The combined organic layer was dried over Na₂SO₄ and concentrated under reduced pressure to obtain the crude. The crude product was purified by chromatography by combi flash using 40 g YMC cartridge with 60-80% ethyl acetate in n-heptane to afford 680 mg, 10.66% yield of the title compound as an off-white solid. 1H NMR (400 MHz, DMSO-d₆): δ 11.64 (br s, 1H), 8.03 (d, J=5.4 Hz, 1H), 7.31 (br s, 1H), 6.97 (d, J=7.8 Hz, 1H), 6.45-6.41 (m, 1H), 6.30-6.27 (m, 2H), 6.20 (br d, J=2.0 Hz, 1H), 5.05 (s, 2H), 1.94 (s, 3H). LCMS: MS (ES)+m/z calc'd for [M+H]⁺ [C₁₄H₁₁N₃O₃+H]⁺: 240.11.09 found 240.0, t_{R}=0.960 mins. [Method: C].

### Synthesis of 6-fluoro-5-(5-methyl-1H-pyrrolo[2,3-b]pyridin-3-ylmethyl)pyridin-2-ylamine [Intermediate 48]

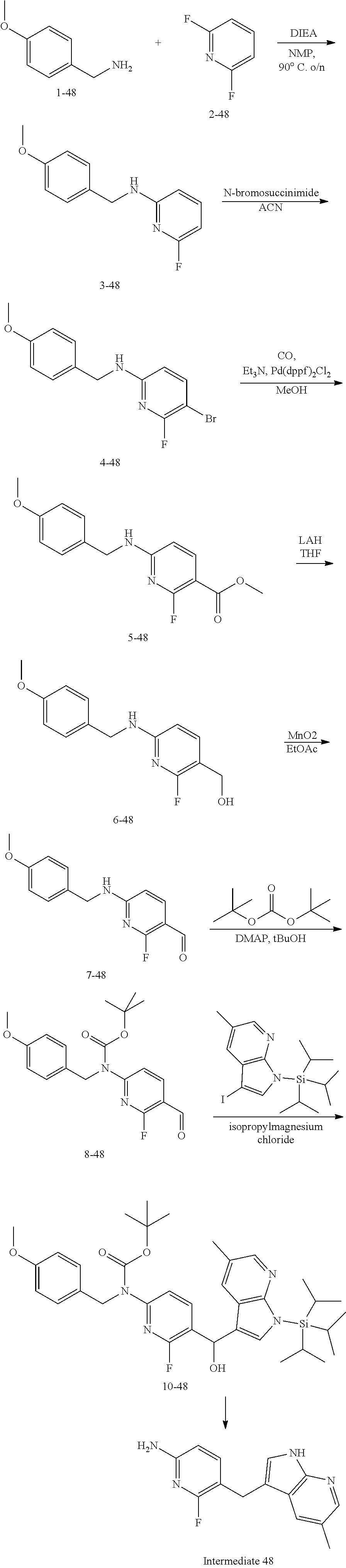

### 6-fluoro-5-(5-methyl-1H-pyrrolo[2,3-b]pyridin-3-ylmethyl)pyridin-2-ylamine [Intermediate 48]

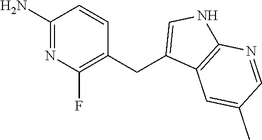 [SMILES: Cc1cnc2[nH]cc(Cc3ccc(N)nc3F)c2c1]

To a stirred solution of tert-butyl N-[6-fluoro-5-[hydroxy-(5-methyl-1-triisopropylsilyl-pyrrolo[2,3-b]pyridin-3-yl)methyl]-2-pyridyl]-N-[(4-methoxyphenyl)methyl]carbamate (2.06 g, 3.17 mmol, 1.00 eq) in acetonitrile at rt was added triethylsilane (5.1 mL, 31.7 mmol, 10.0 eq) and trifluoroacetic acid (2.4 mL, 31.7 mmol, 10.0 eq). The resulting reaction mixture was stirred at 50° C. for 16 h. Then solvent was removed and the DCM (10 mL) was added to the crude followed by addition of TFA (10 mL). The mixture was stirred at 55° C. for 4 hr. The mixture was evaporated and the residue was partitioned between ethyl acetate and Sat. NaHCO₃. The aqueous was extracted with ethyl acetate (3×50 mL). The combined organic layers were washed with brine, dried with Na₂SO₄, filtered and concentrated to give the crude which was purified by flash column chromatography over silica gel, ISCO, CombiFlash, 40 g cartridge (eluting with 85%-95% ethyl acetate in Hexane) to afforded 0.27 g, 31% yield of the title compound as a purple solid. ¹H NMR (500 MHz, DMSO-d₆) δ 11.19 (s, 1H), 8.04-7.97 (m, 1H), 7.64-7.56 (m, 1H), 7.40-7.27 (m, 1H), 7.18-7.11 (m, 1H), 6.28-6.14 (m, 1H), 6.04 (d, J=8.0 Hz, 2H), 3.78 (d, J=7.6 Hz, 2H), 2.33 (d, J=7.7 Hz, 3H). MS (ES⁺) m/z calcd. for [M+H]⁺ [C₁₄H₁₃FN₄+H]⁺: 257.1 found 257.2, LCMS t_{R}=1.15 min [Method B].

### {6-Fluoro-5-[hydroxy-(5-methyl-1-triisopropylsilanyl-1H-pyrrolo[2,3-b]pyridin-3-yl)methyl]pyridin-2-yl}-(4-methoxybenzyl)carbamic acid tert-butyl ester [10-48]

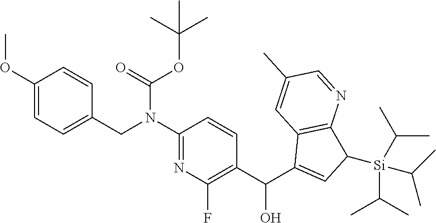 [SMILES: COc1ccc(CN(C(=O)OC(C)(C)C)c2ccc(C(O)C3=CC([Si](C(C)C)(C(C)C)C(C)C)c4ncc(C)cc43)c(F)n2)cc1]

To a stirred solution of (3-iodo-5-methy-pyrrolo[2,3-b]pyridin-1-yl)-triisopropyl-silane (126 mg, 0.305 mmol, 2.20 eq) in 2 ml THF at −78° C. was added butyllithium (0.22 mL, 0.555 mmol, 4.00 eq). The reaction mixture was stirred at −78° C. for 1 hour, then a solution of tert-butyl N-(6-fluoro-5-formyl-2-pyridyl)-N-[(4-methoxyphenyl)methyl]carbamate (50 mg, 0.139 mmol, 1.00 eq) in 1 ml THF. The resulting reaction mixture was stirred at −78° C., then was allowed to warm to −30° C. over a period of 1 hr. LCMS showed the reaction was complete. The reaction was quenched with aq. NH₄Cl, extracted with ethyl acetate, washed with brine, dried with anhydrous NaSO4. filtration and concentration on a rotavapor afforded a crude, which was purified by flash column chromatography over silica gel, ISCO, CombiFlash, 4 g cartridge (eluting with 0-20% ethyl acetate in hexane) to afforded 73 mg, 81% yield of the title compound as a white solid. ¹H NMR (500 MHz, Chloroform-d) δ 8.10 (d, J=2.1 Hz, 1H), 7.91 (dd, J=10.0, 8.2 Hz, 1H), 7.71-7.55 (m, 2H), 7.23 (d, J=8.5 Hz, 2H), 7.15 (s, 1H), 6.80 (d, J=8.6 Hz, 2H), 6.25 (s, 1H), 5.09 (d, J=4.9 Hz, 2H), 3.77 (s, 2H), 2.36 (s, 3H), 1.81 (p, J=7.5, 7.5, 7.5, 7.5 Hz, 3H), 1.45 (s, 10H), 1.10 (dd, J=7.6, 1.8 Hz, 19H); MS (ES⁺) m/z calc'd for [M+H]⁺[C₃₆H₄₉FN₄O₄Si+H]⁺: 649.4 found 649.7. LCMS: t_{R}=9.54 min. [Method: A-12]

### (6-Fluoro-5-formylpyridin-2-yl)(4-methoxybenzyl)carbamic acid tert-butyl ester [8-48]

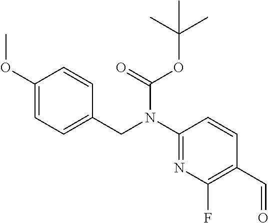 [SMILES: COc1ccc(CN(C(=O)OC(C)(C)C)c2ccc(C=O)c(F)n2)cc1]

To a stirred solution of 2-fluoro-6-[(4-methoxyphenyl)methylamino]pyridine-3-carbaldehyde (1.68 g, 6.13 mmol, 1.00 eq) in tert-butanol (15 mL, 0.4088 M) was added Di-tert-butyl dicarbonate (2.01 g, 9.20 mmol, 1.50 eq) and 4-Dimethylaminopyridine (0.075 g, 0.613 mmol, 0.100 eq). The reaction was stirred at 30° C. overnight and then concentrated under vacuum. The crude material was purified by flash column chromatography over silica gel, ISCO, CombiFlash, 40 g cartridge (eluting with 0-10% ethyl acetate in hexane) to provide 1.40 g, 60.18% yield of the title compound as a colorless oil. ¹H NMR (500 MHz, DMSO-d₆) δ 10.03 (s, 1H), 8.27 (t, J=9.2, 9.2 Hz, 1H), 7.89 (dd, J=8.5, 1.5 Hz, 1H), 7.19 (d, J=8.4 Hz, 2H), 6.87 (d, J=8.7 Hz, 1H), 5.07 (s, 2H), 3.70 (s, 2H), 1.41 (s, 5H); MS (ES⁺) m/z calc'd for [M+H]⁺[C₁₉H₂₁FN₂O₄+H]⁺: 361.1 found. 361.0 LCMS: t_{R}=6.29 min. [Method: A-12]

### 2-Fluoro-6-[(4-methoxyphenyl)methylamino]pyridine-3-carbaldehyde[7-48]

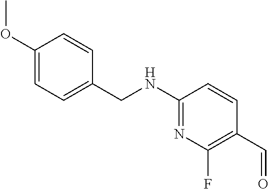 [SMILES: COc1ccc(CNc2ccc(C=O)c(F)n2)cc1]

To [2-fluoro-6-[(4-methoxyphenyl)methylamino]-3-pyridyl]methanol (1.68 g, 6.10 mmol) in ethyl acetate (32 ml), dioxomanganese (7.96 g, 91.5 mmol) was added. The reaction was stirred overnight at room temperature, then filtered through celite and the celite rinsed with ethyl acetate. The combined filtrates were concentrated under vacuum and the resulting solid was triturated with hexane. The solid was collected by filtration and dried under vacuum to provide 1.68 g, 96% yield of the title compound as a white solid. 1H NMR (500 MHz, DMSO-d₆) δ 9.83 (s, 1H), 8.61 (d, J=6.0 Hz, 1H), 7.82 (t, J=9.6, 9.6 Hz, 1H), 7.25 (d, J=8.4 Hz, 2H), 6.89 (d, J=8.3 Hz, 2H), 6.51 (d, J=8.6 Hz, 1H), 4.45 (d, J=5.4 Hz, 2H), 3.72 (s, 3H); MS(ES⁺) m/z calc'd for [M+H]⁺ [C₁₅H₁₃FN₂O₂+H]⁺: 261.1 found 261.1, LCMS t_{R}=4.47 min [Method: A-12]

### [2-fluoro-6-[(4-methoxyphenyl)methylamino]-3-pyridyl]methanol [6-48]

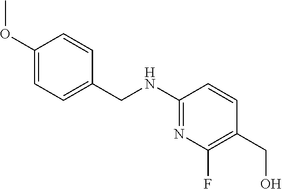 [SMILES: COc1ccc(CNc2ccc(CO)c(F)n2)cc1]

To a stirred solution of methyl 2-fluoro-6-[(4-methoxyphenyl)methylamino]pyridine-3-carboxylate (2.35 g, 7.92 mmol) in THF (12 ml), LAH (1M in THF, 12 ml, 11.9 mmol) was added dropwise while cooling. The reaction was stirred at room temperature for 2 hours, then quenched with dropwise addition of 2 mL of H₂O, 2 mL of 15% aqueous sodium hydroxide, and 2 mL of H₂O, sequentially. The mixture was extracted with ethyl acetate (3×50 ml). The combined organic layers were concentrated under vacuum. The crude material was purified by flash column chromatography over silica gel, ISCO, CombiFlash, 40 g cartridge (eluting with 10-40% ethyl acetate in hexane) to give 1.71 g, 82% yield of the title compound as a white solid. ¹H NMR (500 MHz, DMSO-d₆) 67.47 (dd, J=10.4, 8.1 Hz, 1H), 7.28 (s, 1H), 7.24-7.18 (m, 2H), 6.91-6.81 (m, 2H), 6.33 (dd, J=8.2, 1.8 Hz, 1H), 4.93 (t, J=5.6, 5.6 Hz, 1H), 4.31 (d, J=5.9 Hz, 2H), 4.28 (d, J=5.6 Hz, 2H), 3.70 (s, 3H); MS(ES⁺) m/z calc'd for [M+H]⁺ [C₁₄H₁₅FN₂O₂+H]⁺: 262.1 found 262.2, LCMS t_{R}=4.82 min [Method: A-12]

### Methyl 2-fluoro-6-[(4-methoxyphenyl)methylamino]pyridine-3-carboxylate [5-48]

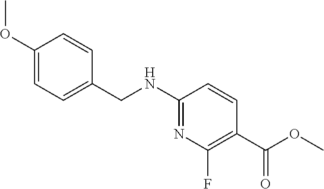 [SMILES: COC(=O)c1ccc(NCc2ccc(OC)cc2)nc1F]

To (5-bromo-6-fluoro-pyridin-2-yl)-(4-methoxy-benzyl)-amine (2.32 g, 7.45 mmol) in MeOH (60 ml) in a Parr flask with a stir bar, triethylamine (2.1 mL, 14.9 mmol) and [1,1′-bis(diphenylphosphino)ferrocene] dichloropalladium(II) (0.27 g, 0.37 mmol) were added. The reaction was heated at 100° C. under 100 psi of carbon monoxide overnight. The reaction was cooled and filtered through celite and the filtrate was concentrated under vacuum. The crude material was purified by flash column chromatography over silica gel, ISCO, CombiFlash, 40 g cartridge (eluting with 0-20% ethyl acetate in hexane) to give 1.82 g, 82% yield of the title compound as a white solid. ¹H NMR (500 MHz, DMSO-d₆) δ 8.23 (t, J=6.0, 6.0 Hz, 1H), 7.91 (t, J=9.3, 9.3 Hz, 1H), 7.26-7.22 (m, 2H), 6.91-6.86 (m, 2H), 6.44 (d, J=8.5 Hz, 1H), 4.40 (d, J=5.7 Hz, 2H), 3.73 (s, 3H), 3.71 (s, 3H); MS(ES⁺) m/z calc'd for [M+H]⁺ [C₁₄H₁₅FN₂03+H]⁺: 263.1 found 263.1, LCMS t_{R}=3.60 min [Method: A-12]

### Synthesis of 6-(5-amino-2-methyl-phenyl)-8-methyl-2-(methylamino) pyrido[2,3-d] pyrimidin-7-one [Intermediate 49]

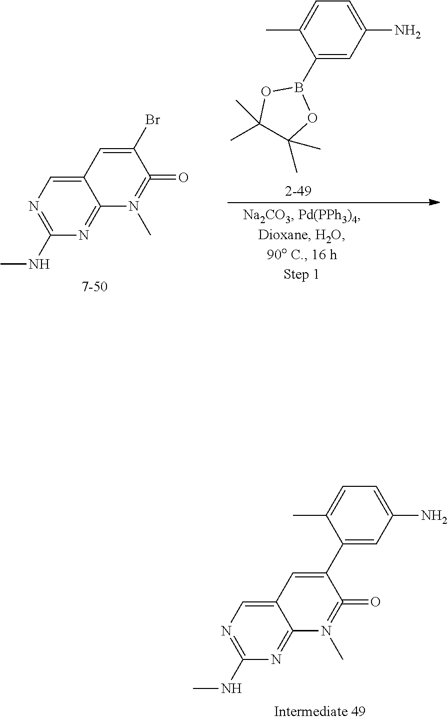

### Step 1: Synthesis of 6-(5-amino-2-methyl-phenyl)-8-methyl-2-(methylamino) pyrido[2,3-d] pyrimidin-7-one [Intermediate 49]

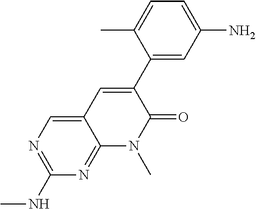 [SMILES: CNc1ncc2cc(-c3cc(N)ccc3C)c(=O)n(C)c2n1]

To a stirred solution of 6-bromo-8-methyl-2-(methylamino) pyrido[2,3-d]pyrimidin-7-one (1.5 g, 5.5741 mmol, 1.00 eq.) in 1,4-Dioxane (15 mL) and Water (5 mL) were added 5-amino-2-methylphenylboronic acid pinacol ester (1.9491 g, 8.3612 mmol, 1.5 eq.) and Na₂CO₃ (1.4771 g, 13.935 mmol, 2.5 eq.) at RT under nitrogen atmosphere. The reaction mixture was degassed with Nitrogen (g) for 10 min followed by the addition of tetrakis(triphenyl)phosphine) palladium (0.6441 g, 0.5574 mmol, 0.1000 eq.). The reaction mixture was heated to 90° C. and stirred for 16 h. The progress of the reaction was monitored by TLC. After the consumption of starting material, the reaction mixture was filtered on celite pad, and the solvent evaporated under reduced pressure to obtain the crude. The crude product was purified by chromatography on Sepaflash 24 g YMC cartridge with a gradient 0-80% of EtOAc in n-heptane to yield 1 g, 59% yield of the title compound as an off-white solid. 1H NMR (400 MHz, DMSO-d6): δ 8.70-8.55 (m, 1H), 7.92-7.70 (m, 1H), 7.65 (s, 1H), 7.15 (br d, J=8.3 Hz, 1H), 6.84-6.76 (m, 1H), 5.52 (br s, 2H), 3.64-3.46 (m, 3H), 2.89 (br s, 3H); MS (ES)+m/z calc'd for [M+H]⁺ [C₁₆H₁₇N₅O+H]⁺: 296.15 found 295.90, and t_{R}=1.157 mins. LCMS: [Method: C].

### 6-(5-amino-2-chlorophenyl)-8-methyl-2-(methylamino)pyrido[2,3-d]pyrimidin-7(8H)-one [Intermediate 50]

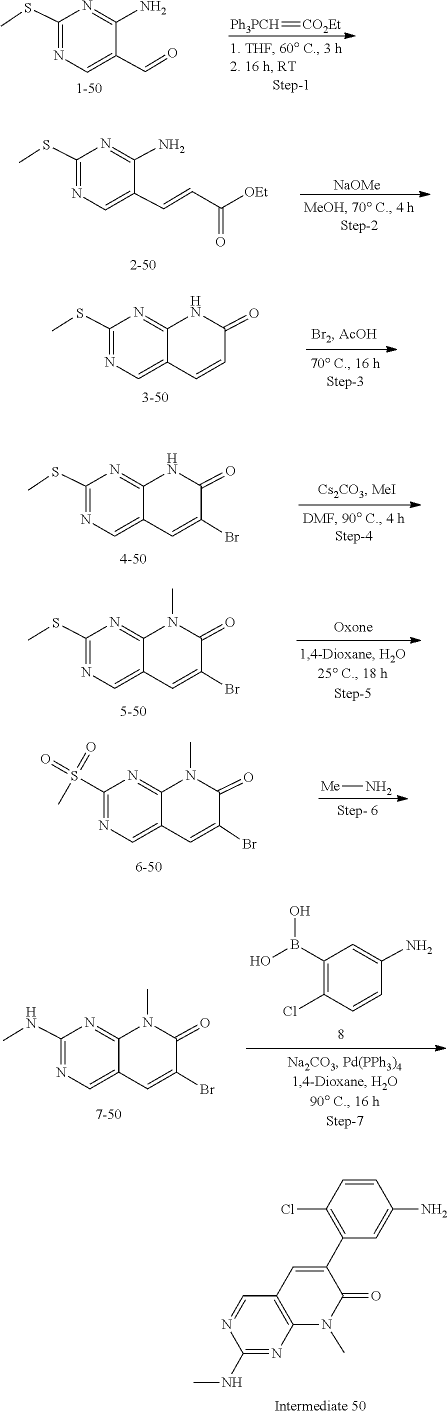

### Synthesis of ethyl (E)-3-(4-amino-2-(methylthio)pyrimidin-5-yl)acrylate [2-50]

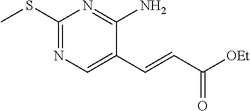 [SMILES: CCOC(=O)/C=C/c1cnc(SC)nc1N]

To a stirred solution of 4-amino-2-(methylthio)pyrimidine-5-carbaldehyde (10 g, 59.102 mmol, 1.00 eq.) in THF (70 mL) was added ethyl(triphenylphosphoranylidene)acetate (30.887 g, 88.652 mmol, 1.50 eq.) at room temperature. The reaction mixture heated to 75° C. and stirred for 3 h, followed by stirred at room temperature for 16 h. The reaction mixture was concentrated under reduced pressure to afford crude. The crude was purified by combi flash with 80 g YMC cartridge and 25-30% of ethyl acetate in heptane as an eluent to afford 8.2 g, 57.98% yield of the title compound as an off-white solid. ¹H NMR (400 MHz, CDCl₃): δ 8.55-8.22 (m, 1H), 7.60-7.49 (m, 1H), 6.36 (br d, J=16.1 Hz, 1H), 5.74 (br s, 2H), 4.33-4.22 (m, 2H), 2.64-2.51 (m, 3H), 1.34 (t, J=7.1 Hz, 3H); MS(ES⁺) m/z: [M+H]⁺ calculated for [C₁₀H₁₃N₃O₂S+H]⁺=240.07, found 240.2 t_{R}=1.36 min.[Method: C]

### Synthesis of 2-(methylthio)pyrido[2,3-d]pyrimidin-7(8H)-one [3-50]

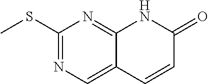

To a stirred solution of ethyl (E)-3-(4-amino-2-methylsulfanyl-pyrimidin-5-yl)prop-2-enoate (20.00 g, 83.6 mmol, 1.00 eq.) in methanol (150 mL, 0.5572 M) was added NaOMe (4.51 g, 83.6 mmol, 1.00 eq.) at room temperature. The reaction mixture heated to 70° C. and stirred for 4 h. The reaction mixture was cooled to room temperature, concentrated under reduced pressure to obtain residue. The residue was treated with water (200 mL) and the pH was adjusted to 8 with 2N HCl. The obtained precipitate was filtered, washed with water and dried under reduced pressure to afford 15 g, 90.11% yield of the title compound as an off-white solid. MS (ES)+m/z calc'd for [M+H]⁺ [C₈H₇N₃OS+H]⁺:194.03 found 193.80, t_{R}=1.122 min. [Method: C].

### Synthesis of 6-bromo-2-(methylthio)pyrido[2,3-d]pyrimidin-7(8H)-one [4-50]

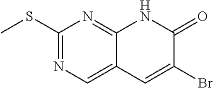

To a stirred solution of 2-methylsulfanyl-8H-pyrido[2,3-d]pyrimidin-7-one (10.00 g, 51.8 mmol, 1.00 eq.) in Acetic acid (60 mL, 0.8625 M) was added bromine (2.7 mL, 51.8 mmol, 1.0 eq.) dropwise at room temperature. The reaction mixture was heated to 70° C. and stirred for 16 h. The reaction mixture was cooled to room temperature and the precipitate was filtered, washed with DCM (50 mL), dried under reduced pressure to obtain 8 g, 56.8% yield of the crude title compound as pale yellow solid. The crude was used in the next step without further purification. ¹H NMR (400 MHz, DMSO-d₆): δ 12.89 (br s, 1H), 8.84 (s, 1H), 8.47 (s, 1H), 2.56 (s, 3H).

### Synthesis of 6-bromo-8-methyl-2-(methylthio)pyrido[2,3-d]pyrimidin-7(8H)-one [5-50]

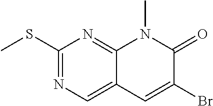

To a stirred solution of 6-bromo-2-methylsulfanyl-8H-pyrido[2,3-d]pyrimidin-7-one (8.00 g, 29.4 mmol, 1.00 eq.) in DMF (100 mL, 0.2940 M) was added Cs₂CO₃ (9.55 g, 29.4 mmol, 1.00 eq.) and Mel (1.8 mL, 29.4 mmol, 1.00 eq.) at room temperature. The reaction mixture was heated to 90° C. and stirred for 4 h. The reaction mixture was cooled to room temperature and treated with water (100 mL) to obtain precipitate. The precipitate was filtered and washed with water (30 mL), dried under reduced pressure to afford 5.0 g, 39.2% yield of the title compound as a pale yellow solid. MS (ES)⁺ m/z calc'd for [M+H]⁺[C₉H₈BrN₃OS+H]⁺:285.96 found 282.85. t_{R}=1.65 min. [Method: C]

### Synthesis of 6-bromo-2-(methylsulfonyl)pyrido[2,3-d]pyrimidin-7(8H)-one [6-50]

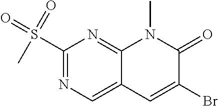 [SMILES: Cn1c(=O)c(Br)cc2cnc(S(C)(=O)=O)nc21]

To a solution of 6-bromo-8-methyl-2-methylsulfanyl-pyrido[2,3-d]pyrimidin-7-one (5.00 g, 17.5 mmol, 1.00 eq.) in 1,4-dioxane (80 mL, 0.1664 M) was added Oxone (5.32 g, 34.9 mmol, 2.00 eq.) in Water (25 mL, 0.1664 M) at room temperature. The reaction mixture was stirred for 18 h. The reaction mixture was quenched with 1N NaHCO₃ solution (100 mL) and extracted with dichloromethane (3×100 mL). The combined organic layer was dried over anhydrous sodium sulphate, concentrated under reduced pressure to afford 3.5 g, 43.4% yield of the title compound as a yellow solid. MS (ES)⁺ m/z calc'd for [M+H]⁺[C₉H₈BrN₃O₃S+H]⁺: 317.95 found 319.70, t_{R}=1.452 mins. [Method: C].

### Synthesis of 6-bromo-8-methyl-2-(methylamino)pyrido[2,3-d]pyrimidin-7-one [7-50]

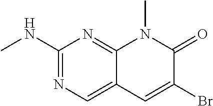

To a stirred solution of 6-bromo-8-methyl-2-methylsulfonyl-pyrido[2,3-d]pyrimidin-7-one (17 g, 53.434 mmol, 1 eq.) in THF (150 mL) was added Methylamine, 33% in EtOH (150 mL) at room temperature. The reaction mixture was heated to 60° C. and stirred for 16 h. The reaction mixture was cooled to room temperature and poured into ice cold water (100 mL) to obtain precipitate. The precipitate was filtered and washed with water to afford 11.2 g, 56% yield of the title compound as a white solid. 1H NMR (400 MHz, DMSO-d6): δ 8.68-8.54 (m, 1H), 8.28 (s, 1H), 7.97-7.79 (m, 1H), 3.66-3.47 (m, 3H), 2.89 (s, 3H); MS (ES)⁺ m/z calc'd for [M+H]⁺ [C₉H₉BrN₄O+H]⁺: 269 found 270.90, t_{R}=1.409 mins. [Method: C].

### Synthesis of 6-(5-amino-2-chloro-phenyl)-8-methyl-2-(methylamino) pyrido[2,3-d]pyrimidin-7-one [Intermediate 50]

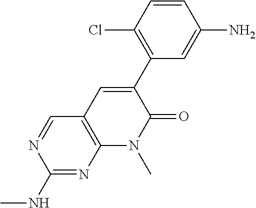 [SMILES: CNc1ncc2cc(-c3cc(N)ccc3Cl)c(=O)n(C)c2n1]

To a stirred solution of 6-bromo-8-methyl-2-(methylamino) pyrido[2,3-d]pyrimidin-7-one (1.5 g, 5.5741 mmol, 1.00 eq.) in 1,4-dioxane (10 mL) and Water (4 mL) was added 5-amino-2-chloro-phenyl) boronic acid (1.9107 g, 11.148 mmol, 2 eq.) and Na₂CO₃ (1.4771 g, 13.935 mmol, 3.00 eq.) at room temperature under nitrogen. The reaction mixture was degassed with nitrogen for 10 min and added Pd(PPh₃)₄ (0.6441 g, 0.5574 mmol, 0.1000 eq.). The reaction mixture was heated to 90° C. and stirred for 16 h. The reaction mixture was filtered through celite pad, and the filtrate was concentrated under reduced pressure to afford the crude. The crude was purified by combi flash with 24 g YMC cartridge and 0-10% MeOH in DCM to yield 370 mg, 20% yield of the titled compound as an off-white solid. 1H NMR (400 MHz, DMSO-d₆): δ 8.72-8.57 (m, 1H), 7.80 (br s, 1H), 7.70-7.54 (m, 2H), 7.09 (br d, J=8.3 Hz, 1H), 6.62-6.48 (m, 2H), 5.66-5.01 (m, 2H), 3.65-3.48 (m, 3H), 2.91 (br s, 3H). LCMS: MS (ES)⁺ m/z calc'd for [M+H]⁺ [C₁₅H₁₄ClN₅O+H]⁺: 316.1 found 315.85, and t_{R}=2.233 min. LCMS: [Method: C].

### SCA Intermediates

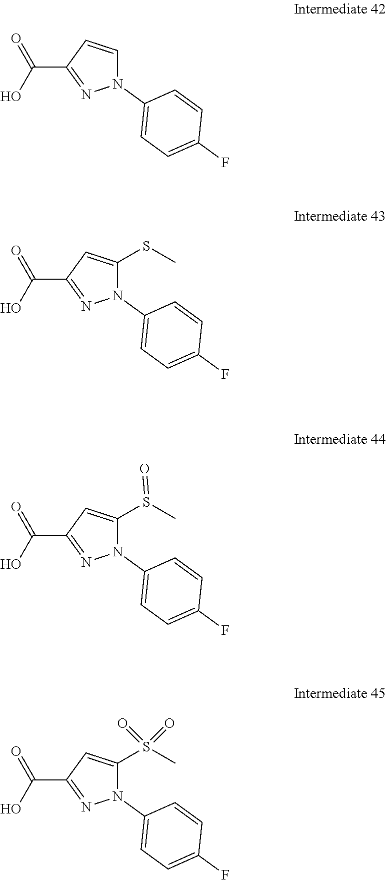 [SMILES: O=C(O)c1ccn(-c2ccc(F)cc2)n1 ; CSc1cc(C(=O)O)nn1-c1ccc(F)cc1 ; CS(=O)c1cc(C(=O)O)nn1-c1ccc(F)cc1 ; CS(=O)(=O)c1cc(C(=O)O)nn1-c1ccc(F)cc1]

### Synthesis of 1-(4-fluorophenyl)-1H-pyrazole-3-carboxylic acid [Intermediate 42]

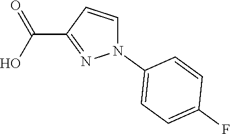 [SMILES: O=C(O)c1ccn(-c2ccc(F)cc2)n1]

Available from Commercial Sources

### Synthesis of 1-(4-fluorophenyl)-5-(methylthio)-1H-pyrazole-3-carboxylic acid [Intermediate 43], 1-(4-fluorophenyl)-5-(methylsulfinyl)-1H-pyrazole-3-carboxylic acid [Intermediate 44], and 1-(4-fluorophenyl)-5-(methylsulfonyl)-1H-pyrazole-3-carboxylic acid [Intermediate 45]

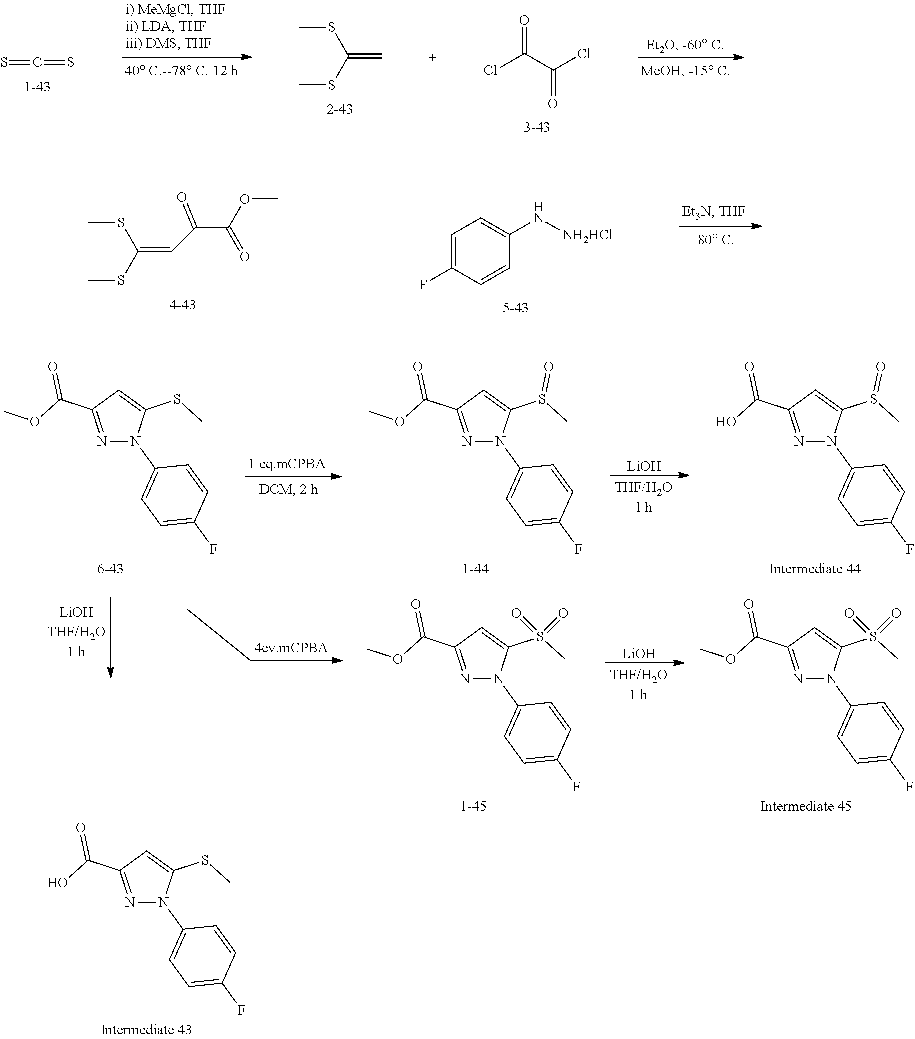

### 1,1-Bis(methylthio)ethylene [2-43]

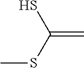

Methyl magnesium chloride (426 mL, 1278 mmol) in anhydrous THF (852 ml) was added to a mechanically stirred 5 L round bottom flask. To this was added carbon disulfide (116 ml, 1917 mmol) in 116 ml of anhydrous THF with an addition rate such that the internal temperature did not exceed 40° C. The reaction mixture was stirred at 40° C. for 2 h. The reaction was then cooled to −78° C. using an acetone/dry ice bath, and to this was added LDA 2.0 M solution in ethyl benzene (639 ml, 1278 mmol) in 640 ml of THF. The reaction was then stirred at −78° C. for 2 h. To this was added a solution of dimethyl sulfate (243 mL, 2556 mmol,) in 246 ml of THF, and the reaction was allowed to warm to room temperature and stirred for 17 h. Then 700 ml of Et₂O was added, and the reaction was stirred for 10 min and allowed to stand during which time the inorganic salts precipitated. The supernatant was decanted, and the salts were rinsed with Et₂O (500 ml). The combined Et₂₀ fractions were then washed with H₂O (2×1500 ml), brine (1000 ml), dried over Na₂SO₄, filtered, and concentrated under reduced pressure to get a dark brown oil. The crude oil was then distilled at 50° C., 3 Torr, to afford 1,1-bis(methylthio)ethylene 2 (60.00 g, 37.09%) as a yellow oil. ¹H NMR (500 MHz, Chloroform-d) δ 5.24 (s, 2H), 2.36 (s, 6H). LCMS: No ionization or UV signal observed.

### methyl 4,4-bis(methylthio)-2-oxo-but-3-enoate [4-43]

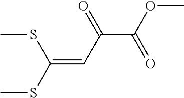

A stirred solution of 1,1-bis(methylthio)ethylene (60.0 g, 474 mmol) in anhydrous Et₂O (600 ml) was cooled to −60° C. using an Et₂O/dry ice bath. To this solution was added oxalyl chloride (50 mL, 569 mmol) dropwise. During the addition of oxalyl chloride the solution turned into a yellow suspension. The suspension was allowed to warm up to −15° C., and MeOH (115 ml, 2859 mmol) was added. The reaction was then allowed to warm to room temperature and stirred for 2 h. Et₂O (400 ml) was added, and the precipitate obtained was filtered and dried in vacuo to give 49.00 g, 47.60% yield of the title compound as a yellow solid. ¹H NMR (500 MHz, chloroform-d) δ 6.83 (s, 1H), 3.86 (s, 3H), 2.56 (s, 3H), 2.54 (s, 3H). LCMS: No ionization or UV signal observed.

### methyl 1-(4-fluorophenyl)-5-(methylthio)-1H-pyrazole-3-carboxylate [6-43]

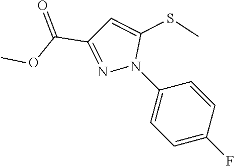 [SMILES: COC(=O)c1cc(SC)n(-c2ccc(F)cc2)n1]

To a stirred solution of 4-fluorophenylhydrazine hydrochloride (22.00 g, 135 mmol) in a dry 500 ml round-bottom flask was added THF (150 ml) and Et₃N (19 ml, 135 mmol). The solution was stirred 10 min after which was added a solution of methyl 4,4-bis(methylthio)-2-oxo-but-3-enoate 4 (28.60 g, 132 mmol) in THF (150 mL). Molecular sieves (3 Å, 8-12 mesh) were added, and the reaction was heated at reflux (80° C.) for 5 h. The reaction was cooled and then filtered through Celite. The Celite was washed with EtOAc (200 ml), and the combined organic layers were evaporated in vacuo to get a crude yellow solid. This solid was further purified using ISCO, Combiflash companion, Siliasep 330 g column, (dry loaded in DCM), eluting 0 to 50% hexane/EtOAc gradient over 60 min). The product fractions were combined and concentrated in vacuo to give 13.00 g, 36.08% yield of the title compound. ¹H NMR (500 MHz, DMSO-d₆) δ 13.00 (s, 1H), 7.64-7.57 (m, 2H), 7.44-7.37 (m, 2H), 6.92 (s, 1H), 2.47 (s, 3H). MS (ES⁺) m/z calcd for [M+H]⁺ [Cl₂H₁₁FN₂O₂S+H]⁺: 252.03 found 253.0, LCMS t_{R}=1.95 min. [Analytical Method: B]

### 1-(4-fluorophenyl)-5-(methylthio)-1H-pyrazole-3-carboxylic acid [Intermediate 43]

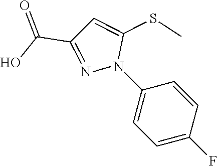 [SMILES: CSc1cc(C(=O)O)nn1-c1ccc(F)cc1]

To a stirred suspension of methyl 1-(4-fluorophenyl)-5-(methylthio)-1H-pyrazole-3-carboxylate 1 (20.3 g, 76.22 mmol, 1.00 eq) in THF (250 mL) and water (250 mL) at rt was added Lithium hydroxide (6.4 g, 152.46 mmol, 2.00 eq). The reaction mixture was stirred at rt for 1 h. THF was removed, aqueous was acidified to pH 4 by adding 1N—HCl. Solids were filtered, washed with water followed by hexanes and dried under reduced pressure to afford 1-(4-fluorophenyl)-5-(methylthio)-1H-pyrazole-3-carboxylic acid as white solid (16.2 g, 84% yield). ¹H NMR (500 MHz, DMSO-d6) δ13.00 (s, 1H), 7.64-7.57 (m, 2H), 7.44-7.37 (m, 2H), 6.92 (s, 1H), 2.47 (s, 3H). MS (ES⁺) m/z calcd for [M+H]⁺ [C₁₁H₉FN₂O₂S+H]⁺: 253.0 found 253.0, LCMS t_{R}=2.03 min. [Analytical Method: B]

### methyl 1-(4-fluorophenyl)-3-(methylsulfinyl)-1H-pyrazole-5-carboxylate [1-44]

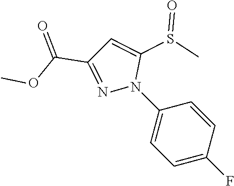 [SMILES: COC(=O)c1cc(S(C)=O)n(-c2ccc(F)cc2)n1]

To a stirred solution of methyl 1-(4-fluorophenyl)-5-(methylthio)-1H-pyrazole-3-carboxylate (13.00 g, 48.8 mmol) in DCM (200 ml) was added 3-chloroperoxybenzoic acid (12.00 g, 48.7 mmol), and the reaction was stirred at room temperature for 2 h. The reaction mixture was then washed with saturated NaHCO₃ solution (2×200 ml), brine (100 ml), dried over Na₂SO₄, filtered, and concentrated to dryness in vacuo to provide 11.50 g, 79.28% yield of the title compound as an orange solid. ¹H NMR (500 MHz, DMSO-d₆) δ 7.76-7.69 (m, 2H), 7.68 (s, 1H), 7.47 (t, J=8.7, 8.7 Hz, 2H), 3.87 (s, 3H), 3.06 (s, 3H). MS (ES⁺) m/z calcd for [M+H]⁺ [Cl₂H₁₁FN₂O₃S+H]⁺: 282.29 found 283.6, LCMS t_{R}=4.91 min. [Analytical Method: A-6]

### 1-(4-fluorophenyl)-5-(methylsulfinyl)-1H-pyrazole-3-carboxylic acid [Intermediate 44]

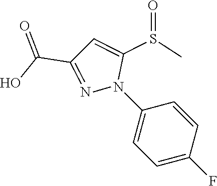 [SMILES: CS(=O)c1cc(C(=O)O)nn1-c1ccc(F)cc1]

To a stirred solution/suspension of methyl 1-(4-fluorophenyl)-5-(methylsulfinyl)-1H-pyrazole-3-carboxylate (11.50 g, 40.7 mmol) in THF (100 ml) was added a solution of LiOH (1.90 g, 79.3 mmol) in H₂O (100 ml). The reaction was stirred for 1 h. After completion, 5% HCl (100 ml) was added to the reaction mixture until pH=1. The mixture was then extracted with EtOAc (500 ml). The organic layer was washed with brine (200 ml), dried over Na₂SO₄, filtered, and concentrated in vacuo to give a yellow oil. The oil was triturated with EtOAc:hexane (1:3), then filtered to afford 9.20 g, 80.65% of the title compound as an orange solid. ¹H NMR (500 MHz, DMSO-d6) δ 13.2-13.4 (s, 1H), δ 7.75-7.68 (m, 2H), 7.60 (d, J=0.9 Hz, 1H), 7.46 (t, J=8.8, 8.8 Hz, 2H), 3.04 (s, 3H).MS (ES⁺) m/z calcd for [M+H]⁺ [C₁₁H₉FN₂O₃S+H]⁺: 268.02 found 268.9, LCMS t_{R}=1.17 min. [Analytical Method: B]

### methyl 1-(4-fluorophenyl)-5-(methylsulfonyl)-1H-pyrazole-3-carboxylate [1-45]

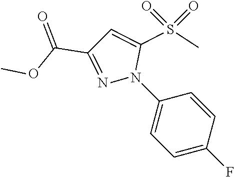 [SMILES: COC(=O)c1cc(S(C)(=O)=O)n(-c2ccc(F)cc2)n1]

To a stirred solution of methyl 1-(4-fluorophenyl)-5-(methylthio)-1H-pyrazole-3-carboxylate (17.00 g, 63.8 mmol) in DCM (500 mL, 0.1277 M) was added 3-chloroperoxybenzoic acid (62.95 g, 255 mmol) and the reaction was stirred at room temperature for 2 h. The mixture was then washed with saturated NaHCO₃ solution (2×200 ml), brine (100 ml), dried over Na₂SO₄, filtered, and evaporated to give 12.50 g, 62.36% yield of the title compound as a yellow solid. ¹H NMR (500 MHz, DMSO-d6) δ 7.73-7.66 (m, 2H), 7.60 (s, 1H), 7.42 (t, J=8.7, 8.7 Hz, 2H), 3.86 (s, 3H), 3.33 (s, 3H). MS (ES⁺) m/z calcd for [M+H]⁺ [C₁₂H₁₁FN₂O₄S+H]⁺: 298.29 found 299.50, LCMS t_{R}=5.42 min. [Analytical Method: A]

### 1-(4-fluorophenyl)-5-(methylsulfonyl)-1H-pyrazole-3-carboxylic acid [Intermediate 45]

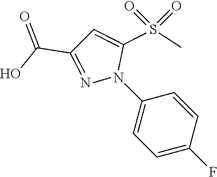 [SMILES: CS(=O)(=O)c1cc(C(=O)O)nn1-c1ccc(F)cc1]

To a stirred solution of methyl 1-(4-fluorophenyl)-5-(methylsulfonyl)-1H-pyrazole-3-carboxylate (11.00 g, 35.0 mmol) in THF (100 ml) was added a solution of LiOH (1.68 g, 70.1 mmol) in H₂O (100 ml). The reaction was allowed to stir for 2 h. After completion, 2N HCL was added to the reaction mixture, which was then extracted with EtOAc (500 ml). The organic layer was washed with brine (200 ml), dried over Na₂SO₄, filtered, and evaporated to get a yellowish oil. The oil was triturated with EtOAc:hexane (1:3) to afford 8.60 g, 28.5 mmol, 81.35% yield of the title compound as a pale yellow solid. ¹H NMR (500 MHz, DMSO-d6) δ 7.68 (ddd, J=8.9, 4.7, 1.8 Hz, 2H), 7.52 (d, J=1.9 Hz, 1H), 7.42 (td, J=8.8, 8.7, 1.9 Hz, 2H), 3.31 (s, 3H). MS (ES⁺) m/z calcd for [M+H]⁺ [C₁₁H₉FN₂O₄S+H]⁺: 284.3 found 285.4, LCMS t_{R}=2.02 min. [Analytical Method: B]

### Biochemical Assays

### 1. Kinase Panel

The disclosed compounds were tested for activity against a panel of at least 300 kinases. Kinase panel screening was conducted by Nanosyn (Santa Clara, CA 95051) using an enzymatic inhibition assay accepted as valid by those skilled in the art (e.g., the Caliper LabChip® mobility shift assay, an ADP detection assay, or time-resolved fluorescence detection technology. Compounds were screened at a concentration of 5 μM using an ATP concentration at the Km for each of the respective kinases and a 30-minute pre-incubation time-point.

A selection of kinases from that panel in which one or more of the disclosed compounds showed inhibition of kinase activity is shown below in Tables 1-4. In Table 1 and 2, kinase inhibition is classified by: A=95% or greater, B=90%-94%, C=80%-89%, and D=79% and less with a compound concentration of 5 JIM. In Table 3 and 4, kinase inhibition is reported in terms of IC50 values corresponding to A=less than 500 nm, B=500 nm to less than 1 μM, C=1 μM to less than 10 μM, and D=10 μM or greater using a compound concentration of 5 μM.

TABLE 1

| Kinase | Ex4 | Ex5 | Ex6 | Ex7 | Ex8 | Ex9 | Ex10 | Ex11 | Ex12 |
|---|---|---|---|---|---|---|---|---|---|
| Abl1 | D | D | D | D | D | D | D | D | D |
| Abl2 | D | D | A | D | D | D | D | D | D |
| ARAF | A | A | D | D | D | A | A | A | A |
| BLK | D | D | C | D | D | D | D | D | D |
| BRAF | A | A | A | B | B | A | A | A | A |
| BRK | D | D | C | D | D | D | D | D | D |
| CLK4 | D | D | D | D | C | D | D | D | D |
| COT1 | D | D | D | D | B | D | D | D | D |
| CRAF | A | A | A | A | A | A | A | A | A |
| CSF1R | D | D | B | D | D | D | D | D | D |
| CSK | D | D | C | D | D | D | D | D | D |
| DDR1 | D | B | A | D | D | A | A | D | C |
| DDR2 | A | D | D | D | D | B | C | D | D |
| EPH | D | D | D | D | D | D | D | D | D |
| ERBB4 | D | D | D | D | D | D | D | D | D |
| FGR | D | D | C | D | D | D | D | D | D |
| FYN | D | D | A | D | D | D | D | D | D |
| HCK | D | D | A | D | D | D | D | D | D |
| HGK | D | D | D | D | C | D | D | D | D |
| KDR | D | D | D | D | D | D | D | D | D |
| KIT | B | C | B | D | D | D | D | D | D |
| LCK | D | B | A | D | D | D | D | D | D |
| LYNa | D | A | B | D | D | D | D | D | D |
| LYNb | D | B | A | D | D | D | D | D | D |
| MAP4K2 | D | D | D | D | C | D | D | D | D |
| MNK2 | D | D | D | D | C | D | D | D | D |
| p38a | C | A | B | D | D | D | D | A | D |
| p38b | D | C | C | D | D | C | D | D | D |
| PDGFRa | D | D | A | D | D | D | D | D | D |
| PDGFRb | C | D | A | D | D | D | D | D | D |
| PIKfyve | D | D | B | D | D | D | D | D | D |
| PKCb1 | D | D | D | D | C | D | D | D | D |
| RET | B | D | B | D | D | D | D | D | D |
| SGK2 | D | D | B | D | D | D | D | D | D |
| SGK3 | D | D | D | D | C | D | D | D | D |
| SPHK2 | D | D | D | D | D | D | D | D | D |
| SRC | D | D | B | D | D | D | D | D | D |
| TAOK2 | D | D | D | D | D | A | B | D | C |
| TNIK | D | D | D | D | B | D | D | D | D |
| YES | D | D | C | D | D | D | D | D | D |
| ZAK | D | D | D | D | D | D | D | D | D |

TABLE 2

| Kinase | Ex13 | Ex14 | Ex15 | Ex17 | Ex18 |
|---|---|---|---|---|---|
| Abl1 | A | D | A | D | A |
| Abl2 | A | A | A | D | C |
| ARAF | A | A | D | D | D |
| BLK | D | C | D | D | D |
| BRAF | A | A | A | D | B |
| BRK | D | D | D | A | D |
| CLK4 | D | D | D | D | D |
| COT1 | D | D | D | D | D |
| CRAF | D | A | A | D | A |
| CSF1R | A | B | A | D | C |
| CSK | D | C | C | D | D |
| DDR1 | A | A | A | C | A |
| DDR2 | A | A | A | D | A |
| EPH | D | D | D | D | D |
| ERBB4 | C | D | D | D | D |
| FGR | D | C | C | D | C |
| FYN | D | B | A | D | A |
| HCK | D | B | C | D | A |
| HGK | D | D | D | D | D |
| KDR | D | B | C | D | D |
| KIT | A | B | A | D | C |
| LCK | B | A | A | D | A |
| LYNa | A | A | A | D | A |
| LYNb | A | A | A | D | D |
| MAP4K2 | D | D | D | D | D |
| MNK2 | D | D | D | D | D |
| p38a | D | D | A | D | A |
| p38b | D | D | C | D | C |
| PDGFRa | A | D | A | D | B |
| PDGFRb | A | B | A | D | B |
| PIKfyve | D | C | D | D | D |
| PKCb1 | D | D | D | D | D |
| RET | D | C | A | D | C |
| SGK2 | D | D | D | D | D |
| SGK3 | D | D | D | D | D |
| SPHK2 | C | D | D | D | C |
| SRC | D | D | B | D | C |
| TAOK2 | D | D | D | D | D |
| TNIK | D | D | D | D | D |
| YES | D | D | A | D | B |
| ZAK | D | C | D | D | D |

TABLE 3

| Kinase | Ex69 | Ex70 | Ex71 | Ex72 | Ex73 | Ex74 | Ex75 | Ex76 | Ex77 | Ex78 |
|---|---|---|---|---|---|---|---|---|---|---|
| AXL | | | A | A | A | | | | | |
| DDR1 | | | A | | A | | | | | |
| DDR2 | | | A | | A | | | | | |
| ITK | | | D | | C | | D | | | |
| JAK3 | | | D | D | D | | | | | |
| LCK | | | A | B | B | | | | | |
| MER | | | A | B | A | | | | | |
| PAK1 | | | | | | D | | D | | |
| PAK2 | | | | | | | | D | | |
| PAK4 | | | | | | | | C | | |
| PLK1 | | | D | D | D | | | | | |
| PLK4 | | | C | B | A | | | | | |
| RIPK1 | | | D | | | | | | | |
| RIPK2 | | | B | | | | | | | |
| RIPK3 | | | C | | | | | | | |
| SYK | | | D | C | D | C | | | D | C |
| TAK1-TAB1 | | | D | D | D | | | | | |
| TYK2 | | | D | C | | | | | | |
| TYRO3 | | | A | | C | | | | A | A |
| WEE1 | D | D | | | | D | | D | | |

TABLE 4

| Kinase | Ex79 | Ex80 | Ex81 | Ex82 | Ex83 | Ex84 | Ex85 | Ex86 | Ex87 |
|---|---|---|---|---|---|---|---|---|---|
| AXL | | | | | | | | | |
| DDR1 | | | | | | | | | |
| DDR2 | | | | | | | | | |
| ITK | | | | | | | | | |
| JAK3 | | | | | | | | | D |
| LCK | | | | | | | | | |
| MER | | | | | | | | | |
| PAK1 | | | | | | | D | | |
| PAK2 | | | | | | | D | | |
| PAK4 | | | | | | | | | |
| PLK1 | | | | | | | | | |
| PLK4 | | | | | | D | | A | |
| RIPK1 | A | D | | | | | | | |
| RIPK2 | A | D | | | | | | | |
| RIPK3 | | | | | | | | | |
| SYK | | | C | | | | | | |
| TAK1- | | | | | D | | | | |
| TAB1 | | | | | | | | | |
| TYK2 | | | | | | | | | D |
| TYRO3 | | | | | | | | | |
| WEE1 | | | | D | | | | | |

While we have described a number of embodiments, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A compound having the Formula I: 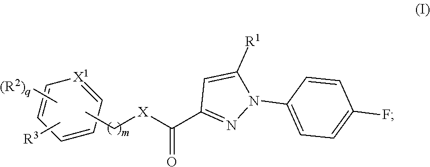
or a pharmaceutically acceptable salt thereof, wherein
R¹ is hydrogen, —SO₂R^{a}, —SOR^{a}, or —SR^{a};
X is —NR^{b} or O;
m is 0, 1, or 2;
X¹ is CH or N;
R² is halogen, hydroxyl, (C₁-C₆)alkyl, cyano, (C₁-C₆)alkoxy, halo(C₁-C₆)alkyl, —NR^{b}R^{d}, and halo(C₁-C₆)alkoxy;
R³ is —OR⁴, —NHR⁵, —N(C₁-C₆)alkylR⁵, —CCHR⁶, —NHCOR⁷, —N(C₁-C₆)alkylCOR⁷, —C(O)R⁷, phenyl, —(C₁-C₆)alkyl[phenyl], heteroaryl, —(C₁-C₆)alkyl[heteroaryl], heterocyclyl, or —(C₁-C₆)alkyl[heterocyclyl] wherein said phenyl, heteroaryl and heterocyclyl alone or part of —(C₁-C₆)alkyl[phenyl], —(C₁-C₆)alkyl[heteroaryl], and —(C₁-C₆)alkyl[heterocyclyl] are each optionally substituted with 1 to 3 groups selected from R⁸; or R³ is taken together with one R² to form a 4- to 6-membered heteroaryl optionally substituted with a heteroaryl which is optionally substituted with 1 to 3 groups selected from R⁹;
R⁴ is heteroaryl optionally substituted with 1 to 3 groups selected from R¹⁰;
R⁵ is heteroaryl or heterocyclyl, each of which are optionally substituted with 1 to 3 groups selected from R¹¹;
R⁶ and R⁷ are each independently heteroaryl optionally substituted with 1 to 3 groups selected from R¹²;
R⁸ is selected from halo, (C₁-C₆)alkyl, halo(C₁-C₆)alkyl, (C₁-C₆)alkoxy, halo(C₁-C₆)alkoxy, oxo, 4- to 6-membered heterocyclyl, —O(C₁-C₆)hydroxyalkyl, —NR^{b}C(O)R^{b}, and NR^{b}R^{c};
R⁹ is selected from halo, (C₁-C₆)alkyl, halo(C₁-C₆)alkyl, (C₁-C₆)alkoxy, and halo(C₁-C₆)alkoxy;
R¹⁰ is selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, halo, (C₁-C₆)alkoxy, halo(C₁-C₆)alkoxy, (C₁-C₆)alkyl, halo(C₁-C₆)alkyl, heterocyclyl, and heteroaryl, wherein said heterocyclyl and heteroaryl group are each optionally substituted with 1 to 3 groups selected from oxo, halo, (C₁-C₆)alkyl halo(C₁-C₆)alkyl, (C₁-C₆)alkoxy, halo(C₁-C₆)alkoxy, —C(O)NR^{b}R^{c}, —NR^{b}R^{c}, —C(O)ONR^{b}R^{c}, —C(O)R^{b}, —C(O)OR^{b}, —NR^{b}C(O)R^{b}, and —(C₁-C₆)alkylNH(C₁-C₆)hydroxyalkyl;
R¹¹ is selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, (C₁-C₆)alkoxy, halo(C₁-C₆)alkoxy, oxo, halo, (C₁-C₆)alkyl, halo(C₁-C₆)alkyl, heterocyclyl, and heteroaryl wherein said heterocyclyl and heteroaryl are each optionally substituted with 1 to 3 groups selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, (C₁-C₆)alkoxy, halo(C₁-C₆)alkoxy, oxo, halo, (C₁-C₆)alkyl, and halo(C₁-C₆)alkyl;
R¹² is selected from —C(O)NR^{b}R^{c}, —NR^{b}R^{c}, —C(O)ONR^{b}R^{c}, —C(O)R^{b}, —C(O)OR^{b}, —NR^{b}C(O)R^{b}, (C₁-C₆)alkoxy, halo(C₁-C₆)alkoxy, oxo, halo, (C₁-C₆)alkyl, and halo(C₁-C₆)alkyl;
q is 0, 1, 2, or 3;
R^{a} is (C₁-C₆)alkyl;
R^{b} and R^{d} are each independently hydrogen or (C₁-C₆)alkyl; and
R^{c} is selected from hydrogen, (C₁-C₆)alkyl, and 4- to 6-membered heterocyclyl.

2. The compound of claim 1, wherein the compound is of the Formula II: 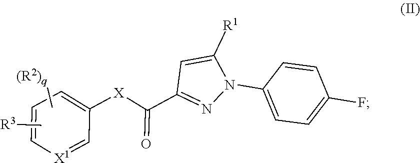
or a pharmaceutically acceptable salt thereof.

3. The compound of claim 1 or 2, wherein the compound is of the Formula III: 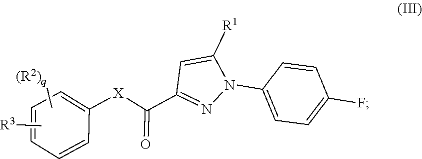
or a pharmaceutically acceptable salt thereof.

4. The compound of any one of claims 1 to 3, wherein the compound is of the Formula IV: 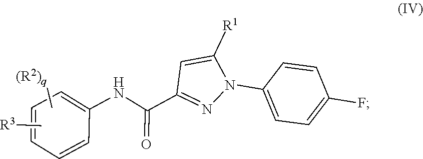
or a pharmaceutically acceptable salt thereof.

5. The compound of any one of claims 1 to 4, or a pharmaceutically acceptable salt thereof, wherein R¹ is —SO₂R^{a}, —SOR^{a}, or —SR^{a}.

6. The compound of any one of claims 1 to 5, or a pharmaceutically acceptable salt thereof, wherein R^{a} is (C₁-C₃)alkyl.

7. The compound of any one of claims 1 to 6, or a pharmaceutically acceptable salt thereof, wherein R¹ is —SO₂CH₃, —SCH₃, or —SOCH₃.

8. The compound of any one of claims 1 to 7, or a pharmaceutically acceptable salt thereof, wherein R² is halogen, —NR^{b}R^{d}, hydroxyl, or (C₁-C₃)alkyl.

9. The compound of any one of claims 1 to 8, or a pharmaceutically acceptable salt thereof, wherein R² is hydroxyl, fluoro, bromo, methyl or NH₂.

10. The compound of any one of claims 1 to 9, or a pharmaceutically acceptable salt thereof, wherein q is 0, 1, or 2.

11. The compound of any one of claims 1 to 10, or a pharmaceutically acceptable salt thereof, wherein q is 1 or 2.

12. The compound of any one of claims 1 to 11, or a pharmaceutically acceptable salt thereof, wherein R³ is —OR⁴, NHR⁵, —CCHR⁶, —NHCOR⁷, —C(O)R⁷, phenyl, heteroaryl, —(C₁-C₆)alkyl[heteroaryl], or heterocyclyl, wherein said phenyl, heteroaryl, heterocyclyl, and the heteroaryl on —(C₁-C₆)alkyl[heteroaryl] are each optionally substituted with 1 to 3 groups selected from R⁸; or R³ is taken together with one R² to form a 4- to 6-membered heteroaryl optionally substituted with a 9- or 10-membered fused-bicyclic heteroaryl which is optionally substituted with 1 to 3 groups selected from R⁹.

13. The compound of any one of claims 1 to 12, or a pharmaceutically acceptable salt thereof, wherein R³ is —OR⁴, NHR⁵, —CCHR⁶, —NHCOR⁷, —C(O)R⁷, phenyl, heteroaryl, or heterocyclyl, wherein said phenyl, heteroaryl and heterocyclyl are each optionally substituted with 1 to 3 groups selected from R⁸; or R³ is taken together with one R² to form a 4- to 6-membered heteroaryl optionally substituted with a 9- or 10-membered fused-bicyclic heteroaryl which is optionally substituted with 1 to 3 groups selected from R⁹.

14. The compound of any one of claims 1 to 13, or a pharmaceutically acceptable salt thereof, wherein R³ is —OR⁴, NHR⁵, —CCHR⁶, —NHCOR⁷, —C(O)R⁷, phenyl, 5- to 6-membered heteroaryl, 9- to 10-membered fused bicyclic heteroaryl, 5- to 6-membered heterocyclyl, or 9- to 10-membered fused bicyclic heterocyclyl, wherein said phenyl, 5- to 6-membered heteroaryl, 9- to 10-membered fused bicyclic heteroaryl, 5- to 6-membered heterocyclyl, and 9- to 10-membered fused bicyclic heterocyclyl are each optionally substituted with 1 to 3 groups selected from R⁸; or R³ is taken together with one R² to form a 5-membered heteroaryl optionally substituted with a 9- or 10-membered fused-bicyclic heteroaryl which is optionally substituted with 1 to 3 groups selected from R⁹.

15. The compound of any one of claims 1 to 12, or a pharmaceutically acceptable salt thereof, wherein R³ is —OR⁴, NHR⁵, —CCHR⁶, —NHCOR⁷, —C(O)R⁷, (C₁-C₄)alkylpyrrolopyridinyl, phenyl, pyridinyl, thienopyrimidinyl, dihydropyridopyrimidinyl, dihydrobenzoimidazolyl imidazopyridinyl, pyridopyrimidinyl, or dihydropyridinyl, wherein said phenyl, pyridinyl, thienopyrimidinyl, dihydropyridopyrimidinyl, dihydrobenzoimidazolyl imidazopyridinyl, pyridopyrimidinyl, dihydropyridinyl, and pyrrolopyridinyl on the (C₁-C₄)alkylpyrrolopyridinyl are each optionally substituted with 1 to 3 groups selected from R⁸; or R³ is taken together with one R² to form a furanyl optionally substituted with imidazopyridazinyl which is optionally substituted with 1 to 3 groups selected from R⁹.

16. The compound of any one of claims 1 to 12, or a pharmaceutically acceptable salt thereof, wherein R³ is (C₁-C₄)alkylpyrrolopyridinyl, phenyl, pyridinyl, thienopyrimidinyl, dihydropyridopyrimidinyl, dihydrobenzoimidazolyl imidazopyridinyl, pyridopyrimidinyl, or dihydropyridinyl, wherein said phenyl, pyridinyl, thienopyrimidinyl, dihydropyridopyrimidinyl, dihydrobenzoimidazolyl imidazopyridinyl, pyridopyrimidinyl, and dihydropyridinyl are each optionally substituted with 1 to 3 groups selected from R⁸; or R³ is taken together with one R² to form a furanyl optionally substituted with imidazopyridazinyl which is optionally substituted with 1 to 3 groups selected from R⁹.

17. The compound of any one of claims 1 to 16, or a pharmaceutically acceptable salt thereof, wherein R⁴ is 5- to 6-membered heteroaryl or 9- to 10-membered fused bicyclic heteroaryl, each of which are optionally substituted with 1 to 3 groups selected from R¹⁰.

18. The compound of any one of claims 1 to 17, or a pharmaceutically acceptable salt thereof, wherein R⁴ is pyridinyl, dihydroquinazolinyl, dihydropyridopyrimidinyl, thiazolopyridinyl, quinolinyl, pyrrolopyridinyl, or tetrahydronaphthyridinyl, each of which are optionally substituted with 1 to 3 groups selected from R¹⁰.

19. The compound of any one of claims 1 to 18, or a pharmaceutically acceptable salt thereof, wherein R⁴ is pyridinyl, dihydroquinazolinyl, dihydropyridopyrimidinyl, thiazolopyridinyl, quinolinyl, or tetrahydronaphthyridinyl, each of which are optionally substituted with 1 to 3 groups selected from R¹⁰.

20. The compound of any one of claims 1 to 19, or a pharmaceutically acceptable salt thereof, wherein R⁵ is 5- to 6-membered heteroaryl, 9- to 10-membered fused bicyclic heteroaryl, 5- to 6-membered heterocyclyl, or 9- to 10-membered fused bicyclic heterocyclyl, each of which are optionally substituted with 1 to 3 groups selected from R¹¹.

21. The compound of any one of claims 1 to 20, or a pharmaceutically acceptable salt thereof, wherein R⁵ is pyridinyl, pyrimidinyl, dihydroquinazolinyl, thiazolyl, dihydropyridopyrimidinyl, or imidazopyridazinyl, each of which are optionally substituted with 1 to 3 groups selected from R¹¹.

22. The compound of any one of claims 1 to 21, or a pharmaceutically acceptable salt thereof, wherein R⁶ is a 9- to 10-membered fused bicyclic heteroaryl optionally substituted with 1 to 3 groups selected from R¹².

23. The compound of any one of claims 1 to 22, or a pharmaceutically acceptable salt thereof, wherein R⁶ is imidazopyridazinyl or thienopyrimidinyl, each of which are optionally substituted with 1 to 3 groups selected from R¹².

24. The compound of any one of claims 1 to 23, or a pharmaceutically acceptable salt, wherein R⁷ is a 5- to 6-membered heteroaryl or a 9- to 10-membered heteroaryl each optionally substituted with 1 to 3 groups selected from R¹².

25. The compound of any one of claims 1 to 24, or a pharmaceutically acceptable salt, wherein R⁷ is a 5- to 6-membered heteroaryl optionally substituted with 1 to 3 groups selected from R¹².

26. The compound of any one of claims 1 to 24, or a pharmaceutically acceptable salt, wherein R⁷ is a 9- to 10-membered heteroaryl optionally substituted with 1 to 3 groups selected from R¹².

27. The compound of any one of claims 1 to 24, or a pharmaceutically acceptable salt, wherein R⁷ is pyrrolopyridinyl.

28. The compound of any one of claims 1 to 27, or a pharmaceutically acceptable salt, wherein R⁸ is selected from (C₁-C₆)alkyl, oxo, morpholinyl, —O(C₁-C₆)hydroxyalkyl, and —NR^{b}C(O)R^{b}, NR^{b}R^{c}.

29. The compound of any one of claims 1 to 28, or a pharmaceutically acceptable salt, wherein R⁹ is halo.

30. The compound of any one of claims 1 to 29, or a pharmaceutically acceptable salt, wherein R¹⁰ is —C(O)NR^{b}R^{c}, —NR^{b}R^{c}, (C₁-C₆)alkoxy, and pyridinyl, wherein said pyridinyl is optionally substituted with —(C₁-C₆)alkylNH(C₁-C₆)hydroxyalkyl.

31. The compound of any one of claims 1 to 30, or a pharmaceutically acceptable salt, wherein R¹¹ is oxo, (C₁-C₆)alkyl, and heteroaryl, wherein said heteroaryl is optionally substituted with 1 to 3 groups selected from NR^{b}R^{c}, halo, (C₁-C₆)alkyl, —NR^{b}C(O)R^{b}, and —NR^{b}C(O)OR^{b}.

32. The compound of any one of claims 1 to 31, or a pharmaceutically acceptable salt, wherein R¹¹ is oxo, (C₁-C₆)alkyl, pyridinyl, thiazolyl, or purinyl, wherein said pyridinyl, thiazolyl, or purinyl are each optionally substituted with 1 to 3 groups selected from NR^{b}R^{c}, halo, (C₁-C₆)alkyl, —NR^{b}C(O)R^{b}, and —NR^{b}C(O)OR^{b}.

33. The compound of any one of claims 1 to 32, or a pharmaceutically acceptable salt, wherein R¹¹ is oxo, (C₁-C₆)alkyl, pyridinyl, thiazolyl, or purinyl, wherein said pyridinyl, thiazolyl, or purinyl are each optionally substituted with 1 to 3 groups selected is optionally substituted with 1 to 3 groups selected from oxo and (C₁-C₆)alkyl.

34. The compound of any one of claims 1 to 33, or a pharmaceutically acceptable salt, wherein R¹² is NR^{b}R^{c} or halo.

35. The compound of any one of claims 1 to 34, or a pharmaceutically acceptable salt, wherein R^{c} is selected from hydrogen, (C₁-C₆)alkyl, and 4- to 6-membered heterocyclyl.

36. The compound of claim 1, wherein the compound is selected from 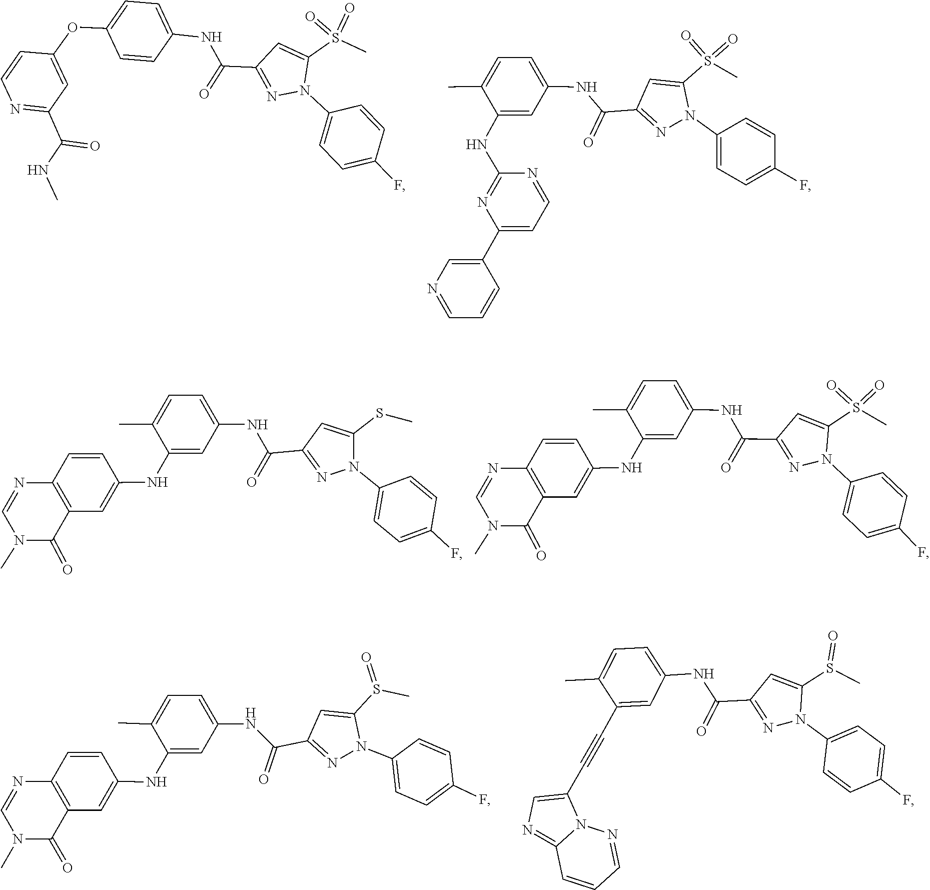 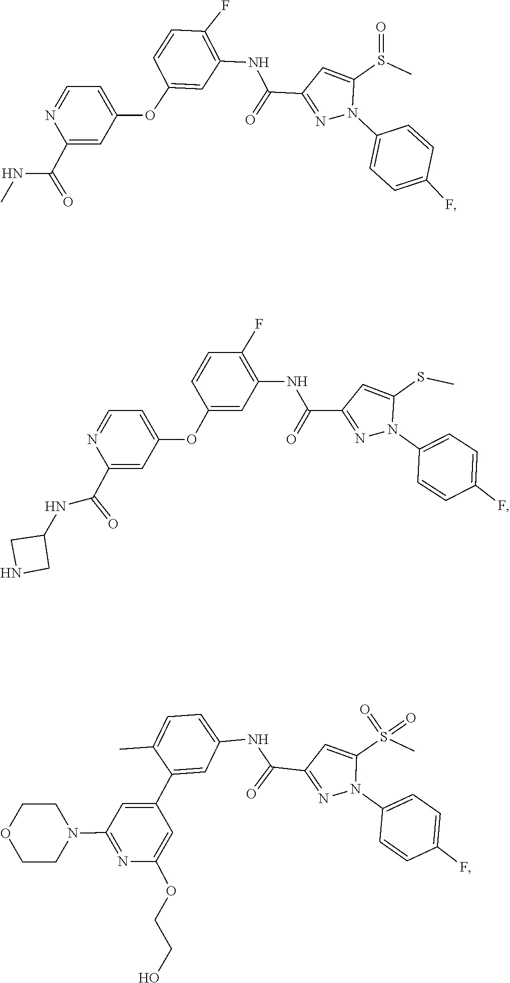 [SMILES: CNC(=O)c1cc(Oc2ccc(F)c(NC(=O)c3cc(S(C)=O)n(-c4ccc(F)cc4)n3)c2)ccn1 ; CSc1cc(C(=O)Nc2cc(Oc3ccnc(C(=O)NC4CNC4)c3)ccc2F)nn1-c1ccc(F)cc1 ; Cc1ccc(NC(=O)c2cc(S(C)(=O)=O)n(-c3ccc(F)cc3)n2)cc1-c1cc(OCCO)nc(N2CCOCC2)c1] 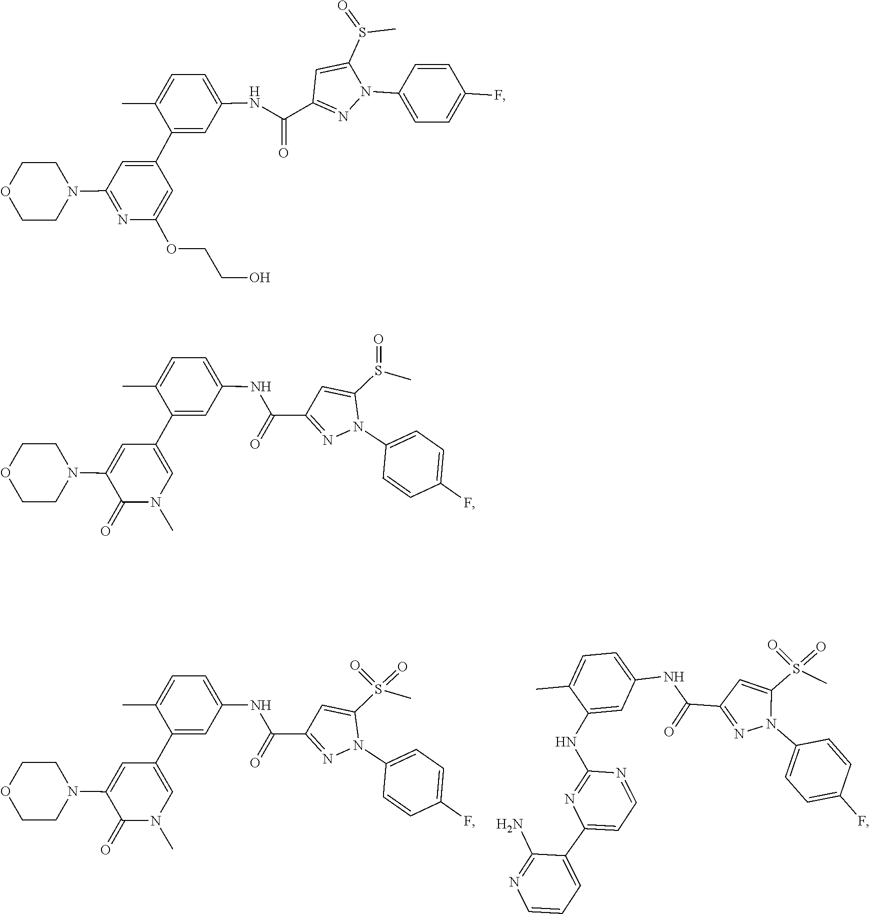 [SMILES: Cc1ccc(NC(=O)c2cc(S(C)=O)n(-c3ccc(F)cc3)n2)cc1-c1cc(OCCO)nc(N2CCOCC2)c1 ; Cc1ccc(NC(=O)c2cc(S(C)=O)n(-c3ccc(F)cc3)n2)cc1-c1cc(N2CCOCC2)c(=O)n(C)c1 ; Cc1ccc(NC(=O)c2cc(S(C)(=O)=O)n(-c3ccc(F)cc3)n2)cc1-c1cc(N2CCOCC2)c(=O)n(C)c1 ; Cc1ccc(NC(=O)c2cc(S(C)(=O)=O)n(-c3ccc(F)cc3)n2)cc1Nc1nccc(-c2cccnc2N)n1] 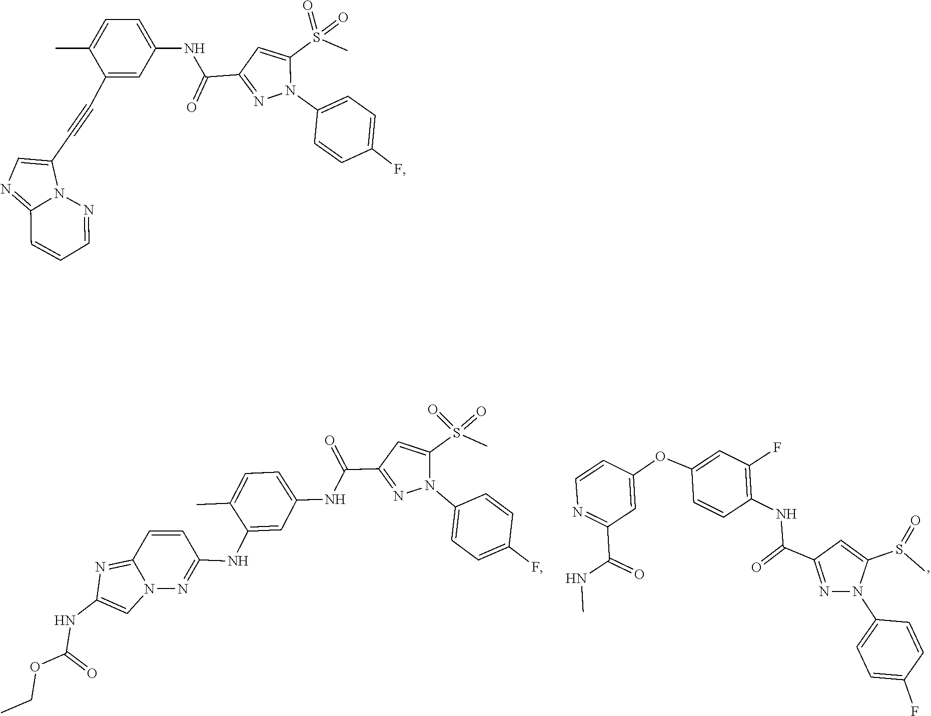 [SMILES: Cc1ccc(NC(=O)c2cc(S(C)(=O)=O)n(-c3ccc(F)cc3)n2)cc1C#Cc1cnc2cccnn12 ; CCOC(=O)Nc1cn2nc(Nc3cc(NC(=O)c4cc(S(C)(=O)=O)n(-c5ccc(F)cc5)n4)ccc3C)ccc2n1 ; CNC(=O)c1cc(Oc2ccc(NC(=O)c3cc(S(C)=O)n(-c4ccc(F)cc4)n3)c(F)c2)ccn1] 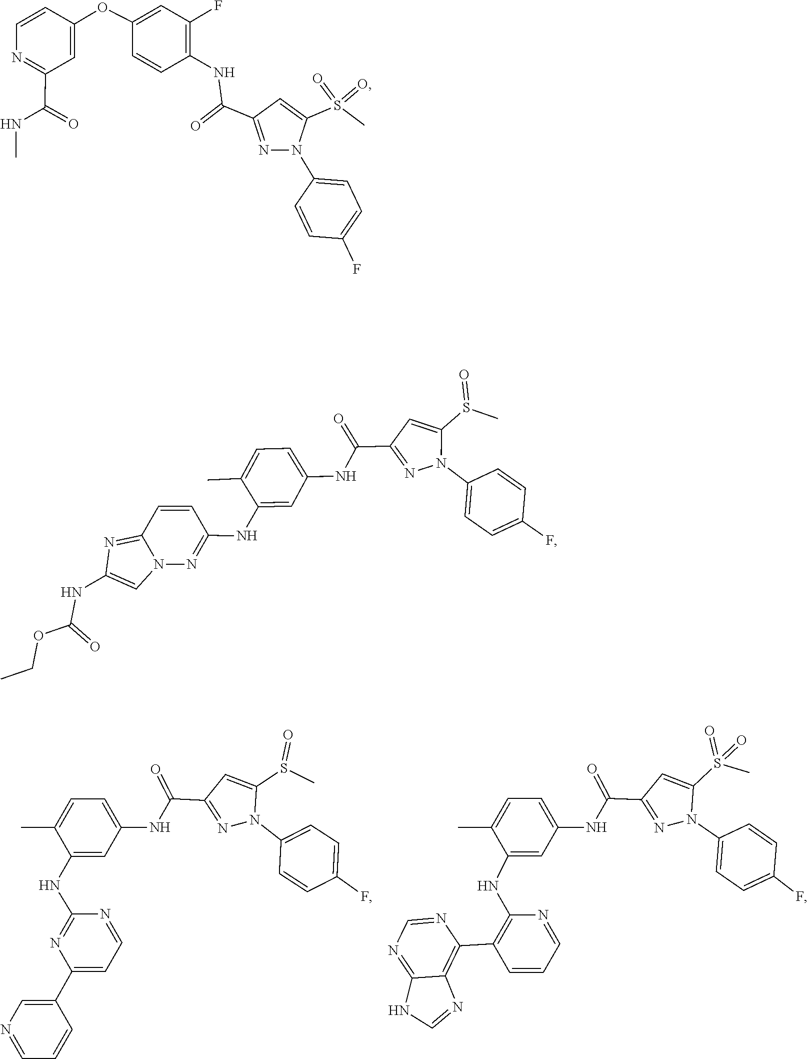 [SMILES: CNC(=O)c1cc(Oc2ccc(NC(=O)c3cc(S(C)(=O)=O)n(-c4ccc(F)cc4)n3)c(F)c2)ccn1 ; CCOC(=O)Nc1cn2nc(Nc3cc(NC(=O)c4cc(S(C)=O)n(-c5ccc(F)cc5)n4)ccc3C)ccc2n1 ; Cc1ccc(NC(=O)c2cc(S(C)=O)n(-c3ccc(F)cc3)n2)cc1Nc1nccc(-c2cccnc2)n1 ; Cc1ccc(NC(=O)c2cc(S(C)(=O)=O)n(-c3ccc(F)cc3)n2)cc1Nc1ncccc1-c1ncnc2[nH]cnc12] 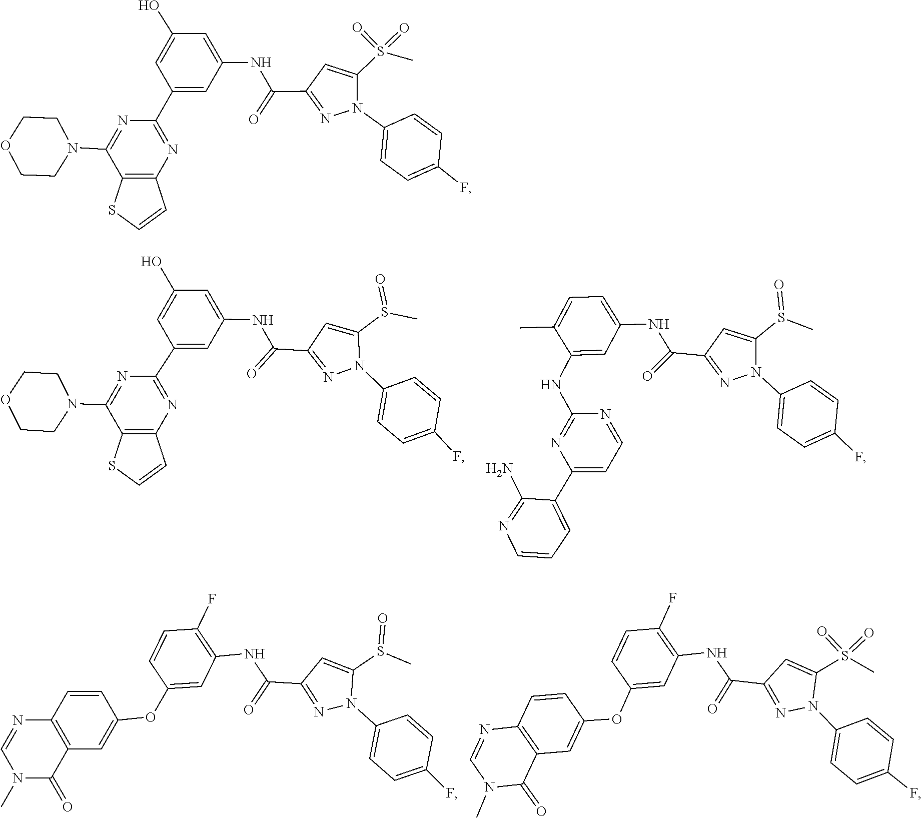 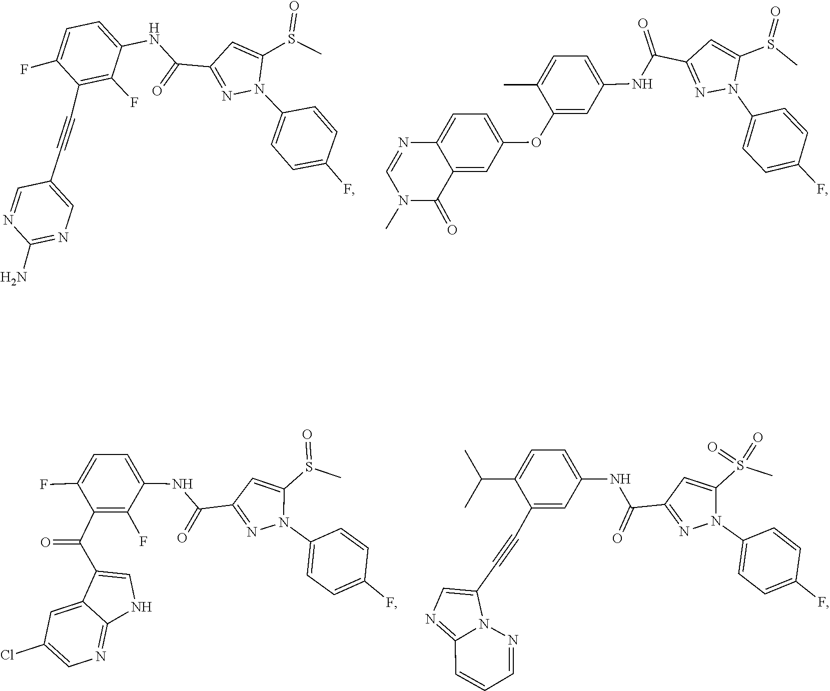 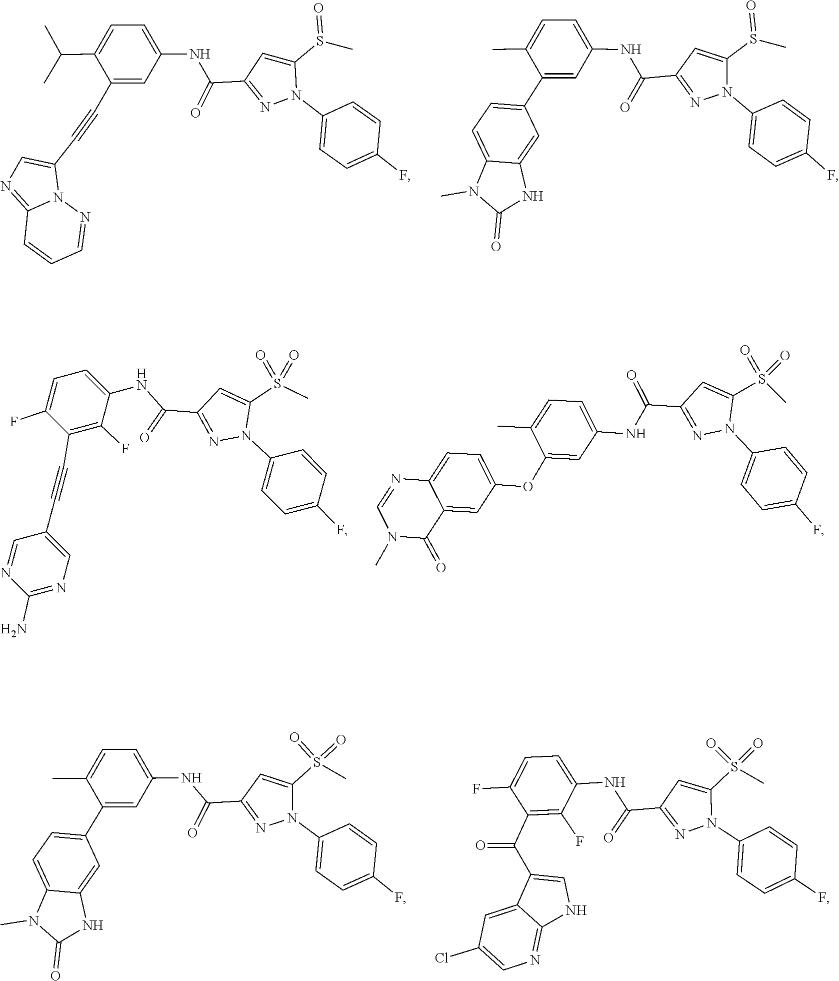 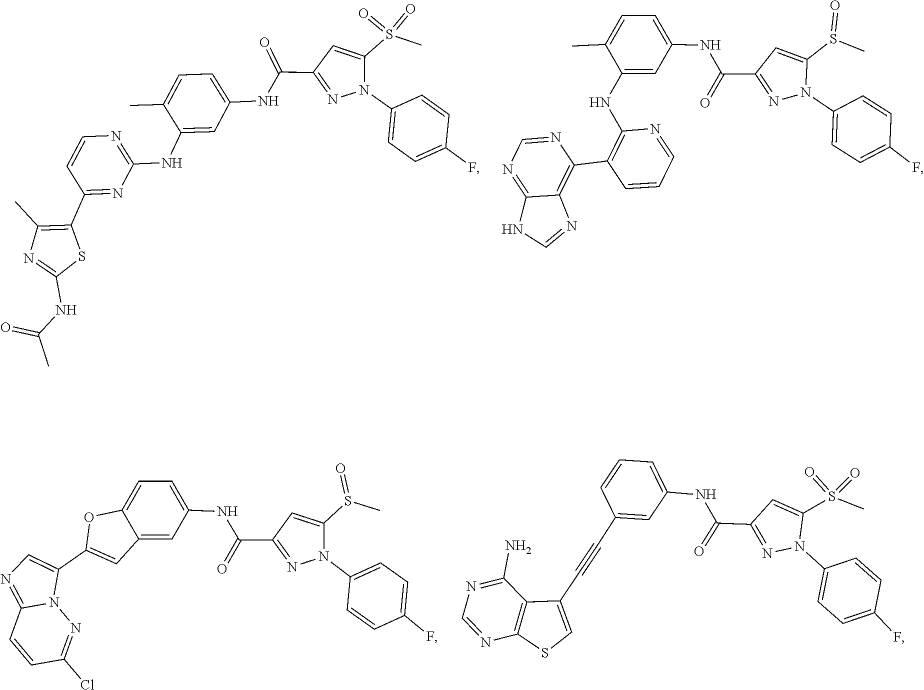 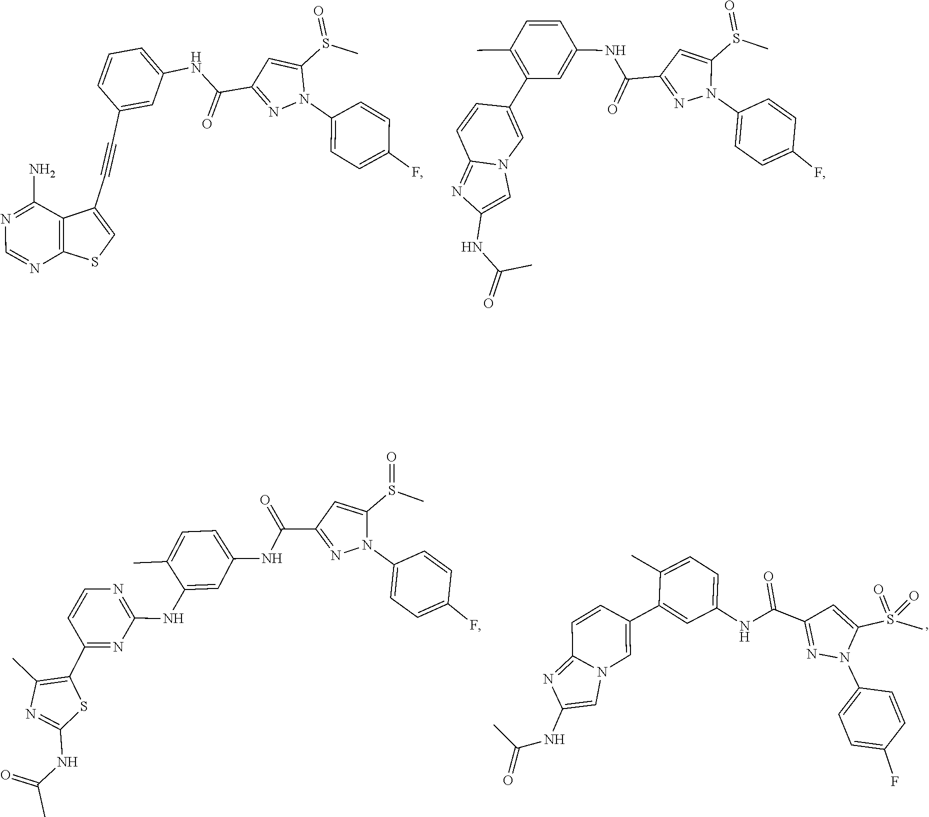 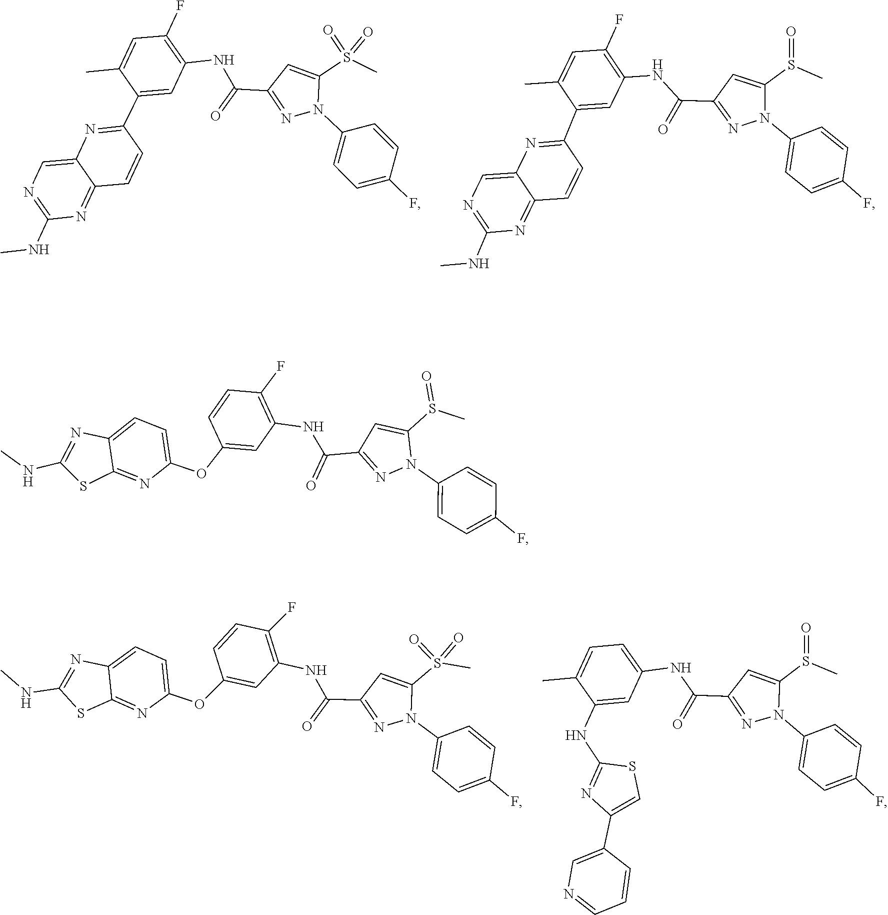 [SMILES: CNc1ncc2nc(-c3cc(NC(=O)c4cc(S(C)(=O)=O)n(-c5ccc(F)cc5)n4)c(F)cc3C)ccc2n1 ; CNc1ncc2nc(-c3cc(NC(=O)c4cc(S(C)=O)n(-c5ccc(F)cc5)n4)c(F)cc3C)ccc2n1 ; CNc1nc2ccc(Oc3ccc(F)c(NC(=O)c4cc(S(C)=O)n(-c5ccc(F)cc5)n4)c3)nc2s1 ; CNc1nc2ccc(Oc3ccc(F)c(NC(=O)c4cc(S(C)(=O)=O)n(-c5ccc(F)cc5)n4)c3)nc2s1 ; Cc1ccc(NC(=O)c2cc(S(C)=O)n(-c3ccc(F)cc3)n2)cc1Nc1nc(-c2cccnc2)cs1] 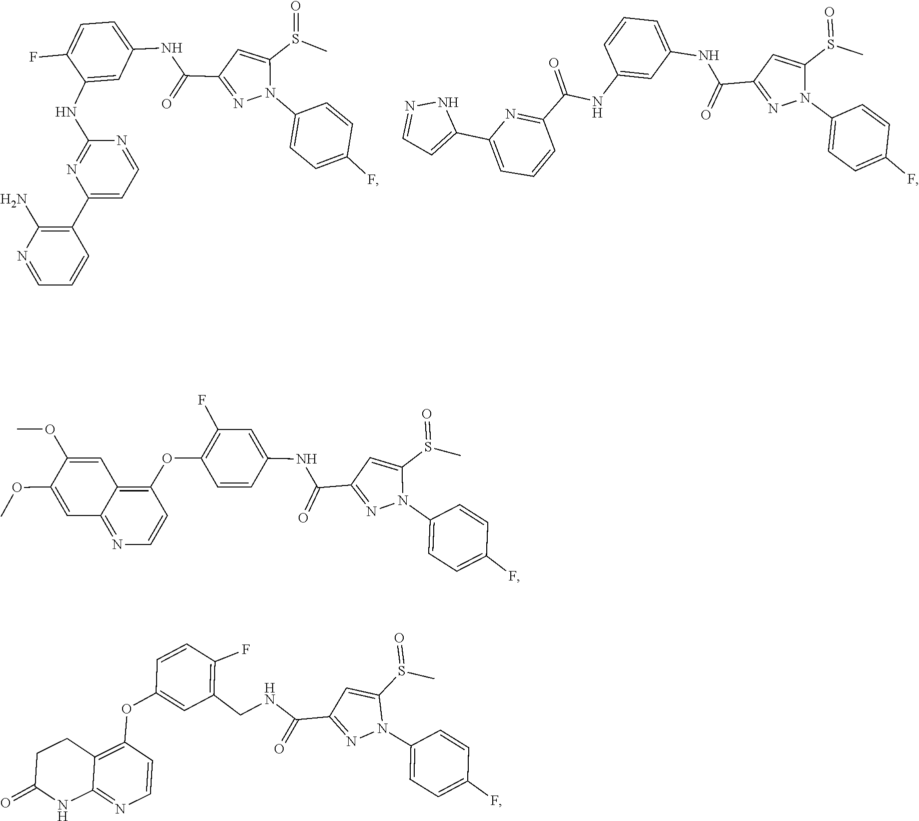 [SMILES: CS(=O)c1cc(C(=O)Nc2ccc(F)c(Nc3nccc(-c4cccnc4N)n3)c2)nn1-c1ccc(F)cc1 ; CS(=O)c1cc(C(=O)Nc2cccc(NC(=O)c3cccc(-c4ccn[nH]4)n3)c2)nn1-c1ccc(F)cc1 ; COc1cc2nccc(Oc3ccc(NC(=O)c4cc(S(C)=O)n(-c5ccc(F)cc5)n4)cc3F)c2cc1OC ; CS(=O)c1cc(C(=O)NCc2cc(Oc3ccnc4c3CCC(=O)N4)ccc2F)nn1-c1ccc(F)cc1] 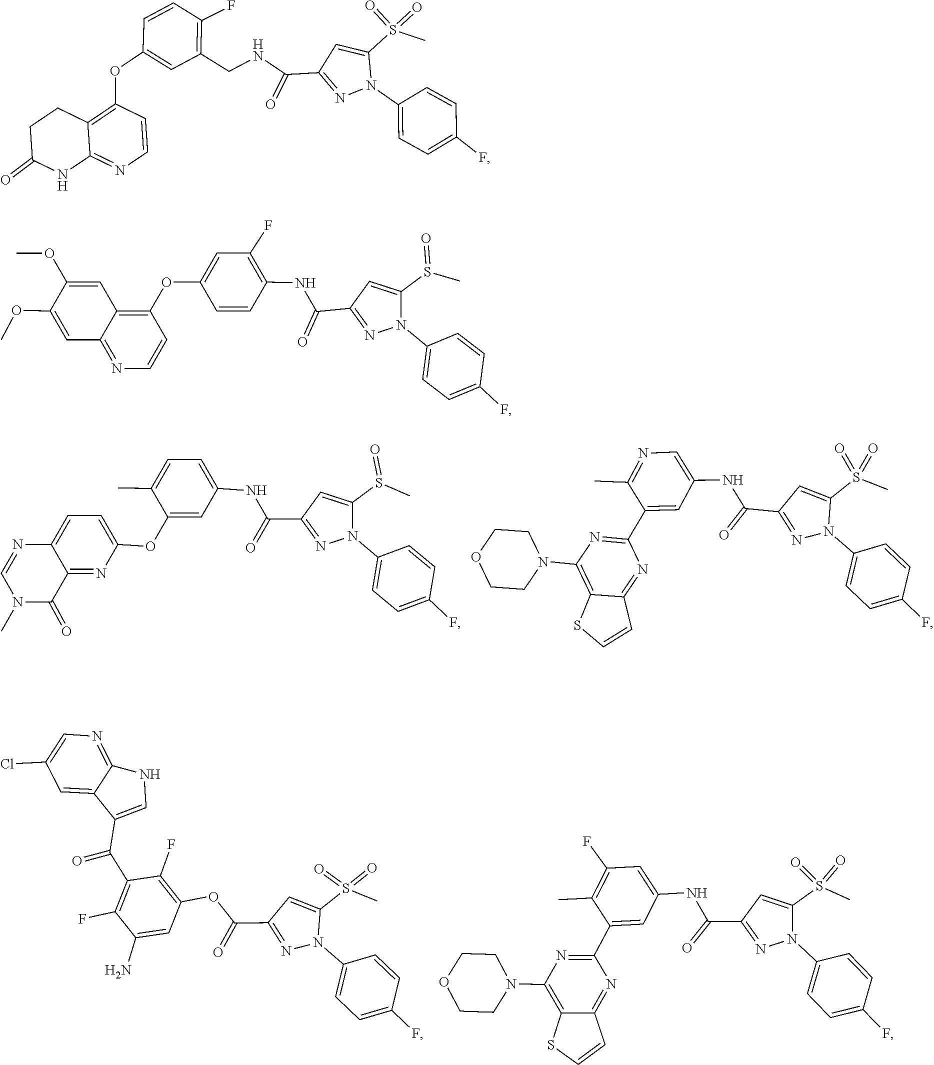 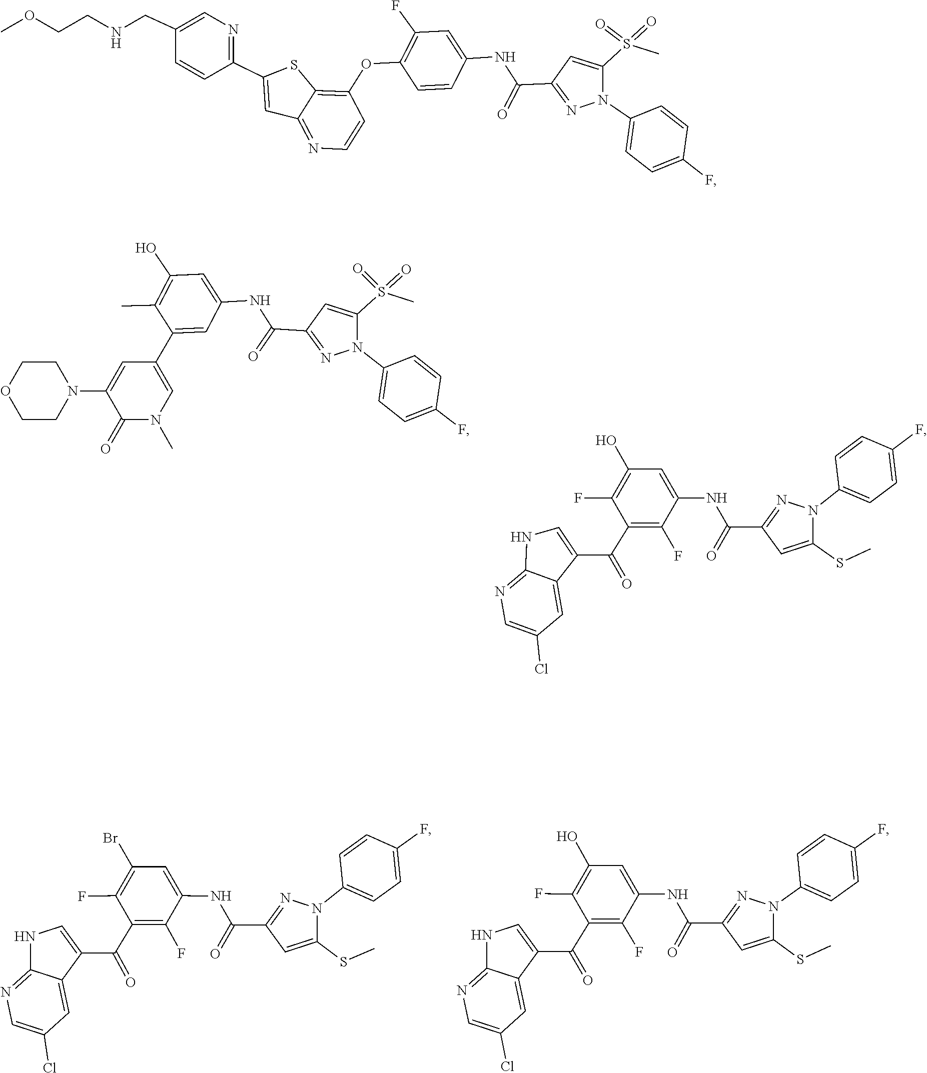 [SMILES: COCCNCc1ccc(-c2cc3nccc(Oc4ccc(NC(=O)c5cc(S(C)(=O)=O)n(-c6ccc(F)cc6)n5)cc4F)c3s2)nc1 ; Cc1c(O)cc(NC(=O)c2cc(S(C)(=O)=O)n(-c3ccc(F)cc3)n2)cc1-c1cc(N2CCOCC2)c(=O)n(C)c1 ; CSc1cc(C(=O)Nc2cc(O)c(F)c(C(=O)c3c[nH]c4ncc(Cl)cc34)c2F)nn1-c1ccc(F)cc1 ; CSc1cc(C(=O)Nc2cc(Br)c(F)c(C(=O)c3c[nH]c4ncc(Cl)cc34)c2F)nn1-c1ccc(F)cc1 ; CSc1cc(C(=O)Nc2cc(O)c(F)c(C(=O)c3c[nH]c4ncc(Cl)cc34)c2F)nn1-c1ccc(F)cc1] 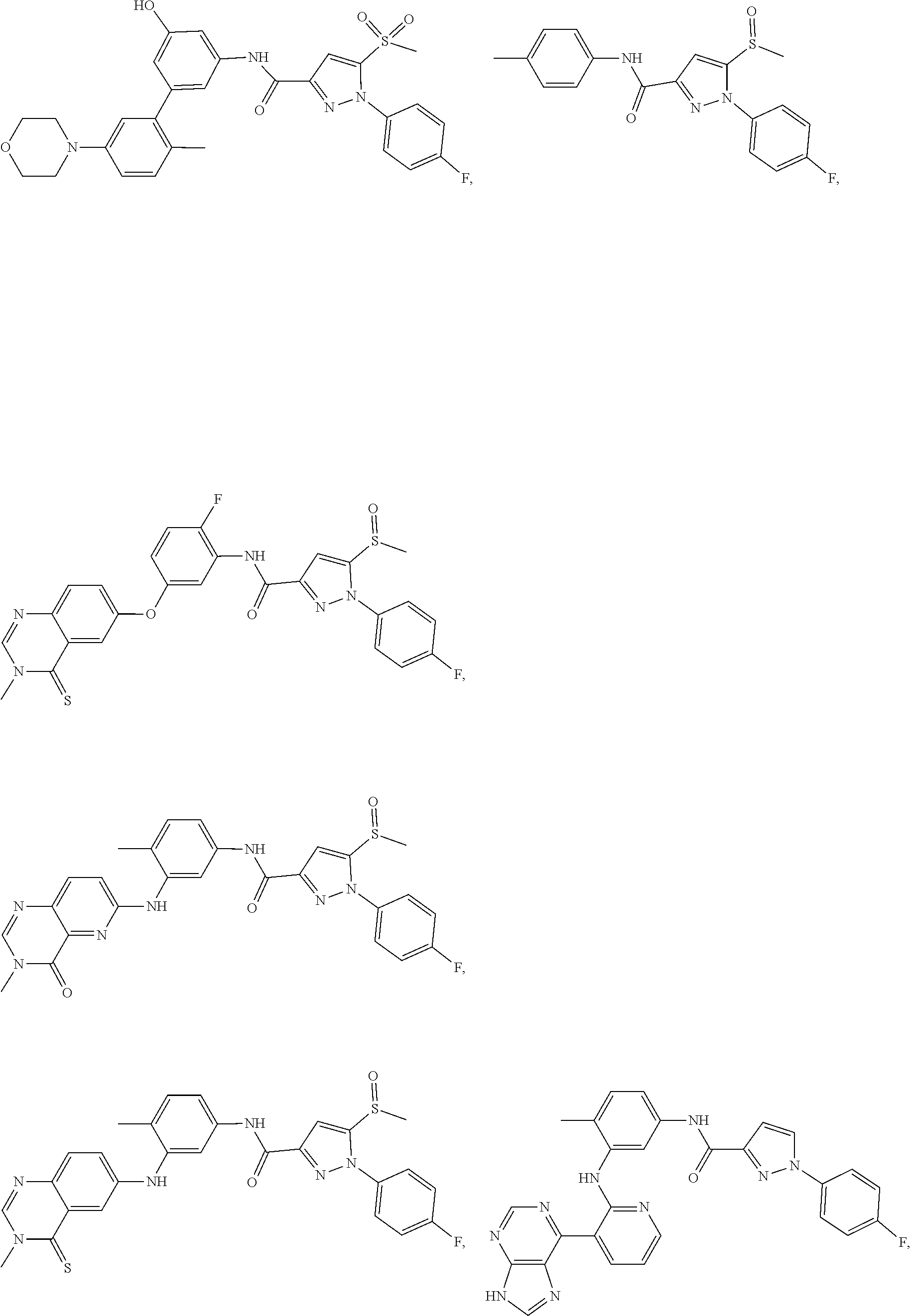 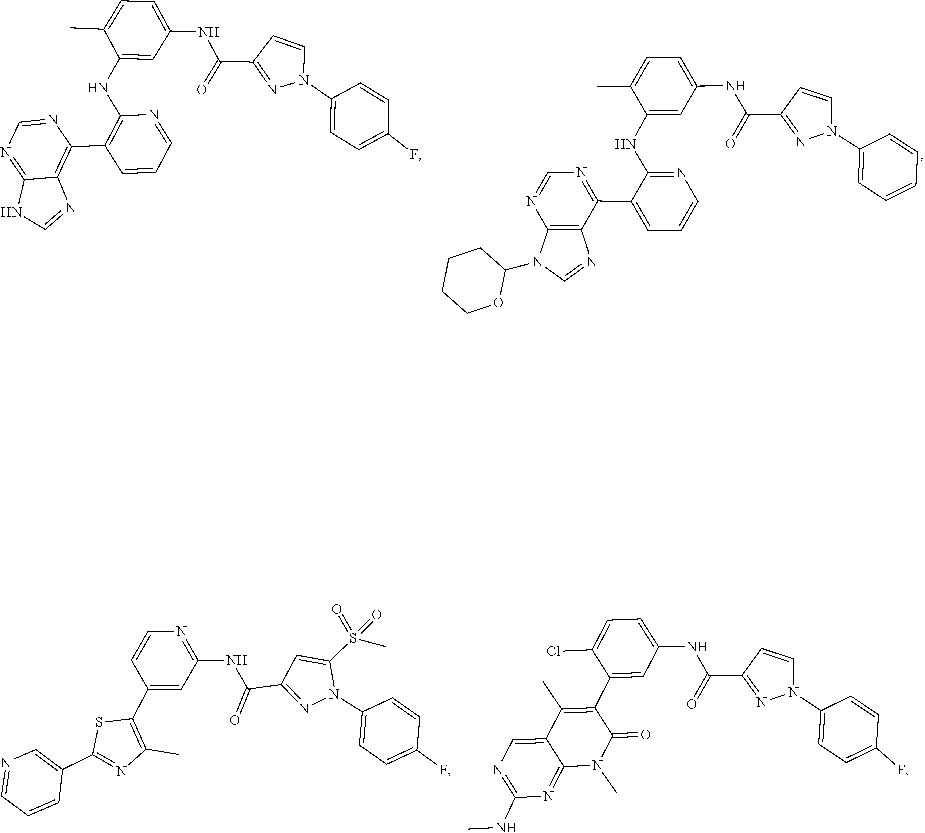 [SMILES: Cc1ccc(NC(=O)c2ccn(-c3ccc(F)cc3)n2)cc1Nc1ncccc1-c1ncnc2[nH]cnc12 ; Cc1ccc(NC(=O)c2ccn(-c3ccccc3)n2)cc1Nc1ncccc1-c1ncnc2c1ncn2C1CCCCO1 ; Cc1nc(-c2cccnc2)sc1-c1ccnc(NC(=O)c2cc(S(C)(=O)=O)n(-c3ccc(F)cc3)n2)c1 ; CNc1ncc2c(C)c(-c3cc(NC(=O)c4ccn(-c5ccc(F)cc5)n4)ccc3Cl)c(=O)n(C)c2n1] 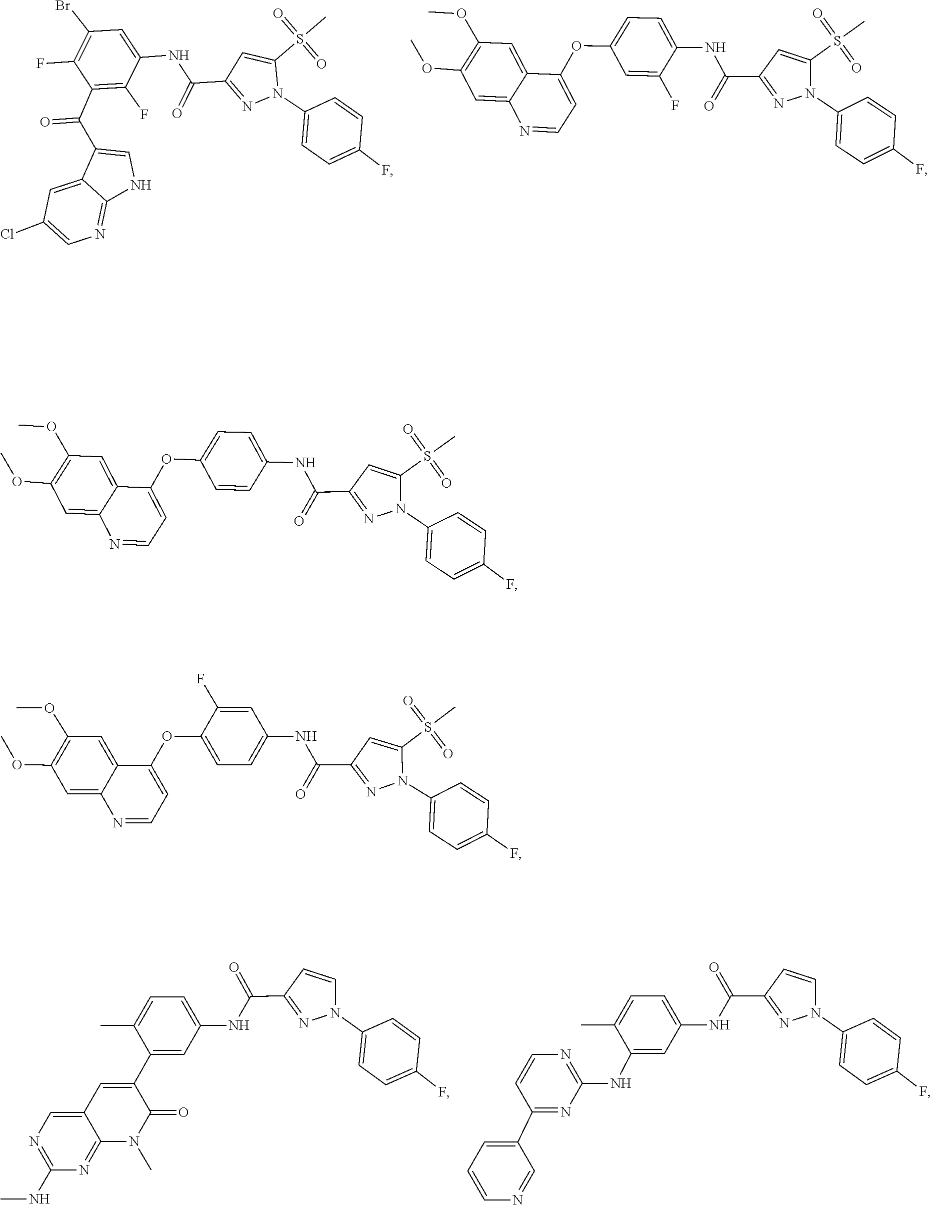 [SMILES: CS(=O)(=O)c1cc(C(=O)Nc2cc(Br)c(F)c(C(=O)c3c[nH]c4ncc(Cl)cc34)c2F)nn1-c1ccc(F)cc1 ; COc1cc2nccc(Oc3ccc(NC(=O)c4cc(S(C)(=O)=O)n(-c5ccc(F)cc5)n4)c(F)c3)c2cc1OC ; COc1cc2nccc(Oc3ccc(NC(=O)c4cc(S(C)(=O)=O)n(-c5ccc(F)cc5)n4)cc3)c2cc1OC ; COc1cc2nccc(Oc3ccc(NC(=O)c4cc(S(C)(=O)=O)n(-c5ccc(F)cc5)n4)cc3F)c2cc1OC ; CNc1ncc2cc(-c3cc(NC(=O)c4ccn(-c5ccc(F)cc5)n4)ccc3C)c(=O)n(C)c2n1 ; Cc1ccc(NC(=O)c2ccn(-c3ccc(F)cc3)n2)cc1Nc1nccc(-c2cccnc2)n1] 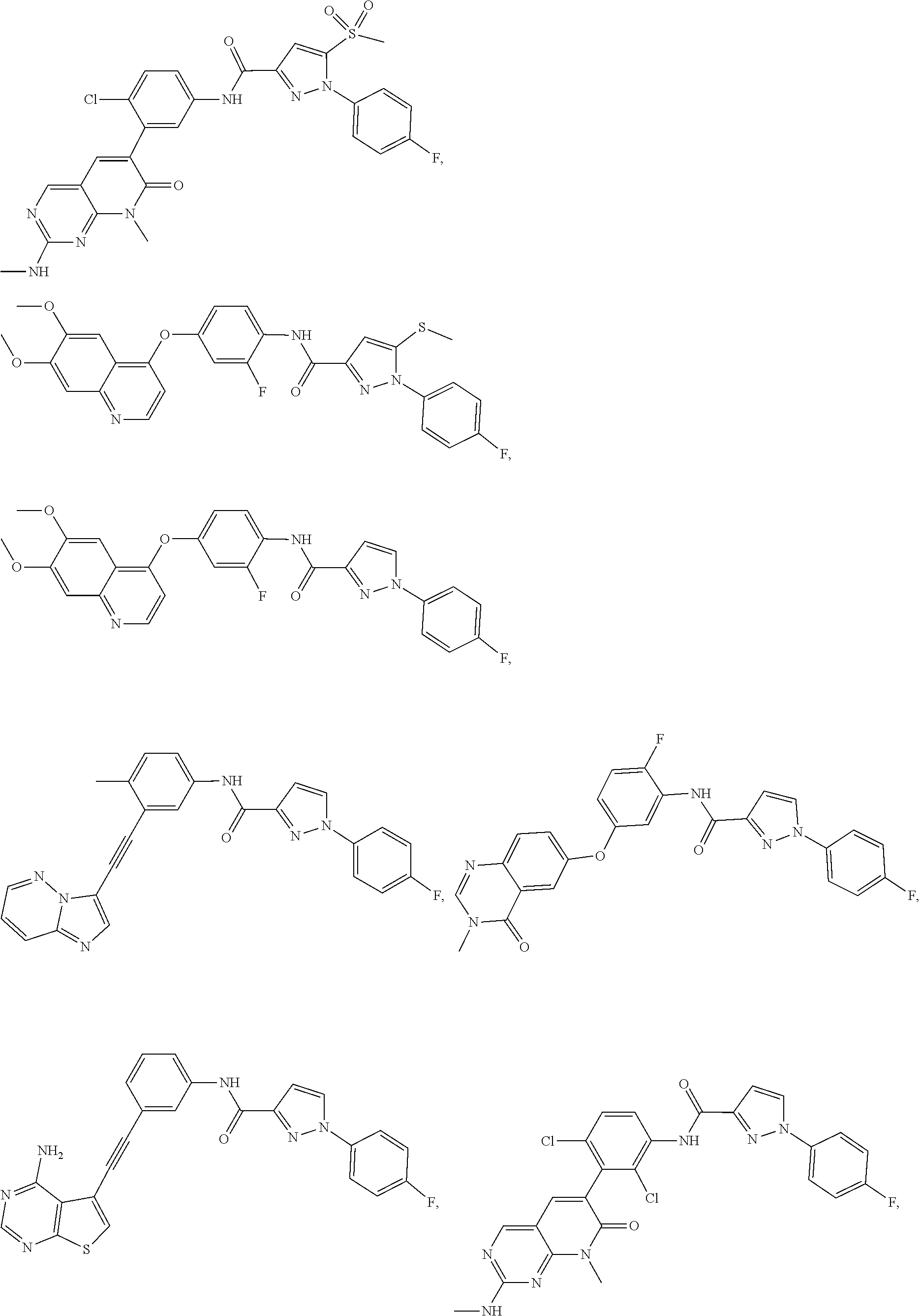 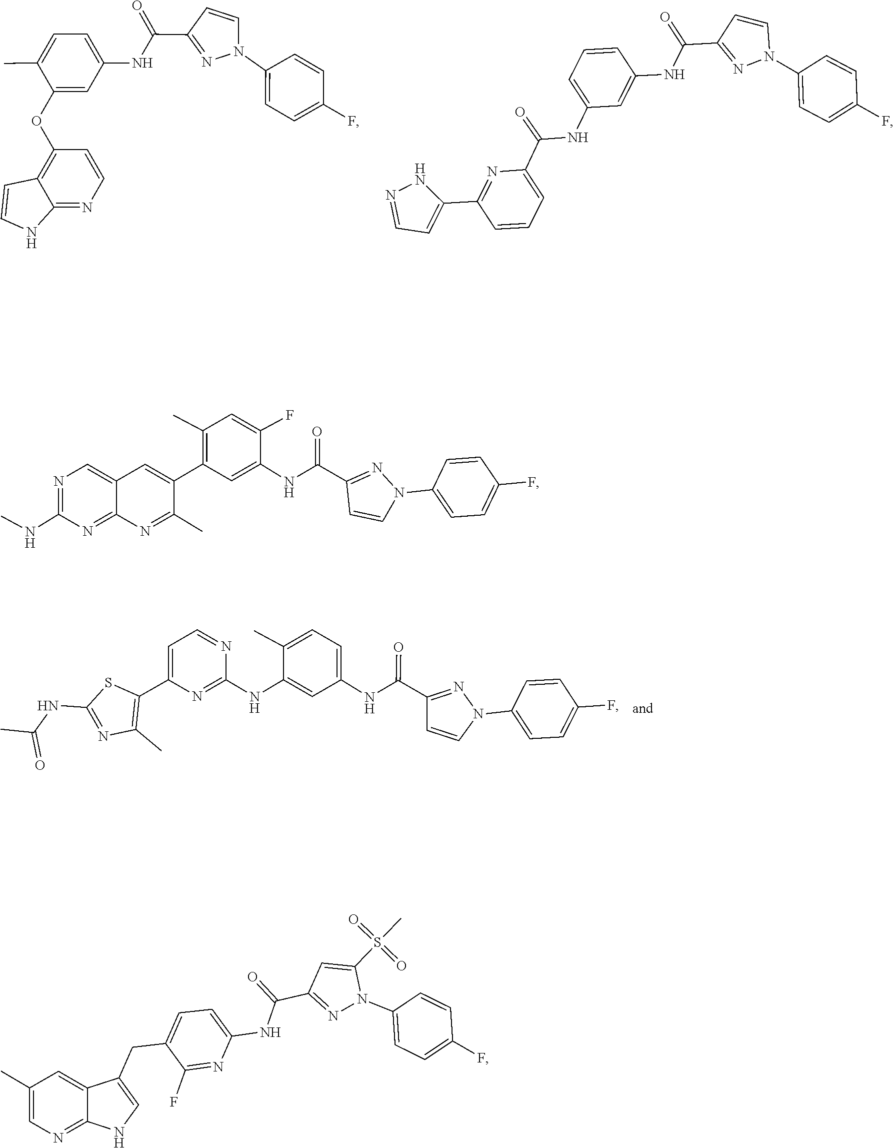 [SMILES: Cc1ccc(NC(=O)c2ccn(-c3ccc(F)cc3)n2)cc1Oc1ccnc2[nH]ccc12 ; O=C(Nc1cccc(NC(=O)c2ccn(-c3ccc(F)cc3)n2)c1)c1cccc(-c2ccn[nH]2)n1 ; CNc1ncc2cc(-c3cc(NC(=O)c4ccn(-c5ccc(F)cc5)n4)c(F)cc3C)c(C)nc2n1 ; CC(=O)Nc1nc(C)c(-c2ccnc(Nc3cc(NC(=O)c4ccn(-c5ccc(F)cc5)n4)ccc3C)n2)s1 ; Cc1cnc2[nH]cc(Cc3ccc(NC(=O)c4cc(S(C)(=O)=O)n(-c5ccc(F)cc5)n4)nc3F)c2c1]
or a pharmaceutically acceptable salt of any of the foregoing.

37. A pharmaceutically acceptable composition comprising the compound of any one of claims 1 to 36, or a pharmaceutically acceptable salt thereof; and a pharmaceutically acceptable carrier.

38. A method of treating a condition responsive to kinase modulation in a subject in need thereof, comprising administering to the subject a therapeutically effective amount of the compound of any one of claims 1 to 36, of the composition of claim 37.
